(19) United States
(12) Patent Application Publication
(10) Pub. No.: US 2009/0111805 A1
(43) Pub. Date: Apr. 30, 2009

(54) BICYCLIC HETEROAROMATIC DERIVATIVES USEFUL AS ANTICANCER AGENTS

(75) Inventors: Joel Morris, East Lyme, CT (US); Blaise Scott Lippa, Mystic, CT (US); Gonghua Pan, Old Lyme, CT (US); Goss Stryker Kauffman, Ledyard, CT (US); Chao Li, East Lyme, CT (US)

Correspondence Address: PFIZER INC, 10555 SCIENCE CENTER DRIVE, SAN DIEGO, CA, 92121, US

(73) Assignee: PFIZER INC.

(21) Appl. No.: 11/816,509

(22) PCT Filed: Feb. 15, 2006
(86) PCT No.: PCT/IB06/00406

§ 371 Date: May 19, 2008

Related U.S. Application Data
(60) Provisional application No. 60/656,467, filed on Feb. 24, 2005.

(51) Int. Cl. A61K 31/5377; C07D 487/04; A61K 31/519; C07D 473/00; A61K 31/52; A61K 31/438; C07D 471/10

(52) U.S. Cl. 514/234.2; 544/230; 514/265.1; 514/263.22; 546/15; 514/278; 546/18; 544/70

(57) ABSTRACT

The invention relates to compounds of the formula I:

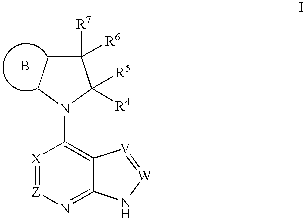

and to pharmaceutically acceptable salts and solvates thereof, wherein X, Z, V, W, R⁴, R⁵, R⁶, R⁷, and ring B are as defined herein. The invention also relates to methods of treating abnormal cell growth in mammals by administering the compounds of formula I and to pharmaceutical compositions for treating such disorders which contain the compounds of formula I. The invention also relates to methods of preparing the compounds of formula I.

This application is the national stage filing under 35 U.S.C. 371, of Patent Cooperation Treaty Patent Application No. PCT/IB2006/000406, filed Feb. 15, 2006, which claims the benefit of U.S. Provisional Patent Application No. 60/656,467 filed Feb. 24, 2005, the disclosures of which are incorporated herein by reference in their entireties.

### BACKGROUND OF THE INVENTION

This invention relates to novel bicyclic heteroaromatic derivatives that are useful in the treatment of abnormal cell growth, such as cancer, in mammals. This invention also relates to a method of using such compounds in the treatment of abnormal cell growth in mammals, especially humans, and to pharmaceutical compositions containing such compounds.

It is known that a cell may become cancerous by virtue of the transformation of a portion of its DNA into an oncogene (i.e., a gene which, on activation, leads to the formation of malignant tumor cells). Many oncogenes encode proteins that are aberrant tyrosine kinases capable of causing cell transformation. Alternatively, the overexpression of a normal proto-oncogenic tyrosine kinase may also result in proliferative disorders, sometimes resulting in a malignant phenotype.

Receptor tyrosine kinases are enzymes which span the cell membrane and possess an extracellular binding domain for growth factors such as epidermal growth factor, a transmembrane domain, and an intracellular portion which functions as a kinase to phosphorylate specific tyrosine residues in proteins and hence to influence cell proliferation. Other receptor tyrosine kinases include c-erbB-2, c-met, tie-2, PDGFr, FGFr, VEGF and TGF-β. When activated, these receptor kinases reportedly induce intracellular events such as intracellular signaling (see J. Dancer et al., Nature Reviews, 2:296-313 (2003)).

The targeted angiogenesis inhibitor Avastin® (Genetech) that prevents the formation of blood vessels by binding to the vascular endothelial growth factor (VEGF) has been approved in the United States for the treatment of colon cancer with combination with chemotherapy regimen that includes 5-fluorouracil (5-FU) and Camptosar® (Irinotecan). Additionally, a second targeted monoclonal antibody Erbitux® (cetuximab) (Imclone) that is believed to bind to the epidermal growth factor receptor (EGFR) was also recently approved for the treatment of colon cancer. A large number of other targeted agents are in clinical development for a variety of cancers.

Intracellular protein kinases such as serine/threonine kinases are reportedly involved in intracellular signaling pathways (see Nature Reviews, 2:296-313, 2003). These serine/threonine kinases are also reported to play a role in cancer. For example, it is reported that serine/threonine kinases are involved in uncontrolled cell proliferation and reduced cell death in tumor cells (see C. Sachsenmaier, Onkologie, 24:346-355, 2001). Examples of serine/threonine kinases reported to be involved in human cancer include protein kinase B (Akt) cyclin-dependent kinases (CDKs), mammalian target of rapamycin (mTor), mitogen-activated protein kinase kinase (MEK), and protein kinase C (PKC) (see Nature Reviews, 2:296-313, 2003).

Akt is a serine/threonine, intracellular kinase, which is reported to be a component of multiple signal transduction pathways involving cell proliferation, apoptosis, angiogenesis, and diabetes. It is reported that the Akt activation pathway can be activated by receptor tyrosine kinases, Ras, G protein-coupled receptors (GPCR), or inactivation of the tumor suppressor phosphatase and tensin homolog deleted on chromosome ten (PTEN) (see, e.g., West et al., Drug Resist. Updates, 5:234-248, 2002). It is also reported that Akt can be activated by cellular stress including heat shock, administration of ultraviolet light, ischemia, hypoxia, hypoglycemia, and oxidative stress (see West et al., Drug Resist. Updates, 5:234-248, 2002).

It is also reported that Akt is overexpressed in tumor cells (see, e.g., E. S. Kandel et. al., Exp. Cell Res., 253:21-229, 1999; Nicholson et. al., Cellular Signalling 14:381-395, 2002; and West et. al., Drug Resist. Updates, 5:234-248, 2002). Thus, overexpression of Akt in tumor cells provides an attractive target for drug intervention and the potential for a significant opportunity for controlling cell division in many types of cancer, and in particular for lung cancer, prostate cancer, colon cancer and breast cancer.

Applicants have identified novel heteroaromatic Akt kinase inhibitors that are able to modulate (reduce) that activity of the Akt kinase directly and in cancer cells, and thereby such agents are useful in effecting tumor growth.

### SUMMARY OF THE INVENTION

The present invention relates to a compound of formula I:

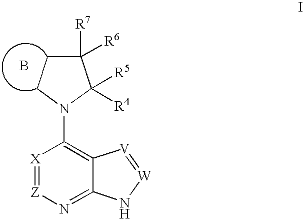

wherein X, Z, V and W are independently selected from the group consisting of N or CR¹;

each R¹ is independently selected from H, halo, cyano, nitro, azido, trifluoromethyl, trifluoromethoxy, —(CH₂)ₙNR⁸R⁹, —(CH₂)ₙOC(O)NR⁸R⁹, —NHC(═NCN)NHR¹⁰, —(C₁-C₁₀)alkyl, —(C₂-C₆)alkenyl, —(C₂-C₆)alkynyl, —O(CH₂)ₙR¹¹, —O(CH₂)ₙNR⁸R⁹, —(CH₂)ₙC(O)R¹⁰, —(CH₂)ₙNR¹⁰C(O)R¹⁰, —(CH₂)ₙNR¹⁰SO₂R¹⁰, —(CH₂)ₙC(O)OR¹⁰, —(CH₂)ₙOC(O)R¹¹, —(CH₂)ₙC(O)NR⁸R⁹, —(CH₂)ₙSO₂NR⁸R⁹, —(CH₂)ₙS(O)ⱼR¹, —(CH₂)ₙNR¹⁰C(O)NR⁸R⁹, —(CH₂)ₙNR¹⁰C(O)OR¹⁰—(CR¹¹R¹²)ₜ(C₆-C₁₀)aryl, —(CR¹¹R¹²)ₜ(4 to 10 membered heterocyclic), —(CR¹¹R¹²)_{q}C(O)(CR¹¹R¹²)ₜ(C₆-C₁₀)aryl, —(CR¹¹R¹²)_{q}C(O)(CR¹¹R¹²)ₜ(4 to 10 membered heterocyclic), —(CR¹¹R¹²)ₜO(CR¹¹R¹²)_{q}(C₆-C₁₀)aryl, —(CR¹¹R¹²)ₜO(CR¹¹R¹²)_{q}(4 to 10 membered heterocyclic), —(CR¹¹R¹²)_{q}S(O)ⱼ(CR¹¹R¹²)ₜ(C₆-C₁₀)aryl, and —(CR¹¹R¹²)_{q}S(O)ⱼ(CR¹¹R¹²)ₜ(4 to 10 membered heterocyclic), wherein 1 or 2 ring carbon atoms of the heterocyclic moieties of the foregoing R¹ groups are optionally substituted with an oxo moiety, and the alkyl, alkenyl, alkynyl, aryl and heterocyclic moieties of the foregoing R¹ groups are optionally substituted with 1 to 3 substituents independently selected from halo, hydroxy, cyano, nitro, trifluoromethyl, trifluoromethoxy, azido, —OR¹⁰, —C(O)R¹⁰, —C(O)OR¹⁰, —OC(O)R¹⁰, —NR¹⁰C(O)R¹⁰—C(O)NR¹⁰R¹¹, —NR⁸R⁹, —NR¹⁰R¹⁰, (C₁-C₁₀)alkyl, (C₂-C₆)alkenyl, (C₂-C₆)alkynyl, —(CR¹¹R¹²)ₜ(C₆-C₁₀)aryl, and —(CR¹¹R¹²)ₜ(4 to 10 membered heterocyclic);

n is an integer selected from 0 to 4;

j is an integer selected from 0 to 2;

q and t are each independently an integer from 0 to 5;

R⁴ is selected from H, —(C₁-C₁₁)alkyl, —(CR¹¹R¹²)ₜ(C₆-C₁₀)aryl, —(CR¹¹R¹²)ₜ(4 to 10 membered heterocyclic), wherein the alkyl, aryl and heterocyclic moieties of the foregoing R⁴ groups are optionally substituted with 1 to 3 substituents independently selected from halo, cyano, nitro, trifluoromethyl, trifluoromethoxy, azido, —OR¹³, —C(O)R¹³, —C(O)OR¹³, —OC(O)R¹³, —NR¹³C(O)R¹³, —C(O)NR¹⁴R¹⁵, —NR¹²OR¹², —(C₁-C₁₀)alkyl, —(C₂-C₆)alkenyl, —(C₂-C₆)alkynyl, —(CR¹¹R¹²)ₜ(C₆-C₁₀)aryl, and —(CR¹¹R¹²)ₜ(4 to 10 membered heterocyclic);

R⁵ is selected from H, —(C₁-C₁₀)alkyl, or wherein R⁴ and R⁵ when taken together form an oxo moiety;

R⁶ and R⁷ are taken together to form a 4 to 10-membered cyclic, bicyclic, heterocyclic or heterobicyclic ring system, said heterocyclic and heterobicyclic ring system containing 1 to 3 heteroatoms independently selected from N, O, or S, wherein each N atom present in the heterocyclic and heterobicyclic ring system is optionally substituted with a substituent selected from —(C₁-C₁₀)alkyl, —R¹⁰—C(O)R¹⁰—SO₂R¹⁰—C(O)NR¹⁰C(O)R¹⁰—C(O)NR¹⁰C(O)OR¹⁰, —C(O)NR⁸R⁹, —C(O)OR¹⁰ and each carbon atom in the heterocyclic and heterobicyclic ring system is independently optionally substituted by 1 to 2 substituents selected from R¹, and each carbon atom in the cyclic and bicyclic ring system is independently optionally substituted by 1 to 2 substituents selected from R¹;

R⁸ and R⁹ are independently selected from H, —(C₁-C₁₀)alkyl, —(CR¹¹R¹²)ₜ(C₆-C₁₀)aryl, —(CR¹¹R¹²)ₜ(4 to 10 membered heterocyclic), or wherein R⁸ and R⁹ when attached to the same N may be taken together to form a 3 to 11 membered mono or bicyclic ring containing an additional 1 to 2 heteroatoms independently selected from N, S or O, wherein each carbon atom of mono or bicyclic ring are optionally substituted with 1 to 2-(C₁-C₁₀)alkyl groups, or an oxo moiety and each additional N atom of the mono or bicyclic ring when present is optionally substituted with a substituent selected from —(C₁-C₁₀)alkyl, —R¹⁰, —C(O)R¹⁰, —SO₂R¹⁰, —C(O)NR¹¹R¹², —C(O)OR¹⁰, wherein 1 or 2 ring carbon atoms of the heterocyclic moieties of the foregoing R⁸ and R⁹ groups are optionally substituted with an oxo moiety, and the alkyl, aryl and heterocyclic moieties of the foregoing R⁸ and R⁹ groups are optionally substituted with 1 to 3 substituents independently selected from halo, cyano, nitro, trifluoromethyl, trifluoromethoxy, azido, —OR¹², —C(O)R¹², —C(O)OR¹², —OC(O)R¹², —NR¹²C(O)R¹², —C(O)NR¹⁴R¹⁵, —(CH₂)ₙNR C(O)NR¹⁴R¹⁵, O(CH₂)ₙNR¹⁴R¹⁵, —NR¹⁴R¹⁵, —NR¹²OR¹²—(CH₂)ₙSOⱼR¹⁰, —(C₁-C₁₀)alkyl, —(C₂-C₆)alkenyl —(C₂-C₆)alkynyl —(CR¹¹R¹²)ₜ(C₆-C₁₀)aryl, and —(CR¹¹R¹²)ₜ(4 to 10 membered heterocyclic);

R¹⁰ is selected from H, —(C₁-C₁₁)alkyl, —(CR¹¹R¹²)ₜ(C₆-C₁₀)aryl, —(CR¹¹R¹²)ₜ(4 to 10 membered heterocyclic), —(CR¹¹R¹²)_{q}S(O)ⱼ(CR¹¹R¹²)ₜ(C₆-C₁₀)aryl, and —(CR¹¹R¹²)_{q}S(O)ⱼ(CR¹¹R¹²)ₜ(4 to 10 membered heterocyclic), wherein the alkyl, aryl and heterocyclic moieties of the foregoing R¹⁰ groups are optionally substituted with 1 to 3 substituents independently selected from halo, cyano, nitro, trifluoromethyl, trifluoromethoxy, azido, —OR¹², —C(O)R¹², —C(O)OR¹², —OC(O)R¹²—NR¹²C(O)R¹², —C(O)NR¹⁴R¹⁵, —O(CH₂)ₙNR¹⁴R¹⁵, —NR¹⁴R¹⁵, —NR¹²OR¹², —(C₁-C₁₀)alkyl, —(C₂-C₆)alkenyl, —(C₂-C₆)alkynyl, —(CR¹¹R¹²)ₜ(C₆-C₁₀)aryl, and —(CR¹¹R¹²)ₜ(4 to 10 membered heterocyclic);

R¹¹ and R¹² are independently selected from H and —(C₁-C₁₀)alkyl;

R¹³ is selected from H, —(C₁-C₁₀)alkyl, —(CR¹¹R¹²)ₜ(C₆-C₁₀ aryl), and —(CR¹¹R¹²)ₜ(4 to 10 membered heterocyclic);

R¹⁴ and R¹⁵ are independently selected from H and —(C₁-C₁₀)alkyl or R¹⁴ and R¹⁵ may be taken together with the N atom they are attached to form a 3 to 11 membered mono- or bicyclic ring optionally containing 1 to 2 additional heteroatoms independently selected from N, O or S(O)ⱼ, wherein the C atoms of said mono- or bicyclic ring are optionally substituted with a substituent selected from oxo or —(C₁-C₁₀)alkyl, and wherein each N atom present in the mono- or bicyclic ring is optionally substituted with a substituent independently selected from —(C₁-C₁₀)alkyl;

B represents a fused 5 or 6-membered aromatic ring containing 0 to 2 heteroatoms, independently selected from N, O or S(O)ₚ, with the proviso the fused ring B does not contain two adjacent O or S(O)ⱼ atoms, wherein the carbon atoms of the fused ring B may be optionally substituted with 1 to 3 substituents independently selected from R¹, wherein the N atoms of the fused ring B may be optionally substituted with 1 to 2 substituents independently selected from R¹⁰, and ring B may optionally be fused to ring C;

C represents a 5 to 7-membered mono or bicyclic ring, optionally containing 0 to 3 heteroatoms, independently selected from N, O, and S(O)ⱼ, with the proviso the fused ring C does not contain two adjacent O or S(O)ⱼ atoms, and wherein the carbon atoms of fused ring C are optionally substituted with 1 to 3 substituents independently selected from R¹³, wherein the N atoms of the fused ring C may be optionally substituted with 1 to 2 substituents independently selected from R¹¹; or the pharmaceutically acceptable salts, solvates or prodrugs thereof.

In one embodiment of the present invention X is N in the compound of formula I.

In another embodiment of the present invention X is CR¹ in the compound of formula I.

In another embodiment of the present invention Z is CR¹ in the compound of formula I.

In another embodiment of the present invention Z is N in the compound of formula I.

In another embodiment of the present invention V is N in the compound of formula I.

In another embodiment of the present invention V is CR¹ in the compound of formula I.

In another embodiment of the present invention W is N in the compound of formula I.

In another embodiment of the present invention W is CR¹ in the compound of formula I.

In one preferred embodiment of the present invention X is N and Z, V and W are CR¹ in the compound of formula I.

In another embodiment of the present invention X and V are N in the compound of formula I.

In another embodiment of the present invention Z and W are CR¹ in the compound of formula I.

In another preferred embodiment of the present invention V are N and Z and W are CR¹ in the compound of formula I.

In another embodiment of the present invention X and Z are CR¹ in the compound of formula I.

In another embodiment of the present invention V and W are CR¹ in the compound of formula I.

In another preferred embodiment of the present invention X, Z, V and W are CR¹ in the compound of formula I.

In another embodiment of the present invention X is N and Z is CR¹ in the compound of formula I.

In another embodiment of the present invention V is CR¹ in the compound of formula I.

In another embodiment of the present invention W is N in the compound of formula I.

In another preferred embodiment of the present invention X and W are N and Z and V are CR¹ in the compound of formula I.

In one embodiment of the present invention each R¹ in formula I is independently selected from H, halo, cyano, nitro, azido, trifluoromethyl, trifluoromethoxy, —(CH₂)ₙNR⁸R⁹, —(CH₂)ₙOC(O)NR⁸R⁹, —NHC(═NCN)NHR¹⁰, —(C₁-C₁₀)alkyl, —(C₂-C₆)alkenyl, —(C₂-C₆)alkynyl, —O(CH₂)ₙR¹⁰, —O(CH₂)ₙNR⁸R⁹, —(CH₂)ₙC(O)R¹¹, —(CH₂)ₙNR¹⁰C(O)R¹⁰, —(CH₂)ₙNR¹⁰SO₂R¹⁰, —(CH₂)ₙC(O)OR¹⁰, —(CH₂)ₙOC(O)R¹⁰, —(CH₂)ₙC(O)NR⁸R⁹, —(CH₂)ₙSO₂NR⁸R⁹, —(CH₂)ₙSOⱼR¹⁰, —(CH₂)ₙNR¹⁰C(O)NR⁸R⁹, —(CH₂)ₙNR¹⁰C(O)OR¹⁰, —(CR¹¹R¹²)ₜ(C₆-C₁₀)aryl, —(CR¹¹R¹²)ₜ(4 to 10 membered heterocyclic), —(CR¹¹R¹²)_{q}C(O)(CR¹¹R¹²)ₜ(C₆-C₁₀)aryl, —(CR¹¹R¹²)_{q}C(O)(CR¹¹R¹²)ₜ(4 to 10 membered heterocyclic), —(CR¹¹R¹²)ₜO(CR¹¹R¹²)_{q}(C₆-C₁₀)aryl, —(CR¹¹R¹²)ₜO(CR¹¹R¹²)_{q}(4 to 10 membered heterocyclic), —(CR¹¹R¹²)_{q}S(O)ⱼ(CR¹¹R¹²)ₜ(C₆-C₁₀)aryl, and —(CR¹¹R¹²)_{q}S(O)ⱼ(CR¹¹R¹²)ₜ(4 to 10 membered heterocyclic), wherein 1 or 2 ring carbon atoms of the heterocyclic moieties of the foregoing R¹ groups are optionally substituted with an oxo moiety, and the alkyl, alkenyl, alkynyl, aryl and heterocyclic moieties of the foregoing R¹ groups are optionally substituted with 1 to 3 substituents independently selected from halo, hydroxy, cyano, nitro, trifluoromethyl, trifluoromethoxy, azido, —OR¹⁰, —C(O)R¹⁰, —C(O)OR¹⁰, —OC(O)R¹⁰, —NR¹⁰C(O)R¹⁰—C(O)NR¹⁰R¹¹, —NR⁸R⁹, —NR¹⁰R¹⁰, —(C₁-C₁₀)alkyl, —(C₂-C₆)alkenyl, —(C₂-C₆)alkynyl, —(CR¹¹R¹²)ₜ(C₆-C₁₀)aryl, and —(CR¹¹R¹²)ₜ(4 to 10 membered heterocyclic).

In another embodiment of the present invention each R¹ in formula I is independently selected from H, halo, cyano, nitro, azido, trifluoromethyl, trifluoromethoxy, —(CH₂)ₙNR⁸R⁹, —(CH₂)ₙOC(O)NR⁸R⁹, —NHC(═NCN)NHR¹⁰, —(C₁-C₁₀)alkyl, —(C₂-C₆)alkenyl, —(C₂-C₆)alkynyl, —O(CH₂)ₙR¹⁰, —O(CH₂)ₙNR⁸R⁹, —(CH₂)ₙC(O)R¹¹, —(CH₂)ₙNR¹⁰C(O)R¹—(CH₂)ₙNR¹¹SO₂R¹⁰, —(CH₂)ₙC(O)OR¹⁰, —(CH₂)ₙOC(O)R¹⁰, —(CH₂)ₙC(O)NR⁸R⁹, —(CH₂)ₙSO₂NR⁸R⁹, —(CH₂)ₙSOⱼR¹⁰, —(CH₂)ₙNR¹⁰C(O)NR⁸R⁹, —(CH₂)ₙNR¹⁰C(O)OR¹⁰ wherein the alkyl, alkenyl, alkynyl moieties of the foregoing R¹ groups are optionally substituted with 1 to 3 substituents independently selected from halo, hydroxy, cyano, nitro, trifluoromethyl, trifluoromethoxy, azido, —OR¹⁰, —C(O)R¹⁰, —C(O)OR¹⁰, —OC(O)R¹⁰, —NR¹⁰C(O)R¹⁰—C(O)NR¹⁰R¹¹, —NR⁸R⁹, —NR¹⁰OR¹⁰, —(C₁-C₁₀)alkyl, —(C₂-C₆)alkenyl, —(C₂-C₆)alkynyl, —(CR¹¹R¹²)ₜ(C₆-C₁₀)aryl, and —(CR¹¹R¹²)ₜ(4 to 10 membered heterocyclic).

In another embodiment of the present invention each R¹ in formula I is independently selected from H, halo, cyano, nitro, azido, trifluoromethyl, trifluoromethoxy, —(CH₂)ₙNR⁸R⁹, —(CH₂)ₙOC(O)NR⁸R⁹, —NHC(═NCN)NHR¹⁰, —(C₁-C₁₀)alkyl, —(C₂-C₆)alkenyl, —(C₂-C₆)alkynyl, wherein the alkyl, alkenyl, alkynyl moieties of the foregoing R¹ groups are optionally substituted with 1 to 3 substituents independently selected from halo, hydroxy, cyano, nitro, trifluoromethyl, trifluoromethoxy, azido, —OR¹⁰, —C(O)R¹⁰, —C(O)OR¹⁰, —OC(O)R¹⁰, —NR¹⁰C(O)R¹⁰—C(O)NR¹⁰R¹¹, —NR⁸R⁹, —NR¹⁰R¹⁰, —(C₁-C₁₀)alkyl, —(C₂-C₆)alkenyl, —(C₂-C₆)alkynyl, —(CR¹¹R¹²)ₜ(C₆-C₁₀)aryl, and —(CR¹¹R¹²)ₜ(4 to 10 membered heterocyclic).

In another embodiment of the present invention each R¹ in formula I is independently selected from H, halo, cyano, nitro, azido, trifluoromethyl, trifluoromethoxy and —(C₁-C₁₀)alkyl.

In another embodiment of the present invention each R¹ in formula I is independently selected from H, halo, cyano, trifluoromethyl and —(C₁-C₁₀)alkyl.

In a preferred embodiment of the present invention each R¹ in formula I is H.

In another embodiment of the present invention each R¹ in formula I is independently selected from —(CR¹¹R¹²)ₜ(C₆-C₁₀)aryl, —(CR¹¹R¹²)ₜ(4 to 10 membered heterocyclic), —(CR¹¹R¹²)_{q}C(O)(CR¹¹R¹²)ₜ(C₆-C₁₀)aryl, —(CR¹¹R¹²)_{q}C(O)(CR¹¹R¹²)ₜ(4 to 10 membered heterocyclic), —(CR¹¹R¹²)ₜO(CR¹¹R¹²)_{q}(C₆-C₁₀)aryl, —(CR¹¹R¹²)ₜO(CR¹¹R¹²)_{q}(4 to 10 membered heterocyclic), —(CR¹¹R¹²)_{q}S(O)ⱼ(CR¹¹R¹²)ₜ(C₆-C₁₀)aryl, and —(CR¹¹R¹²)_{q}S(O)ⱼ(CR¹¹R¹²)ₜ(4 to 10 membered heterocyclic), wherein 1 or 2 ring carbon atoms of the heterocyclic moieties of the foregoing R¹ groups are optionally substituted with an oxo moiety, and the aryl and heterocyclic moieties of the foregoing R¹ groups are optionally substituted with 1 to 3 substituents independently selected from halo, hydroxy, cyano, nitro, trifluoromethyl, trifluoromethoxy, azido, —OR¹⁰, —C(O)R¹⁰, —C(O)OR¹⁰, —OC(O)R¹⁰, —NR¹⁰C(O)R¹⁰—C(O)NR¹⁰R¹¹, —NR⁸R⁹, —NR¹⁰OR¹⁰, —(C₁-C₁₀)alkyl —(C₂-C₆)alkenyl —(C₂-C₆)alkynyl —(CR¹¹R¹²)ₜ(C₆-C₁₀)aryl, and —(CR¹¹R¹²)ₜ(4 to 10 membered heterocyclic).

In another embodiment of the present invention each R¹ in formula I is independently selected from —(CR¹¹R¹²)ₜ(C₆-C₁₀)aryl, and —(CR¹¹R¹²)ₜ(4 to 10 membered heterocyclic), wherein 1 or 2 ring carbon atoms of the heterocyclic moiety of the foregoing R¹ group is optionally substituted with an oxo moiety, and the aryl and heterocyclic moieties of the foregoing R¹ groups are optionally substituted with 1 to 3 substituents independently selected from halo, hydroxy, cyano, nitro, trifluoromethyl, trifluoromethoxy, azido, —OR¹⁰, —C(O)R¹⁰, —C(O)OR¹⁰, —OC(O)R¹⁰—NR¹⁰C(O)R¹⁰—C(O)NR¹⁰R¹¹, —NR⁸R⁹, —NR¹⁰R¹⁰, —(C₁-C₁₀)alkyl, —(C₂-C₆)alkenyl, —(C₂-C₆)alkynyl, —(CR¹¹R¹²)ₜ(C₆-C₁₀)aryl, and —(CR¹¹R¹²)ₜ(4 to 10 membered heterocyclic).

In one embodiment of the present invention ring B in formula I represents a fused 5-membered aromatic ring containing 0 to 2 heteroatoms, independently selected from N, O or S(O)ⱼ, with the proviso the fused ring B does not contain two adjacent O or S(O)ⱼ atoms, wherein the carbon atoms of the fused ring B may be optionally substituted with 1 to 3 substituents independently selected from R¹, wherein the N atoms of the fused ring B may be optionally substituted with 1 to 2 substituents independently selected from R¹⁰, and ring B may optionally be fused to ring C.

In another embodiment of the present invention ring B in formula I represents a fused 5-membered aromatic ring containing 0 to 1 heteroatom, independently selected from N, O or S(O)ⱼ, with the proviso the fused ring B does not contain two adjacent O or S(O)ⱼ atoms, wherein the carbon atoms of the fused ring B may be optionally substituted with 1 to 3 substituents independently selected from R¹, wherein the N atoms of the fused ring B may be optionally substituted with 1 to 2 substituents independently selected from R¹⁰, and ring B may optionally be fused to ring C.

In another embodiment of the present invention ring B in formula I represents a fused 5-membered aromatic ring containing 1 heteroatom, independently selected from N, O or S(O)ⱼ, with the proviso the fused ring B does not contain two adjacent O or S(O)ⱼ atoms, wherein the carbon atoms of the fused ring B may be optionally substituted with 1 to 3 substituents independently selected from R¹, wherein the N atoms of the fused ring B may be optionally substituted with 1 to 2 substituents independently selected from R¹⁰, and ring B may optionally be fused to ring C.

In another embodiment of the present invention ring B in formula I represents a fused 5-membered aromatic ring containing 0 heteroatom, wherein the carbon atoms of the fused ring B may be optionally substituted with 1 to 3 substituents independently selected from R¹, and ring B may optionally be fused to ring C.

In another embodiment of the present invention ring B in formula I represents 6-membered aromatic ring containing 0 to 2 heteroatoms, independently selected from N, O or S(O)ⱼ, with the proviso the fused ring B does not contain two adjacent O or S(O)ⱼ atoms, wherein the carbon atoms of the fused ring B may be optionally substituted with 1 to 3 substituents independently selected from R¹, wherein the N atoms of the fused ring B may be optionally substituted with 1 to 2 substituents independently selected from R¹⁰, and ring B may optionally be fused to ring C.

In another embodiment of the present invention ring B in formula I represents 6-membered aromatic ring containing 0 to 1 heteroatom, independently selected from N, O or S(O)ⱼ, with the proviso the fused ring B does not contain two adjacent O or S(O)ⱼ atoms, wherein the carbon atoms of the fused ring B may be optionally substituted with 1 to 3 substituents independently selected from R¹, wherein the N atoms of the fused ring B may be optionally substituted with 1 to 2 substituents independently selected from R¹⁰, and ring B may optionally be fused to ring C.

In another embodiment of the present invention ring B in formula I represents 6-membered aromatic ring containing 0 heteroatom, wherein the carbon atoms of the fused ring B may be optionally substituted with 1 to 3 substituents independently selected from R¹, and ring B may optionally be fused to ring C.

In a preferred embodiment of the present invention ring B in formula I is selected from the following rings:

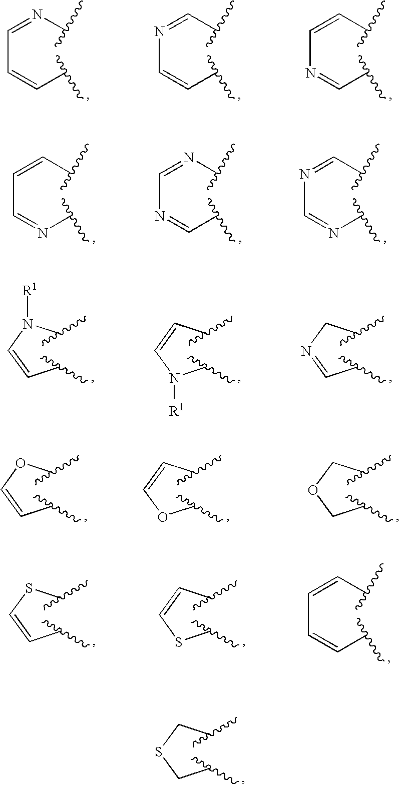

and wherein each substitutable ring carbon of said fused ring is independently substituted by 1 to 2 R¹ substituents.

In another preferred embodiment of the present invention ring B in formula I is selected from the following rings:

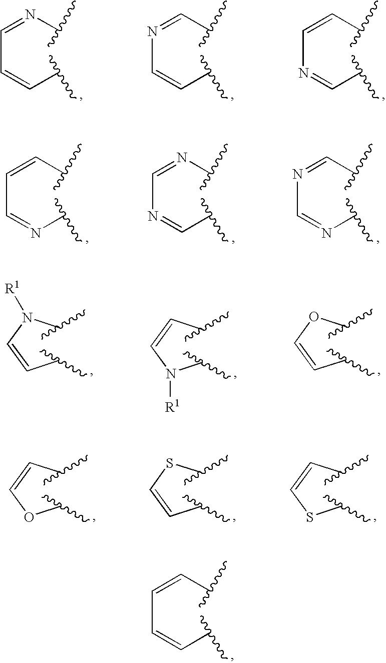

and wherein each substitutable ring carbon of said fused ring is independently substituted by 1 to 2 R¹ substituents

In more preferred embodiment of the present invention ring B in formula I is selected from the following rings:

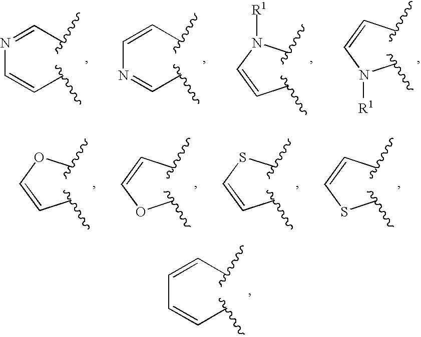

and wherein each substitutable ring carbon of said fused ring is independently substituted by 1 to 2 R¹ substituents.

In another more preferred embodiment of the present invention ring B in formula I is selected from the following rings:

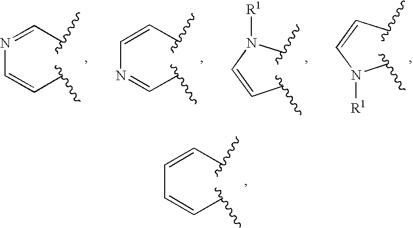

and wherein each substitutable ring carbon of said fused ring is independently substituted by 1 to 2 R¹ substituents.

In another more preferred embodiment of the present invention ring B in formula I is selected from the following rings:

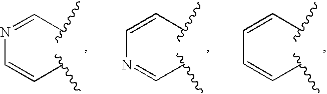

and wherein each substitutable ring carbon of said fused ring is independently substituted by 1 to 2 R¹ substituents.

In a most preferred embodiment of the present invention ring B in formula I is selected from the following rings:

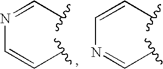

and wherein each substitutable ring carbon of said fused ring is independently substituted by 1 to 2 R¹ substituents.

In another most preferred embodiment of the present invention ring B in formula I is selected from the following ring:

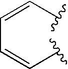

wherein each substitutable ring carbon of said fused ring is independently substituted by 1 to 2 R¹ substituents.

In another preferred embodiment of the present invention ring B in formula I is selected from the following rings:

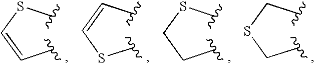

and wherein each substitutable ring carbon of said fused ring is independently substituted by 1 to 2 R¹ substituents.

In another most preferred embodiment of the present invention ring B in formula I is selected from the following rings:

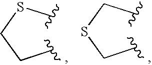

and wherein each substitutable ring carbon of said fused ring is independently substituted by 1 to 2 R¹ substituents.

In another most preferred embodiment of the present invention ring B in formula I is selected from the following rings:

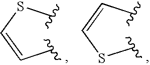

and wherein each substitutable ring carbon of said fused ring is independently substituted by 1 to 2 R¹ substituents.

In another most preferred embodiment of the present invention ring B in formula I is selected from the following rings:

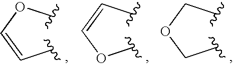

and wherein each substitutable ring carbon of said fused ring is independently substituted by 1 to 2 R¹ substituents.

In one embodiment of the present invention ring C in formula I is a 5-membered mono or bicyclic ring, optionally containing 0 to 3 heteroatoms, independently selected from N, O, and S(O), with the proviso the fused ring C does not contain two adjacent O or S(O)ⱼ atoms, and wherein the carbon atoms of fused ring C are optionally substituted with 1 to 3 substituents independently selected from R¹³ wherein the N atoms of the fused ring C may be optionally substituted with 1 to 2 substituents independently selected from R¹¹.

In another embodiment of the present invention ring C in formula I is a 6-membered mono or bicyclic ring, optionally containing 0 to 3 heteroatoms, independently selected from N, O, and S(O)ⱼ, with the proviso the fused ring C does not contain two adjacent O or S(O)ⱼ atoms, and wherein the carbon atoms of fused ring C are optionally substituted with 1 to 3 substituents independently selected from R¹³ wherein the N atoms of the fused ring C may be optionally substituted with 1 to 2 substituents independently selected from R¹¹.

In another embodiment of the present invention ring C in formula I is a 7-membered mono or bicyclic ring, optionally containing 0 to 3 heteroatoms, independently selected from N, O, and S(O)ⱼ, with the proviso the fused ring C does not contain two adjacent O or S(O)ⱼ atoms, and wherein the carbon atoms of fused ring C are optionally substituted with 1 to 3 substituents independently selected from R¹³ wherein the N atoms of the fused ring C may be optionally substituted with 1 to 2 substituents independently selected from R¹¹.

In another embodiment of the present invention ring C in formula I is a 5 to 7-membered monocyclic ring, optionally containing 0 to 3 heteroatoms, independently selected from N, O, and S(O)ⱼ, with the proviso the fused ring C does not contain two adjacent O or S(O)ⱼ atoms, and wherein the carbon atoms of fused ring C are optionally substituted with 1 to 3 substituents independently selected from R¹³ wherein the N atoms of the fused ring C may be optionally substituted with 1 to 2 substituents independently selected from R¹¹.

In another embodiment of the present invention ring C in formula I is a 5 to 7-membered bicyclic ring, optionally containing 0 to 3 heteroatoms, independently selected from N, O, and S(O)ₚ, with the proviso the fused ring C does not contain two adjacent O or S(O)ⱼ atoms, and wherein the carbon atoms of fused ring C are optionally substituted with 1 to 3 substituents independently selected from R¹³, wherein the N atoms of the fused ring C may be optionally substituted with 1 to 2 substituents independently selected from R¹¹.

In one embodiment of the present invention R⁴ in formula I is selected from H and (C₁-C₁₀)alkyl, wherein the alkyl moiety of the foregoing R⁴ group is optionally substituted with 1 to 3 substituents independently selected from halo, cyano, nitro, trifluoromethyl, trifluoromethoxy, azido, —OR¹³, —C(O)R¹³, —C(O)OR¹³, —OC(O)R¹³, —NR¹³C(O)R¹³, —C(O)NR¹⁴R¹⁵, —NR¹²OR¹², —(C₁-C₁₀)alkyl, —(C₂-C₆)alkenyl, —(C₂-C₆)alkynyl, —(CR¹¹R¹²)ₜ(C₆-C₁₀)aryl, and —(CR¹¹R¹²)ₜ(4 to 10 membered heterocyclic).

In another embodiment of the present invention R⁴ in formula I is selected from —(CR¹¹R¹²)ₜ(C₆-C₁₀)aryl, —(CR¹¹R¹²)ₜ(4 to 10 membered heterocyclic), wherein the aryl and heterocyclic moieties of the foregoing R⁴ groups are optionally substituted with 1 to 3 substituents independently selected from halo, cyano, nitro, trifluoromethyl, trifluoromethoxy, azido, —OR¹³, —C(O)R¹³, —C(O)OR¹³ OC(O)R¹³, —NR¹³C(O)R¹³, —C(O)NR¹⁴R¹⁵, —NR¹²OR¹² (C₁-C₁₀)alkyl, —(C₂-C₆)alkenyl, —(C₂-C₆)alkynyl, —(CR¹¹R¹²)ₜ(C₆-C₁₀)aryl, and —(CR¹¹R¹²)ₜ(4 to 10 membered heterocyclic).

In one embodiment of the present invention R⁵ in formula I is —(C₁-C₁₀)alkyl.

In another embodiment of the present invention R⁵ in formula I is H.

In one embodiment of the present invention R⁶ and R⁷ in formula I are taken together to form a 4 to 10-membered cyclic or bicyclic and each carbon atom in the cyclic and bicyclic ring system is independently optionally substituted by 1 to 2 substituents selected from R¹.

In another embodiment of the present invention R⁶ and R⁷ in formula I are taken together to form a 4 to 8-membered cyclic or bicyclic and each carbon atom in the cyclic and bicyclic ring system is independently optionally substituted by 1 to 2 substituents selected from R¹.

In another embodiment of the present invention R⁶ and R⁷ in formula I are taken together to form a 4 to 6-membered cyclic or bicyclic and each carbon atom in the cyclic and bicyclic ring system is independently optionally substituted by 1 to 2 substituents selected from R¹.

In another embodiment of the present invention R⁶ and R⁷ in formula I are taken together to form a 6-membered cyclic or bicyclic and each carbon atom in the cyclic and bicyclic ring system is independently optionally substituted by 1 to 2 substituents selected from R¹.

In another embodiment of the present invention R⁶ and R⁷ in formula I are taken together to form a 4 to 10-membered heterocyclic or heterobicyclic ring system, said heterocyclic and heterobicyclic ring system containing 1 to 3 heteroatoms independently selected from N, O, or S, wherein each N atom present in the heterocyclic and heterobicyclic ring system is optionally substituted with a substituent selected from —(C₁-C₁₀)alkyl, —R¹⁰, —C(O)R¹⁰—SO₂R¹⁰, —C(O)NR¹⁰C(O)R¹⁰, —C(O)NR¹⁰C(O)OR¹⁰, —C(O)NR⁸R⁹, —C(O)OR¹⁰ and each carbon atom in the heterocyclic and heterobicyclic ring system is independently optionally substituted by 1 to 2 substituents selected from R¹.

In another embodiment of the present invention R⁶ and R⁷ in formula I are taken together to form a 4 to 8-membered heterocyclic or heterobicyclic ring system, said heterocyclic and heterobicyclic ring system containing 1 to 3 heteroatoms independently selected from N, O, or S, wherein each N atom present in the heterocyclic and heterobicyclic ring system is optionally substituted with a substituent selected from —(C₁-C₁₀)alkyl, —R¹⁰, —C(O)R¹⁰, —SO₂R¹⁰, —C(O)NR¹⁰C(O)R¹⁰, —C(O)NR¹⁰C(O)OR¹⁰, —C(O)NR⁸R⁹, —C(O)OR¹⁰ and each carbon atom in the heterocyclic and heterobicyclic ring system is independently optionally substituted by 1 to 2 substituents selected from R¹.

In another embodiment of the present invention R⁶ and R⁷ in formula I are taken together to form a 4 to 6-membered heterocyclic or heterobicyclic ring system, said heterocyclic and heterobicyclic ring system containing 1 to 3 heteroatoms independently selected from N, O, or S, wherein each N atom present in the heterocyclic and heterobicyclic ring system is optionally substituted with a substituent selected from —(C₁-C₁₀)alkyl, —R¹⁰, —C(O)R¹⁰, —SO₂R¹⁰, —C(O)NR¹⁰C(O)R¹⁰, —C(O)NR¹⁰C(O)OR¹⁰, —C(O)NR⁸R⁹, —C(O)OR¹⁰ and each carbon atom in the heterocyclic and heterobicyclic ring system is independently optionally substituted by 1 to 2 substituents selected from R¹.

In another embodiment of the present invention R⁶ and R⁷ in formula I are taken together to form a 4 to 10-membered heterocyclic or heterobicyclic ring system, said heterocyclic and heterobicyclic ring system containing 1 to 2 heteroatoms independently selected from N, O, or S, wherein each N atom present in the heterocyclic and heterobicyclic ring system is optionally substituted with a substituent selected from —(C₁-C₁₀)alkyl, —R¹⁰, —C(O)R¹⁰—SO₂R¹⁰, —C(O)NR¹⁰C(O)R¹⁰, —C(O)NR¹⁰C(O)OR¹⁰, —C(O)NR⁸R⁹, —C(O)OR¹⁰ and each carbon atom in the heterocyclic and heterobicyclic ring system is independently optionally substituted by 1 to 2 substituents selected from R¹.

In another embodiment of the present invention R⁶ and R⁷ in formula I are taken together to form a 4 to 10-membered heterocyclic or heterobicyclic ring system, said heterocyclic and heterobicyclic ring system containing 1 heteroatom selected from N, O, or S, wherein each N atom present in the heterocyclic and heterobicyclic ring system is optionally substituted with a substituent selected from —(C₁-C₁₀)alkyl, —R¹⁰, —C(O)R¹⁰, —SO₂R¹⁰, —C(O)NR¹⁰C(O)R¹⁰, —C(O)NR¹⁰C(O)OR¹⁰, —C(O)NR⁸R⁹, —C(O)OR¹⁰ and each carbon atom in the heterocyclic and heterobicyclic ring system is independently optionally substituted by 1 to 2 substituents selected from R¹.

In another embodiment of the present invention R⁶ and R⁷ in formula I are taken together to form a 4 to 10-membered heterocyclic or heterobicyclic ring system, said heterocyclic and heterobicyclic ring system containing 1 to 2 heteroatoms independently selected from N or S, wherein each N atom present in the heterocyclic and heterobicyclic ring system is optionally substituted with a substituent selected from —(C₁-C₁₀)alkyl, —R¹⁰, —C(O)R¹⁰, —SO₂R¹⁰, —C(O)NR¹⁰C(O)R¹⁰, —C(O)NR¹⁰C(O)OR¹⁰, —C(O)NR⁸R⁹, —C(O)OR¹⁰ and each carbon atom in the heterocyclic and heterobicyclic ring system is independently optionally substituted by 1 to 2 substituents selected from R¹.

In another embodiment of the present invention R⁶ and R⁷ in formula I are taken together to form a 4 to 8-membered heterocyclic or heterobicyclic ring system, said heterocyclic and heterobicyclic ring system containing 1 to 2 heteroatoms independently selected from N or S, wherein each N atom present in the heterocyclic and heterobicyclic ring system is optionally substituted with a substituent selected from —(C₁-C₁₀)alkyl, —R¹⁰, —C(O)R¹⁰, —SO₂R¹⁰, —C(O)NR¹⁰C(O)R¹⁰, —C(O)NR¹⁰C(O)OR¹⁰, —C(O)NR⁸R⁹, —C(O)OR¹⁰ and each carbon atom in the heterocyclic and heterobicyclic ring system is independently optionally substituted by 1 to 2 substituents selected from R¹.

In another embodiment of the present invention R⁶ and R⁷ in formula I are taken together to form a 4 to 6-membered heterocyclic or heterobicyclic ring system, said heterocyclic and heterobicyclic ring system containing 1 to 2 heteroatoms independently selected from N or S, wherein each N atom present in the heterocyclic and heterobicyclic ring system is optionally substituted with a substituent selected from —(C₁-C₁₀)alkyl, —R¹⁰, —C(O)R¹⁰, —SO₂R¹⁰, —C(O)NR¹⁰C(O)R¹⁰, —C(O)NR¹⁰C(O)OR¹⁰, —C(O)NR⁸R⁹, —C(O)OR¹⁰ and each carbon atom in the heterocyclic and heterobicyclic ring system is independently optionally substituted by 1 to 2 substituents selected from R¹.

In another embodiment of the present invention R⁶ and R⁷ in formula I are taken together to form a 4 to 10-membered heterocyclic or heterobicyclic ring system, said heterocyclic and heterobicyclic ring system containing 1 heteroatom independently selected from N, wherein each N atom present in the heterocyclic and heterobicyclic ring system is optionally substituted with a substituent selected from —(C₁-C₁₀)alkyl, —R¹⁰, —C(O)R¹⁰, —SO₂R¹⁰, —C(O)NR¹⁰C(O)R¹⁰, —C(O)NR¹⁰C(O)OR¹⁰, —C(O)NR⁸R⁹, —C(O)OR¹⁰ and each carbon atom in the heterocyclic and heterobicyclic ring system is independently optionally substituted by 1 to 2 substituents selected from R¹.

In another embodiment of the present invention R⁶ and R⁷ in formula I are taken together to form a 4 to 8-membered heterocyclic or heterobicyclic ring system, said heterocyclic and heterobicyclic ring system containing 1 to 2 heteroatoms selected from N, wherein each N atom present in the heterocyclic and heterobicyclic ring system is optionally substituted with a substituent selected from —(C₁-C₁₀)alkyl, —R¹⁰, —C(O)R¹⁰, —SO₂R¹⁰, —C(O)NR¹⁰C(O)R¹⁰, —C(O)NR¹⁰C(O)OR¹⁰, —C(O)NR⁸R⁹, —C(O)OR¹⁰ and each carbon atom in the heterocyclic and heterobicyclic ring system is independently optionally substituted by 1 to 2 substituents selected from R¹.

In another embodiment of the present invention R⁶ and R⁷ in formula I are taken together to form a 4 to 6-membered heterocyclic or heterobicyclic ring system, said heterocyclic and heterobicyclic ring system containing 1 to 2 heteroatoms independently selected from N, wherein each N atom present in the heterocyclic and heterobicyclic ring system is optionally substituted with a substituent selected from —(C₁-C₁₀)alkyl, —R¹⁰, —C(O)R¹⁰, —SO₂R¹⁰, —C(O)NR¹⁰C(O)R¹⁰, —C(O)NR¹⁰C(O)OR¹⁰, —C(O)NR⁸R⁹, —C(O)OR¹⁰ and each carbon atom in the heterocyclic and heterobicyclic ring system is independently optionally substituted by 1 to 2 substituents selected from R¹.

In another embodiment of the present invention R⁶ and R⁷ in formula I are taken together to form a 7 membered heterocyclic ring system, said heterocyclic ring system containing 1 to 2 heteroatoms independently selected from N and O wherein each N atom present in the ring system is optionally substituted with a substituent selected from —(C₁-C₁₀)alkyl, —R¹⁰—C(O)R¹⁰—SO₂R¹⁰, —C(O)NR¹⁰C(O)R¹⁰, —C(O)NR¹⁰C(O)OR¹, —C(O)NR⁸R⁹, —C(O)OR¹⁰ and each carbon atom in the ring is independently optionally substituted by 1 to 2 substituents selected from R¹.

In another embodiment of the present invention R⁶ and R⁷ in formula I are taken together to form a 4 membered heterocyclic ring system, said heterocyclic ring system containing 1 to 2 N heteroatoms, wherein each N atom present in the ring system is optionally substituted with a substituent selected from —(C₁-C₁₀)alkyl, —R¹⁰, —C(O)R¹⁰, —SO₂R¹⁰, —C(O)NR¹⁰C(O)R¹⁰, —C(O)NR¹⁰C(O)OR¹⁰, —C(O)NR⁸R⁹, —C(O)OR¹⁰ and each carbon atom in the ring is independently optionally substituted by 1 to 2 substituents selected from R¹.

In another embodiment of the present invention R⁶ and R⁷ in formula I are taken together to form a 5 membered heterocyclic ring system, said heterocyclic ring system containing 1 to 2 N heteroatoms, wherein each N atom present in the ring system is optionally substituted with a substituent selected from —(C₁-C₁₀)alkyl, —R¹⁰—C(O)R¹⁰, —SO₂R¹⁰, —C(O)NR¹⁰C(O)R¹⁰, —C(O)NR¹⁰C(O)OR¹⁰, —C(O)NR⁸R⁹, —C(O)OR¹⁰ and each carbon atom in the ring is independently optionally substituted by 1 to 2 substituents selected from R¹.

In another embodiment of the present invention R⁶ and R⁷ in formula I are taken together to form a 6 membered heterocyclic ring system, said heterocyclic ring system containing 1 to 2 N heteroatoms, wherein each N atom present in the ring system is optionally substituted with a substituent selected from —(C₁-C₁₀)alkyl, —R¹⁰, —C(O)R¹⁰, —SO₂R¹⁰, —C(O)NR¹⁰C(O)R¹⁰, —C(O)NR¹⁰C(O)OR¹⁰, —C(O)NR⁸R⁹, —C(O)OR¹⁰ and each carbon atom in the ring is independently optionally substituted by 1 to 2 substituents selected from R¹.

In another embodiment of the present invention R⁶ and R⁷ in formula I are taken together to form a 7 membered heterocyclic ring system, said heterocyclic ring system containing 1 to 2 N heteroatoms, wherein each N atom present in the ring system is optionally substituted with a substituent selected from —(C₁-C₁₀)alkyl, —R¹⁰, —C(O)R¹⁰, —SO₂R¹⁰, —C(O)NR¹⁰C(O)R¹⁰, —C(O)NR¹⁰C(O)OR¹⁰, —C(O)NR⁸R⁹, —C(O)OR¹⁰ and each carbon atom in the ring is independently optionally substituted by 1 to 2 substituents selected from R¹.

In another embodiment of the present invention R⁶ and R⁷ in formula I are taken together to form a 4 membered heterocyclic ring system, said heterocyclic ring system containing 1 N heteroatom, wherein said N atom present in the ring system is optionally substituted with a substituent selected from —(C₁-C₁₀)alkyl, —R¹⁰, —C(O)R¹⁰, —SO₂R¹⁰, —C(O)NR¹⁰C(O)R¹⁰, —C(O)NR¹⁰C(O)OR¹⁰, —C(O)NR⁸R⁹, —C(O)OR¹⁰ and each carbon atom in the ring is independently optionally substituted by 1 to 2 substituents selected from R¹.

In another embodiment of the present invention R⁶ and R⁷ in formula I are taken together to form a 5 membered heterocyclic ring system, said heterocyclic ring system containing 1 N heteroatom, wherein said N atom present in the ring system is optionally substituted with a substituent selected from —(C₁-C₁₀)alkyl, —R¹⁰, —C(O)R¹⁰, —SO₂R¹⁰, —C(O)NR¹⁰C(O)R¹⁰, —C(O)NR¹⁰C(O)OR¹⁰, —C(O)NR⁸R⁹, —C(O)OR¹⁰ and each carbon atom in the ring is independently optionally substituted by 1 to 2 substituents selected from R¹.

In another embodiment of the present invention R⁶ and R⁷ in formula I are taken together to form a 6 membered heterocyclic ring system, said heterocyclic ring system containing 1 N heteroatom, wherein said N atom present in the ring system is optionally substituted with a substituent selected from —(C₁-C₁₀)alkyl, —R¹⁰, —C(O)R¹⁰, —SO₂R¹⁰, —C(O)NR¹⁰C(O)R¹⁰, —C(O)NR¹⁰C(O)OR¹⁰, —C(O)NR⁸R⁹, —C(O)OR¹⁰ and each carbon atom in the ring is independently optionally substituted by 1 to 2 substituents selected from R¹.

In another embodiment of the present invention R⁶ and R⁷ in formula I are taken together to form a 7 membered heterocyclic ring system, said heterocyclic ring system containing 1 N heteroatom, wherein said N atom present in the ring system is optionally substituted with a substituent selected from —(C₁-C₁₀)alkyl, —R¹⁰, —C(O)R¹⁰, —SO₂R¹⁰, —C(O)NR¹⁰C(O)R¹⁰, —C(O)NR¹⁰C(O)OR¹⁰, —C(O)NR⁸R⁹, —C(O)OR¹⁰ and each carbon atom in the ring is independently optionally substituted by 1 to 2 substituents selected from R¹.

In another embodiment of the present invention R⁶ and R⁷ in formula I are taken together to form a 4 to 7 membered heterocyclic ring system, said heterocyclic ring system containing 1 to 2 S heteroatoms, wherein each carbon atom in the ring are independently optionally substituted by 1 to 2 substituents selected from R¹.

In a preferred embodiment of the present invention R⁶ and R⁷ in formula I are taken together to form a ring system selected from the group consisting of:

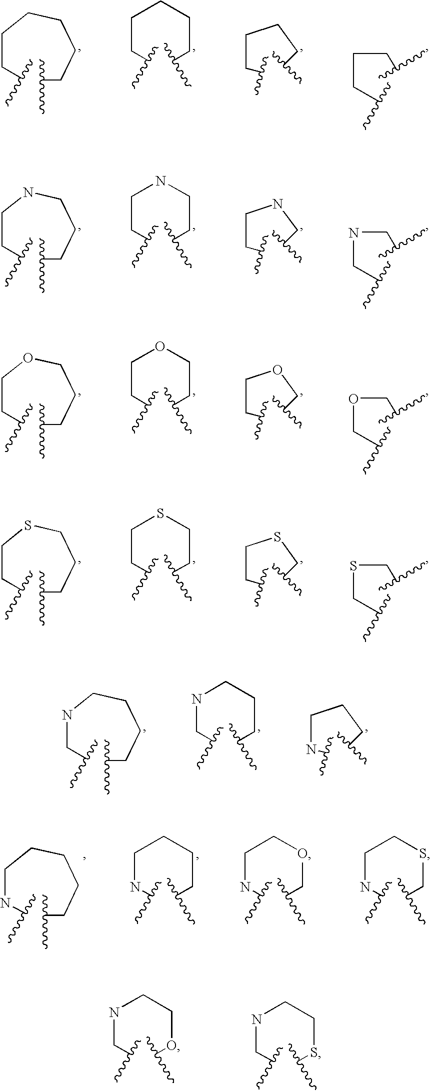

and wherein each substitutable N atom present in the heterocyclic ring systems above is optionally substituted with a substituent selected from —(C₁-C₁₀)alkyl, —R¹⁰, —C(O)R¹⁰, —SO₂R¹⁰, —C(O)NR¹⁰C(O)R¹⁰, —C(O)NR¹⁰C(O)OR¹⁰, —C(O)NR⁸R⁹, —C(O)OR¹⁰ and each carbon atom in the heterocyclic ring systems above is independently optionally substituted by 1 to 2 substituents selected from R¹, and each carbon atom in the cyclic ring systems above is independently optionally substituted by 1 to 2 substituents selected from R¹.

In a more preferred embodiment of the present invention R⁶ and R⁷ in formula I are taken together to form a ring system selected from the group consisting of:

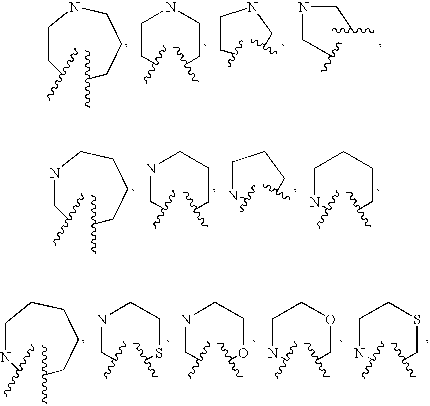

and wherein each substitutable N atom present in the heterocyclic ring system above is optionally substituted with a substituent selected from —(C₁-C₁₀)alkyl, —R¹⁰, —C(O)R¹⁰, —SO₂R¹⁰, —C(O)NR¹⁰C(O)R¹⁰, —C(O)NR¹⁰C(O)OR¹⁰, —C(O)NR⁸R⁹, —C(O)OR¹⁰ and each carbon atom in the heterocyclic ring systems above is independently optionally substituted by 1 to 2 substituents selected from R¹.

In a most preferred embodiment of the present invention R⁶ and R⁷ in formula I are taken together to form a ring system selected from the group consisting of:

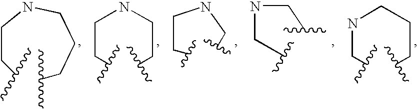

and wherein each substitutable N atom present in the heterocyclic ring systems above is optionally substituted with a substituent selected from —(C₁-C₁₀)alkyl, —R¹⁰, —C(O)R¹⁰, —SO₂R¹⁰, —C(O)NR¹⁰C(O)R¹⁰, —C(O)NR¹⁰C(O)OR¹⁰, —C(O)NR⁸R⁹, —C(O)OR¹⁰ and each carbon atom in the heterocyclic ring systems above is independently optionally substituted by 1 to 2 substituents selected from R¹.

In another most preferred embodiment of the present invention R⁶ and R⁷ in formula I are taken together to form a ring system selected from the group consisting of:

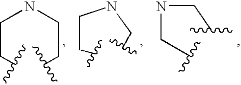

and wherein each substitutable N atom present in the heterocyclic ring systems above is optionally substituted with a substituent selected from —(C₁-C₁₀)alkyl, —R¹⁰, —C(O)R¹⁰, —SO₂R¹⁰, —C(O)NR¹⁰C(O)R¹⁰, —C(O)NR¹⁰C(O)OR¹⁰, —C(O)NR⁸R⁹, —C(O)OR¹⁰ and each carbon atom in the heterocyclic ring systems above is independently optionally substituted by 1 to 2 substituents selected from R¹.

Specific embodiments of the compounds of formula I include those selected from the group consisting of:
(3R)-1-(1H-pyrrolo[2,3-b]pyridin-4-yl)-1,2-dihydrospiro[indole-3,3′-pyrrolidine];
(3R)-1′-(3-furylmethyl)-1-(7H-pyrrolo[2,3-d]pyrimidin-4-yl)-1,2-dihydrospiro[indole-3,3′-pyrrolidine];
(3R)-1′-(3-methylbutyl)-1-(7H-pyrrolo[2,3-d]pyrimidin-4-yl)-1,2-dihydrospiro[indole-3,3′-pyrrolidine];
(3R)-1′-(4-chlorobenzyl)-1-(7H-pyrrolo[2,3-d]pyrimidin-4-yl)-1,2-dihydrospiro[indole-3,3′-pyrrolidine];
(3R)-1-(5-chloro-7H-pyrrolo[2,3-d]pyrimidin-4-yl)-1′-(cyclopropylmethyl)-1,2-dihydrospiro[indole-3,3′-pyrrolidine];
(3R)-1-(5-chloro-7H-pyrrolo[2,3-d]pyrimidin-4-yl)-1,2-dihydrospiro[indole-3,3′-pyrrolidine];
(3R)-1-(5-chloro-7H-pyrrolo[2,3-d]pyrimidin-4-yl)-1′-ethyl-1,2-dihydrospiro[indole-3,3′-pyrrolidine];
(3R)-1-(5-chloro-7H-pyrrolo[2,3-d]pyrimidin-4-yl)-1′-methyl-1,2-dihydrospiro[indole-3,3′-pyrrolidine];
(3R)-1-(5-methyl-7H-pyrrolo[2,3-d]pyrimidin-4-yl)-1,2-dihydrospiro[indole-3,3′-pyrrolidine];
(3R)-1-(7H-pyrrolo[2,3-d]pyrimidin-4-yl)-1,2-dihydrospiro[indole-3,3′-pyrrolidine];
(3R)-1′-(cyclopropylmethyl)-1-(7H-pyrrolo[2,3-d]pyrimidin-4-yl)-1,2-dihydrospiro[indole-3,3′-pyrrolidine];
(3R)-1′-butyl-1-(5-chloro-7H-pyrrolo[2,3-d]pyrimidin-4-yl)-1,2-dihydrospiro[indole-3,3′-pyrrolidine];
(3R)-1′-butyl-1-(7H-pyrrolo[2,3-d]pyrimidin-4-yl)-1,2-dihydrospiro[indole-3,3′-pyrrolidine];
(3R)-1′-ethyl-1-(7H-pyrrolo[2,3-d]pyrimidin-4-yl)-1,2-dihydrospiro[indole-3,3′-pyrrolidine];
(3R)-1′-methyl-1-(7H-pyrrolo[2,3-d]pyrimidin-4-yl)-1,2-dihydrospiro[indole-3,3′-pyrrolidine];
(3R)-1′-propyl-1-(7H-pyrrolo[2,3-d]pyrimidin-4-yl)-1,2-dihydrospiro[indole-3,3′-pyrrolidine];
(3S)-1-(5-chloro-7H-pyrrolo[2,3-d]pyrimidin-4-yl)-1,2-dihydrospiro[indole-3,3′-pyrrolidine];
(3S)-1-(7H-pyrrolo[2,3-d]pyrimidin-4-yl)-1,2-dihydrospiro[indole-3,3′-pyrrolidine];
(3S)-1-(7H-pyrrolo[2,3-d]pyrimidin-4-yl)-1,2-dihydrospiro[indole-3,3′-pyrrolidine];
(3S)-1′-methyl-1-(7H-pyrrolo[2,3-d]pyrimidin-4-yl)-1,2-dihydrospiro[indole-3,3′-pyrrolidine];
1-(1H-pyrrolo[2,3-b]pyridin-4-yl)-1,2-dihydrospiro[indole-3,4′-piperidine];
1-(3-chloro-1H-pyrrolo[2,3-b]pyridin-4-yl)-1,2-dihydrospiro[indole-3,4′-piperidine];
1′-(4-chlorobenzyl)-5-methoxy-1-(7H-pyrrolo[2,3-d]pyrimridin-4-yl)-1,2-dihydrospiro[indole-3,4′-piperidine];
1-(5-chloro-7H-pyrrolo[2,3-d]pyrimidin-4-yl)-1,2-dihydrospiro[indole-3,4′-piperidine];
1-(5-chloro-7H-pyrrolo[2,3-d]pyrimidin-4-yl)-5-fluoro-1′-(morpholin-4-ylcarbonyl)-1,2-dihydrospiro[indole-3,4′-piperidine];
1-(5-chloro-7H-pyrrolo[2,3-d]pyrimidin-4-yl)-5-fluoro-1,2-dihydrospiro[indole-3,4′-piperidine];
1-(5-chloro-7H-pyrrolo[2,3-d]pyrimidin-4-yl)-5-methyl-1,2-dihydrospiro[indole-3,4′-piperidine];
1-(5-chloro-7H-pyrrolo[2,3-d]pyrimidin-4-yl)-5-methyl-1′-[(4-methyl piperazin-1-yl)carbonyl]-1,2-dihydrospiro[indole-3,4′-piperidine];
1-(5-methyl-7H-pyrrolo[2,3-d]pyrimidin-4-yl)-1,2-dihydrospiro[indole-3,4′-piperidine];
1-(7H-pyrrolo[2,3-d]pyrimidin-4-yl)-1,2-dihydrospiro[indole-3,4′-piperidine];
1-(7H-pyrrolo[2,3-d]pyrimidin-4-yl)-1,2-dihydrospiro[indole-3,4′-piperidine]-4-carbonitrile;
1-(7H-pyrrolo[2,3-d]pyrimidin-4-yl)-1,2-dihydrospiro[indole-3,4′-piperidine]-5-carbonitrile;
1-(7H-pyrrolo[2,3-d]pyrimidin-4-yl)-1,2-dihydrospiro[indole-3,4′-piperidine]-5-sulfonamide;
1-(7H-pyrrolo[2,3-d]pyrimidin-4-yl)-5-(2-thienyl)-1,2-dihydrospiro[indole-3,4′-piperidine];
1-(7H-pyrrolo[2,3-d]pyrimidin-4-yl)-5-(3-thienyl)-1,2-dihydrospiro[indole-3,4′-piperidine];
1-(7H-pyrrolo[2,3-d]pyrimidin-4-yl)-5-(trifluoromethyl)-1,2-dihydrospiro[indole-3,4′-piperidine];
1-(7H-pyrrolo[2,3-d]pyrimidin-4-yl)-N-[3-(trifluoromethoxy)benzyl]-1,2-dihydrospiro[indole-3,4′-piperidin]-5-amine;
1-(9H-purin-6-yl)-1,2-dihydrospiro[indole-3,4′-piperidine];
1′-(isopropylsulfonyl)-1-(7H-pyrrolo[2,3-d]pyrimidin-4-yl)-1,2-dihydrospiro[indole-3,4′-piperidine];
1′,5-dimethyl-1-(7H-pyrrolo[2,3-d]pyrimidin-4-yl)-1,2-dihydrospiro[indole-3,4′-piperidine];
1′-[1-(4-chlorophenyl)ethyl]-5-fluoro-1-(7H-pyrrolo[2,3-d]pyrimidin-4-yl)-1,2-dihydrospiro[indole-3,4′-piperidine];
1′-methyl-1-(1H-pyrrolo[2,3-b]pyridin-4-yl)-1,2-dihydrospiro[indole-3,4′-piperidine];
2-[1-(7H-pyrrolo[2,3-d]pyrimidin-4-yl)-1,2-dihydrospiro[indole-3,4′-piperidin]-5-yl]benzonitrile;
2-cyclopropyl-1-(1H-pyrazolo[3,4-d]pyrimidin-4-yl)-1,2-dihydrospiro[indole-3,4′-piperidine];
2-methyl-1-(7H-pyrrolo[2,3-d]pyrimidin-4-yl)-1,2-dihydrospiro[indole-3,4′-piperidine];
2-methyl-1-(9H-purin-6-yl)-1,2-dihydrospiro[indole-3,4′-piperidine];
2-phenyl-1-(1H-pyrazolo[3,4-d]pyrimidin-4-yl)-1,2-dihydrospiro[indole-3,4′-piperidine];
2-propyl-1-(1H-pyrazolo[3,4-d]pyrimidin-4-yl)-1,2-dihydrospiro[indole-3,4′-piperidine];
3-(7H-pyrrolo[2,3-d]pyrimidin-4-yl)-2,3-dihydrospiro[benzo[e]indole-1,4′-piperidine];
3-[1-(5-chloro-7H-pyrrolo[2,3-d]pyrimidin-4-yl)-5-methyl-1,2-dihydro-1′H-spiro[indole-3,4′-piperidin]-1′-yl]-N,N,2,2-tetramethylpropan-1-amine;
3-[1-(7H-pyrrolo[2,3-d]pyrimidin-4-yl)-1,2-dihydrospiro[indole-3,4′-piperidin]-5-yl]benzonitrile;
4-(5-chlorospiro[indole-3,4′-piperidin]-1(2H)-yl)-7H-pyrrolo[2,3-d]pyrimidine-5-carbonitrile;
4-(5-fluorospiro[indole-3,4′-piperidin]-1(2H)-yl)-7H-pyrrolo[2,3-d]pyrimidine-5-carbonitrile;
4,5-dichloro-1-(7H-pyrrolo[2,3-d]pyrimidin-4-yl)-1,2-dihydrospiro[indole-3,4′-piperidine];
4,5-dimethyl-1-(7H-pyrrolo[2,3-d]pyrimidin-4-yl)-1,2-dihydrospiro[indole-3,4′-piperidine];
4-chloro-1-(7H-pyrrolo[2,3-d]pyrimidin-4-yl)-1,2-dihydrospiro[indole-3,4′-piperidine];
4-chloro-5-methyl-1-(7H-pyrrolo[2,3-d]pyrimidin-4-yl)-1,2-dihydrospiro[indole-3,4′-piperidine];
4-fluoro-1-(7H-pyrrolo[2,3-d]pyrimidin-4-yl)-1,2-dihydrospiro[indole-3,4′-piperidine];
4-methyl-1-(7H-pyrrolo[2,3-d]pyrimidin-4-yl)-1,2-dihydrospiro[indole-3,4′-piperidine];
4-spiro[indole-3,4′-piperidin]-1(2H)-yl-7H-pyrrolo[2,3-d]pyrimidine-5-carbonitrile;
5-(1,3-benzodioxol-5-yl)-1-(7H-pyrrolo[2,3-d]pyrimidin-4-yl)-1,2-dihydrospiro[indole-3,4′-piperidine];
5-(2-methylphenyl)-1-(7H-pyrrolo[2,3-d]pyrimidin-4-yl)-1,2-dihydrospiro[indole-3,4′-piperidine];
5-(2-phenoxyphenyl)-1-(7H-pyrrolo[2,3-d]pyrimidin-4-yl)-1,2-dihydrospiro[indole-3,4′-piperidine];
5-(3,4-dihydroquinolin-1(2H)-yl)-1-(7H-pyrrolo[2,3-d]pyrimidin-4-yl)-1,2-dihydrospiro[indole-3,4′-piperidine];
5-(3,5-dimethylisoxazol-4-yl)-1-(7H-pyrrolo[2,3-d]pyrimidin-4-yl)-1,2-dihydrospiro[indole-3,4′-piperidine];
5-(3-furyl)-1-(7H-pyrrolo[2,3-d]pyrimidin-4-yl)-1,2-dihydrospiro[indole-3,4′-piperidine];
5-(4-methylpiperazin-1-yl)-1-(7H-pyrrolo[2,3-d]pyrimidin-4-yl)-1,2-dihydrospiro[indole-3,4′-piperidine];
5-(5-methyl-2-furyl)-1-(7H-pyrrolo[2,3-d]pyrimidin-4-yl)-1,2-dihydrospiro[indole-3,4′-piperidine];
5-(methylsulfonyl)-1-(7H-pyrrolo[2,3-d]pyrimidin-4-yl)-1,2-dihydrospiro[indole-3,4′-piperidine];
5-biphenyl-2-yl-1-(7H-pyrrolo[2,3-d]pyrimidin-4-yl)-1,2-dihydrospiro[indole-3,4′-piperidine];
5-chloro-1-(5-chloro-7H-pyrrolo[2,3-d]pyrimidin-4-yl)-1′-(1H-imidazol-4-ylmethyl)-1,2-dihydrospiro[indole-3,4′-piperidine];
5-chloro-1-(5-chloro-7H-pyrrolo[2,3-d]pyrimidin-4-yl)-1,2-dihydrospiro[indole-3,4′-piperidine];
5-chloro-1-(7H-pyrrolo[2,3-d]pyrimidin-4-yl)-1,2-dihydrospiro[indole-3,4′-piperidine];
5-fluoro-1-(1H-pyrrolo[2,3-b]pyridin-4-yl)-1,2-dihydrospiro[indole-3,4′-piperidine];
5-fluoro-1-(3-methyl-1H-pyrazolo[3,4-d]pyrimidin-4-yl)-1,2-dihydrospiro[indole-3,4′-piperidine];
5-fluoro-1-(5-methyl-7H-pyrrolo[2,3-d]pyrimidin-4-yl)-1,2-dihydrospiro[indole-3,4′-piperidine];
5-fluoro-1-(7H-pyrrolo[2,3-d]pyrimidin-4-yl)-1,2-dihydrospiro[indole-3,4′-piperidine];
5-fluoro-1′-methyl-1-(7H-pyrrolo[2,3-d]pyrimidin-4-yl)-1,2-dihydrospiro[indole-3,4′-piperidine];
5-isopropyl-1-(7H-pyrrolo[2,3-d]pyrimidin-4-yl)-1,2-dihydrospiro[indole-3,4′-piperidine];
5-methoxy-1-(7H-pyrrolo[2,3-d]pyrimidin-4-yl)-1,2-dihydrospiro[indole-3,4′-piperidine];
5-methyl-1-(5-methyl-7H-pyrrolo[2,3-d]pyrimidin-4-yl)-1,2-dihydrospiro[indole-3,4′-piperidine];
5-methyl-1-(9H-purin-6-yl)-1,2-dihydrospiro[indole-3,4′-piperidine];
5-morpholin-4-yl-1-(7H-pyrrolo[2,3-d]pyrimidin-4-yl)-1,2-dihydrospiro[indole-3,4′-piperidine];
5-phenoxy-1-(7H-pyrrolo[2,3-d]pyrimidin-4-yl)-1,2-dihydrospiro[indole-3,4′-piperidine];
5-phenyl-1-(7H-pyrrolo[2,3-d]pyrimidin-4-yl)-1,2-dihydrospiro[indole-3,4′-piperidine];
5-pyridin-3-yl-1-(7H-pyrrolo[2,3-d]pyrimidin-4-yl)-1,2-dihydrospiro[indole-3,4′-piperidine];
5-pyridin-4-yl-1-(7H-pyrrolo[2,3-d]pyrimidin-4-yl)-1,2-dihydrospiro[indole-3,4′-piperidine];
5-pyrimidin-5-yl-1-(7H-pyrrolo[2,3-d]pyrimidin-4-yl)-1,2-dihydrospiro[indole-3,4′-piperidine];
6-(3-methyl-1H-pyrazolo[3,4-d]pyrimidin-4-yl)-6,7-dihydro-3H-spiro[imidazo[4,5-e]indole-8,4′-piperidine];
6-(7H-pyrrolo[2,3-d]pyrimidin-4-yl)-6,7-dihydro-3H-spiro[imidazo[4,5-e]indole-8,4′-piperidine];
6-chloro-5-methyl-1-(7H-pyrrolo[2,3-d]pyrimidin-4-yl)-1,2-dihydrospiro[indole-3,4′-piperidine];
methyl 4-{[1-(7H-pyrrolo[2,3-d]pyrimidin-4-yl)-1,2-dihydro-1′H-spiro[indole-3,4′-piperidin]-1′-yl]methyl}benzoate;
N-(2,2-dimethylpropyl)-1-(7H-pyrrolo[2,3-d]pyrimidin-4-yl)-1,2-dihydrospiro[indole-3,4′-piperidin]-5-amine;
N-(2-fluorobenzyl)-1-(7H-pyrrolo[2,3-d]pyrimidin-4-yl)-1,2-dihydrospiro[indole-3,4′-piperidin]-5-amine;
N-(2-methoxybenzyl)-1-(7H-pyrrolo[2,3-d]pyrimidin-4-yl)-1,2-dihydrospiro[indole-3,4′-piperidin]-5-amine;
N-(2-methoxyethyl)-1-(7H-pyrrolo[2,3-d]pyrimidin-4-yl)-1,2-dihydrospiro[indole-3,4′-piperidin]-5-amine;
N-(2-methyl benzyl)-1-(7H-pyrrolo[2,3-d]pyrimidin-4-yl)-1,2-dihydrospiro[indole-3,4′-piperidin]-5-amine;
N-(2-phenylethyl)-1-(7H-pyrrolo[2,3-d]pyrimidin-4-yl)-1,2-dihydrospiro[indole-3,4′-piperidin]-5-amine;
N-(3-chlorophenyl)-1-(7H-pyrrolo[2,3-d]pyrimidin-4-yl)-1,2-dihydrospiro[indole-3,4′-piperidin]-5-amine;
N-(3-methoxybenzyl)-1-(7H-pyrrolo[2,3-d]pyrimidin-4-yl)-1,2-dihydrospiro[indole-3,4′-piperidin]-5-amine;
N-(3-methyl benzyl)-1-(7H-pyrrolo[2,3-d]pyrimidin-4-yl)-1,2-dihydrospiro[indole-3,4′-piperidin]-5-amine;
N-(3-methylphenyl)-1-(7H-pyrrolo[2,3-d]pyrimidin-4-yl)-1,2-dihydrospiro[indole-3,4′-piperidin]-5-amine;
N-(4-chlorobenzyl)-1-(7H-pyrrolo[2,3-d]pyrimidin-4-yl)-1,2-dihydrospiro[indole-3,4′-piperidin]-5-amine;
N-(4-chlorophenyl)-1-(7H-pyrrolo[2,3-d]pyrimidin-4-yl)-1,2-dihydrospiro[indole-3,4′-piperidin]-5-amine;
N-(4-methyl benzyl)-1-(7H-pyrrolo[2,3-d]pyrimidin-4-yl)-1,2-dihydrospiro[indole-3,4′-piperidin]-5-amine;
N-(4-methylphenyl)-1-(7H-pyrrolo[2,3-d]pyrimidin-4-yl)-1,2-dihydrospiro[indole-3,4′-piperidin]-5-amine;
N-(4-phenoxybenzyl)-1-(7H-pyrrolo[2,3-d]pyrimidin-4-yl)-1,2-dihydrospiro[indole-3,4′-piperidin]-5-amine;
N-(biphenyl-3-ylmethyl)-1-(7H-pyrrolo[2,3-d]pyrimidin-4-yl)-1,2-dihydrospiro[indole-3,4′-piperidin]-5-amine;
N-(biphenyl-4-ylmethyl)-1-(7H-pyrrolo[2,3-d]pyrimidin-4-yl)-1,2-dihydrospiro[indole-3,4′-piperidin]-5-amine;
N,N′-dimethyl-N-[1-(7H-pyrrolo[2,3-d]pyrimidin-4-yl)-1,2-dihydrospiro[indole-3,4′-piperidin]-5-yl]ethane-1,2-diamine;
N-[3-(1H-pyrazol-1-yl)benzyl]-1-(7H-pyrrolo[2,3-d]pyrimidin-4-yl)-1,2-dihydrospiro[indole-3,4′-piperidin]-5-amine;
N-[4-(1H-pyrazol-1-yl)benzyl]-1-(7H-pyrrolo[2,3-d]pyrimidin-4-yl)-1,2-dihydrospiro[indole-3,4′-piperidin]-5-amine;
N-{4-[1-(7H-pyrrolo[2,3-d]pyrimidin-4-yl)-1,2-dihydrospiro[indole-3,4′-piperidin]-5-yl]phenyl}acetamide;
N-benzyl-1-(7H-pyrrolo[2,3-d]pyrimidin-4-yl)-1,2-dihydrospiro[indole-3,4′-piperidin]-5-amine;
N-cyclobutyl-1-(7H-pyrrolo[2,3-d]pyrimidin-4-yl)-1,2-dihydrospiro[indole-3,4′-piperidin]-5-amine;
N-cyclopropyl-1-(7H-pyrrolo[2,3-d]pyrimidin-4-yl)-1,2-dihydrospiro[indole-3,4′-piperidine]-5-carboxamide;
N-phenyl-1-(7H-pyrrolo[2,3-d]pyrimidin-4-yl)-1,2-dihydrospiro[indole-3,4′-piperidin]-5-amine;
N-phenyl-1-(7H-pyrrolo[2,3-d]pyrimidin-4-yl)-1,2-dihydrospiro[indole-3,4′-piperidine]-5-carboxamide; and the pharmaceutically acceptable salts and solvates of the foregoing compounds.

More preferred embodiments of the compounds of formula I include those selected from the group consisting of:
(3R)-1-(5-methyl-7H-pyrrolo[2,3-d]pyrimidin-4-yl)-1,2-dihydrospiro[indole-3,3′-pyrrolidine];
(3R)-1-(7H-pyrrolo[2,3-d]pyrimidin-4-yl)-1,2-dihydrospiro[indole-3,3′-pyrrolidine];
(3S)-1-(5-chloro-7H-pyrrolo[2,3-d]pyrimidin-4-yl)-1,2-dihydrospiro[indole-3,3′-pyrrolidine];
(3S)-1-(7H-pyrrolo[2,3-d]pyrimidin-4-yl)-1,2-dihydrospiro[indole-3,3′-pyrrolidine];
(3S)-1′-methyl-1-(7H-pyrrolo[2,3-d]pyrimidin-4-yl)-1,2-dihydrospiro[indole-3,3′-pyrrolidine];
1-(1H-pyrrolo[2,3-b]pyridin-4-yl)-1,2-dihydrospiro[indole-3,4′-piperidine];
1-(3-chloro-1H-pyrrolo[2,3-b]pyridin-4-yl)-1,2-dihydrospiro[indole-3,4′-piperidine];
1-(5-chloro-7H-pyrrolo[2,3-d]pyrimidin-4-yl)-5-fluoro-1-(morpholin-4-ylcarbonyl)-1,2-dihydrospiro[indole-3,4′-piperidine];
1-(5-methyl-7H-pyrrolo[2,3-d]pyrimidin-4-yl)-1,2-dihydrospiro[indole-3,4′-piperidine];
1-(7H-pyrrolo[2,3-d]pyrimidin-4-yl)-1,2-dihydrospiro[indole-3,4′-piperidine];
1-(7H-pyrrolo[2,3-d]pyrimidin-4-yl)-1,2-dihydrospiro[indole-3,4′-piperidine]-4-carbonitrile;
1-(7H-pyrrolo[2,3-d]pyrimidin-4-yl)-1,2-dihydrospiro[indole-3,4′-piperidine]-5-carbonitrile;
1-(7H-pyrrolo[2,3-d]pyrimidin-4-yl)-5-(trifluoromethyl)-1,2-dihydrospiro[indole-3,4′-piperidine];
1-(9H-purin-6-yl)-1,2-dihydrospiro[indole-3,4′-piperidine]; 1′-(isopropylsulfonyl)-1-(7H-pyrrolo[2,3-d]pyrimidin-4-yl)-1,2-dihydrospiro[indole-3,4′-piperidine];
1′-[1-(4-chlorophenyl)ethyl]-5-fluoro-1-(7H-pyrrolo[2,3-d]pyrimidin-4-yl)-1,2-dihydrospiro[indole-3,4′-piperidine];
1′-methyl-1-(1H-pyrrolo[2,3-b]pyridin-4-yl)-1,2-dihydrospiro[indole-3,4′-piperidine];
2-methyl-1-(7H-pyrrolo[2,3-d]pyrimidin-4-yl)-1,2-dihydrospiro[indole-3,4′-piperidine];
2-methyl-1-(9H-purin-6-yl)-1,2-dihydrospiro[indole-3,4′-piperidine];
3-(7H-pyrrolo[2,3-d]pyrimidin-4-yl)-2,3-dihydrospiro[benzo[e]indole-1,4′-piperidine];
3-[1-(5-chloro-7H-pyrrolo[2,3-d]pyrimidin-4-yl)-5-methyl-1,2-dihydro-1′H-spiro[indole-3,4′-piperidin]-1′-yl]-N,N,2,2-tetramethylpropan-1-amine;
4-(5-chlorospiro[indole-3,4′-piperidin]-1(2H)-yl)-7H-pyrrolo[2,3-d]pyrimidine-5-carbonitrile;
4-(5-fluorospiro[indole-3,4′-piperidin]-1(2H)-yl)-7H-pyrrolo[2,3-d]pyrimidine-5-carbonitrile;
4-spiro[indole-3,4′-piperidin]-1(2H)-yl-7H-pyrrolo[2,3-d]pyrimidine-5-carbonitrile;
5-(3,5-dimethylisoxazol-4-yl)-1-(7H-pyrrolo[2,3-d]pyrimidin-4-yl)-1,2-dihydrospiro[indole-3,4′-piperidine];
5-(4-methylpiperazin-1-yl)-1-(7H-pyrrolo[2,3-d]pyrimidin-4-yl)-1,2-dihydrospiro[indole-3,4′-piperidine];
5-chloro-1-(5-chloro-7H-pyrrolo[2,3-d]pyrimidin-4-yl)-1′-(1H-imidazol-4-ylmethyl)-1,2-dihydrospiro[indole-3,4′-piperidine];
5-chloro-1-(5-chloro-7H-pyrrolo[2,3-d]pyrimidin-4-yl)-1,2-dihydrospiro[indole-3,4′-piperidine];
5-chloro-1-(7H-pyrrolo[2,3-d]pyrimidin-4-yl)-1,2-dihydrospiro[indole-3,4′-piperidine];
5-fluoro-1-(1H-pyrrolo[2,3-b]pyridin-4-yl)-1,2-dihydrospiro[indole-3,4′-piperidine];
5-fluoro-1-(7H-pyrrolo[2,3-d]pyrimidin-4-yl)-1,2-dihydrospiro[indole-3,4′-piperidine];
5-methyl-1-(9H-purin-6-yl)-1,2-dihydrospiro[indole-3,4′-piperidine];
5-phenoxy-1-(7H-pyrrolo[2,3-d]pyrimidin-4-yl)-1,2-dihydrospiro[indole-3,4′-piperidine];
6-(3-methyl-1H-pyrazolo[3,4-d]pyrimidin-4-yl)-6,7-dihydro-3H-spiro[imidazo[4,5-e]indole-8,4′-piperidine];
6-(7H-pyrrolo[2,3-d]pyrimidin-4-yl)-6,7-dihydro-3H-spiro[imidazo[4,5-e]indole-8,4′-piperidine];
methyl 4-{[1-(7H-pyrrolo[2,3-d]pyrimidin-4-yl)-1,2-dihydro-1′H-spiro[indole-3,4′-piperidin]-1′-yl]methyl}benzoate;
N-(3-chlorophenyl)-1-(7H-pyrrolo[2,3-d]pyrimidin-4-yl)-1,2-dihydrospiro[indole-3,4′-piperidin]-5-amine;
N-cyclopropyl-1-(7H-pyrrolo[2,3-d]pyrimidin-4-yl)-1,2-dihydrospiro[indole-3,4′-piperidine]-5-carboxamide; and the pharmaceutically acceptable salts and solvates of the foregoing compounds.

Most preferred embodiments of the compounds of formula I include those selected from the group consisting of:
(3S)-1-(7H-pyrrolo[2,3-d]pyrimidin-4-yl)-1,2-dihydrospiro[indole-3,3′-pyrrolidine];
1-(1H-pyrrolo[2,3-b]pyridin-4-yl)-1,2-dihydrospiro[indole-3,4′-piperidine];
1-(7H-pyrrolo[2,3-d]pyrimidin-4-yl)-1,2-dihydrospiro[indole-3,4′-piperidine]-5-carbonitrile;
1′-[1-(4-chlorophenyl)ethyl]-5-fluoro-1-(7H-pyrrolo[2,3-d]pyrimidin-4-yl)-1,2-dihydrospiro[indole-3,4′-piperidine];
4-(5-fluorospiro[indole-3,4′-piperidin]-1(2H)-yl)-7H-pyrrolo[2,3-d]pyrimidine-5-carbonitrile;
4-spiro[indole-3,4′-piperidin]-1(2H)-yl-7H-pyrrolo[2,3-d]pyrimidine-5-carbonitrile;
5-(3,5-dimethylisoxazol-4-yl)-1-(7H-pyrrolo[2,3-d]pyrimidin-4-yl)-1,2-dihydrospiro[indole-3,4′-piperidine];
5-chloro-1-(7H-pyrrolo[2,3-d]pyrimidin-4-yl)-1,2-dihydrospiro[indole-3,4′-piperidine];
5-fluoro-1-(7H-pyrrolo[2,3-d]pyrimidin-4-yl)-1,2-dihydrospiro[indole-3,4′-piperidine];
6-(3-methyl-1H-pyrazolo[3,4-d]pyrimidin-4-yl)-6,7-dihydro-3H-spiro[imidazo[4,5-e]indole-8,4′-piperidine];
N-(3-chlorophenyl)-1-(7H-pyrrolo[2,3-d]pyrimidin-4-yl)-1,2-dihydrospiro[indole-3,4′-piperidin]-5-amine; and the pharmaceutically acceptable salts and solvates of the foregoing compounds.

This invention also relates to a method for the treatment of abnormal cell growth in a mammal, including a human, comprising administering to said mammal an amount of a compound of the formula I, as defined above, or a pharmaceutically acceptable salt or solvate thereof, that is effective in treating abnormal cell growth.

In one embodiment of this method, the abnormal cell growth is cancer, including, but not limited to, mesothelioma, hepatobilliary (hepatic and billiary duct), a primary or secondary CNS tumor, a primary or secondary brain tumor, lung cancer (NSCLC and SCLC), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, non hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In a preferred embodiment of the method of the present invention the abnormal cell growth is cancer selected from lung cancer (NSCLC and SCLC), cancer of the head or neck, ovarian cancer, colon cancer, rectal cancer, cancer of the anal region, stomach cancer, breast cancer, prostate cancer, cancer of the kidney or ureter, renal cell carcinoma, carcinoma of the renal pelvis, neoplasms of the central nervous system (CNS), primary CNS lymphoma, non hodgkins's lymphoma, spinal axis tumors, or a combination of one or more of the foregoing cancers.

In a more preferred embodiment of the method of the present invention the abnormal cell growth is cancer selected selected from lung cancer (NSCLC and SCLC), ovarian cancer, colon cancer, breast cancer, prostate cancer, rectal cancer, cancer of the anal region, or a combination of one or more of the foregoing cancers.

In an even more preferred embodiment of the method of the present invention the abnormal cell growth is cancer selected selected from lung cancer (NSCLC and SCLC), ovarian cancer, colon cancer, breast cancer, prostate cancer, rectal cancer, or a combination of one or more of the foregoing cancers.

In a most preferred embodiment of the method of the present invention the abnormal cell growth is cancer selected selected from lung cancer (NSCLC and SCLC), colon cancer, breast cancer, prostate cancer, rectal cancer, or a combination of one or more of the foregoing cancers.

In the most preferred embodiment of the method of the present invention the abnormal cell growth is cancer selected from lung cancer (NSCLC and SCLC), colon cancer, rectal cancer, colorectal cancer, breast cancer, and prostate cancer.

In another embodiment of said method, said abnormal cell growth is a benign proliferative disease, including, but not limited to, psoriasis, benign prostatic hypertrophy or restinosis.

This invention also relates to a method for the treatment of abnormal cell growth in a mammal which comprises administering to said mammal an amount of a compound of formula I, or a pharmaceutically acceptable salt or solvate thereof, that is effective in treating abnormal cell growth in combination with an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, antibodies, cytotoxics, anti-hormones, and anti-androgens.

This invention also relates to a pharmaceutical composition for the treatment of abnormal cell growth in a mammal, including a human, comprising an amount of a compound of the formula I, as defined above, or a pharmaceutically acceptable salt or solvate thereof, that is effective in treating abnormal cell growth, and a pharmaceutically acceptable carrier. In one embodiment of said composition, said abnormal cell growth is cancer, including, but not limited to, mesothelioma, hepatobilliary (hepatic and billiary duct), a primary or secondary CNS tumor, a primary or secondary brain tumor, lung cancer (NSCLC and SCLC), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, non hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers. In another embodiment of said pharmaceutical composition, said abnormal cell growth is a benign proliferative disease, including, but not limited to, psoriasis, benign prostatic hypertrophy or restinosis.

The invention also relates to a pharmaceutical composition for the treatment of abnormal cell growth in a mammal, including a human, which comprises an amount of a compound of formula I, as defined above, or a pharmaceutically acceptable salt or solvate thereof, that is effective in treating abnormal cell growth in combination with a pharmaceutically acceptable carrier and an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, and anti-androgens.

The invention also relates to a method for the treatment of a hyperproliferative disorder in a mammal which comprises administering to said mammal a therapeutically effective amount of a compound of formula I, or a pharmaceutically acceptable salt or hydrate thereof, in combination with an anti-tumor agent selected from the group consisting antiproliferative agents, kinase inhibitors, angiogenesis inhibitors, growth factor inhibitors, cox-I inhibitors, cox-II inhibitors, mitotic inhibitors, alkylating agents, anti-metabolites, intercalating antibiotics, growth factor inhibitors, radiation, cell cycle inhibitors, enzymes, topoisomerase inhibitors, biological response modifiers, antibodies, cytotoxics, anti-hormones, statins, and anti-androgens.

In one embodiment of the present invention provides for a method for preparing a compound of formula I which comprises reacting a compound of formula 9 with a compound of formula 20 as shown below:

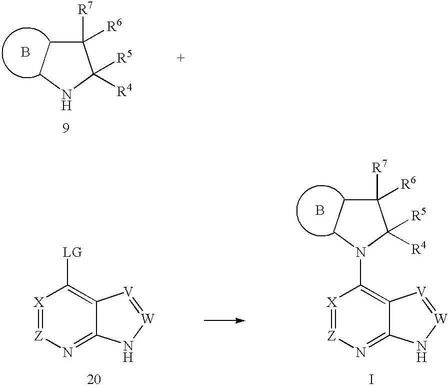

wherein LG is a leaving group in formula 20 and X, Z, V, W, B, R⁴, R⁵, R⁶, R⁷ are as defined hereinabove.

In one embodiment of the method of preparing the compound of formula I the leaving group LG in formula 20 is a halogen, preferably a chloride.

In one embodiment of the method for preparing the compound of formula I the reaction is carried out in the absence of a solvent.

In another embodiment of the method for preparing the compound of formula I the reaction is carried out in a solvent selected from the group consisting of ethyl acetate, DMF and NMP.

In one embodiment of the method for preparing the compound of formula I the reaction is carried out at a temperature between 60° C. and 140° C., for a period of 1 to 48 hrs.

In one embodiment of the method for preparing the compound of formula I the reaction is carried out in the presence of TFA or phosphorous acid.

In another embodiment of the method for preparing the compound of formula I the reaction is carried out in a buffered system with K₂PO₄.

In one embodiment of the present invention the anti-tumor agent used in conjunction with a compound of formula I and pharmaceutical compositions described herein is an anti-angiogenesis agent, kinase inhibitor, pan kinase inhibitor or growth factor inhibitor.

Preferred pan kinase inhibitors include SUTENT® (SU-11248), described in U.S. Pat. No. 6,573,293 (Pfizer, Inc, NY, USA).

Anti-angiogenesis agents, include but are not limited to the following agents, such as EGF inhibitor, EGFR inhibitors, VEGF inhibitors, VEGFR inhibitors, TIE2 inhibitors, IGF1R inhibitors, COX-II (cyclooxygenase 11) inhibitors, MMP-2 (matrix-metalloprotienase 2) inhibitors, and MMP-9 (matrix-metalloprotienase 9) inhibitors.

Preferred VEGF inhibitors, include for example, Avastin (bevacizumab), an anti-VEGF monoclonal antibody of Genentech, Inc. of South San Francisco, Calif.

Additional VEGF inhibitors include CP-547,632 (Pfizer Inc., NY, USA), AG13736 (Pfizer Inc.), ZD-6474 (AstraZeneca), AEE788 (Novartis), AZD-2171), VEGF Trap (Regeneron/Aventis), Vatalanib (also known as PTK-787, ZK-222584: Novartis & Schering AG), Macugen (pegaptanib octasodium, NX-1838, EYE-001, Pfizer Inc./Gilead/Eyetech), IM862 (Cytran Inc. of Kirkland, Wash., USA); and angiozyme, a synthetic ribozyme from Ribozyme (Boulder, Colo.) and Chiron (Emeryville, Calif.) and combinations thereof. VEGF inhibitors useful in the practice of the present invention are disclosed in U.S. Pat. Nos. 6,534,524 and 6,235,764, both of which are incorporated in their entirety for all purposed.

Particularly preferred VEGF inhibitors include CP-547,632, AG13736, Vatalanib, Macugen and combinations thereof.

Additional VEGF inhibitors are described in, for example in WO 99/24440 (published May 20, 1999), PCT International Application PCT/IB99/00797 (filed May 3, 1999), in WO 95/21613 (published Aug. 17, 1995), WO 99/61422 (published Dec. 2, 1999), U.S. Pat. No. 6,534,524 (discloses AG13736), U.S. Pat. No. 5,834,504 (issued Nov. 10, 1998), WO 98/50356 (published Nov. 12, 1998), U.S. Pat. No. 5,883,113 (issued Mar. 16, 1999), U.S. Pat. No. 5,886,020 (issued Mar. 23, 1999), U.S. Pat. No. 5,792,783 (issued Aug. 11, 1998), U.S. Pat. No. 6,653,308 (issued Nov. 25, 2003), WO 99/10349 (published Mar. 4, 1999), WO 97/32856 (published Sep. 12, 1997), WO 97/22596 (published Jun. 26, 1997), WO 98/54093 (published Dec. 3, 1998), WO 98/02438 (published Jan. 22, 1998), WO 99/16755 (published Apr. 8, 1999), and WO 98/02437 (published Jan. 22, 1998), all of which are herein incorporated by reference in their entirety.

Other antiproliferative agents that may be used with the compounds of the present invention include inhibitors of the enzyme farnesyl protein transferase and inhibitors of the receptor tyrosine kinase PDGFr, including the compounds disclosed and claimed in the following U.S. patent application Ser. Nos. 09/221,946 (filed Dec. 28, 1998); 09/454,058 (filed Dec. 2, 1999); 09/501,163 (filed Feb. 9, 2000); 09/539,930 (filed Mar. 31, 2000); 09/202,796 (filed May 22, 1997); 09/384,339 (filed Aug. 26, 1999); and 09/383,755 (filed Aug. 26, 1999); and the compounds disclosed and claimed in the following U.S. provisional patent applications: 60/168,207 (filed Nov. 30, 1999); 60/170,119 (filed Dec. 10, 1999); 60/177,718 (filed Jan. 21, 2000); 60/168,217 (filed Nov. 30, 1999), and 60/200,834 (filed May 1, 2000). Each of the foregoing patent applications and provisional patent applications is herein incorporated by reference in their entirety.

PDGRr inhibitors include but not limited to those disclosed international patent application publication number WO01/40217, published Jul. 7, 2001 and international patent application publication number WO2004/020431, published Mar. 11, 2004, the contents of which are incorporated in their entirety for all purposes.

Preferred PDGFr inhibitors include Pfizer's CP-673,451 and CP-868,596 and its pharmaceutically acceptable salts.

Preferred GARF inhibitors include Pfizer's AG-2037 (pelitrexol and its pharmaceutically acceptable salts. GARF inhibitors useful in the practice of the present invention are disclosed in U.S. Pat. No. 5,608,082 which is incorporated in its entirety for all purposed.

Examples of useful COX-11 inhibitors which can be used in conjunction with a compound of formula I and pharmaceutical compositions described herein include CELEBREX™ (celecoxib), parecoxib, deracoxib, ABT-963, MK-663 (etoricoxib), COX-189 (Lumiracoxib), BMS 347070, RS 57067, NS-398, Bextra (valdecoxib), paracoxib, SD-8381,4-Methyl-2-(3,4-dimethylphenyl)-1-(4-sulfamoyl-phenyl)-1H-pyrrole, 2-(4-Ethoxyphenyl)-4-methyl-1-(4-sulfamoylphenyl)-1H-pyrrole, T-614, JTE-522, S-2474, SVT-2016, CT-3, SC-58125 and Arcoxia (etoricoxib). Additionally, COX-II inhibitors are disclosed in U.S. patent application Ser. Nos. 10/801,446 and 10/801,429, the contents of which are incorporated in their entirety for all purposes.

In one preferred embodiment the anti-tumor agent is celecoxib as disclosed in U.S. Pat. No. 5,466,823, the contents of which are incorporated by reference in its entirety for all purposes. The structure for Celecoxib is shown below:

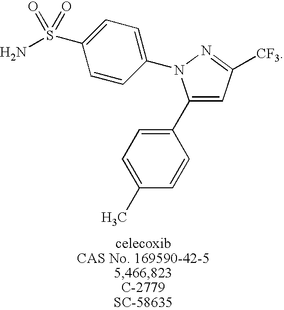

In one preferred embodiment the anti-tumor agent is valecoxib as disclosed in U.S. Pat. No. 5,633,272, the contents of which are incorporated by reference in its entirety for all purposes. The structure for valdecoxib is shown below:

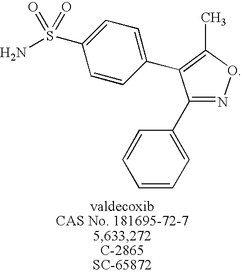

In one preferred embodiment the anti-tumor agent is parecoxib as disclosed in U.S. Pat. No. 5,932,598, the contents of which are incorporated by reference in its entirety for all purposes. The structure for paracoxib is shown below:

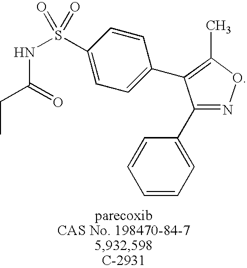

In one preferred embodiment the anti-tumor agent is deracoxib as disclosed in U.S. Pat. No. 5,521,207, the contents of which are incorporated by reference in its entirety for all purposes. The structure for deracoxib is shown below:

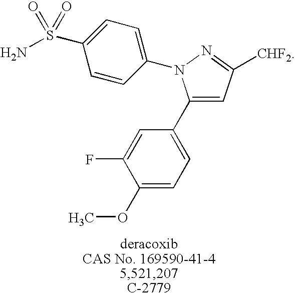

In one preferred embodiment the anti-tumor agent is SD-8381 as disclosed in U.S. Pat. No. 6,034,256, the contents of which are incorporated by reference in its entirety for all purposes. The structure for SD-8381 is shown below:

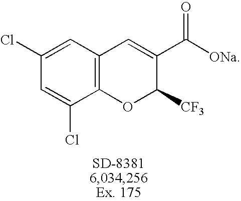

In one preferred embodiment the anti-tumor agent is ABT-963 as disclosed in International Publication Number WO 2002/24719, the contents of which are incorporated by reference in its entirety for all purposes. The structure for ABT-963 is shown below:

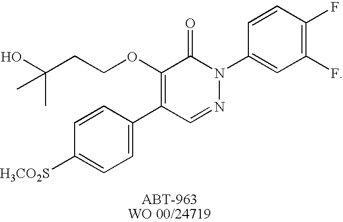

In one preferred embodiment the anti-tumor agent is MK-663 (etoricoxib) as disclosed in International Publication Number WO 1998/03484, the contents of which are incorporated by reference in its entirety for all purposes. The structure for etoricoxib is shown below:

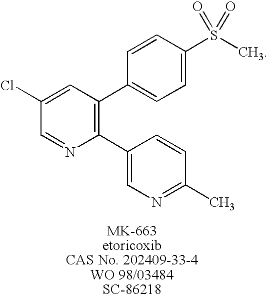

In one preferred embodiment the anti-tumor agent is COX-189 (Lumiracoxib) as disclosed in International Publication Number WO 1999/11605, the contents of which are incorporated by reference in its entirety for all purposes. The structure for Lumiracoxib is shown below:

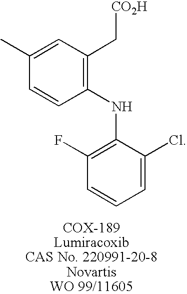

In one preferred embodiment the anti-tumor agent is BMS-347070 as disclosed in U.S. Pat. No. 6,180,651, the contents of which are incorporated by reference in its entirety for all purposes. The structure for BMS-347070 is shown below:

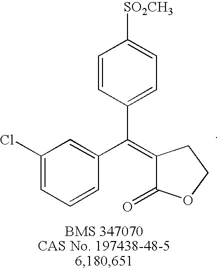

In one preferred embodiment the anti-tumor agent is NS-398 (CAS 123653-11-2). The structure for NS-398 (CAS 123653-11-2) is shown below:

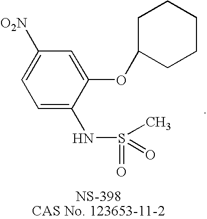

In one preferred embodiment the anti-tumor agent is RS 57067 (CAS 17932-91-3). The structure for RS-57067 (CAS 17932-91-3) is shown below:

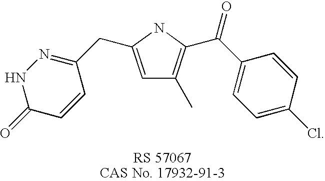

In one preferred embodiment the anti-tumor agent is 4-Methyl-2-(3,4-dimethylphenyl)-1-(4-sulfamoyl-phenyl)-1H-pyrrole. The structure for 4-Methyl-2-(3,4-dimethylphenyl)-1-(4-sulfamoyl-phenyl)-1H-pyrrole is shown below:

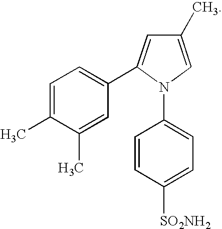

In one preferred embodiment the anti-tumor agent is 2-(4-Ethoxyphenyl)-4-methyl-1-(4-sulfamoylphenyl)-1H-pyrrole. The structure for 2-(4-Ethoxyphenyl)-4-methyl-1-(4-sulfamoylphenyl)-1H-pyrrole is shown below:

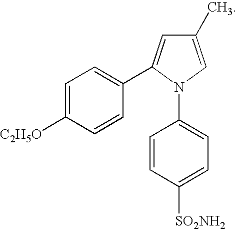

In one preferred embodiment the anti-tumor agent is meloxicam. The structure for meloxicam is shown below:

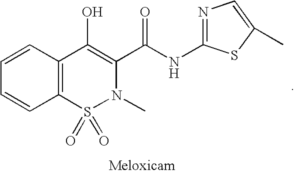

Other useful inhibitors as anti-tumor agents used in conjunction with a compound of formula I and pharmaceutical compositions described herein include aspirin, and non-steroidal anti-inflammatory drugs (NSAIDs) which inhibit the enzyme that makes prostaglandins (cyclooxygenase 1 and 11), resulting in lower levels of prostaglandins, include but are not limited to the following, Salsalate (Amigesic), Diflunisal (Dolobid), Ibuprofen (Motrin), Ketoprofen (Orudis), Nabumetone (Relafen), Piroxicam (Feldene), Naproxen (Aleve, Naprosyn), Diclofenac (Voltaren), Indomethacin (Indocin), Sulindac (Clinoril), Tolmetin (Tolectin), Etodolac (Lodine), Ketorolac (Toradol), Oxaprozin (Daypro) and combinations thereof.

Preferred COX-1 inhibitors include ibuprofen (Motrin), nuprin, naproxen (Aleve), indomethacin (Indocin), nabumetone (Relafen) and combinations thereof.

Targeted agents used in conjunction with a compound of formula I and pharmaceutical compositions described herein include EGFr inhibitors such as Iressa (gefitinib, AstraZeneca), Tarceva (erlotinib or OSI-774, OSI Pharmaceuticals Inc.), Erbitux (cetuximab, Imclone Pharmaceuticals, Inc.), EMD-7200 (Merck AG), ABX-EGF (Amgen Inc. and Abgenix Inc.), HR3 (Cuban Government), IgA antibodies (University of Erlangen-Nuremberg), TP-38 (IVAX), EGFR fusion protein, EGF-vaccine, anti-EGFr immunoliposomes (Hermes Biosciences Inc.) and combinations thereof.

Preferred EGFr inhibitors include Iressa, Erbitux, Tarceva and combinations thereof.

The present invention also relates to anti-tumor agents selected from pan erb receptor inhibitors or ErbB2 receptor inhibitors, such as CP-724,714 (Pfizer, Inc.), CI-1033 (canertinib, Pfizer, Inc.), Herceptin (trastuzumab, Genentech Inc.), Omitarg (2C4, pertuzumab, Genentech Inc.), TAK-165 (Takeda), GW-572016 (Ionafarnib, GlaxoSmithKline), GW-282974 (GlaxoSmithKline), EKB-569 (Wyeth), PKI-166 (Novartis), dHER2 (HER2Vaccine, Corixa and GlaxoSmithKline), APC8024 (HER2Vaccine, Dendreon), anti-HER2/neu bispecific antibody (Decof Cancer Center), B7.her2.IgG3 (Agensys), AS HER2 (Research Institute for Rad Biology & Medicine), trifunctional bispecific antibodies (University of Munich) and mAB AR-209 (Aronex Pharmaceuticals Inc) and mAB 2B-1 (Chiron) and combinations thereof.

Preferred erb selective anti-tumor agents include Herceptin, TAK-165, CP-724,714, ABX-EGF, HER3 and combinations thereof.

Preferred pan erbb receptor inhibitors include GW572016, CI-1033, EKB-569, and Omitarg and combinations thereof.

Additional erbB2 inhibitors include those described in WO 98/02434 (published Jan. 22, 1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995), U.S. Pat. No. 5,587,458 (issued Dec. 24, 1996), and U.S. Pat. No. 5,877,305 (issued Mar. 2, 1999), each of which is herein incorporated by reference in its entirety. ErbB2 receptor inhibitors useful in the present invention are also described in U.S. Pat. Nos. 6,465,449, and 6,284,764, and International Application No. WO 2001/98277 each of which are herein incorporated by reference in their entirety.

Additionally, other anti-tumor agents may be selected from the following agents, BAY-43-9006 (Onyx Pharmaceuticals Inc.), Genasense (augmerosen, Genta), Panitumumab (Abgenix/Amgen), Zevalin (Schering), Bexxar (Corixa/GlaxoSmithKline), Abarelix, Alimta, EPO 906 (Novartis), discodermolide (XAA-296), ABT-510 (Abbott), Neovastat (Aeterna), enzastaurin (Eli Lilly), Combrestatin A4P (Oxigene), ZD-6126 (AstraZeneca), flavopiridol (Aventis), CYC-202 (Cyclacel), AVE-8062 (Aventis), DMXAA (Roche/Antisoma), Thymitaq (Eximias), Temodar (temozolomide, Schering Plough) and Revilimd (Celegene) and combinations thereof.

Other anti-tumor agents may be selected from the following agents, CyPat (cyproterone acetate), Histerelin (histrelin acetate), Plenaixis (abarelix depot), Atrasentan (ABT-627), Satraplatin (JM-216), thalomid (Thalidomide), Theratope, Temilifene (DPPE), ABI-007 (paclitaxel), Evista (raloxifene), Atamestane (Biomed-777), Xyotax (polyglutamate paclitaxel), Targetin (bexarotine) and combinations thereof.

Additionally, other anti-tumor agents may be selected from the following agents, Trizaone (tirapazamine), Aposyn (exisulind), Nevastat (AE-941), Ceplene (histamine dihydrochloride), Orathecin (rubitecan), Virulizin, Gastrimmune (G17DT), DX-8951f (exatecan mesylate), Onconase (ranpirnase), BEC2 (mitumoab), Xcytrin (motexafin gadolinium) and combinations thereof.

Further anti-tumor agents may selected from the following agents, CeaVac (CEA), NeuTrexin (trimetresate glucuronate) and combinations thereof.

Additional anti-tumor agents may selected from the following agents, OvaRex (oregovomab), Osidem (IDM-1), and combinations thereof.

Additional anti-tumor agents may selected from the following agents, Advexin (ING 201), Tirazone (tirapazamine), and combinations thereof.

Additional anti-tumor agents may selected from the following agents, RSR13 (efaproxiral), Cotara (131I chTNT 1/b), NBI-3001 (IL-4) and combinations thereof.

Additional anti-tumor agents may selected from the following agents, Canvaxin, GMK vaccine, Oncophage (HSPPC-96), PEG Interon A, Taxoprexin (DHA/paciltaxel) and combinations thereof.

Other preferred anti-tumor agents include Pfizer's MEK1/2 inhibitor PD325901, Array Biopharm's MEK inhibitor ARRY-142886, Bristol Myers' CDK2 inhibitor BMS-387,032, Pfizer's CDK inhibitor PD0332991 and AstraZeneca's AXD-5438 and combinations thereof.

Additionally, mTOR inhibitors may also be utilized such as CCl-779 (Wyeth) and rapamycin derivatives RAD001 (Novartis) and AP-23573 (Ariad), HDAC inhibitors SAHA (Merck Inc./Aton Pharmaceuticals) and combinations thereof.

Additional anti-tumor agents include aurora 2 inhibitor VX-680 (Vertex), Chk1/2 inhibitor XL844 (Exilixis).

The following cytotoxic agents, e.g., one or more selected from the group consisting of epirubicin (Ellence), docetaxel (Taxotere), paclitaxel, Zinecard (dexrazoxane), rituximab (Rituxan) imatinib mesylate (Gleevec), and combinations thereof, may be used in conjunction with a compound of formula I and pharmaceutical compositions described herein.

The invention also contemplates the use of the compounds of the present invention together with hormonal therapy, including but not limited to, exemestane (Aromasin, Pfizer Inc.), leuprorelin (Lupron or Leuplin, TAP/Abbott/Takeda), anastrozole (Arimidex, Astrazeneca), gosrelin (Zoladex, AstraZeneca), doxercalciferol, fadrozole, formestane, tamoxifen citrate (tamoxifen, Nolvadex, AstraZeneca), Casodex (AstraZeneca), Abarelix (Praecis), Trelstar, and combinations thereof.

The invention also relates to hormonal therapy agents such as anti-estrogens including, but not limited to fulvestrant, toremifene, raloxifene, lasofoxifene, letrozole (Femara, Novartis), anti-androgens such as bicalutamide, flutamide, mifepristone, nilutamide, Casodex® (4′-cyano-3-(4-fluorophenylsulphonyl)-2-hydroxy-2-methyl-3′-(trifluoromethyl) propionanilide, bicalutamide) and combinations thereof.

Further, the invention provides a compound of the present invention alone or in combination with one or more supportive care products, e.g., a product selected from the group consisting of Filgrastim (Neupogen), ondansetron (Zofran), Fragmin, Procrit, Aloxi, Emend, or combinations thereof.

Particularly preferred cytotoxic agents include Camptosar, Erbitux, Iressa, Gleevec, Taxotere and combinations thereof.

The following topoisomerase I inhibitors may be utilized as anti-tumor agents camptothecin, irinotecan HCl (Camptosar), edotecarin, orathecin (Supergen), exatecan (Daiichi), BN-80915 (Roche) and combinations thereof.

Particularly preferred toposimerase II inhibitors include epirubicin (Ellence).

The compounds of the invention may be used with antitumor agents, alkylating agents, antimetabolites, antibiotics, plant-derived antitumor agents, camptothecin derivatives, tyrosine kinase inhibitors, antibodies, interferons, and/or biological response modifiers.

Alkylating agents include, but are not limited to, nitrogen mustard N-oxide, cyclophosphamide, ifosfamide, melphalan, busulfan, mitobronitol, carboquone, thiotepa, ranimustine, nimustine, temozolomide, AMD-473, altretamine, AP-5280, apaziquone, brostallicin, bendamustine, carmustine, estramustine, fotemustine, glufosfamide, ifosfamide, KW-2170, mafosfamide, and mitolactol; platinum-coordinated alkylating compounds include but are not limited to, cisplatin, Paraplatin (carboplatin), eptaplatin, lobaplatin, nedaplatin, Eloxatin (oxaliplatin, Sanofi) or satrplatin and combinations thereof.

Particularly preferred alkylating agents include Eloxatin (oxaliplatin).

Antimetabolites include but are not limited to, methotrexate, 6-mercaptopurine riboside, mercaptopurine, 5-fluorouracil (5-FU) alone or in combination with leucovorin, tegafur, UFT, doxifluridine, carmofur, cytarabine, cytarabine ocfosfate, enocitabine, S-1, Alimta (premetrexed disodium, LY231514, MTA), Gemzar (gemcitabine, Eli Lilly), fludarabin, 5-azacitidine, capecitabine, cladribine, clofarabine, decitabine, eflornithine, ethynylcytidine, cytosine arabinoside, hydroxyurea, TS-1, melphalan, nelarabine, nolatrexed, ocfosfate, disodium premetrexed, pentostatin, pelitrexol, raltitrexed, triapine, trimetrexate, vidarabine, vincristine, vinorelbine; or for example, one of the preferred anti-metabolites disclosed in European Patent Application No. 239362 such as N-(5-[N-(3,4-dihydro-2-methyl-4-oxoquinazolin-6-ylmethyl)-N-methylamino]-2-thenoyl)-L-glutamic acid and combinations thereof.

Antibiotics include intercalating antibiotics but are not limited to: aclarubicin, actinomycin D, amrubicin, annamycin, adriamycin, bleomycin, daunorubicin, doxorubicin, elsamitrucin, epirubicin, galarubicin, idarubicin, mitomycin C, nemorubicin, neocarzinostatin, peplomycin, pirarubicin, rebeccamycin, stimalamer, streptozocin, valrubicin, zinostatin and combinations thereof.

Plant derived anti-tumor substances include for example those selected from mitotic inhibitors, for example vinblastine, docetaxel (Taxotere), paclitaxel and combinations thereof.

Cytotoxic topoisomerase inhibiting agents include one or more agents selected from the group consisting of aclarubicn, amonafide, belotecan, camptothecin, 10-hydroxycamptothecin, 9-aminocamptothecin, diflomotecan, irinotecan HCl (Camptosar), edotecarin, epirubicin (Ellence), etoposide, exatecan, gimatecan, lurtotecan, mitoxantrone, pirarubicin, pixantrone, rubitecan, sobuzoxane, SN-38, tafluposide, topotecan, and combinations thereof.

Preferred cytotoxic topoisomerase inhibiting agents include one or more agents selected from the group consisting of camptothecin, 10-hydroxycamptothecin, 9-aminocamptothecin, irinotecan HCl (Camptosar), edotecarin, epirubicin (Ellence), etoposide, SN-38, topotecan, and combinations thereof.

Immunologicals include interferons and numerous other immune enhancing agents. Interferons include interferon alpha, interferon alpha-2a, interferon, alpha-2b, interferon beta, interferon gamma-1a, interferon gamma-1b (Actimmune), or interferon gamma-ni and combinations thereof. Other agents include filgrastim, lentinan, sizofilan, TheraCys, ubenimex, WF-10, aldesleukin, alemtuzumab, BAM-002, dacarbazine, daclizumab, denileukin, gemtuzumab ozogamicin, ibritumomab, imiquimod, lenograstim, lentinan, melanoma vaccine (Corixa), molgramostim, OncoVAX-CL, sargramostim, tasonermin, tecleukin, thymalasin, tositumomab, Virulizin, Z-100, epratuzumab, mitumomab, oregovomab, pemtumomab (Y-muHMFG1), Provenge (Dendreon) and combinations thereof.

Biological response modifiers are agents that modify defense mechanisms of living organisms or biological responses, such as survival, growth, or differentiation of tissue cells to direct them to have anti-tumor activity. Such agents include krestin, lentinan, sizofuran, picibanil, ubenimex and combinations thereof.

Other anticancer agents include alitretinoin, ampligen, atrasentan bexarotene, bortezomib. Bosentan, calcitriol, exisulind, finasteride, fotemustine, ibandronic acid, miltefosine, mitoxantrone, 1-asparaginase, procarbazine, dacarbazine, hydroxycarbamide, pegaspargase, pentostatin, tazarotne, Telcyta (TLK-286, Telik Inc.), Velcade (bortemazib, Millenium), tretinoin, and combinations thereof.

Other anti-angiogenic compounds include acitretin, fenretinide, thalidomide, zoledronic acid, angiostatin, aplidine, cilengtide, combretastatin A-4, endostatin, halofuginone, rebimastat, removab, Revlimid, squalamine, ukrain, Vitaxin and combinations thereof.

Platinum-coordinated compounds include but are not limited to, cisplatin, carboplatin, nedaplatin, oxaliplatin, and combinations thereof.

Camptothecin derivatives include but are not limited to camptothecin, 10-hydroxycamptothecin, 9-aminocamptothecin, irinotecan, SN-38, edotecarin, topotecan and combinations thereof.

Other antitumor agents include mitoxantrone, 1-asparaginase, procarbazine, dacarbazine, hydroxycarbamide, pentostatin, tretinoin and combinations thereof.

Anti-tumor agents capable of enhancing antitumor immune responses, such as CTLA4 (cytotoxic lymphocyte antigen 4) antibodies, and other agents capable of blocking CTLA4 may also be utilized, such as MDX-010 (Medarex) and CTLA4 compounds disclosed in U.S. Pat. No. 6,682,736; and anti-proliferative agents such as other farnesyl protein transferase inhibitors, for example the farnesyl protein transferase inhibitors. Additional, specific CTLA4 antibodies that can be used in the present invention include those described in U.S. Provisional Application 60/113,647 (filed Dec. 23, 1998), U.S. Pat. No. 6,682,736 both of which are herein incorporated by reference in their entirety.

Specific IGF1R antibodies that can be used in the present invention include those described in International Patent Application No. WO 2002/053596, which is herein incorporated by reference in its entirety.

Specific CD40 antibodies that can be used in the present invention include those described in International Patent Application No. WO 2003/040170 which is herein incorporated by reference in its entirety.

Gene therapy agents may also be employed as anti-tumor agents such as TNFerade (GeneVec), which express TNFalpha in response to radiotherapy.

In one embodiment of the present invention statins may be used in conjunction with a compound of formula I and pharmaceutical compositions. Statins (HMG-CoA reducatase inhibitors) may be selected from the group consisting of Atorvastatin (Lipitor, Pfizer Inc.), Provastatin (Pravachol, Bristol-Myers Squibb), Lovastatin (Mevacor, Merck Inc.), Simvastatin (Zocor, Merck Inc.), Fluvastatin (Lescol, Novartis), Cerivastatin (Baycol, Bayer), Rosuvastatin (Crestor, AstraZeneca), Lovostatin and Niacin (Advicor, Kos Pharmaceuticals), derivatives and combinations thereof.

In a preferred embodiment the statin is selected from the group consisting of Atovorstatin and Lovastatin, derivatives and combinations thereof.

Other agents useful as anti-tumor agents include Caduet.

In one preferred embodiment radiation can be used in conjunction with a compound of formula I and pharmaceutical compositions described herein. Radiation may be administered in a variety of fashions. For example, radiation may be electromagnetic or particulate in nature. Electromagnetic radiation useful in the practice of this invention includes, but is not limited, to X-rays and gamma rays. In a preferable embodiment, supervoltage X-rays (X-rays>=4 MeV) may be used in the practice of this invention. Particulate radiation useful in the practice of this invention includes, but is not limited to, electron beams, protons beams, neutron beams, alpha particles, and negative pi mesons. The radiation may be delivered using conventional radiological treatment apparatus and methods, and by intraoperative and stereotactic methods. Additional discussion regarding radiation treatments suitable for use in the practice of this invention may be found throughout Steven A. Leibel et al., Textbook of Radiation Oncology (1998) (publ. W. B. Saunders Company), and particularly in Chapters 13 and 14. Radiation may also be delivered by other methods such as targeted delivery, for example by radioactive “seeds,” or by systemic delivery of targeted radioactive conjugates. J. Padawer et al., Combined Treatment with Radioestradiol lucanthone in Mouse C3HBA Mammary Adenocarcinoma and with Estradiol lucanthone in an Estrogen Bioassay, Int. J. Radiat. Oncol. Biol. Phys. 7:347-357 (1981). Other radiation delivery methods may be used in the practice of this invention.

The amount of radiation delivered to the desired treatment volume may be variable. In a preferable embodiment, radiation may be administered in amount effective to cause the arrest or regression of the cancer, in combination with a compound of formula I and pharmaceutical compositions described herein.

In a more preferable embodiment, radiation is administered in at least about 1 Gray (Gy) fractions at least once every other day to a treatment volume, still more preferably radiation is administered in at least about 2 Gray (Gy) fractions at least once per day to a treatment volume, even more preferably radiation is administered in at least about 2 Gray (Gy) fractions at least once per day to a treatment volume for five consecutive days per week.

In a more preferable embodiment, radiation is administered in 3 Gy fractions every other day, three times per week to a treatment volume.

In yet another more preferable embodiment, a total of at least about 20 Gy, still more preferably at least about 30 Gy, most preferably at least about 60 Gy of radiation is administered to a host in need thereof.

In one more preferred embodiment of the present invention 14 GY radiation is administered.

In another more preferred embodiment of the present invention 10 GY radiation is administered.

In another more preferred embodiment of the present invention 7 GY radiation is administered.

In a most preferable embodiment, radiation is administered to the whole brain of a host, wherein the host is being treated for metastatic cancer.

Examples of useful matrix metalloproteinase inhibitors used in conjunction with a compound of formula I and pharmaceutical compositions described herein are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931,788 (published Jul. 28, 1999), WO 90/05719 (published May 331, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain patent application number 9912961.1 (filed Jun. 3, 1999), U.S. Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are herein incorporated by reference in their entirety.

Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13).

Some specific examples of MMP inhibitors useful in combination with the compounds of the present invention are AG-3340, RO 32-3555, RS 13-0830, and the compounds recited in the following list:
3-[[4-(4-fluoro-phenoxy)-benzenesulfonyl]-(1-hydroxycarbamoyl-cyclopentyl)-amino]-propionic acid;
3-exo-3-[4-(4-fluoro-phenoxy)-benzenesulfonylamino]-8-oxa-bicyclo[3.2.1]octane-3-carboxylic acid hydroxyamide;
(2R,3R) 1-[4-(2-chloro-4-fluoro-benzyloxy)-benzenesulfonyl]-3-hydroxy-3-methyl-piperidine-2-carboxylic acid hydroxyamide;
4-[4-(4-fluoro-phenoxy)-benzenesulfonylamino]-tetrahydro-pyran-4-carboxylic acid hydroxyamide;
3-[[4-(4-fluoro-phenoxy)-benzenesulfonyl]-(1-hydroxycarbamoyl-cyclobutyl)-amino]-propionic acid;
4-[4-(4-chloro-phenoxy)-benzenesulfonylamino]-tetrahydro-pyran-4-carboxylic acid hydroxyamide;
3-[4-(4-chloro-phenoxy)-benzenesulfonylamino]-tetrahydro-pyran-3-carboxylic acid hydroxyamide;
(2R,3R) 1-[4-(4-fluoro-2-methyl-benzyloxy)-benzenesulfonyl]-3-hydroxy-3-methyl-piperidine-2-carboxylic acid hydroxyamide;
3-[[4-(4-fluoro-phenoxy)-benzenesulfonyl]-(1-hydroxycarbamoyl-1-methyl-ethyl)-amino]-propionic acid;
3-[[4-(4-fluoro-phenoxy)-benzenesulfonyl]-(4-hydroxycarbamoyl-tetrahydro-pyran-4-yl)-amino]-propionic acid;
3-exo-3-[4-(4-chloro-phenoxy)-benzenesulfonylamino]-8-oxa-bicyclo[3.2.1]octane-3-carboxylic acid hydroxyamide;
3-endo-3-[4-(4-fluoro-phenoxy)-benzenesulfonylamino]-8-oxa-bicyclo[3.2.1]octane-3-carboxylic acid hydroxyamide; and
3-[4-(4-fluoro-phenoxy)-benzenesulfonylamino]-tetrahydro-furan-3-carboxylic acid hydroxyamide;

and pharmaceutically acceptable salts, solvates and prodrugs of said compounds.

Various other compounds, such as styrene derivatives, have also been shown to possess tyrosine kinase inhibitory properties, and some tyrosine kinase inhibitors have been identified as erbB2 receptor inhibitors. More recently, five European patent publications, namely EP 0 566 226 A1 (published Oct. 20, 1993), EP 0 602 851 A1 (published Jun. 22, 1994), EP 0 635 507 A1 (published Jan. 25, 1995), EP 0 635 498 A1 (published Jan. 25, 1995), and EP 0 520 722 A1 (published Dec. 30, 1992), refer to certain bicyclic derivatives, in particular quinazoline derivatives, as possessing anti-cancer properties that result from their tyrosine kinase inhibitory properties. Also, World Patent Application WO 92/20642 (published Nov. 26, 1992), refers to certain bis-mono and bicyclic aryl and heteroaryl compounds as tyrosine kinase inhibitors that are useful in inhibiting abnormal cell proliferation. World Patent Applications WO96/16960 (published Jun. 6, 1996), WO 96/09294 (published Mar. 6, 1996), WO 97/30034 (published Aug. 21, 1997), WO 98/02434 (published Jan. 22, 1998), WO 98/02437 (published Jan. 22, 1998), and WO 98/02438 (published Jan. 22, 1998), also refer to substituted bicyclic heteroaromatic derivatives as tyrosine kinase inhibitors that are useful for the same purpose. Other patent applications that refer to anti-cancer compounds are World Patent Application WO00/44728 (published Aug. 3, 2000), EP 1029853A1 (published Aug. 23, 2000), and WO01/98277 (published Dec. 12, 2001) all of which are incorporated herein by reference in their entirety.

“Abnormal cell growth”, as used herein, unless otherwise indicated, refers to cell growth that is independent of normal regulatory mechanisms (e.g., loss of contact inhibition). This includes the abnormal growth of: (1) tumor cells (tumors) that proliferate by expressing a mutated tyrosine kinase or overexpression of a receptor tyrosine kinase; (2) benign and malignant cells of other proliferative diseases in which aberrant tyrosine kinase activation occurs; and (4) any tumors that proliferate by receptor tyrosine kinases.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

The term “halo”, as used herein, unless otherwise indicated, means fluoro, chloro, bromo or iodo. Preferred halo groups are fluoro, chloro and bromo.

The term “alkyl”, as used herein, unless otherwise indicated, includes saturated monovalent hydrocarbon radicals having straight, branched, or cyclic moieties (including fused and bridged bicyclic and spirocyclic moieties), or a combination of the foregoing moieties. For an alkyl group to have cyclic moieties, the group must have at least three carbon atoms; for an alkyl group to have bicyclic moieties, the group must have at least four carbon atoms.

The term “alkenyl”, as used herein, unless otherwise indicated, includes alkyl moieties having at least one carbon-carbon double bond wherein alkyl is as defined above and including E and Z isomers of said alkenyl moiety.

The term “alkynyl”, as used herein, unless otherwise indicated, includes alkyl moieties having at least one carbon-carbon triple bond wherein alkyl is as defined above.

The term “alkoxy”, as used herein, unless otherwise indicated, includes O-alkyl groups wherein alkyl is as defined above.

The term “aryl”, as used herein, unless otherwise indicated, includes an organic radical derived from an aromatic hydrocarbon by removal of one hydrogen, such as phenyl or naphthyl.

The term “4-10 membered heterocyclic”, as used herein, unless otherwise indicated, includes aromatic and non-aromatic heterocyclic and heterobicyclic groups containing one to four heteroatoms each selected from O, S and N, wherein each heterocyclic group has from 4-10 atoms in its ring system, and with the proviso that the ring of said group does not contain two adjacent O or S atoms. Non-aromatic heterocyclic groups include groups having at least 4 atoms in their ring system and aromatic heterocyclic groups have at least 5 atoms in their ring system. The heterocyclic groups include benzo-fused ring systems. An example of a 4 membered heterocyclic group is azetidinyl (derived from azetidine). An example of a 5 membered heterocyclic group is thiazolyl and an example of a 10 membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazol idinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached). An example of a heterocyclic group wherein 2 ring carbon atoms are substituted with oxo (═O) moieties is 1,1-dioxo-thiomorpholinyl.

The phrase “pharmaceutically acceptable salt(s)”, as used herein, unless otherwise indicated, includes salts of acidic or basic groups that may be present in the compounds of formula I. The compounds of formula I that are basic in nature are capable of forming a wide variety of salts with various inorganic and organic acids. The acids that may be used to prepare pharmaceutically acceptable acid addition salts of such basic compounds of formula I are those that form non-toxic acid addition salts, i.e., salts containing pharmacologically acceptable anions, such as the acetate, adipate, aspartate, benzenesulfonate, benzoate, besylate, bicarbonate/carbonate, bisulphate/sulphate, bitartrate, borate, bromide, calcium edetate, camsylate, carbonate, chloride, clavulanate, citrate, cyclamate, dihydrochloride, edetate, edislyate, estolate, esylate, ethylsuccinate, formate, fumarate, gluceptate, gluconate, glucuronate, glutamate, glycollylarsanilate, hexylresorcinate, hexafluorophosphate, hibenzate, hydrabamine, hydrobromide, hydrochloride, hydroiodide, iodide, isethionate, lactate, lactobionate, laurate, malate, maleate, malonate, mandelate, mesylate, methylsulfate, mucate, naphthylate, nitrate, 2-napsylate, nicotinate, nitrate, orotate, oleate, oxalate, pamoate (embonate), palmitate, pantothenate, phosphate/hydrogen phosphate/dihydrogen, phosphate phospate/diphosphate, polygalacturonate, salicylate, stearate, subacetate, succinate, tannate, tartrate, teoclate, tosylate, triethiodode, trifluoroacetate, valerate and xinofoate salts.

Pharmaceutically acceptable salts of the compounds of formula I include the acid and base salts thereof.

Suitable base salts are formed from bases that form non-toxic salts. Examples include the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

For a review on suitable salts, see Handbook of Pharmaceutical Salts: Properties, Selection, and Use by Stahl and Wermuth (Wiley-VCH, 2002).

Pharmaceutically acceptable salts of compounds of formula I may be prepared by one or more of three methods:

(i) by reacting the compound of formula I with the desired acid or base;

(ii) by removing an acid- or base-labile protecting group from a suitable precursor of the compound of formula I or by ring-opening a suitable cyclic precursor, for example, a lactone or lactam, using the desired acid or base; or

(iii) by converting one salt of the compound of formula I to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterised by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterised by a phase change, typically first order (‘melting point’).

The compounds of the invention may also exist in unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

Also included within the scope of the invention are multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents that are bound together through non-covalent interactions, but could also be a complex of a neutral molecule with a salt. Co-crystals may be prepared by melt crystallisation, by recrystallisation from solvents, or by physically grinding the components together—see Chem Commun, 17, 1889-1896, by 0. Almarsson and M. J. Zaworotko (2004). For a general review of multi-component complexes, see J Pharm Sci, 64 (8), 1269-1288, by Haleblian (August 1975).

The compounds of the invention may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO—Na+, —COO—K+, or —SO₃—Na+) or non-ionic (such as —N—N+(CH₃)₃) polar head group. For more information, see Crystals and the Polarizing Microscope by N. H. Hartshorne and A. Stuart, 4th Edition (Edward Arnold, 1970).

Hereinafter all references to compounds of formula I include references to salts, solvates, multi-component complexes and liquid crystals thereof and to solvates, multi-component complexes and liquid crystals of salts thereof.

The compounds of the invention include compounds of formula I as hereinbefore defined, including all polymorphs and crystal habits thereof, prodrugs and isomers thereof (including optical, geometric and tautomeric isomers) as hereinafter defined and isotopically-labeled compounds of formula I.

The compounds of the invention include compounds of formula I as hereinbefore defined, including all polymorphs and crystal habits thereof, prodrugs and isomers thereof (including optical, geometric and tautomeric isomers) as hereinafter defined and isotopically-labeled compounds of formula I.

As indicated, so-called ‘prodrugs’ of the compounds of formula I are also within the scope of the invention. Thus certain derivatives of compounds of formula I which may have little or no pharmacological activity themselves can, when administered into or onto the body, be converted into compounds of formula I having the desired activity, for example, by hydrolytic cleavage. Such derivatives are referred to as ‘prodrugs’. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (Ed. E. B. Roche, American Pharmaceutical Association).

Prodrugs in accordance with the invention can, for example, be produced by replacing appropriate functionalities present in the compounds of formula I with certain moieties known to those skilled in the art as ‘pro-moieties’ as described, for example, in Design of Prodrugs by H. Bundgaard (Elsevier, 1985).

Some examples of prodrugs in accordance with the invention include

(i) where the compound of formula I contains a carboxylic acid functionality (—COOH), an ester thereof, for example, a compound wherein the hydrogen of the carboxylic acid functionality of the compound of formula I is replaced by an alkyl;

(ii) where the compound of formula I contains an alcohol functionality, an ether thereof, for example, a compound wherein the hydrogen of the alcohol functionality of the compound of formula I is replaced by an alkanoyloxymethyl; and

(iii) where the compound of formula I contains a primary or secondary amino functionality, an amide thereof, for example, a compound wherein, as the case may be, one or both hydrogens of the amino functionality of the compound of formula I is/are replaced by an alkanoyl.

Further examples of replacement groups in accordance with the foregoing examples and examples of other prodrug types may be found in the aforementioned references.

Moreover, certain compounds of formula I may themselves act as prodrugs of other compounds of formula I.

Also included within the scope of the invention are metabolites of compounds of formula I, that is, compounds formed in vivo upon administration of the drug. Some examples of metabolites in accordance with the invention include

(i) where the compound of formula I contains a methyl group, an hydroxymethyl derivative thereof:

(ii) where the compound of formula I contains an alkoxy group, an hydroxy derivative thereof;

(iii) where the compound of formula I contains a tertiary amino group, a secondary amino derivative thereof;

(iv) where the compound of formula I contains a secondary amino group, a primary derivative thereof;

(v) where the compound of formula I contains a phenyl moiety, a phenol derivative thereof; and

(vi) where the compound of formula I contains an amide group, a carboxylic acid derivative thereof.

Compounds of formula I containing one or more asymmetric carbon atoms can exist as two or more stereoisomers. Where a compound of formula I contains an alkenyl or alkenylene group, geometric cis/trans (or Z/E) isomers are possible. Where structural isomers are interconvertible via a low energy barrier, tautomeric isomerism (‘tautomerism’) can occur. This can take the form of proton tautomerism in compounds of formula I containing, for example, an imino, keto, or oxime group, or so-called valence tautomerism in compounds that contain an aromatic moiety. It follows that a single compound may exhibit more than one type of isomerism.

Included within the scope of the present invention are all stereoisomers, geometric isomers and tautomeric forms of the compounds of formula I including compounds exhibiting more than one type of isomerism, and mixtures of one or more thereof. Also included are acid addition or base salts wherein the counterion is optically active, for example, d-lactate or I-lysine, or racemic, for example, dl-tartrate or dl-arginine.

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of formula I contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture.

When any racemate crystallises, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

While both of the crystal forms present in a racemic mixture have identical physical properties, they may have different physical properties compared to the true racemate. Racemic mixtures may be separated by conventional techniques known to those skilled in the art—see, for example, Stereochemistry of Organic Compounds by E. L. Eliel and S. H. Wilen (Wiley, 1994).

The present invention includes all pharmaceutically acceptable isotopically-labelled compounds of formula I wherein one or more atoms are replaced by atoms having the same atomic number, but an atomic mass or mass number different from the atomic mass or mass number which predominates in nature.

Examples of isotopes suitable for inclusion in the compounds of the invention include isotopes of hydrogen, such as 2H and 3H, carbon, such as 11C, 13C and 14C, chlorine, such as 36Cl, fluorine, such as 18F, iodine, such as 123I and 125I, nitrogen, such as 13N and 15N, oxygen, such as 15O, 17O and 18O, phosphorus, such as 32P, and sulphur, such as 35S.

Certain isotopically-labelled compounds of formula I for example, those incorporating a radioactive isotope, are useful in drug and/or substrate tissue distribution studies. The radioactive isotopes tritium, i.e. 3H, and carbon-14, i.e. 14C, are particularly useful for this purpose in view of their ease of incorporation and ready means of detection.

Substitution with heavier isotopes such as deuterium, i.e. 2H, may afford certain therapeutic advantages resulting from greater metabolic stability, for example, increased in vivo half-life or reduced dosage requirements, and hence may be preferred in some circumstances.

Substitution with positron emitting isotopes, such as 11C, 18F, 15O and 13N, can be useful in Positron Emission Topography (PET) studies for examining substrate receptor occupancy.

Isotopically-labeled compounds of formula I can generally be prepared by conventional techniques known to those skilled in the art or by processes analogous to those described in the accompanying Examples and Preparations using an appropriate isotopically-labeled reagent in place of the non-labeled reagent previously employed.

Pharmaceutically acceptable solvates in accordance with the invention include those wherein the solvent of crystallization may be isotopically substituted, e.g. D₂O, d₆-acetone, d₆-DMSO.

Also within the scope of the invention are intermediate compounds of formula I as hereinbefore defined, all salts, solvates and complexes thereof and all solvates and complexes of salts thereof as defined hereinbefore for compounds of formula I. The invention includes all polymorphs of the aforementioned species and crystal habits thereof.

### DETAILED DESCRIPTION OF THE INVENTION

The compounds of the present invention of formula I are prepared according to the following schemes 1 to 8 described in detailed below. The groups and substituents shown in the following schemes 1 to 8, such as B, X, Z, V, W, R¹, R⁴, R⁵, R⁶, and R⁷, are as defined in definitions for Formula I in the detailed description of the invention.

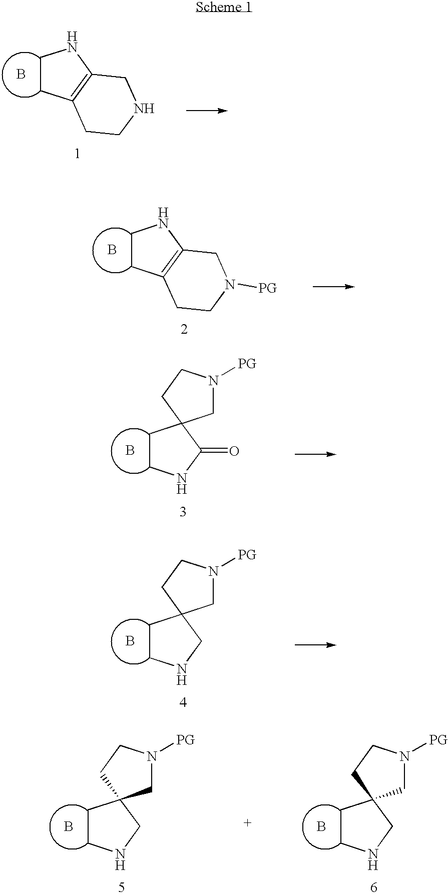

Scheme 1 illustrates a method to synthesize intermediates of the formula 5 and 6. Optionally substituted carbolines (1) are protected with a suitable protecting group (PG), preferably a carbamate, most preferably a benzyl carbamate. The protection takes place in an aprotic solvent, preferably dichloromethane, with an electrophilic protecting group, preferably benzyl chloroformate, with a base, preferably triethylamine, at a temperature between 0 and 60° C., preferably room temperature for a time between 0.5-1.5 hrs, preferably 1 hour. The resulting product 2 is then treated with N-chlorosuccinamide, in a polar aprotic solvent, preferably THF, with a base, preferably triethylamine, at a temperature between −15 and 15° C., preferably 0° C., for a time between 0.5-1.5 hrs, preferably 1 hour. The resulting spiroindilinone 3 is then reduced in an aprotic solvent, preferably THF, with a reducing agent, preferably sodium borohydride in the presence of iodine, at a temperature between −20 and 22° C., for a time between 4-8 hrs to provide compound 4 as a racemic mixture. The enantiomers of 4 can be separated into 5 and 6 through forming a salt with one of the enantiomers of a chiral acid, preferably Di-P-toluoyl-tartaric acid followed by selective crystallization. Alternatively, the enantiomers 5 and 6 can be separated through the use of chiral prep-HPLC.

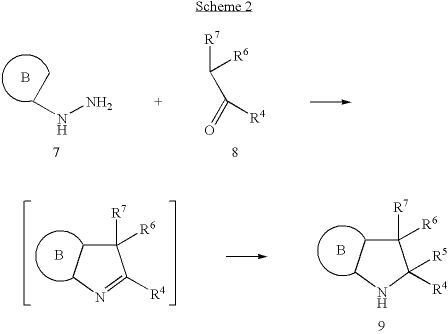

Scheme 2 shows a method to synthesize intermediates of the formula 9. Optionally substituted aryl or heteroaryl hydrazines of the formula 7, and aldehydes or ketones of the formula 8 are available commercially or can be synthesized by those skilled in the art. 7 and 8 are combined in an aprotic solvent, preferably dichloromethane, in the presence of 0.5-30% (by volume) of an acid, preferably trifluoroacetic acid, at temperatures between 10° C. and 110° C. for between 1 and 48 hrs. In the case that 8 is an aldehyde (R⁴═H), an intermediate imine of 9 is formed, which can either be isolated or directly reduced with a reducing agent, preferably sodium borohydride, in the presence of acid, preferably trifluoroacetic acid, at a temperature between 10° C. and 60° C. for 30 min. to 24 hrs. to form 9. If the imine of 9 was isolated, R⁴ nucleophiles, preferably alkyl magnesium bromides, can be added to this imine in polar aprotic solvents, preferably THF, at temperatures ranging from 10° C. to 110° C. for between 1 and 96 hrs. to provide 9 where R⁴ is an optionally substituted carbon atom.

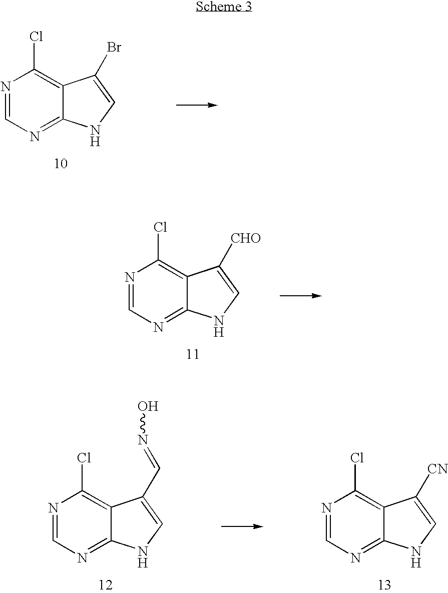

Scheme 3 illustrates a method to synthesize intermediates of the formula 13. 4-Chloro-5-bromo-pyrrolopyrimidine (10) was prepared by the method of Townsend (J. Med. Chem. 1990, 33 (7), 1984). Compound 10 is dissolved in a polar, aprotic solvent, preferably THF, at a temperature of −78° C., and an alkyl lithium reagent is added, preferably n-BuLi. The reaction is treated with dimethylformamide and stirred at −78° C. to room temperature for 0.5-2.5 hrs, preferably 1.5 hrs to form the aldehyde 11. This compound is dissolved in a polar solvent, preferably ethanol, and hydroxylamine is added, followed by a base, preferably sodium hydroxide. The reaction is stirred from room temperature to 50° C. for 5 hrs to provide 12 as a mixture of isomers. This product is then dissolved in a non-polar solvent, preferably methylene chloride, and treated with a dehydrating reagent, preferably thionyl chloride, at a temperature between room temperature and 45° C., for a period of 1 hour to provide intermediate 13.

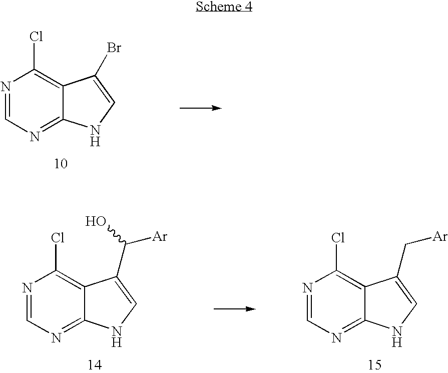

Scheme 4 illustrates a method to synthesize intermediates of the formula 14 and 15. The compound of formula 10 was prepared as described in Scheme 3 above. Compound 10 is dissolved in a polar, aprotic solvent, preferably THF, at a temperature of −78° C., and an alkyl lithium reagent is added, preferably n-butyl lithium. The reaction is treated with an electrophile, preferably an aryl aldehyde. The reaction is stirred at −78° C. to room temperature for 2 hrs, to form intermediate 14. This intermediate can be coupled directly to amines as shown in Scheme 6, or can be deoxygenated to form intermediate 15. This deoxygenation can occur by dissolving 14 in a non-polar solvent, preferably methylene chloride, in the presence of an acid, preferably trifluoroacetic acid, and a hydride donating reagent, preferably triethylsilane, and stirring at room temperature for a period of 21 hrs to produce 15.

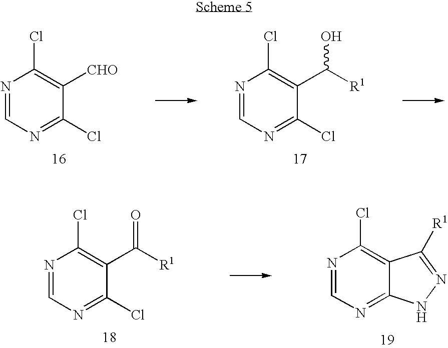

Scheme 5 illustrates a method to synthesize intermediates of the formula 19. 4,6-dichloro-5-formylpyrimidine (16) was synthesized using the method of: J. Med. Chem. 2002, 45, 3639. Compound 16 is then dissolved in a polar, aprotic solvent, preferably ethyl ether, and a nucleophile is added, preferably an alkyl magnesium reagent, at room temperature for 2 hrs to form intermediate 17. This intermediate is then treated with an oxidizing reagent, preferably chromium trioxide, in a polar aprotic solvent, preferably acetone, from 0° C. to room temperature for 2.5 hrs to form ketone 18. This intermediate is then treated with hydrazine in the presence of a base, preferably triethylamine, in a polar aprotic solvent, preferably dioxane, at room temperature for 18 hrs. to form intermediate 19.

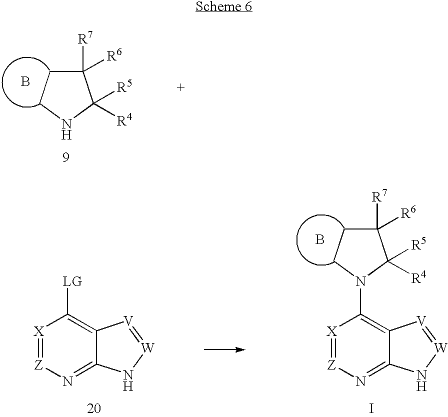

Scheme 6 details the synthesis of compounds of the formula I. The synthesis of several intermediates of the formula 9 are shown in schemes 1 and 2, and other compounds of the formula 9 are known in the literature or can readily be synthesized by those skilled in the art. Compound 9 can be coupled to heterocycles such as 20, where V, W, X, Y and Z are defined above in Formula I, and LG is a leaving group, preferably a halogen, most preferably a chloride, and the NH group of 20 is optionally protected. The coupling is done by mixing 9 with 20 without solvent or with a solvent, preferably ethyl acetate, DMF, DMSO, or NMP, at a temperature between 60° C. and 140° C., for a period of 1-48 hrs. In some cases, acid can be added to this reaction to increase the yield and speed of the reaction, preferably trifluoroacetic acid, p-toluenesulfonic acid or phosphoric acid. Compounds of the formula I can then be purified by utilizing standard methods, and can be further elaborated using methods known by those skilled in the art. One example of further elaboration is the removal of protecting groups on N of the R⁶R⁷ group. A preferred protecting group is the CBZ group, which can be removed through the use of a strong acid, preferably TFA, at temperatures ranging from 40° C. to 120° C., preferably 70° C., for 30 min. to 6 hours, preferably for 1 hr. The CBZ group can also be removed through hydrogenation with a catalyst, preferably Pd/C, at temperatures between 0° C. and 80° C., preferably room temperature, in a polar solvent, preferably methanol, optionally with the addition of catalytic acid, for a period of 1-48 hrs. Another preferred protecting group is the t-Boc group. This group can be deprotected at room temperature with acid, preferably HCl or trifluoroacetic acid, in a non-polar aprotic solvent, preferably dichloromethane, for a period of 0.5-6 hrs. Further examples of how compounds of the formula I can be elaborated are provided in the schemes below.

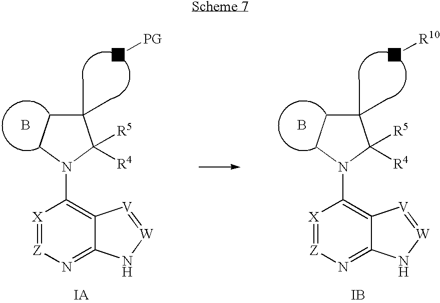

A preferred sub-class of compound of formula I wherein R⁶, R⁷ forms a cyclic amine appended to the indoline ring, as shown in scheme 7. The synthesis of compounds of this type (scheme 6) often utilizes a protecting group for this cyclic amine, preferably a carbonate protecting group such as t-Boc or CBZ. Once compound IA is synthesized, the protecting group can be removed with acid or through a different method as described in scheme 6. The resulting unprotected amines can then be further elaborated to a large number of derivatives through methods known to those skilled in the art. For example, amides can be synthesized through the coupling of acid chlorides or acids, ureas can be synthesized through the coupling of isocyanates or isocyanate isosteres, sulfonamides can be synthesized through coupling to sulfonyl chlorides, and alkyl derivatives can be synthesized through reaction with an aldehyde in the presence of a reducing agent. Alternatively, to obtain the product 1B where R¹⁰Me, the CBZ derivative can be treated directly with a hydride reagent, preferably lithium aluminum hydride, in a polar aprotic solvent, preferably THF, at a temperature between 0° C. and 70° C., preferably 20° C., for a period of 1-8 hrs.

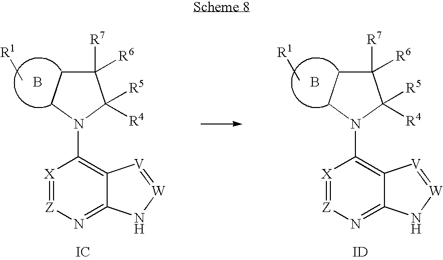

Scheme 8 exemplifies how substituents on the B ring of compounds of formula I can be replaced to produce other preferred compounds. For instance, if R¹ on 1C is a halogen, preferably a bromide, it is possible to synthesize the derivatives 1D where R¹ is a substituted amine or a substituted carbon. In the case where R¹ of 1D is a substituted amine, 1C is treated with a metal, preferably Pd₂ dba₃ in an aprotic solvent, preferably THF, with a phosphine ligand, preferably XPHOS, with an amine, and a base, preferably LHMDS, at temperatures ranging from 20° C. to 110° C., preferably 65° C., for a time of 1-48 hours. In the case where R¹ of 1D is a substituted carbon, 1C(R¹=Br) is treated with a metal, preferably Pd(PPh₃)₄, with an aryl or heteroaryl boronic acid or ester, in a polar solvent mixture, preferably DME, water and ethanol, with a base, preferably potassium carbonate, at a temperature of 150° C.-250° C., for a time of 1 minute-24 hours. Separately, if R¹ on 1C is a carboxylic acid, it is possible to synthesize ID where R¹ is a substituted amide. Thus, the IC (R¹=—CO₂H) is dissolved in a polar aprotic solvent, preferably DMF, and treated with an amine in the presence of a coupling reagent, preferably HATU, at a temperature of 0° C.-110° C., preferably room temperature, for a time of 1-48 hours to provide ID where R¹ is a substituted amide. In all cases, protecting groups may be employed elsewhere on the molecule. These groups, if employed, can be deprotected as described previously or by methods known in the art.

The compounds of the present invention may have asymmetric carbon atoms. Diasteromeric mixtures can be separated into their individual diastereomers on the basis of their physical chemical differences by methods known to those skilled in the art, for example, by chromatography or fractional crystallization. Enantiomers can be separated by converting the enantiomeric mixtures into a diastereomeric mixture by reaction with an appropriate optically active compound (e.g., alcohol), separating the diastereomers and converting (e.g., hydrolyzing) the individual diastereomers to the corresponding pure enantiomers. All such isomers, including diastereomeric mixtures and pure enantiomers are considered as part of the invention.

The compounds of formula I that are basic in nature are capable of forming a wide variety of different salts with various inorganic and organic acids. Although such salts must be pharmaceutically acceptable for administration to animals, it is often desirable in practice to initially isolate the compound of formula I from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained. The desired acid salt can also be precipitated from a solution of the free base in an organic solvent by adding to the solution an appropriate mineral or organic acid.

Those compounds of formula I that are acidic in nature are capable of forming base salts with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of this invention are those which form non-toxic base salts with the acidic compounds of formula I. Such non-toxic base salts include those derived from such pharmacologically acceptable cations as sodium, potassium calcium and magnesium, etc. These salts can easily be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum yields of the desired final product.

The compounds of the present invention are useful in the prevention and treatment of a variety of human hyperproliferative disorders such as malignant and benign tumors of the liver, kidney, bladder, breast, gastric, ovarian, colorectal, prostate, pancreatic, lung, vulval, thyroid, hepatic carcinomas, sarcomas, glioblastomas, head and neck, and other hyperplastic conditions such as benign hyperplasia of the skin (e.g., psoriasis) and benign hyperplasia of the prostate (e.g., BPH). It is, in addition, expected that a compound of the present invention may possess activity against a range of leukemias and lymphoid malignancies.

The compounds of the present invention may also be useful in the treatment of additional disorders in which aberrant expression ligand/receptor interactions or activation or signalling events related to various protein tyrosine kinases, are involved. Such disorders may include those of neuronal, glial, astrocytal, hypothalamic, and other glandular, macrophagal, epithelial, stromal, and blastocoelic nature in which aberrant function, expression, activation or signalling of the erbB tyrosine kinases are involved. In addition, the compounds of the present invention may have therapeutic utility in inflammatory, angiogenic and immunologic disorders involving both identified and as yet unidentified tyrosine kinases that are inhibited by the compounds of the present invention.

The in vitro activity of the compounds of formula I may be determined by the following procedure.

The Akt1 kinase assay is based on the measurement of fluorescence polarization using IMAP technology (Molecular Devices Corporation). Four microliters of inhibitor compounds diluted to a concentration of 10 millimolar are added to the bottom row of a polypropylene 96-well plate containing 200 microliters of 100% DMSO. The various test compounds are serially diluted up the plate by pipetting 20 microliters of compounds into wells containing 60 microliters of 100% DMSO. The components of the wells are mixed and 15 microliters of each well are transferred to another 96-well plate already containing 60 microliters of reaction buffer (RB: 10 mM Tris-HCl, pH 7.5, 10 mM MgCl₂, 0.1 mM EGTA, 0.01% Triton-X100 (Sigma #X-100), freshly added 1 mM DTT). After mixing, the Akt reactions are assembled. First, five microliters of the above compound/reaction buffer mixture is transferred to the bottom of a 96-well black polystyrene reaction plate (Costar, #3694). Next, ten microliters of a solution containing 4 micromolar ATP and 40 nanomolar fluorescent-labeled Crosstide (Tamara-labeled GRPRTSSFAEG peptide) are added. Then, 5 microliters of Akt protein in RB are added. The version of Akt used in these studies lacks the pleckstrin homology (PH) domain, and contains an aspartic acid residue in place of a serine residue in position 473 within the Akt1 hydrophobic motif. The Akt1 protein contains a polyhistidine tag at the amino terminus and is prephosphorylated on threonine at position 308 in order to activate latent kinase activity. Once the reaction components and inhibitors are assembled, the plates are gently tapped, covered with foil, and then incubated at ambient temperature for 90 minutes. IMAP beads (Molecular Devices) are then added (60 microliters of a 1:400 dilution of beads in RB). Plates are read on a Victor Plate Reader with the following settings: CW lamp filter: 544 nm, emission filter: 615 nm. Control values from wells lacking Akt protein are subtracted from the gross readings, and IC₅₀ values are calculated using XLDA.

Administration of the compounds of the present invention (hereinafter the “active compound(s)”) can be effected by any method that enables delivery of the compounds to the site of action. These methods include oral routes, intraduodenal routes, parenteral injection (including intravenous, subcutaneous, intramuscular, intravascular or infusion), topical, and rectal administration.

The amount of the active compound administered will be dependent on the subject being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to about 7 g/day, preferably about 0.1 to about 2.5 g/day. In some instances, dosage levels below the lower limit of the aforesaid range may be more than adequate, while in other cases still larger doses may be employed without causing any harmful side effect, provided that such larger doses are first divided into several small doses for administration throughout the day.

The active compound may be applied as a sole therapy or may involve one or more other anti-tumour substances, for example those selected from, for example, mitotic inhibitors, for example vinblastine; alkylating agents, for example cis-platin, carboplatin and cyclophosphamide; anti-metabolites, for example 5-fluorouracil, cytosine arabinoside and hydroxyurea, or, for example, one of the preferred anti-metabolites disclosed in European Patent Application No. 239362 such as N-(5-[N-(3,4-dihydro-2-methyl-4-oxoquinazolin-6-ylmethyl)-N-methylamino]-2-thenoyl)-L-glutamic acid; growth factor inhibitors; cell cycle inhibitors; intercalating antibiotics, for example adriamycin and bleomycin; enzymes, for example interferon; and anti-hormones, for example anti-estrogens such as Nolvadex™ (tamoxifen) or, for example anti-androgens such as Casodex™ (4′-cyano-3-(4-fluorophenylsulphonyl)-2-hydroxy-2-methyl-3′-(trifluoromethyl)propionanilide). Such conjoint treatment may be achieved by way of the simultaneous, sequential or separate dosing of the individual components of the treatment.

The pharmaceutical composition may, for example, be in a form suitable for oral administration as a tablet, capsule, pill, powder, sustained release formulations, solution suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compounds in sterile aqueous solutions, for example, aqueous propylene glycol or dextrose solutions. Such dosage forms can be suitably buffered, if desired.

Suitable pharmaceutical carriers include inert diluents or fillers, water and various organic solvents. The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials, therefor, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Methods of preparing various pharmaceutical compositions with a specific amount of active compound are known, or will be apparent, to those skilled in this art. For examples, see Remington's Pharmaceutical Sciences, Mack Publishing Company, Easter, Pa., 15th Edition (1975).

The examples and preparations provided below further illustrate and exemplify the compounds of the present invention and methods of preparing such compounds. It is to be understood that the scope of the present invention is not limited in any way by the scope of the following examples and preparations. In the following examples, “Ac” means acetyl, “Et” means ethyl, “Me” means methyl, and “Bu” means butyl.

Where HPLC chromatography is referred to in the preparations and examples below, the general conditions used, unless otherwise indicated, are as detailed by HPLC methods A through K as shown in in the following table:

| HPLC | | |
|---|---|---|
| methods | Column | Gradient |
| A | Symmetry C18 (4.6 × 50 mm; 3.5 um), 2.0 mL/min | H₂O/CH₃CN/1% TFA in H₂O |
| | | (85:10:5 at 0 min and 0:95:5 at |
| | | 5 min) |
| B | Symmetry C8 (4.6 × 50 mm; 3.5 um), 2.0 mL/min | H₂O/CH₃CN/1% TFA in H₂O |
| | | (90:5:5 at 0 min and 0:95:5 at |
| | | 5 min) |
| C | Symmetry C18 (4.6 × 50 mm; 3.5 um), 2.0 mL/min | H₂O/CH₃CN/1% TFA in H₂O |
| | | (75:20:5 at 0 min and 0:95:5 at |
| | | 5 min) |
| D | Symmetry C18 (4.6 × 100 mm, 5 um), 2.0 mL/min | H₂O/CH₃CN/1% TFA in H₂O |
| | | (90:5:5 at 0 min and 0:95:5 at |
| | | 7.5 min) |
| E | XTerra MS C8 (4.6 × 50 mm; 3.5 um), 2.0 mL/min | H₂O/CH₃CN/2% NH₃ in H₂O |
| | | (90:5:5 at 0 min and 0:95:5 at |
| | | 5 min) |
| F | Symmetry C8 (4.6 × 50 mm; 3.5 um), 2.0 mL/min | H₂O/CH₃CN/1% TFA in H₂O |
| | | (90:5:5 at 0 min and 15:80:5 at |
| | | 5 min) |
| G | Xterra MS C18 (4.6 × 50 mm; 3.5 um), 2.0 mL/min | H₂O/CH₃CN/2% NH₃ in H₂O |
| | | (90:5:5 at 0 min and 0:95:5 at |
| | | 5 min) |
| H | Zorbax 5B-C18, 5 micron, 4.6 × 150 mm, | 0.2 M ammonium acetate/acetic |
| | 3.00 mL/min | acid aqueous buffer/acetonitrile |
| | | (100:0 at 0 min, 0:100 at 10 min) |
| I | Polaris 5 micron C18-A 20 × 2.0 mm, | 94.952% water, 4.998% |
| | 1 mL/min | acetonitrile, 0.05% formic acid/ |
| | | 0.05% formic acid in acetonitrile |
| | | (95:5 at 0 min, 80:20 at 1.05 min, |
| | | 50:50 at 2.30 min, 0:100 at |
| | | 3.55 min) |
| J | Polaris 5 micron C18-A, 20 × 2.0 mm, 1 mL/min | 94.952% water, 4.998% |
| | | acetonitrile, 0.05% formic acid/ |
| | | 0.05% formic acid in acetonitrile |
| | | (95:5 at 0 min, 80:20 at 1.05 min, |
| | | 50:50 at 2.30 min, 0:100 at |
| | | 3.55 min) |
| K | Waters Xterra MS C18, 5 mm, 3.0 × 50 mm, | 0.1% TFA/acetonitrile (100:0 at |
| | 1.5 mL/min | 0 min, 100:0 at 1 min, 0:100 at 6 min) |

### EXAMPLE 1

### Preparation of 5-chloro-1-(7H-pyrrolo[2,3-d]pyrimidin-4-yl)-1,2-dihydrospiro[indole-3,4′-piperidine]

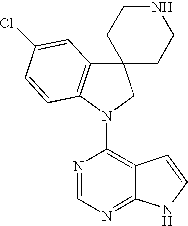

Step 1: 4-Chloro-7H-pyrrolo[2,3-d]pyrimidine was prepared by the method of Townsend (J. Med. Chem. 1990, 33 (7), 1984) or Ugarkar (J. Med. Chem. 2000, 43 (15), 2883).

Step 2: tert-Butyl 5-chloro-1,2-dihydro-1′H-spiro[indole-3,4′-piperidine]-1′-carboxylate was prepared by the method of Houghton (Tetrahedron 53 (32), 10983, 1997).

Step 3: 0.4 M stock solutions of 4-Chloro-7H-pyrrolo[2,3-d]pyrimidine and tert-butyl 5-chloro-1,2-dihydro-1′H-spiro[indole-3,4′-piperidine]-1′-carboxylate were prepared. 4-chloro-7H-pyrrolo[2,3-d]pyrimidine (0.4 M, 500 ul, 200 umol) and tert-butyl 5-chloro-1,2-dihydro-1′H-spiro[indole-3,4′-piperidine]-1′-carboxylate (0.4 M, 500 ul, 200 umol) (a suspension) in DMSO were mixed together. DIEA (neat, 70 ul, 401 umol) was added. The contents were concentrated to dryness in a Genevac. Ethyl acetate (200 ul) was added to the reaction vial and the vial was capped and heated at 105° C. for 2.5 h. Methanol (0.5 ml) was added, followed by 4 M HCl in dioxane (0.25 ml, 1000 umol). The reaction mixture was shaken at room temperature for 20 h. 2 M Ammonia in methanol (0.6 ml, 1200 umol) was added to quench the reaction and neutralize the HCl. The resulting mixture was concentrated in the Genevac to dryness and the title compound was isolated. LRMS (M+340.23), T_{R} 1.84 min, HPLC conditions F.

### EXAMPLE 2

### Preparation of 1-(5-methyl-7H-pyrrolo[2,3-d]pyrimidin-4-yl)-1,2-dihydrospiro[indole-3,4′-piperidine]

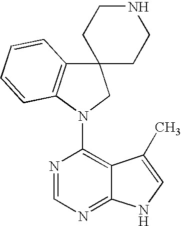

Step 1: 4-Chloro-5-methyl-7H-pyrrolo[2,3-d]pyrimidine was prepared by the method of Townsend (J. Med. Chem. 1990, 33 (7), 1984).

Step 2: Benzyl 1,2-dihydro-1′H-spiro[indole-3,4′-piperidine]-1′-carboxylate was prepared by the method described in Example 1.

Step 3: A suspension of 4-Chloro-5-methyl-7H-pyrrolo[2,3-d]pyrimidine (0.05 g, 0.30 mmol) and benzyl 1,2-dihydro-1′H-spiro[indole-3,4′-piperidine]-1′-carboxylate (0.0961 g, 0.3 mmol) in ethyl acetate was heated to 100° C., boiling off the ethyl acetate. The mixture was heated at 100° C. for 48 h. The cooled reaction was taken up in ethyl acetate and saturated sodium bicarbonate. The layers were separated and the aqueous layer was extracted three times with ethyl acetate, and the combined organics were washed with brine, dried over sodium sulfate and evaporated. The crude material was absorbed on silica gel and chromatographed with 1:1 ethyl acetate/hexanes to afford 0.693 g (51%) of benzyl 1-(5-methyl-7H-pyrrolo[2,3-d]pyrimidin-4-yl)-1,2-dihydro-1′H-spiro[indole-3,4′-piperidine]-1′-carboxylate; T_{R} 7.80 min (HPLC conditions H).

Step 4: A solution of benzyl 1-(5-methyl-7H-pyrrolo[2,3-d]pyrimidin-4-yl)-1,2-dihydro-1′H-spiro[indole-3,4′-piperidine]-1′-carboxylate (0.062 g, 0.14 mmol) in 1.5 mL of trifluoroacetic acid was heated at 70° C. for 1.5 h. The reaction mixture was evaporated to dryness. The residue was taken up in ethyl acetate and saturated sodium bicarbonate. The layers were separated and the aqueous layer was extracted three times with ethyl acetate and the combined organics were washed with brine, dried over sodium sulfate and evaporated. The crude material (0.495 g) was chromatographed on silica gel (eluting with 5-5.5% methanol/1% ammonium hydroxide/dichloromethane to afford 0.0281 g (64%) of the title compound; T_{R} 3.89 (HPLC conditions H), LRMS (M+320.2).

### EXAMPLE 3

### Preparation of 4-spiro[indole-3,4′-piperidin]-1(2H)-yl-7H-pyrrolo[2,3-d]pyrimidine-5-carbonitrile

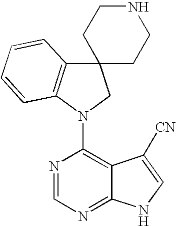

Step 1: 5-Bromo-4-chloro-7H-pyrrolo[2,3-d]pyrimidine was prepared by the method of Townsend (J. Med. Chem. 33 (7), 1984, 1990).

Step 2: A solution of 5-bromo-4-chloro-7H-pyrrolo[2,3-d]pyrimidine (4 g, 17.2 mmol) in 170 mL of anhydrous THF was cooled to −78° C. A solution of nBuLi (15.14 mL, 37.8 mmol, 2.2 eq) in hexanes was added slowly over 10 min. The reaction mixture was stirred at −78° C. for 1 hr, and DMF (1.465 mL, 18.9 mmol, 1.1 eq) was added dropwise to the yellow suspension/slurry over 10 min. The reaction mixture was stirred at −78° C. for 30 min and warmed to rt. After 1 hr, the reaction mixture was quenched with 2 mL of water and the THF was removed in vacuo. The slurry was taken up in ethyl acetate and water and saturated NH₄Cl was added. The layers were separated and the aqueous layer was extracted four times with ethyl acetate. After the last extraction, a precipitate crashed out of the water layer. The precipitate was filtered, washed with water and dried in vacuo to give 2.44 g (78%) of 4-chloro-7H-pyrrolo[2,3-d]pyrimidine-5-carbaldehyde. ¹H NMR (DMSOd₆) δ 10.23 (s, 1H), 8.75 (s, 1H), 8.61 (s, 1H) ppm.

Step 3: A sample of 4-chloro-7H-pyrrolo[2,3-d]pyrimidine-5-carbaldehyde (1.6755 g, 9.22 mmol) was crushed by mortar and pestle and was suspended in 25 mL of EtOH. Hydroxylamine hydrochloride (0.7694 g, 11.1 mmol, 1.2 eq) was added as a solid. A solution of aqueous 2M NaOH (5.45 mL, 10.9 mmol, 1.18 eq) was added to the suspension. After stirring for 3 h at rt, the material was diluted with EtOH to allow stirring and the mixture was heated at 50° C. for 2 h. The material was filtered and washed with water. The solid was dried to afford 1.7160 g, 94.6% of 4-chloro-7H-pyrrolo[2,3-d]pyrimidine-5-carbaldehyde oxime as a mixture of isomers. ¹H NMR (DMSOd₆) δ 13.03 and 12.96 (m, 1H), 11.92 and 11.05 (s, 1H), 8.63 and 8.59 (s, 1H), 8.54 and 8.48 (s, 1H), 8.05 and 7.99 (s, 1H) ppm.

Step 4: A sample of 4-chloro-7H-pyrrolo[2,3-d]pyrimidine-5-carbaldehyde oxime (diastereomeric mixture) (1.71 g, 8.7 mmol) was suspended in methylene chloride and thionyl chloride (10.38 g, 87 mmol, 10 eq) was added dropwise. After 5 h stirring at rt, another 2 mL of SOCl₂ was added and the reaction was stirred overnight at rt. The reaction was heated at 45° C. for 1 hr, the mixture was cooled to rt and was evaporated to dryness in vacuo. The mixture was taken up in ethyl acetate, water and saturated sodium bicarbonate. The precipitate that formed in the seperatory funnel was filtered. The filtrate was extracted with ethyl acetate and the combined organics were washed with brine, dried over Na₂SO₄, filtered and concentrated in vacuo to yield 0.5 g of the desired compound. The isolated precipitate (1.02 g) was stirred with aqueous ammonium chloride and ethyl acetate. The layers were separated and the aqueous layer was extracted with ethyl acetate. The combined organics were evaporated to give an additional 0.89 g of 4-chloro-7H-pyrrolo[2,3-d]pyrimidine-5-carbonitrile (Total yield is 1.39 g, 89.4%). ¹H NMR (DMSOd₆) δ13.70 (brs, 1H), 8.78 (s, 1H), 8.70 (s, 1H) ppm.

Step 5: A solution of 4-chloro-7H-pyrrolo[2,3-d]pyrimidine-5-carbonitrile (1.0 g, 5.6 mmol), benzyl 1,2-dihydro-1′H-spiro[indole-3,4′-piperidine]-1′-carboxylate (1.80 g, 5.6 mmol), phosphoric acid (0.11 g, 1.1 mmol), and potassium dihydrogen phosphate (0.76 g, 5.6 mmol) in 3 mL of dimethyl sulfoxide has heated at 80° C. for 12 h. The mixture was poured into 25% sodium bicarbonate in water (by weight) and extracted 3× with ethyl acetate. The combined organics were dried over sodium sulfate. Chromatography on silica gel, eluting with 62-65% ethyl acetate/hexanes afforded 1.94 g (74%) of benzyl 1-(5-cyano-7H-pyrrolo[2,3-d]pyrimidin-4-yl)-1,2-dihydro-1′H-spiro[indole-3,4′-piperidine]-1′-carboxylate; T_{R} 7.54 min (HPLC Conditions H).

Step 6: A solution of benzyl 1-(5-cyano-7H-pyrrolo[2,3-d]pyrimidin-4-yl)-1,2-dihydro-1′H-spiro[indole-3,4′-piperidine]-1′-carboxylate (0.374 g, 0.81 mmol) in 2 mL of trifluoroacetic acid was heated at 70° C. for 2.5 h. The mixture was evaporated and the residue taken up in 5 mL of methanol and cooled to 0° C. 2N ammonia in methanol (3 mL) was added. The precipitate that formed was filtered and washed with methanol affording 0.277 g (77%) of the title compound as the trifluoroacetate salt; LRMS (M+330.2), T_{R} 3.86 min (HPLC Conditions H).

### EXAMPLE 4

### Preparation of 5-chloro-1-(5-chloro-7H-pyrrolo[2,3-d]pyrimidin-4-yl)-1,2-dihydrospiro[indole-3,4′-piperidine]

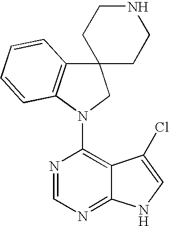

Step 1: 4,5-Dichloropyrrolopyrimidine was prepared by the method of Townsend (J. Med. Chem. 1988, 31, 2086).

Step 2: The title compound was prepared by the coupling of 4,5-dichloropyrrolopyrimidine and tert-butyl 5-chloro-1,2-dihydro-1′H-spiro[indole-3,4′-piperidine]-1′-carboxylate as described in Example 1; LRMS (M+374.19), T_{R} 2.13 (HPLC conditions F).

### EXAMPLE 5

### Preparation of 1-(5-benzyl-7H-pyrrolo[2,3-d]pyrimidin-4-yl)-1,2-dihydrospiro[indole-3,4′-piperidine]

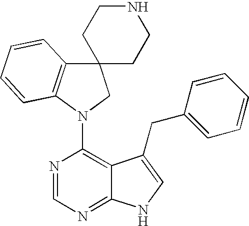

Step 1: A solution of 5-bromo-4-chloro-7H-pyrrolo[2,3-d]pyrimidine (2.49 g, 10.7 mmol) in 100 mL of tetrahydrofuran at −78° C. was treated dropwise with a 2.5 M hexanes solution of n-butyl lithium (9.45 mL, 23.7 mmol). After stirring at −78° C. for 1 h, the mixture was treated dropwise with benzaldehyde (1.35 mL, 13.4 mmol). The cooling bath was removed after 10 min and the mixture was stirred while warming to rt for 2 h. Water (1 mL) was carefully added and the tetrahydrofuran was evaporated. The residue was taken up in ethyl acetate and half-saturated ammonium chloride. The layers were separated and the aqueous layer was extracted four times with ethyl acetate and the combined organics were washed with brine, dried over sodium sulfate and evaporated. The crude material was chromatographed on silica gel, eluting with 2.2-3.4% methanol/methylene chloride to afford 2.13 g (76%) of (4-chloro-7H-pyrrolo[2,3-d]pyrimidin-5-yl)(phenyl)methanol; T_{R} 4.29 min (HPLC conditions H).

Step 2: A suspension of (4-chloro-7H-pyrrolo[2,3-d]pyrimidin-5-yl)(phenyl)methanol (2.05 g, 7.9 mmol) in 30 mL of methylene chloride was treated sequentially with triethylsilane (1.10 g, 9.5 mmol) and trifluoroacetic acid 1.21 mL, 15.8 mmol) and stirred at rt for 21 h. The mixture was evaporated to dryness and taken up in ethyl acetate and water. Sodium carbonate was added to neutralize residual trifluoroacetic acid, the layers were separated and the aqueous layer was extracted three times with ethyl acetate. The combined organics were washed with brine and dried over sodium sulfate giving 2.39 g of crude material. The crude product was chromatographed on silica gel, eluting with 1% methanol/methylene chloride to afford 1.60 g of 5-benzyl-4-chloro-7H-pyrrolo[2,3-d]pyrimidine; T_{R} 6.18 min (HPLC conditions H).

Step 3: A suspension of 5-benzyl-4-chloro-7H-pyrrolo[2,3-d]pyrimidine (0.0683 g, 0.28 mmol) and benzyl 1,2-dihydro-1′H-spiro[indole-3,4′-piperidine]-1′-carboxylate (0.0904 g, 0.28 mmol) in ethyl acetate was heated to 100° C., boiling off the ethyl acetate. The mixture was heated at 100° C. for 24 h. The mixture was taken up in 4:1 dichloromethane/methanol and 2M ammonia in methanol was added until neutral. The mixture was evaporated to dryness and chromatographed on silica gel, eluting with 2-2.3% methanol/methylene chloride to afford 0.075 g (50%) of benzyl 1-(5-benzyl-7H-pyrrolo[2,3-d]pyrimidin-4-yl)-1,2-dihydro-1′H-spiro[indole-3,4′-piperidine]-1′-carboxylate; T_{R} 8.62 min (HPLC conditions H).

Step 4: A solution of benzyl 1-(5-benzyl-7H-pyrrolo[2,3-d]pyrimidin-4-yl)-1,2-dihydro-1′H-spiro[indole-3,4′-piperidine]-1′-carboxylate (0.072 g, 0.14 mmol) in 1.5 mL of trifluoroacetic acid was heated at 70° C. for 1.5 h. The reaction mixture was evaporated to dryness. The mixture was taken up in 4:1 dichloromethane/methanol and 2M ammonia in methanol was added until neutral. The mixture was evaporated to dryness and chromatographed on silica gel, eluting with 7-7.5% methanol/methylene chloride/1% ammonium hydroxide to afford 0.0492 g (92%) of the title compound; T_{R} 4.76 (HPLC conditions H), LRMS (M+1, 396.4).

### EXAMPLE 6

### Preparation of 5-pyridin-3-yl-1-(7H-pyrrolo[2,3-d]pyrimidin-4-yl)-1,2-dihydrospiro[indole-3,4′-piperidine]

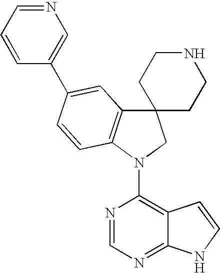

Step 1: Benzyl 5-bromo-1-(7H-pyrrolo[2,3-d]pyrimidin-4-yl)-1,2-dihydro-1′H-spiro[indole-3,4′-piperidine]-1′-carboxylate (synthesized through the method of example 1) (100 mg, 0.192 mmol) was slurried in DME (1.2 mL) and EtOH (0.8 mL). 3-pyridylboronic acid (48 mg, 0.384 mmol) was charged to the reaction vial. PS-PPh₃-Pd(0) (80 mg, 0.008 mmol) was added to the reaction mixture and finally potassium carbonate (40 mg, 0.289 mmol) was added to the reaction in 0.32 mL water. The Emrys vial was then sealed with a crimper and placed in the microwave for 20 min. at 160° C. The reaction was then concentrated to dryness and placed on a Waters Oasis MCX resin cartridge with methanol. The cartridge was eluted with 25 mL of methanol followed by 25 mL of 1N NH₃/MeOH. The alkaline methanol solution was then stripped to dryness to recover 83 mg of 5-pyridin-3-yl-1-(7H-pyrrolo[2,3-d]pyrimidin-4-yl)-1,2-dihydrospiro[indole-3,4′-1′-benzoyloxypiperidine] as a tan solid that was used without further purification. Crude yield=84%; T_{R}=2.24 min (HPLC method I); LRMS (M+): 517.4.

Step 2: 5-Pyridin-3-yl-1-(7H-pyrrolo[2,3-d]pyrimidin-4-yl)-1,2-dihydrospiro[indole-3,4′-1′-benzoyloxypiperidine] (83 mg, 0.161 mmol) was dissolved and heated in trifluoroacetic acid (1 mL) for 1 h at 70° C. The reaction solution was taken to dryness under high vacuum and redissolved in 1 mL of DMSO before chromatography on reverse phase preparative HPLC. The clean fractions are isolated to give 16 mg of the title compound. T_{R}=0.98 min (HPLC method I); LRMS (M+): 383.3; ¹H NMR (400 MHz, CD₃OD) [symbol] 8.84 (1H, s), 8.62 (1H, d, J=8.81 Hz), 8.51 (1H, s), 8.40 (1H, s), 8.27 (2H, s), 8.13 (1H, d, J=8.56), 7.63-7.60 (2H, m), 7.54 (1H, dd, J=5.2, J=10.6), 7.31 (1H, s), 6.96 (1H, s), 4.63 (2H, s), 3.53 (2H, d, J=13.0) 3.39 (2H, t, J=13.5), 2.28 (2H, t, J=13.7 Hz), 2.10 (1H, d, J=14.5 Hz).

### EXAMPLE 7

### Preparation of N-(3-methylbenzyl)-1-(7H-pyrrolo[2,3-d]pyrimidin-4-yl)-1,2-dihydrospiro[indole-3,4′-piperidin]-5-amine

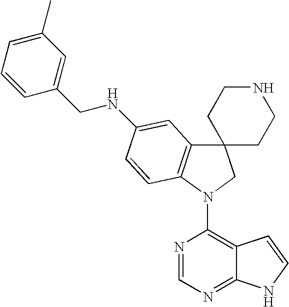

Step 1: Benzyl 5-bromo-1-(7H-pyrrolo[2,3-d]pyrimidin-4-yl)-1,2-dihydro-1′H-spiro[indole-3,4′-piperidine]-1′-carboxylate (made through the method of example 1) (75 mg, 0.145 mmol) was dissolved in dioxane (1 mL) under nitrogen. X-PHOS (14 mg, 0.029 mmol, 20 mol %) and Pd₂(dba)₃ (6.6 mg, 0.0075 mmol, 5 mol %) were added, followed by lithium hexamethyldisilazide (522 μL, 0.522 mmol) and 3-methylbenzylamine (46 μL, 0.36 mmol). The resulting mixture was stirred at 65° C. for 2 hours. The solvent was removed by rotary evaporation to afford benzyl 5-[(3-methylbenzyl)amino]-1-(7H-pyrrolo[2,3-d]pyrimidin-4-yl)-1,2-dihydro-1′H-spiro[indole-3,4′-piperidine]-1′-carboxylate which was used directly in next step. LRMS (M+): 559.5; t_{R} (HPLC method I): 2.9 min.

Step 2: The Cbz group of benzyl 5-[(3-methylbenzyl)amino]-1-(7H-pyrrolo[2,3-d]pyrimidin-4-yl)-1,2-dihydro-1′H-spiro[indole-3,4′-piperidine]-1′-carboxylate was deprotected utilizing the method described in example 6 to provide 10 mg. (13% yield) of N-(3-methylbenzyl)-1-(7H-pyrrolo[2,3-d]pyrimidin-4-yl)-1,2-dihydrospiro[indole-3,4′-piperidin]-5-amine. ¹H NMR (500 MHz, Methanol-d₄) [symbol] 1.95 (2H, d, J=14.5 Hz), 2.12 (2H, m), 2.33 (3H, s), 3.27 (2H, m), 3.45 (2H, d, J=13 Hz), 4.30 (2H, s), 4.44 (2H, s), 6.61 (2H, d, J=8.5 Hz), 6.86 (1H, d, J=3.5 Hz), 7.05 (1H, d, J=7.5 Hz), 7.19 (4H, m), 8.19 (1H, d, J=8.5 Hz), 8.25 (1H, s); LRMS (M+): 425.2; t_{R} (HPLC method I): 1.34 min.

### EXAMPLE 8

### Preparation of 2-cyclopropyl-1-(1H-pyrazolo[3,4-d]pyrimidin-4-yl)-1,2-dihydrospiro[indole-3,4′-piperidine]

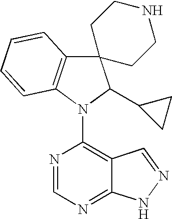

Step 1: In 600 mL of chloroform is dissolved phenylhydrazine (4.07 g, 37.7 mmol) and 8.04 g of tert-butyl 4-formylpiperidine-1-carboxylate (8.04 g, 37.7 mmol) was added to the flask under nitrogen. The pot was cooled to 0° C. and TFA was added dropwise over 15 minutes. The pot was then warmed to 50° C. and stirred overnight. The reaction was then recooled to 0° C. and neutralized with 6% aqueous ammonium hydroxide. The product was extracted with EtOAc (3×100 mL) and washed with brine. The combined organics are dried over Na₂SO₄ and filtered. The filtrate was stripped to a pale orange foam to give 5.06 g (47% yield) of tert-butyl 1′H-spiro[indole-3,4′-piperidine]-1′-carboxylate, which was used without further purification. T_{R}: 2.28 min. (HPLC conditions I); LRMS (M+): 287.3.

Step 2: tert-Butyl 1′H-spiro[indole-3,4′-piperidine]-1′-carboxylate (0.60 g, 2.1 mmol) was added to a dry flask under nitrogen and dissolved in 2 mL of anhydrous tetrahydrofuran. The pot was cooled to 0° C. and 20 mL of 0.5M cyclopropylmagnesium bromide (4.76 eq.) was added dropwise before allowing the pot to warm to room temperature. The reaction was stirred for 20 h and is then recooled to 0° C. before quenching with 10 mL of saturated aqueous ammonium chloride. The product, after extracting with ethyl acetate (3×10 mL), is dried over Na₂SO₄, filtered, stripped and pumped to dryness to provide 0.64 g of tert-butyl 2-cyclopropyl-1-(1H-pyrazolo[3,4-d]pyrimidin-4-yl)-1,2-dihydro-1′H-spiro[indole-3,4′-piperidine]-1′-carboxylate that was used without further purification (93% crude yield). T_{R}: 2.26 min. (HPLC conditions I); LRMS (M+): 329.2.

Step 3: tert-Butyl 2-cyclopropyl-1-(1H-pyrazolo[3,4-d]pyrimidin-4-yl)-1,2-dihydro-1′H-spiro[indole-3,4′-piperidine]-1′-carboxylate (64 mg, 0.198 mmol) was placed in a one dram vial with DMF (75 uL). 4-chloro-1H-pyrazolo[3,4-d]pyrimidine (31 mg, 0.198 mmol) was added and the reaction was heated at 70° C. for 3 h. The reaction is cooled to rt and directly chromatographed (Rf=0.2, 5-10% methanol/chloroform) to give 46 mg of tert-butyl 2-cyclopropyl-1-(1H-pyrazolo[3,4-d]pyrimidin-4-yl)-1,2-dihydro-1′H-spiro[indole-3,4′-piperidine]-1′-carboxylate; T_{R} 2.67 min. (HPLC conditions I); LRMS (M+): 447.4.

Step 4: tert-Butyl 2-cyclopropyl-1-(1H-pyrazolo[3,4-d]pyrimidin-4-yl)-1,2-dihydro-1′H-spiro[indole-3,4′-piperidine]-1′-carboxylate (46 mg) was treated with 1 mL of neat trifluoroacetic acid and was placed on a shaker plate for 30 minutes at 70° C. The reaction was stripped to dryness on the rotovap before being redissolved in ethyl acetate (2 mL) and restripped two additional times. The trifluoroacetic acid salt was then dissolved in DMSO (500 uL) and purified using preparative reverse phase HPLC to recover 14 mg of purified 2-cyclopropyl-1-(1H-pyrazolo[3,4-d]pyrimidin-4-yl)-1,2-dihydrospiro[indole-3,4′-piperidine]. T_{R}: 1.35 min. (HPLC conditions I); LRMS (M+): 347.2; ¹H NMR (400 MHz, CD₃OD) δ 8.47 (1H, s), 8.40 (1H, s), 8.28 (1H, d, J=7.7 Hz), 7.327 (2H, m), 7.16 (1H, t, J=7.5 Hz), 5.13 (1H, d, J=4.6), 3.57-3.66, (1H, m), 3.44 (1H, t, J=12.8 Hz), 3.27-3.40 (2H, m), 1.76-1.82 (2H, m), 1.19-1.27 (1H, m), 0.54-0.61 (1H, m) 0.31-0.38 (1H, m), 0.21-0.27 (1H, m), −0.04-−0.09 (1H, m).

### EXAMPLE 9

### Preparation of N-benzyl-1-(7H-pyrrolo[2,3-d]pyrimidin-4-yl)-1,2-dihydrospiro[indole-3,4′-piperidine]-5-carboxamide

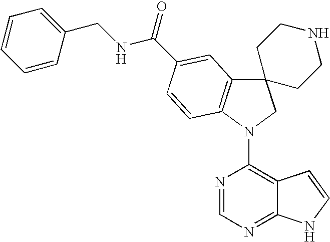

Step 1: 1′-[(Benzyloxy)carbonyl]-1-(7H-pyrrolo[2,3-d]pyrimidin-4-yl)-1,2-dihydrospiro[indole-3,4′-piperidine]-5-carboxylic acid was prepared by the methods described in Example 1,1′-[(Benzyloxy)carbonyl]-1-(7H-pyrrolo[2,3-d]pyrimidin-4-yl)-1,2-dihydrospiro[indole-3,4′-piperidine]-5-carboxylic acid (100 mg, 0.206 mmol) was dissolved in DMF (1 mL). DIPEA (72 μL, 0.206 mmol) and HATU (82 mg, 0.206 mmol) were added, followed by benzylamine (22 μL, 0.206 mmol). The resulting mixture was stirred at room temperature for 2 hours. Saturated aqueous NaHCO₃ was added (7 mL). The mixture was extracted by ethyl acetate (25 mL). The separated organic phase was dried over Na₂SO₄ The solvent was removed by rotary evaporation. The crude product is purified by flash-chromatography (ethyl acetate/hexane) to give 80 mg (68%) of benzyl 5-[(benzylamino)carbonyl]-1-(7H-pyrrolo[2,3-d]pyrimidin-4-yl)-1,2-dihydro-1′H-spiro[indole-3,4′-piperidine]-1′-carboxylate; ¹H NMR (500 MHz, Methanol-d₄) [symbol] 1.82 (2H, m), 1.97 (2H, m), 3.18 (2H, m), 4.24 (2H, d, J=14 Hz), 4.56 (2H, s), 4.60 (2H, d, J=5 Hz), 5.18 (2H, s), 6.88 (1H, d, J=3.5 Hz), 7.33 (11H, m), 7.78 (1H, d, J=1.5 Hz), 7.83 (1H, d, J=1.5 Hz), 8.38 (1H, s), 8.53 (1H, d, J=8.5 Hz); LRMS (M+): 573.4; t_{R} (HPLC method I): 2.64 min.

The Cbz group of benzyl 5-[(benzylamino)carbonyl]-1-(7H-pyrrolo[2,3-d]pyrimidin-4-yl)-1,2-dihydro-1′H-spiro[indole-3,4′-piperidine]-1′-carboxylate was deprotected utilizing the method described in example 6 to provide 44 mg. (76% yield) of N-benzyl-1-(7H-pyrrolo[2,3-d]pyrimidin-4-yl)-1,2-dihydrospiro[indole-3,4′-piperidine]-5-carboxamide; ¹H NMR (400 MHz, Methanol-d₄) δ 2.05 (2H, d, J=14.8 Hz), 2.29 (2H, m), 3.33 (2H, m), 3.49 (2H, d, J=13.2 Hz), 4.59 (4H, d, J=9.2 Hz), 6.95 (1H, d, J=4 Hz), 7.29 (6H, m), 7.81 (2H, m), 8.40 (1H, s), 8.53 (1H, d, J=8.8 Hz); LRMS (M+): 439.3; t_{R} (LCMS standard): 1.20 min.

### EXAMPLE 10

### Preparation of 1′-methyl-1-(7H-pyrrolo[2,3-d]pyrimidin-4-yl)-1,2-dihydrospiro[indole-3,4′-piperidine]

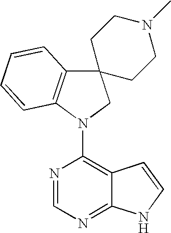

4-Chloro-7H-pyrrolo[2,3-d]pyrimidine (0.4 M, 300 ul, 120 umol) and benzyl 1,2-dihydro-1′H-spiro[indole-3,4′-piperidine]-1′-carboxylate (0.4 M, 300 ul, 120 umol) in DMSO were mixed together. DIEA (neat, 75 ul, 431 umol) was added. The contents were concentrated to dryness in a Genevac and the reaction vial was capped and heated at 100° C. for 25 h, 40 min to form benzyl 1-(7H-pyrrolo[2,3-d]pyrimidin-4-yl)-1,2-dihydro-1′H-spiro[indole-3,4′-piperidine]-1′-carboxylate. THF (0.5 ml) was added to the mixture, followed by lithium aluminum hydride (1 M in THF, 0.24 ml, 240 umol). The resulting solution was shaken at room temperature for 2 h, 20 min. Water (2 ml) and dichloroethane (2 ml) were added to the reaction mixture and the layers were separated. The aqueous layer was re-extracted with dichloroethane (2 ml). The combined organic layers were concentrated to dryness to afford 27.5 mg of a crude product. The material was dissolved in DMSO (1 ml) and purified by HPLC to afford 9.4 mg of the title compound as a TFA salt (18% overall yield after two steps). APCI LCMS: Observed mass: 320.13 (M+1). Retention time: 1.9 min (Method E).

### EXAMPLE 11

### Preparation of 5-chloro-1′-(2-methylbutyl)-1-(7H-pyrrolo[2,3-d]pyrimidin-4-yl)-1,2-dihydrospiro[indole-3,4′-piperidine]

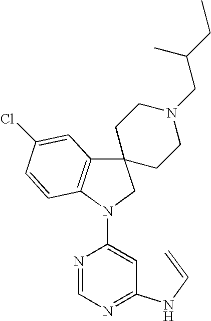

A 1.0 M stock solution of 2-methylbutanal in dichloroethane and a 0.25 M stock solution of sodium triacetoxyborohydride in dichloroethane (a suspension) were prepared. To 5-chloro-1-(7H-pyrrolo[2,3-d]pyrimidin-4-yl)-1,2-dihydrospiro[indole-3,4′-piperidine] as prepared in example 1 was added to the 2-methylbutanal solution (1.0 M, 400 ul, 400 umol) and sodium triacetoxyborohydride solution (0.25 M, 1.6 ml, 400 umol). The resulting reaction mixture was shaken at room temperature for 25 h. Dichloroethane (1 ml) and 10% aqueous ammonia (3 ml) were added. The contents were shaken and centrifuged. The layers were separated and the aqueous layer was re-extracted with dichloroethane (3.5 ml). The combined organic layers were concentrated in the Genevac to dryness to afford 104.8 mg of a crude product. The material was dissolved in DMSO and purified by HPLC to afford 26.9 mg of the title compound as the TFA salt (26% overall yield for three steps). APCI LCMS: Observed mass: 410.05 (M+1). Retention time: 2.02 min (Method B).

### EXAMPLE 12

### Preparation of 1′-(cyclopropylacetyl)-1-(7H-pyrrolo[2,3-d]pyrimidin-4-yl)-1,2-dihydrospiro[indole-3,4′-piperidine]

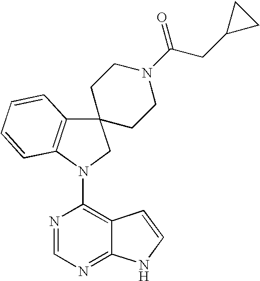

A 0.5 M stock solution of cyclopropyl methyl carboxylic acid in DMF and a 0.25 M stock solution of HBTU in DMF were prepared. To a sample of 1-(7H-pyrrolo[2,3-d]pyrimidin-4-yl)-1,2-dihydrospiro[indole-3,4′-piperidine], prepared on a 120 μmol scale by the methods described in Example 1, was added DMF (300 ul), the cyclopropyl methyl carboxylic acid solution (0.5 M, 300 ul, 150 umol), DIEA (neat, 60 ul, 344 umol) and a HBTU solution (0.25 M, 1.2 ml). The resulting reaction mixture was shaken at room temperature for 22 h. Dichloroethane (3 ml) and 0.4 M NaOH (2 ml) were added. The contents were shaken and centrifuged. The layers were separated and the aqueous layer was re-extracted with dichloroethane (2 ml). The combined organic layers were concentrated to dryness to afford 121.7 mg of a crude product that was dissolved in DMSO (1 ml) and purified by HPLC to afford 7.6 mg of the title compound (13% overall yield after 3 steps). APCI LCMS: Observed mass: 388.08 (M+1). Retention time: 2.00 min (Method C).

### EXAMPLE 13

### Preparation of 1′-(isopropylsulfonyl)-1-(7H-pyrrolo[2,3-d]pyrimidin-4-yl)-1,2-dihydrospiro[indole-3,4′-piperidine]

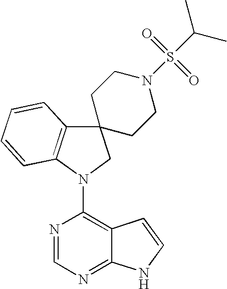

A 0.25 M stock solution of isopropyl sulfonyl chloride in THF was prepared. To a sample of 1-(7H-pyrrolo[2,3-d]pyrimidin-4-yl)-1,2-dihydrospiro[indole-3,4′-piperidine], prepared on a 120 μmol scale by the method described in Example 1, was added DMF (600 ul), TEA (neat, 50 ul, 360 umol) and the isopropyl sulfonyl chloride solution (0.25 M, 600 ul, 150 umol). The reaction mixture was shaken at room temperature for 48 h. Dichloroethane (3 ml) and water (2 ml) were added. The contents were shaken and centrifuged. The aqueous layer was re-extracted with dichloroethane (2 ml). The combined organic layers were concentrated to dryness to afford 45.0 mg of a crude product. It was dissolved in DMSO (1 ml) and purified by HPLC to afford 8.9 mg of the title compound (14% overall yield after 3 steps). APCI LCMS: Observed mass: 412.17 (M+1). Retention time: 2.15 min (Method C).

### EXAMPLE 14

### Preparation of isobutyl 1-(7H-pyrrolo[2,3-d]pyrimidin-4-yl)-1,2-dihydro-1′H-spiro[indole-3,4′-piperidine]-1′-carboxylate

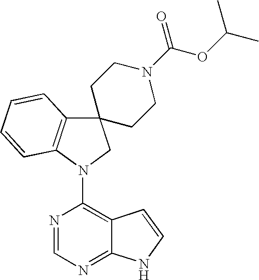

A 0.25 M stock solution of isobutyl chloroformate in dichloroethane and a 1.0 M stock solution of DMAP in DCE were prepared. To a sample of 1-(7H-pyrrolo[2,3-d]pyrimidin-4-yl)-1,2-dihydrospiro[indole-3,4′-piperidine], prepared on a 120 μmol scale by the method described in Example 1, was added dichloroethane (300 ul), the isobutyl chloroformate solution (0.25 M, 600 ul, 150 umol) and a DMAP solution (1.0 M, 375 ul, 375 umol). The resulting reaction mixture was shaken at room temperature for 48 h. Dichloroethane (3 ml) and water (2 ml) were added. The contents were shaken and centrifuged. The layers were separated and the aqueous layer was re-extracted with dichloroethane (2 ml). The combined organic layers were concentrated to dryness to afford 35.7 mg of a crude product. It was dissolved in DMSO (1 ml) and purified by HPLC to afford 7.5 mg of the title compound (12% overall yield after 3 steps). APCI LCMS: Observed mass: 406.06 (M+1). Retention time: 2.48 min (Method C).

### EXAMPLE 15

### Preparation of 1′-(morpholin-4-ylcarbonyl)-1-(7H-pyrrolo[2,3-d]pyrimidin-4-yl)-1,2-dihydrospiro[indole-3,4′-piperidine]

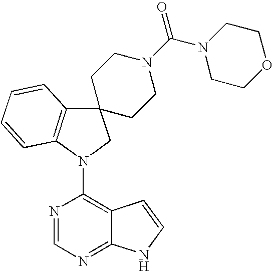

A 0.5 M stock solution of morpholinyl carbonyl chloride in methanol was prepared. To a sample of 1-(7H-pyrrolo[2,3-d]pyrimidin-4-yl)-1,2-dihydrospiro[indole-3,4′-piperidine], prepared on a 120 μmol scale by the method described in Example 1, was added dichloroethane (600 ul), N-methyl morpholine (neat, 40 ul, 364 umol), and the morpholinyl carbonyl chloride solution (0.5 M, 300 ul, 150 umol). The reaction mixture was shaken at 50° C. for 21.5 h. Dichloroethane (2.7 ml) and water (2 ml) were added. The vial was shaken and centrifuged. The layers were separated and the aqueous layer was re-extracted with dichloroethane (2 ml). The combined organic layers were concentrated to dryness to afford 42.3 mg of a crude product that was dissolved in DMSO (1 ml) and purified by HPLC to afford 13.6 mg of the title compound (22% overall yield after 3 steps). APCI LCMS: Observed mass: 419.06 (M+1). Retention time: 1.81 min (Method C).

### EXAMPLE 16

### Preparation of 1-(1H-pyrrolo[2,3-b]pyridin-4-yl)-1,2-dihydrospiro[indole-3,4′-piperidine]

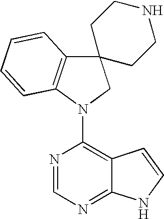

Step 1: Benzyl 1,2-dihydro-1′H-spiro[indole-3,4′-piperidine]-1′-carboxylate (2 g, 6.2 mmol) is mixed with 4-chloro-1H-pyrrolo[2,3-b]pyridine (prepared as described in WO 2003000690), DMF (1 mL) and TFA (0.478 mL). The suspension is heated to 65° C. for 2 days. The reaction is then cooled to room temperature and partitioned between EtOAc and a solution of saturated sodium bicarbonate in water. The EtOAc layer is removed and the water layer is extracted twice more. The combined organics are dried over sodium sulfate, filtered, and the solvent is removed in vacuo. The resulting solid is then purified by column chromatography (99% EtOAc+1% Et₃N) to provide 1.41 g (52% yield) of benzyl 1-(1H-pyrrolo[2,3-b]pyridin-4-yl)-1,2-dihydro-1′H-spiro[indole-3,4′-piperidine]-1′-carboxylate as an orange solid. ¹H NMR (500 MHz, DMSO-d₆) δ 11.56 (1H, br. s), 8.05 (1H, d, J=6.0 Hz), 7.38-7.40 (4H, m), 7.31-7.35 (2H, m), 7.27 (1H, br. d, J=7.0 Hz), 7.12 (1H, td, J=8.0, 1.0 Hz), 7.05 (1H, d, J=8.0 Hz), 6.92 (1H, d, J=6.0 Hz), 6.85 (1H, t, J=7.8 Hz), 6.43-6.44 (1H, m), 5.11 (2H, br. s), 4.18 (2H, s), 3.98-4.06 (2H, m), 2.98-3.19 (2H, m), 1.81 (2H, dt, J=12.9, 4.7 Hz), 1.67-1.75 (2H, m); LRMS (M+): 439.2; HPLC retention time: 1.99 min. (HPLC method 1).

Step 2: Benzyl 1-(1H-pyrrolo[2,3-b]pyridin-4-yl)-1,2-dihydro-1′H-spiro[indole-3,4′-piperidine]-1′-carboxylate (200 mg, 0.457 mmol) was dissolved in TFA (1 mL). The solution is heated to 70° C. for 1.5 hrs. The reaction is then cooled to room temperature and the solvent is removed in vacuo. The resulting brown oil is then purified by column chromatography (47.5% MeOH+47.5% EtOAc+5% Et₃N). The solvent from the resulting fractions is then removed in vacuo and the residue is dissolved in EtOAc and filtered. The mother liquor is then concentrated and the resulting solid is recrystallized from EtOAC to yield 54 mg (39% yield) of the title compound as a white solid. ¹H NMR (500 MHz, DMSO-d₆) δ 11.56 (1H, br. s), 8.04 (1H, d, J=5.5 Hz), 7.32 (1H, t, J=2.8 Hz), 7.22 (1H, d, J=7.5 Hz), 7.12 (1H, dt, J=7.0, 1.5 Hz), 7.05 (1H, d, J=8.0 Hz), 6.92 (1H, d, J=5.0 Hz), 6.87 (1H, dt, J=7.3, 1.0 Hz), 6.42-6.44 (1H, m), 4.14 (2H, br. s), 2.90-2.96 (2H, m), 2.66 (2H, t, J=12.5 Hz), 1.78 (2H, dt, J=12.8, 4.8 Hz), 1.61 (2H, br. d, J=13 Hz); LRMS (M+): 305.3; HPLC retention time: 0.55 min (HPLC Conditions I).

### EXAMPLE 17

### Preparation of 1-(3-chloro-1H-pyrrolo[2,3-b]pyridin-4-yl)-1,2-dihydrospiro[indole-3,4′-piperidine]

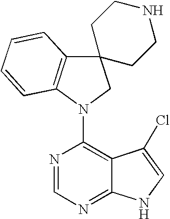

Step 1: Benzyl 1-(1H-pyrrolo[2,3-b]pyridin-4-yl)-1,2-dihydro-1′H-spiro[indole-3,4′-piperidine]-1′-carboxylate (prepared as described in example 16) (0.65 mmol) was dissolved in acetonitrile (5 mL), and triethylamine (91 uL, 0.65 mmol) was added followed by N-chlorosuccinamide (104 mg, 0.78 mmol). The reaction was heated to 80° C. for 3 hrs. The reaction was cooled to room temperature and partitioned between ethyl acetate and water. The ethyl acetate layer was removed and the water layer is extracted twice more. The combined organics were washed with brine, dried over sodium sulfate, filtered, and the solvent was removed in vacuo to provide benzyl 1-(3-chloro-1H-pyrrolo[2,3-b]pyridin-4-yl)-1,2-dihydro-1′H-spiro[indole-3,4′-piperidine]-1′-carboxylate as a brown oil. LRMS (M+): 473.3; HPLC retention time: 2.8 min (HPLC conditions 1).

Step 2: Benzyl 1-(3-chloro-1H-pyrrolo[2,3-b]pyridin-4-yl)-1,2-dihydro-1′H-spiro[indole-3,4′-piperidine]-1′-carboxylate (0.65 mmol) was deprotected using TFA as described in example 16 to yield a crude product that was purified by prep-HPLC to provide 44 mg (20% yield) of the formate salt of the title compound as a white solid. ¹H NMR (500 MHz, DMSO-d₆) δ 12.08 (1H, br. s), 8.22 (1H, d, J=5.0 Hz), 8.17 (1H, s), 7.61 (1H, d, J=2.5 Hz), 7.16 (1H, d, J=8.0 Hz), 7.07 (1H, t, J=7.3 Hz), 7.02 (1H, d, J=6.0 Hz), 6.85 (1H, t, J=7.8 Hz), 6.62 (1H, d, J=7.5 Hz), 4.01 (2H, br. s), 3.30-3.35 (2H, m), 3.04-3.12 (2H, m), 1.98-2.08 (2H, m), 1.90-1.96 (2H, m); LRMS (M+): 339.3; HPLC retention time: 1.02 min. (HPLC conditions 1).

### EXAMPLE 18

### Preparation of 1′-methyl-1-(1H-pyrrolo[2,3-b]pyridin-4-yl)-1,2-dihydrospiro[indole-3,4′-piperidine]

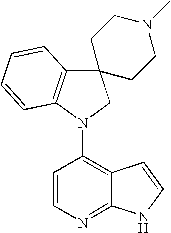

Benzyl 1-(1H-pyrrolo[2,3-b]pyridin-4-yl)-1,2-dihydro-1′H-spiro[indole-3,4′-piperidine]-1′-carboxylate (prepared as described in example 16) (0.65 mmol) was reduced with lithium aluminum hydride as described in example 10 to yield a crude product that was purified by prep-HPLC to provide 77 mg (37% yield) of the trifluoroacetate salt of the title compound as a white solid; ¹H NMR (500 MHz, DMSO-d₆) δ 12.36 (1H, br. s), 9.68 (1H, br. s), 8.14-8.16 (1H, m), 7.50-7.52 (1H, m), 7.38 (1H, d, J=8 Hz), 7.26-7.31 (2H, m), 7.21 (1H, d, J=7.0), 7.10 (1H, t, J=7.5 Hz), 6.83 (1H, m), 4.44 (2H, br. s), 3.46 (2H, br. d, J=12.5 Hz), 3.15-3.27 (2H, m), 2.84 (3H, s), 2.08-2.14 (2H, m), 1.94-2.00 (2H, m); LRMS (M+): 319.3; HPLC retention time: 0.41 min. (HPLC conditions 1).

### EXAMPLE 19

### Preparation of (3R)-1-(1H-pyrrolo[2,3-b]pyridin-4-yl)-1,2-dihydrospiro[indole-3,3′-pyrrolidine]

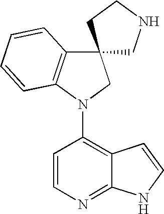

Step 1: In a 2 L RBF was added 1,2,3,4-tetrahydro-9H-pyrido(3,4-B)indole (98%, 50 g, 290.3 mmol) followed by dichloromethane (7.771 moles; 660.0 g; 500.0 mL) and triethylamine (1.200 equiv; 348.4 mmoles; 35.25 g; 48.96 mL). The reaction was cooled from 20° C. to a temperature of −2 to 2° C. and benzyl chloroformate (1.170 equiv; 339.7 mmoles; 57.94 g; 48.69 mL) was added dropwise. The reaction was stirred for 0.5 to 1.5 hr at 18 to 22° C. Water (27.75 moles; 500.0 g; 500.0 mL) was added and stirred for 30 to 40 min at 18 to 22° C. The reaction was transferred to a seperatory funnel and the phases were separated. The aqueous phase was re-extracted with dichloromethane (3.886 moles; 330.0 g; 250.0 mL). The extracted organics are combined and water (12.49 moles; 225.0 g; 225.0 mL) was added followed by hydrochloric acid (37 WT % in water, 0.2359 equiv; 68.50 mmoles; 8.100 g; 6.750 mL). The phases were separated and to the organic phase was added water (13.88 moles; 250.0 g; 250.0 mL) followed by potassium carbonate (0.5608 equiv; 162.8 mmoles; 22.50 g). The phases were separated and the solvent of the organic phase is exchanged by adding tetrahydrofuran (9.153 moles; 660.0 g; 750.0 mL), and distilling the resulting mixture at a temperature of 55 to 75° C. (at 100 mm Hg). The distillation was continued until the pot temperature reached 66° C. and a final volume of 10 ml THF/g of benzyl 1,3,4,9-tetrahydro-2H-beta-carboline-2-carboxylate was reached.

Step 2: The THF solution of benzyl 1,3,4,9-tetrahydro-2H-beta-carboline-2-carboxylate was cooled from 66° C. to a temperature of 18 to 22° C. and water (12.49 moles; 225.0 g; 225.0 mL) was added followed by triethylamine (1.050 equiv; 304.8 mmoles; 30.85 g; 42.84 mL). The mixture was cooled from 20° C. to a temperature of −2 to 2° C. and N-chlorosuccinimide (1.080 equiv; 313.5 mmoles; 41.87 g) was added while keeping the temperature at −15 to 15° C. The reaction was allowed to stir for 0.5 to 1.5 hr at 18 to 22° C., and potassium carbonate was added (1.000 equiv; 290.3 mmoles; 40.12 g) until the pH measured 7.0. The resulting material was concentrated in vacuo. The solvent was exchanged by adding 2-methyltetrahydrofuran (7.489 moles; 645.0 g; 750.0 mL), and the resulting mixture was distilled until all the tetrahydrofuran was removed. Water (24.98 moles; 450.0 g; 450.0 mL) was added followed by potassium carbonate (1.246 equiv; 361.8 mmoles; 50.00 g). This mixture was added to a seperatory funnel and the phases were separated. The organic phase was transferred to a 3 L flask and water (52.73 moles; 950.0 g; 950.0 mL) was added followed by sodium chloride (855.6 mmoles; 50.00 g). After 25 to 35 min., this material was transferred to a seperatory funnel and the phases were separated. The organic layer was filtered and the filtrate was concentrated in vacuo. The solvent was exchanged by adding isopropyl ether, (99%, 1.762 moles; 180.0 g; 250.0 mL), and the resulting mixture was distilled at a temperature of 35 to 55° C. (at 100 mm Hg). The solution was cooled from 45° C. to a temperature of 15 to 25° C. and the mixture was held at this temperature for 6 to 18 hr, cooled from 20° C. to a temperature of −2.5 to 2.5° C. and the resulting suspension was filtered. The filter cake was washed with isopropyl ether, (99%, 704.7 mmoles; 72.00 g; 100.0 mL). The solid was collected and dried at 40 to 50° C. (at 10 mm Hg) for 6 to 18 hr to obtain benzyl 2-oxo-1,2-dihydro-1′H-spiro[indole-3,3′-pyrrolidine]-1′-carboxylate (275.8 mmoles; 88.91 g).

Step 3: In a 1 L RBF was added benzyl 2-oxo-1,2-dihydro-1′H-spiro[indole-3,3′-pyrrolidine]-1′-carboxylate (1.000 equiv [Limiting Reagent]; 155.1 mmoles; 50.00 g) followed by tetrahydrofuran (6.103 moles; 440.0 g; 500.0 mL) and sodium borohydride (5.000 equiv; 775.5 mmoles; 29.34 g). The reaction was cooled to −20 to −10° C. In a separate flask was added tetrahydrofuran (3.051 moles; 220.0 g; 250.0 mL) followed by iodine (2.000 equiv; 310.2 mmoles; 78.73 g). This solution was transferred to an addition funnel and added to the 1 L RBF over a period of 6.5 to 7.5 hr while maintaining the 1 L RBF at a temperature of −10 to 0° C. The reaction was heated from −15° C. to a temperature of 18 to 22° C. over a period of 10 to 14 hr and held for 4 to 8 hr at 18 to 22° C. Water (27.48 moles; 495.0 g; 500.0 mL) was added slowly followed by the slow addition of hydrochloric acid (37 wt % in water, 2.000 equiv; 310.2 mmoles; 36.68 g; 30.57 mL). This mixture was held for 25 to 35 min at 18 to 22° C. and was cooled from 20° C. to a temperature of −5 to 5° C. Tert-butyl methyl ether (2.939 moles; 259.0 g; 350.0 mL) was added followed by water (13.74 moles; 247.5 g; 250.0 mL) and sodium hydroxide (50% solution in water, 2.000 equiv; 310.2 mmoles; 37.47 g; 24.81 mL) to reach a pH of 9-10. The phases were separated and to the organic phase was added sodium sulfate (352.0 mmoles; 50.00 g) and the resulting suspension was agitated for 1.5 to 2 hr at 18 to 22° C. The suspension was filtered and the filter cake was washed with tert-butyl methyl ether (839.6 mmoles; 74.00 g; 100.0 mL). This material was concentrated in vacuo and ethyl acetate (5.051 moles; 445.0 g; 500.0 mL) was added followed by di-p-toluoyl-D-tartaric acid, (made from the unnatural enantiomer of tartaric acid) (97%, 1.000 equiv; 155.1 mmoles; 64.66 g). The mixture was held for 4 hr at 18 to 22° C. and then cooled to a temperature of −0.2 to 0.2° C. The suspension was filtered and then the filter cake was washed with ethyl acetate (1.010 moles; 89.00 g; 100.0 mL) while maintaining the filter at a temperature of −2.5 to 2.5° C. The solid was dried at 35 to 45° C. (at 100 mm Hg) for 8 to 16 hr to provide benzyl (3S)-1,2-dihydro-1′H-spiro[indole-3,3′-pyrrolidine]-1′-carboxylate (108.6 mmoles; 33.48 g).

Step 4: Benzyl (3S)-1,2-dihydro-1′H-spiro[indole-3,3′-pyrrolidine]-1′-carboxylate (1.1 g, 1.6 mmol) was dissolved in ethyl acetate (30 mL) and saturated aqueous sodium bicarbonate (30 mL) was added. The layers were separated and the aqueous layer was re-extracted with ethyl acetate (30 mL). The combined organics were washed with brine (30 mL) and dried over sodium sulfate. The mixture was filtered and then concentrated in vacuo to give the free base of benzyl (3S)-1,2-dihydro-1′H-spiro[indole-3,3′-pyrrolidine]-1′-carboxylate (350 mg, 1.14 mmol) as a colorless oil. An aliquot of this material (200 mg, 0.65 mmol) was combined with 4-chloro-1H-pyrrolo[2,3-b]pyridine and ethyl acetate (0.2 mL). The reaction was heated un-covered at 150° C. for 2.5 days. The reaction was cooled to room temperature and trifluoroacetic acid (0.5 mL) was added and the reaction was heated to 70° C. for 2 hours. The trifluoroacetic acid was removed in vacuo and the product was purified by prep-HPLC (0-20% Acetonitrile/water) isolating the title compound as the trifluoroacetate salt (22 mg, 0.076 mmol) as a light brown solid. ¹H NMR (400 MHz, CD₃OD) δ 8.07 (1H, d, J=6.8 Hz), 7.48-7.50 (2H, m), 7.40 (1H, d, J=3.6 Hz), 7.37 (1H, td, J=7.6, 0.8 Hz), 7.31 (1H, d, J=6.4 Hz), 7.19 (1H, t, J=7.6 Hz), 6.83 (1H, d, J=4.0 Hz), 4.57 (1H, d, J=10.4 Hz), 4.50 (1H, d, J=10.4 Hz), 3.61-3.71 (2H, m), 3.48-3.57 (2H, m), 2.36-2.44 (2H, m); LRMS (M+): 291.3; HPLC retention time: 0.55 min. (HPLC conditions 1).

### EXAMPLE 20

### Preparation of 1-(3-methyl-1H-pyrazolo[3,4-d]pyrimidin-4-yl)-1,2-dihydrospiro[indole-3,4′-piperidine]

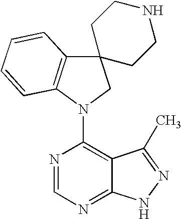

Step 1: Dimethylformamide (31.82 mL, 413 mmol) was added dropwise to phosphorus oxychloride (100 mL, 1.07 mol) at 0° C. To this mixture at 0° C. was added 4, 6-dihydroxypyrimidine (25 g, 223 mmol). The mixture was stirred at rt for 30 min and then at reflux for 2.5 h. The volatiles were removed in vacuo and the mixture was poured over ice water and extracted 6× with ether. The combined organics were washed with aqueous saturated sodium bicarbonate and dried over sodium sulfate to give 22.78 g (58%) of 4,6-dichloro-5-formylpyrimidine (J. Med. Chem. 2002, 45, 3639).

Step 2: A solution of 4,6-dichloro-5-formylpyrimidine (10.0 g, 56.5 mmol) in 100 mL of ether at 0° C. was treated with a solution of methyl magnesium bromide in ether (44.4 mL, 62.2 mmol, 1.4 M). The reaction was warmed to rt over 2 h period, filtered and the precipitate was taken up in a 20% saturated solution of ammonium chloride and water with cooling. The mixture was extracted 3× with ether. The combined organics were washed with brine and dried over sodium sulfate to give 9.5 g (87%) of 1-(4,6-dichloropyrimidin-5-yl)ethanol; HPLC T_{R} 3.62 min (HPLC conditions H).

Step 3: A solution of 1-(4,6-dichloropyrimidin-5-yl)ethanol (8.95 g, 46.4 mmol) in 140 mL of acetone was treated with chromium trioxide (9.27 g, 92.7 mmol). After stirring for 2.5 h, the mixture was quenched with 15 mL of isopropanol and stirred 15 min. The mixture was poured slowly into 500 mL of saturated sodium bicarbonate at 0° C. The mixture was extracted 3× with dichloromethane. The combined organics were dried with sodium sulfate to give 8.02 g of material. Chromatography on silica gel, eluting with 10% ethyl acetate/hexanes gave 7.62 g (86%) of 1-(4,6-dichloropyrimidin-5-yl)ethanone; HPLC T_{R} 4.92 min (HPLC Conditions H).

Step 4: A solution of 1-(4,6-dichloropyrimidin-5-yl)ethanone (3.81 g, 19.9 mmol) in 90 mL of dioxane at 0° C. was treated with triethylamine (2.78 mL, 19.9 mmol) and hydrazine hydrate (1.16 mL, 23.9 mmol). After addition, the reaction was stirred for 18 h at rt. The mixture was filtered and the precipitate washed with dioxane. The combined organics were evaporated and chromatographed on silica gel, eluting with 30-35% ethyl acetate/hexanes to afford 2.98 g, (89%) of 4-chloro-3-methyl-1H-pyrazolo[3,4-d]pyrimidine; ¹H NMR (500 MHz, DMSO-d₆) δ 8.71 (1H), 2.60 (3H) ppm.

Step 5: A mixture of 4-chloro-3-methyl-1H-pyrazolo[3,4-d]pyrimidine (1.30 g, 7.7 mmol) and benzyl 1,2-dihydro-1′H-spiro[indole-3,4′-piperidine]-1′-carboxylate (2.48 g, 7.7 mmol) in ethyl acetate was heated to 100° C., boiling off the ethyl acetate. The mixture was heated at 100° C. for 4 h and then cooled to rt. The mixture was taken up in 4:1 dichloromethane/methanol and 2M ammonia in methanol was added until neutral. The mixture was evaporated to dryness and chromatographed on silica gel, eluting with 60-90% ethyl acetate/hexanes to afford 1.86 g (53%) of benzyl 1-(3-methyl-1H-pyrazolo[3,4-d]pyrimidin-4-yl)-1,2-dihydro-1′H-spiro[indole-3,4′-piperidine]-1′-carboxylate; T_{R} 7.35 min (HPLC conditions H).

Step 6: A solution of benzyl 1-(3-methyl-1H-pyrazolo[3,4-d]pyrimidin-4-yl)-1,2-dihydro-1′H-spiro[indole-3,4′-piperidine]-1′-carboxylate (0.947 g, 2.16 mmol) in 4.3 mL of trifluoroacetic acid was heated at 70° C. for 1 h. The reaction mixture was quenched with 2 M ammonia in methanol and evaporated to dryness. The residue was chromatographed on silica gel (eluting with 5-7% methanol/0.5-0.75% ammonium hydroxide/dichloromethane to afford 0.577 g (87%) of the title compound; T_{R} 3.31 (HPLC conditions H), LRMS (M+320.3).

### EXAMPLE 21

### Preparation of 5-[4-(methylsulfonyl)phenyl]-1-(7H-pyrrolo[2,3-d]pyrimidin-4-yl)-1,2-dihydrospiro[indole-3,4′-piperidine]

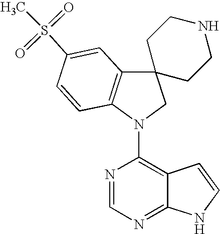

Step 1: 1-(4-(Methylsulfonyl)phenyl)hydrazine hydrochloride (2.30 g, 10.3 mmol) was slurried in dichloromethane (45 mL) in a dry flask under nitrogen. Benzyl 4-formylpiperidine-1-carboxylate (2.03 g, 10.3 mmol) was charged to the pot and cooled to 0° C. Trifluoroacetic acid (5 mL) was added to the flask over 15 minutes in a dropwise fashion and the pot was heated to 40° C. and held for 18 h. The reaction is checked by LC/MS and was complete. The solvent was concentrated down to 10% original volume. The slurry was diluted with 40 mL of toluene, acetonitrile (1 mL) and methanol (2 mL). The solution was cooled down to −5° C. and sodium borohydride (0.456 g, 12.1 mmol) was added portionwise over 30 minutes. The reaction is stirred for an additional hour at 0° C. The reaction was complete by LC/MS and was neutralized with 6% NH₄OH at 0° C. The organic was collected and aqueous phase was extracted with 2×40 mL ethyl acetate. The combined EtOAc phases were washed with brine, dried (Na₂SO₄), and concentrated to dryness. The material was chromatographed using 1:1 ethyl acetate—heptane to give 3.04 g of benzyl 5-[4-(methylsulfonyl)phenyl]-1,2-dihydro-1′H-spiro[indole-3,4′-piperidine]-1′-carboxylate as a tan solid. % yield=74% T_{R} 2.4 min. (HPLC conditions I); LRMS (M+): 401.2; ¹H NMR (400 MHz, DMSO d6) δ 7.28-7.36 (5H, m), 6.63 (1H, s), 6.50 (1H, d, J=8.3 Hz), 5.08 (2H, s), 3.95 (2H, d, J=12.9 Hz), 3.49 (1H, s), 3.02, (3H, s), 2.84-3.04 (2H, m), 1.70 (2H, t, J=13.1 Hz), 1.59 (2H, d, J=13.3 Hz).

Step 2: Benzyl 5-[4-(methylsulfonyl)phenyl]-1,2-dihydro-1′H-spiro[indole-3,4′-piperidine]-1′-carboxylate (361 mg, 0.90 mmol) was charged to a 2-dram vial and 4-chloro-1H-pyrazolo[3,4-d]pyrimidine (137.2 mg, 0.90 mmol) was added. The solids were well mixed and 0.210 mL of dimethylsulfoxide was charged to the reaction with 5 mg of p-toluenesulfonic acid monohydrate(catalytic) and the reaction was set up on shaker plate at 65° C. for 14 h. The reaction temperature was increased to 80° C. and run for an additional 90 minutes to complete the conversion to benzyl 5-(methylsulfonyl)-1-(7H-pyrrolo[2,3-d]pyrimidin-4-yl)-1,2-dihydro-1′H-spiro[indole-3,4′-piperidine]-1′-carboxylate. The reaction mixture was used without further purification. T_{R}=2.4 min. (HPLC conditions I); LRMS (M+): 518.2.

Step 3: The reaction mixture of benzyl 5-(methylsulfonyl)-1-(7H-pyrrolo[2,3-d]pyrimidin-4-yl)-1,2-dihydro-1′H-spiro[indole-3,4′-piperidine]-1′-carboxylate from above (II) is then treated with 2 mL of trifluoroacetic acid at 70° C. in a sealed 2 dram vial for 60 minutes. The reaction was complete by LC/MS. The volatiles were stripped off and the reaction is purified after diluting the reaction with 4 mL of dimethylsulfoxide and using preparative chromatography. (A Water's Xterra C18 reverse phase 30×50 mm column was used with a 5 μm pore size. The flow rate was 40 mL/min. and a linear column gradient of 10% acetonitrile/water to 40% acetonitrile, always with 0.1% formic acid present is used with a 8 minute total run time). 104 mg of the title compound as a white solid was recovered as the mono-trifluoroacetic acid salt (23% yield—two steps) T_{R} 1.0 min. (HPLC conditions I); LRMS (M+): 384.2; ¹H NMR (400 MHz, CD₃OD) δ 8.68 (1H, d, J=8.7 Hz), 8.42 (1H, s), 7.87 (1H, d, J=8.8 Hz), 7.80 (1H, s), 7.33 (1H, d, J=3.7 Hz), 6.94 (1H, d, J=3.3), 4.65, (2H, s), 3.49 (2H, d, J=13.3 Hz), 3.30-3.36 (2H, m), 3.12 (3H, s), 2.19 (2H, t), 2.08 (2H, d, J=14.5 Hz).

### EXAMPLES 22-640

The following compounds listed in Table 1 were prepared according to the Examples described above. Method of Preparation (Method of Prep.) in the second column identifies the example upon which the name compound was prepared.

TABLE 1

| Example | Method of | | Observed | HPLC | HPLC |
|---|---|---|---|---|---|
| No. | Prep. | IUPACNAME | Mass | RT | Method |
| 22 | 1 | 7-bromo-1-(7H-pyrrolo[2,3- | 384.2 | 2.29 | F |
| | | d]pyrimidin-4-yl)-1,2- | | | |
| | | dihydrospiro[indole-3,4′- | | | |
| | | piperidine] | | | |
| 23 | 1 | 5-bromo-1-(7H-pyrrolo[2,3- | 384.2 | 1.88 | F |
| | | d]pyrimidin-4-yl)-1,2- | | | |
| | | dihydrospiro[indole-3,4′- | | | |
| | | piperidine] | | | |
| 24 | 1, 4 | 7-bromo-1-(5-chloro-7H- | 418.16 | 2.59 | F |
| | | pyrrolo[2,3-d]pyrimidin-4-yl)- | | | |
| | | 1,2-dihydrospiro[indole-3,4′- | | | |
| | | piperidine] | | | |
| 25 | 1, 4 | 5-bromo-1-(5-chloro-7H- | 418.16 | 2.16 | F |
| | | pyrrolo[2,3-d]pyrimidin-4-yl)- | | | |
| | | 1,2-dihydrospiro[indole-3,4′- | | | |
| | | piperidine] | | | |
| 26 | 1 | 1-(7H-pyrrolo[2,3-d]pyrimidin- | 306 | 2.4 | J |
| | | 4-yl)-1,2-dihydrospiro[indole- | | | |
| | | 3,4′-piperidine] | | | |
| 27 | 1 | 5-methoxy-1-(7H-pyrrolo[2,3- | 336.3 | 1 | I |
| | | d]pyrimidin-4-yl)-1,2- | | | |
| | | dihydrospiro[indole-3,4′- | | | |
| | | piperidine] | | | |
| 28 | 1 | 1-(7H-pyrrolo[2,3-d]pyrimidin- | 331.3 | 1 | I |
| | | 4-yl)-1,2-dihydrospiro[indole- | | | |
| | | 3,4′-piperidine]-5-carbonitrile | | | |
| 29 | 1 | 1-(7H-pyrrolo[2,3-d]pyrimidin- | 374.1 | 1.6 | I |
| | | 4-yl)-5-(trifluoromethyl)-1,2- | | | |
| | | dihydrospiro[indole-3,4′- | | | |
| | | piperidine] | | | |
| 30 | 1 | 5-methyl-1-(7H-pyrrolo[2,3- | 320.13 | 3.08 | D |
| | | d]pyrimidin-4-yl)-1,2- | | | |
| | | dihydrospiro[indole-3,4′- | | | |
| | | piperidine] | | | |
| 31 | 1, 4 | 1-(5-chloro-7H-pyrrolo[2,3- | 368.1 | 3.7 | D |
| | | d]pyrimidin-4-yl)-5,7-dimethyl- | | | |
| | | 1,2-dihydrospiro[indole-3,4′- | | | |
| | | piperidine] | | | |
| 32 | 1 | 5-fluoro-1-(7H-pyrrolo[2,3- | 324.08 | 2.99 | D |
| | | d]pyrimidin-4-yl)-1,2- | | | |
| | | dihydrospiro[indole-3,4′- | | | |
| | | piperidine] | | | |
| 33 | 1 | 5,7-dimethyl-1-(7H-pyrrolo[2,3- | 334.14 | 3.2 | D |
| | | d]pyrimidin-4-yl)-1,2- | | | |
| | | dihydrospiro[indole-3,4′- | | | |
| | | piperidine] | | | |
| 34 | 1, 4 | 1-(5-chloro-7H-pyrrolo[2,3- | 354.08 | 3.73 | D |
| | | d]pyrimidin-4-yl)-5-methyl-1,2- | | | |
| | | dihydrospiro[indole-3,4′- | | | |
| | | piperidine] | | | |
| 35 | 1, 4 | 1-(5-chloro-7H-pyrrolo[2,3- | 358.03 | 3.73 | D |
| | | d]pyrimidin-4-yl)-5-fluoro-1,2- | | | |
| | | dihydrospiro[indole-3,4′- | | | |
| | | piperidine] | | | |
| 36 | 1, 4 | 1-(5-chloro-7H-pyrrolo[2,3- | 340.07 | 3.49 | D |
| | | d]pyrimidin-4-yl)-1,2- | | | |
| | | dihydrospiro[indole-3,4′- | | | |
| | | piperidine] | | | |
| 37 | 2 | 5-fluoro-1-(5-methyl-7H- | 337.3 | 4.05 | H |
| | | pyrrolo[2,3-d]pyrimidin-4-yl)- | | | |
| | | 1,2-dihydrospiro[indole-3,4′- | | | |
| | | piperidine] | | | |
| 38 | 2 | 5-methyl-1-(5-methyl-7H- | 333.2 | 4.22 | H |
| | | pyrrolo[2,3-d]pyrimidin-4-yl)- | | | |
| | | 1,2-dihydrospiro[indole-3,4′- | | | |
| | | piperidine] | | | |
| 39 | 1 | 5-isopropyl-1-(7H-pyrrolo[2,3- | 348.3 | 2.43 | J |
| | | d]pyrimidin-4-yl)-1,2- | | | |
| | | dihydrospiro[indole-3,4′- | | | |
| | | piperidine] | | | |
| 40 | 1 | 1-(7H-pyrrolo[2,3-d]pyrimidin- | 374.2 | 2.24 | J |
| | | 4-yl)-6-(trifluoromethyl)-1,2- | | | |
| | | dihydrospiro[indole-3,4′- | | | |
| | | piperidine] | | | |
| 41 | 1 | 1-(7H-pyrrolo[2,3-d]pyrimidin- | 374.2 | 2.31 | J |
| | | 4-yl)-4-(trifluoromethyl)-1,2- | | | |
| | | dihydrospiro[indole-3,4′- | | | |
| | | piperidine] | | | |
| 42 | 1 | 6-chloro-1-(7H-pyrrolo[2,3- | 340.2 | 2.31 | J |
| | | d]pyrimidin-4-yl)-1,2- | | | |
| | | dihydrospiro[indole-3,4′- | | | |
| | | piperidine] | | | |
| 43 | 1 | 4-chloro-1-(7H-pyrrolo[2,3- | 340.2 | 2.19 | J |
| | | d]pyrimidin-4-yl)-1,2- | | | |
| | | dihydrospiro[indole-3,4′- | | | |
| | | piperidine] | | | |
| 44 | 1 | 4-methyl-1-(7H-pyrrolo[2,3- | 0.94 | 320.3 | I |
| | | d]pyrimidin-4-yl)-1,2- | | | |
| | | dihydrospiro[indole-3,4′- | | | |
| | | piperidine] | | | |
| 45 | 1 | 6-methyl-1-(7H-pyrrolo[2,3- | 0.94 | 320.3 | I |
| | | d]pyrimidin-4-yl)-1,2- | | | |
| | | dihydrospiro[indole-3,4′- | | | |
| | | piperidine] | | | |
| 46 | 1 | 4,5-dimethyl-1-(7H-pyrrolo[2,3- | 1.23 | 334.1 | I |
| | | d]pyrimidin-4-yl)-1,2- | | | |
| | | dihydrospiro[indole-3,4′- | | | |
| | | piperidine] | | | |
| 47 | 1 | 5,6-dimethyl-1-(7H-pyrrolo[2,3- | 1.23 | 334.1 | I |
| | | d]pyrimidin-4-yl)-1,2- | | | |
| | | dihydrospiro[indole-3,4′- | | | |
| | | piperidine] | | | |
| 48 | 1 | 4-chloro-5-methyl-1-(7H- | 1.4 | 354 | I |
| | | pyrrolo[2,3-d]pyrimidin-4-yl)- | | | |
| | | 1,2-dihydrospiro[indole-3,4′- | | | |
| | | piperidine] | | | |
| 49 | 1 | 6-chloro-5-methyl-1-(7H- | 1.56 | 354 | I |
| | | pyrrolo[2,3-d]pyrimidin-4-yl)- | | | |
| | | 1,2-dihydrospiro[indole-3,4′- | | | |
| | | piperidine] | | | |
| 50 | 1 | 4,5-dichloro-1-(7H-pyrrolo[2,3- | 1.52 | 374 | I |
| | | d]pyrimidin-4-yl)-1,2- | | | |
| | | dihydrospiro[indole-3,4′- | | | |
| | | piperidine] | | | |
| 51 | 1 | 5,6-dichloro-1-(7H-pyrrolo[2,3- | 1.67 | 372 | I |
| | | d]pyrimidin-4-yl)-1,2- | | | |
| | | dihydrospiro[indole-3,4′- | | | |
| | | piperidine] | | | |
| 52 | 1 | 4-fluoro-1-(7H-pyrrolo[2,3- | 1.06 | 324.1 | I |
| | | d]pyrimidin-4-yl)-1,2- | | | |
| | | dihydrospiro[indole-3,4′- | | | |
| | | piperidine] | | | |
| 53 | 1, 8 | 2-methyl-1-(7H-pyrrolo[2,3- | 1 | 320.3 | I |
| | | d]pyrimidin-4-yl)-1,2- | | | |
| | | dihydrospiro[indole-3,4′- | | | |
| | | piperidine] | | | |
| 54 | 1 | 5-phenoxy-1-(7H-pyrrolo[2,3- | 1.73 | 398.3 | I |
| | | d]pyrimidin-4-yl)-1,2- | | | |
| | | dihydrospiro[indole-3,4′- | | | |
| | | piperidine] | | | |
| 55 | 1 | 3-(7H-pyrrolo[2,3-d]pyrimidin- | 1.2 | 356.3 | I |
| | | 4-yl)-2,3- | | | |
| | | dihydrospiro[benzo[e]indole- | | | |
| | | 1,4′-piperidine] | | | |
| 56 | 1, 9 | N-(2-methoxyethyl)-1-(7H- | 407.3 | 0.88 | I |
| | | pyrrolo[2,3-d]pyrimidin-4-yl)- | | | |
| | | 1,2-dihydrospiro[indole-3,4′- | | | |
| | | piperidine]-5-carboxamide | | | |
| 57 | 1, 9 | N-phenyl-1-(7H-pyrrolo[2,3- | 425.3 | 1.31 | I |
| | | d]pyrimidin-4-yl)-1,2- | | | |
| | | dihydrospiro[indole-3,4′- | | | |
| | | piperidine]-5-carboxamide | | | |
| 58 | 1, 9 | N-(2,2-dimethylpropyl)-1-(7H- | 419.4 | 1.6 | I |
| | | pyrrolo[2,3-d]pyrimidin-4-yl)- | | | |
| | | 1,2-dihydrospiro[indole-3,4′- | | | |
| | | piperidine]-5-carboxamide | | | |
| 59 | 1 | 1-(7H-pyrrolo[2,3-d]pyrimidin- | 350.3 | 0.9 | I |
| | | 4-yl)-1,2-dihydrospiro[indole- | | | |
| | | 3,4′-piperidine]-5-carboxylic | | | |
| | | acid | | | |
| 60 | 1, 9 | 5-(morpholin-4-ylcarbonyl)-1- | 419.3 | 0.65 | I |
| | | (7H-pyrrolo[2,3-d]pyrimidin-4- | | | |
| | | yl)-1,2-dihydrospiro[indole-3,4′- | | | |
| | | piperidine] | | | |
| 61 | 1, 9 | N-cyclopropyl-1-(7H- | 389.3 | 0.84 | I |
| | | pyrrolo[2,3-d]pyrimidin-4-yl)- | | | |
| | | 1,2-dihydrospiro[indole-3,4′- | | | |
| | | piperidine]-5-carboxamide | | | |
| 62 | 1, 9 | N-methyl-1-(7H-pyrrolo[2,3- | 363.3 | 0.66 | I |
| | | d]pyrimidin-4-yl)-1,2- | | | |
| | | dihydrospiro[indole-3,4′- | | | |
| | | piperidine]-5-carboxamide | | | |
| 63 | 1, 7 | N-phenyl-1-(7H-pyrrolo[2,3- | 397.4 | 1.52 | I |
| | | d]pyrimidin-4-yl)-1,2- | | | |
| | | dihydrospiro[indole-3,4′- | | | |
| | | piperidin]-5-amine | | | |
| 64 | 1 | 6,8-dichloro-3-(7H-pyrrolo[2,3- | 424.3 | 1.98 | I |
| | | d]pyrimidin-4-yl)-2,3- | | | |
| | | dihydrospiro[benzo[e]indole- | | | |
| | | 1,4′-piperidine] | | | |
| 65 | 1 | 6,9-dichloro-3-(7H-pyrrolo[2,3- | 424.1 | 1.84 | I |
| | | d]pyrimidin-4-yl)-2,3- | | | |
| | | dihydrospiro[benzo[e]indole- | | | |
| | | 1,4′-piperidine] | | | |
| 66 | 1 | 6-(7H-pyrrolo[2,3-d]pyrimidin- | 346.3 | 0.71 | I |
| | | 4-yl)-6,7-dihydro-3H- | | | |
| | | spiro[imidazo[4,5-e]indole-8,4′- | | | |
| | | piperidine] | | | |
| 67 | 1 | 5-(methylsulfonyl)-1-(7H- | 384.2 | 0.9 | I |
| | | pyrrolo[2,3-d]pyrimidin-4-yl)- | | | |
| | | 1,2-dihydrospiro[indole-3,4′- | | | |
| | | piperidine] | | | |
| 68 | 1, 7 | N-benzyl-1-(7H-pyrrolo[2,3- | 411.4 | 1.46 | I |
| | | d]pyrimidin-4-yl)-1,2- | | | |
| | | dihydrospiro[indole-3,4′- | | | |
| | | piperidin]-5-amine | | | |
| 69 | 1, 7 | 5-(4-methylpiperazin-1-yl)-1- | 404.4 | 0.54 | I |
| | | (7H-pyrrolo[2,3-d]pyrimidin-4- | | | |
| | | yl)-1,2-dihydrospiro[indole-3,4′- | | | |
| | | piperidine] | | | |
| 70 | 1, 7 | N-cyclobutyl-1-(7H-pyrrolo[2,3- | 375.2 | 0.98 | I |
| | | d]pyrimidin-4-yl)-1,2- | | | |
| | | dihydrospiro[indole-3,4′- | | | |
| | | piperidin]-5-amine | | | |
| 71 | 1, 7 | N-(2-methoxyethyl)-1-(7H- | 379.2 | 0.81 | I |
| | | pyrrolo[2,3-d]pyrimidin-4-yl)- | | | |
| | | 1,2-dihydrospiro[indole-3,4′- | | | |
| | | piperidin]-5-amine | | | |
| 72 | 1, 7 | 5-morpholin-4-yl-1-(7H- | 391.3 | 1.2 | I |
| | | pyrrolo[2,3-d]pyrimidin-4-yl)- | | | |
| | | 1,2-dihydrospiro[indole-3,4′- | | | |
| | | piperidine] | | | |
| 73 | 1, 7 | 5-piperidin-1-yl-1-(7H- | 389.4 | 0.79 | I |
| | | pyrrolo[2,3-d]pyrimidin-4-yl)- | | | |
| | | 1,2-dihydrospiro[indole-3,4′- | | | |
| | | piperidine] | | | |
| 74 | 1, 7 | N-(2,2-dimethylpropyl)-1-(7H- | 391.4 | 1.53 | I |
| | | pyrrolo[2,3-d]pyrimidin-4-yl)- | | | |
| | | 1,2-dihydrospiro[indole-3,4′- | | | |
| | | piperidin]-5-amine | | | |
| 75 | 1, 7 | 5-(3,4-dihydroquinolin-1(2H)- | 437.2 | 2.1 | I |
| | | yl)-1-(7H-pyrrolo[2,3- | | | |
| | | d]pyrimidin-4-yl)-1,2- | | | |
| | | dihydrospiro[indole-3,4′- | | | |
| | | piperidine] | | | |
| 76 | 1, 7 | N-(4-chlorophenyl)-1-(7H- | 431.2 | 1.75 | I |
| | | pyrrolo[2,3-d]pyrimidin-4-yl)- | | | |
| | | 1,2-dihydrospiro[indole-3,4′- | | | |
| | | piperidin]-5-amine | | | |
| 77 | 1, 7 | N-(3-methylphenyl)-1-(7H- | 411.2 | 1.61 | I |
| | | pyrrolo[2,3-d]pyrimidin-4-yl)- | | | |
| | | 1,2-dihydrospiro[indole-3,4′- | | | |
| | | piperidin]-5-amine | | | |
| 78 | 1, 7 | N-(3-chlorophenyl)-1-(7H- | 431.2 | 1.74 | I |
| | | pyrrolo[2,3-d]pyrimidin-4-yl)- | | | |
| | | 1,2-dihydrospiro[indole-3,4′- | | | |
| | | piperidin]-5-amine | | | |
| 79 | 1, 7 | N-(1-phenylethyl)-1-(7H- | 425.1 | 1.19 | I |
| | | pyrrolo[2,3-d]pyrimidin-4-yl)- | | | |
| | | 1,2-dihydrospiro[indole-3,4′- | | | |
| | | piperidin]-5-amine | | | |
| 80 | 1, 7 | N-(2-phenylethyl)-1-(7H- | 425.2 | 1.12 | I |
| | | pyrrolo[2,3-d]pyrimidin-4-yl)- | | | |
| | | 1,2-dihydrospiro[indole-3,4′- | | | |
| | | piperidin]-5-amine | | | |
| 81 | 1, 6 | 1-(7H-pyrrolo[2,3-d]pyrimidin- | 388.3 | 1.8 | I |
| | | 4-yl)-5-(2-thienyl)-1,2- | | | |
| | | dihydrospiro[indole-3,4′- | | | |
| | | piperidine] | | | |
| 82 | 1, 6 | 1-(7H-pyrrolo[2,3-d]pyrimidin- | 388.3 | 2 | I |
| | | 4-yl)-5-(3-thienyl)-1,2- | | | |
| | | dihydrospiro[indole-3,4′- | | | |
| | | piperidine] | | | |
| 83 | 1, 6 | 5-(3-furyl)-1-(7H-pyrrolo[2,3- | 372.3 | 1.8 | I |
| | | d]pyrimidin-4-yl)-1,2- | | | |
| | | dihydrospiro[indole-3,4′- | | | |
| | | piperidine] | | | |
| 84 | 1, 6 | 5-phenyl-1-(7H-pyrrolo[2,3- | 382.3 | 1.7 | I |
| | | d]pyrimidin-4-yl)-1,2- | | | |
| | | dihydrospiro[indole-3,4′- | | | |
| | | piperidine] | | | |
| 85 | 1, 6 | 5-pyridin-4-yl-1-(7H- | 383.3 | 0.9 | I |
| | | pyrrolo[2,3-d]pyrimidin-4-yl)- | | | |
| | | 1,2-dihydrospiro[indole-3,4′- | | | |
| | | piperidine] | | | |
| 86 | 3 | 4-(5-fluorospiro[indole-3,4′- | 348.2 | 4.1 | H |
| | | piperidin]-1(2H)-yl)-7H- | | | |
| | | pyrrolo[2,3-d]pyrimidine-5- | | | |
| | | carbonitrile | | | |
| 87 | 3 | 4-(5-chlorospiro[indole-3,4′- | 364 | 4.47 | H |
| | | piperidin]-1(2H)-yl)-7H- | | | |
| | | pyrrolo[2,3-d]pyrimidine-5- | | | |
| | | carbonitrile | | | |
| 88 | 1 | 4-nitro-1-(7H-pyrrolo[2,3- | 351.2 | 1.2 | I |
| | | d]pyrimidin-4-yl)-1,2- | | | |
| | | dihydrospiro[indole-3,4′- | | | |
| | | piperidine] | | | |
| 89 | 1 | 1-(7H-pyrrolo[2,3-d]pyrimidin- | 385.1 | 0.74 | I |
| | | 4-yl)-1,2-dihydrospiro[indole- | | | |
| | | 3,4′-piperidine]-5-sulfonamide | | | |
| 90 | 1 | 1-(7H-pyrrolo[2,3-d]pyrimidin- | 331.2 | 1.23 | I |
| | | 4-yl)-1,2-dihydrospiro[indole- | | | |
| | | 3,4′-piperidine]-4-carbonitrile | | | |
| 91 | 1 | 5-(3-methylphenyl)-1-(7H- | 396.4 | 2 | I |
| | | pyrrolo[2,3-d]pyrimidin-4-yl)- | | | |
| | | 1,2-dihydrospiro[indole-3,4′- | | | |
| | | piperidine] | | | |
| 92 | 1 | 5-[2-(benzyloxy)phenyl]-1-(7H- | 488.4 | 2.3 | I |
| | | pyrrolo[2,3-d]pyrimidin-4-yl)- | | | |
| | | 1,2-dihydrospiro[indole-3,4′- | | | |
| | | piperidine] | | | |
| 93 | 1, 6 | 5-(3-isopropylphenyl)-1-(7H- | 424.5 | 2.2 | I |
| | | pyrrolo[2,3-d]pyrimidin-4-yl)- | | | |
| | | 1,2-dihydrospiro[indole-3,4′- | | | |
| | | piperidine] | | | |
| 94 | 1, 6 | N,N-dimethyl-4-[1-(7H- | 425.4 | 1.7 | I |
| | | pyrrolo[2,3-d]pyrimidin-4-yl)- | | | |
| | | 1,2-dihydrospiro[indole-3,4′- | | | |
| | | piperidin]-5-yl]aniline | | | |
| 95 | 1, 6 | 5-biphenyl-2-yl-1-(7H- | 458.5 | 2.2 | I |
| | | pyrrolo[2,3-d]pyrimidin-4-yl)- | | | |
| | | 1,2-dihydrospiro[indole-3,4′- | | | |
| | | piperidine] | | | |
| 96 | 1, 6 | 5-(4-isobutylphenyl)-1-(7H- | 438.5 | 2.5 | I |
| | | pyrrolo[2,3-d]pyrimidin-4-yl)- | | | |
| | | 1,2-dihydrospiro[indole-3,4′- | | | |
| | | piperidine] | | | |
| 97 | 1, 6 | 5-(4-isopropylphenyl)-1-(7H- | 424.5 | 2.3 | I |
| | | pyrrolo[2,3-d]pyrimidin-4-yl)- | | | |
| | | 1,2-dihydrospiro[indole-3,4′- | | | |
| | | piperidine] | | | |
| 98 | 1, 6 | 5-(1-benzothien-3-yl)-1-(7H- | 438.4 | 2.1 | I |
| | | pyrrolo[2,3-d]pyrimidin-4-yl)- | | | |
| | | 1,2-dihydrospiro[indole-3,4′- | | | |
| | | piperidine] | | | |
| 99 | 1, 6 | 5-(3-methoxyphenyl)-1-(7H- | 412.4 | 1.9 | I |
| | | pyrrolo[2,3-d]pyrimidin-4-yl)- | | | |
| | | 1,2-dihydrospiro[indole-3,4′- | | | |
| | | piperidine] | | | |
| 100 | 1, 6 | 5-(1-naphthyl)-1-(7H- | 432.2 | 2.1 | I |
| | | pyrrolo[2,3-d]pyrimidin-4-yl)- | | | |
| | | 1,2-dihydrospiro[indole-3,4′- | | | |
| | | piperidine] | | | |
| 101 | 1, 6 | 5-[3-(3,5-dimethyl-1H-pyrazol- | 476.5 | 2.1 | I |
| | | 1-yl)phenyl]-1-(7H-pyrrolo[2,3- | | | |
| | | d]pyrimidin-4-yl)-1,2- | | | |
| | | dihydrospiro[indole-3,4′- | | | |
| | | piperidine] | | | |
| 102 | 1, 6 | 4-[1-(7H-pyrrolo[2,3- | 407.4 | 1.9 | I |
| | | d]pyrimidin-4-yl)-1,2- | | | |
| | | dihydrospiro[indole-3,4′- | | | |
| | | piperidin]-5-yl]benzonitrile | | | |
| 103 | 1, 6 | 5-[3-(1H-pyrazol-1-yl)phenyl]- | 497.4 | 2.4 | I |
| | | 1-(7H-pyrrolo[2,3-d]pyrimidin- | | | |
| | | 4-yl)-1,2-dihydrospiro[indole- | | | |
| | | 3,4′-piperidine] | | | |
| 104 | 1, 6 | 5-(2,3-dihydro-1,4- | 440.4 | 1.9 | I |
| | | benzodioxin-6-yl)-1-(7H- | | | |
| | | pyrrolo[2,3-d]pyrimidin-4-yl)- | | | |
| | | 1,2-dihydrospiro[indole-3,4′- | | | |
| | | piperidine] | | | |
| 105 | 1, 6 | N-{4-[1-(7H-pyrrolo[2,3- | 439.4 | 1.7 | I |
| | | d]pyrimidin-4-yl)-1,2- | | | |
| | | dihydrospiro[indole-3,4′- | | | |
| | | piperidin]-5- | | | |
| | | yl]phenyl}acetamide | | | |
| 106 | 1, 6 | 3-[1-(7H-pyrrolo[2,3- | 407.4 | 1.9 | I |
| | | d]pyrimidin-4-yl)-1,2- | | | |
| | | dihydrospiro[indole-3,4′- | | | |
| | | piperidin]-5-yl]benzonitrile | | | |
| 107 | 1, 6 | 5-biphenyl-4-yl-1-(7H- | 458.5 | 2.4 | I |
| | | pyrrolo[2,3-d]pyrimidin-4-yl)- | | | |
| | | 1,2-dihydrospiro[indole-3,4′- | | | |
| | | piperidine] | | | |
| 108 | 1, 6 | 5-(1,3-benzodioxol-5-yl)-1- | 426.4 | 1.9 | I |
| | | (7H-pyrrolo[2,3-d]pyrimidin-4- | | | |
| | | yl)-1,2-dihydrospiro[indole-3,4′- | | | |
| | | piperidine] | | | |
| 109 | 1, 6 | 5-(2-phenoxyphenyl)-1-(7H- | 474.5 | 2.3 | I |
| | | pyrrolo[2,3-d]pyrimidin-4-yl)- | | | |
| | | 1,2-dihydrospiro[indole-3,4′- | | | |
| | | piperidine] | | | |
| 110 | 1, 6 | 5-(2-naphthyl)-1-(7H- | 432.5 | 2.3 | I |
| | | pyrrolo[2,3-d]pyrimidin-4-yl)- | | | |
| | | 1,2-dihydrospiro[indole-3,4′- | | | |
| | | piperidine] | | | |
| 111 | 1, 6 | 5-(4-methoxyphenyl)-1-(7H- | 412.4 | 1.9 | I |
| | | pyrrolo[2,3-d]pyrimidin-4-yl)- | | | |
| | | 1,2-dihydrospiro[indole-3,4′- | | | |
| | | piperidine] | | | |
| 112 | 1, 6 | 5-(3,5-dimethylisoxazol-4-yl)- | 401.5 | 1.7 | I |
| | | 1-(7H-pyrrolo[2,3-d]pyrimidin- | | | |
| | | 4-yl)-1,2-dihydrospiro[indole- | | | |
| | | 3,4′-piperidine] | | | |
| 113 | 1, 6 | 5-(1-benzofuran-2-yl)-1-(7H- | 422.4 | 2.2 | I |
| | | pyrrolo[2,3-d]pyrimidin-4-yl)- | | | |
| | | 1,2-dihydrospiro[indole-3,4′- | | | |
| | | piperidine] | | | |
| 114 | 1, 6 | 1-(7H-pyrrolo[2,3-d]pyrimidin- | 433.4 | 1.5 | I |
| | | 4-yl)-5-quinolin-5-yl-1,2- | | | |
| | | dihydrospiro[indole-3,4′- | | | |
| | | piperidine] | | | |
| 115 | 1, 6 | 2-[1-(7H-pyrrolo[2,3- | 461.2 | 1.3 | I |
| | | d]pyrimidin-4-yl)-1,2- | | | |
| | | dihydrospiro[indole-3,4′- | | | |
| | | piperidin]-5- | | | |
| | | yl]benzenesulfonamide | | | |
| 116 | 1, 6 | 5-pyrimidin-5-yl-1-(7H- | 384.4 | 1.3 | I |
| | | pyrrolo[2,3-d]pyrimidin-4-yl)- | | | |
| | | 1,2-dihydrospiro[indole-3,4′- | | | |
| | | piperidine] | | | |
| 117 | 1, 6 | 5-(5-methyl-2-furyl)-1-(7H- | 386.3 | 1.6 | I |
| | | pyrrolo[2,3-d]pyrimidin-4-yl)- | | | |
| | | 1,2-dihydrospiro[indole-3,4′- | | | |
| | | piperidine] | | | |
| 118 | 1, 6 | 5-(1-benzothien-2-yl)-1-(7H- | 438.4 | 2.3 | I |
| | | pyrrolo[2,3-d]pyrimidin-4-yl)- | | | |
| | | 1,2-dihydrospiro[indole-3,4′- | | | |
| | | piperidine] | | | |
| 119 | 1, 6 | 1-(7H-pyrrolo[2,3-d]pyrimidin- | 386.3 | 1.6 | I |
| | | 4-yl)-5-quinolin-8-yl-1,2- | | | |
| | | dihydrospiro[indole-3,4′- | | | |
| | | piperidine] | | | |
| 120 | 1, 6 | 5-(4-tert-butylphenyl)-1-(7H- | 438.5 | 2.4 | I |
| | | pyrrolo[2,3-d]pyrimidin-4-yl)- | | | |
| | | 1,2-dihydrospiro[indole-3,4′- | | | |
| | | piperidine] | | | |
| 121 | 1, 6 | 5-(2-methylphenyl)-1-(7H- | 396.4 | 2 | I |
| | | pyrrolo[2,3-d]pyrimidin-4-yl)- | | | |
| | | 1,2-dihydrospiro[indole-3,4′- | | | |
| | | piperidine] | | | |
| 122 | 1, 6 | 5-[3-(benzyloxy)phenyl]-1-(7H- | 488.5 | 2.3 | I |
| | | pyrrolo[2,3-d]pyrimidin-4-yl)- | | | |
| | | 1,2-dihydrospiro[indole-3,4′- | | | |
| | | piperidine] | | | |
| 123 | 1, 6 | 5-(6-methoxypyridin-3-yl)-1- | 413.4 | 1.8 | I |
| | | (7H-pyrrolo[2,3-d]pyrimidin-4- | | | |
| | | yl)-1,2-dihydrospiro[indole-3,4′- | | | |
| | | piperidine] | | | |
| 124 | 1, 6 | N-{3-[1-(7H-pyrrolo[2,3- | 439.4 | 1.8 | I |
| | | d]pyrimidin-4-yl)-1,2- | | | |
| | | dihydrospiro[indole-3,4′- | | | |
| | | piperidin]-5- | | | |
| | | yl]phenyl}acetamide | | | |
| 125 | 1, 6 | N-{2-[1-(7H-pyrrolo[2,3- | 475.4 | 1.7 | I |
| | | d]pyrimidin-4-yl)-1,2- | | | |
| | | dihydrospiro[indole-3,4′- | | | |
| | | piperidin]-5- | | | |
| | | yl]phenyl}methanesulfonamide | | | |
| 126 | 1, 6 | 2-[1-(7H-pyrrolo[2,3- | 407.4 | 2 | I |
| | | d]pyrimidin-4-yl)-1,2- | | | |
| | | dihydrospiro[indole-3,4′- | | | |
| | | piperidin]-5-yl]benzonitrile | | | |
| 127 | 1, 7 | N-(2-fluorobenzyl)-1-(7H- | 429.32 | 4.276 | K |
| | | pyrrolo[2,3-d]pyrimidin-4-yl)- | | | |
| | | 1,2-dihydrospiro[indole-3,4′- | | | |
| | | piperidin]-5-amine | | | |
| 128 | 1, 7 | N-(2-methoxybenzyl)-1-(7H- | 441.35 | 4.27 | K |
| | | pyrrolo[2,3-d]pyrimidin-4-yl)- | | | |
| | | 1,2-dihydrospiro[indole-3,4′- | | | |
| | | piperidin]-5-amine | | | |
| 129 | 1, 7 | N-(4-chlorobenzyl)-1-(7H- | 445.28 | 4.57 | K |
| | | pyrrolo[2,3-d]pyrimidin-4-yl)- | | | |
| | | 1,2-dihydrospiro[indole-3,4′- | | | |
| | | piperidin]-5-amine | | | |
| 130 | 1, 7 | N-(4-fluorobenzyl)-1-(7H- | 429.32 | 4.26 | K |
| | | pyrrolo[2,3-d]pyrimidin-4-yl)- | | | |
| | | 1,2-dihydrospiro[indole-3,4′- | | | |
| | | piperidin]-5-amine | | | |
| 131 | 1, 7 | N,N′-dimethyl-N-[1-(7H- | 392.34 | 4.47 | K |
| | | pyrrolo[2,3-d]pyrimidin-4-yl)- | | | |
| | | 1,2-dihydrospiro[indole-3,4′- | | | |
| | | piperidin]-5-yl]ethane-1,2- | | | |
| | | diamine | | | |
| 132 | 1, 7 | N-(biphenyl-2-ylmethyl)-1-(7H- | 487.37 | 4.82 | K |
| | | pyrrolo[2,3-d]pyrimidin-4-yl)- | | | |
| | | 1,2-dihydrospiro[indole-3,4′- | | | |
| | | piperidin]-5-amine | | | |
| 133 | 1, 7 | N-[2-(methylthio)benzyl]-1- | 457.34 | 4.46 | K |
| | | (7H-pyrrolo[2,3-d]pyrimidin-4- | | | |
| | | yl)-1,2-dihydrospiro[indole-3,4′- | | | |
| | | piperidin]-5-amine | | | |
| 134 | 1, 7 | N-(4-methylphenyl)-1-(7H- | 411.34 | 4.48 | K |
| | | pyrrolo[2,3-d]pyrimidin-4-yl)- | | | |
| | | 1,2-dihydrospiro[indole-3,4′- | | | |
| | | piperidin]-5-amine | | | |
| 135 | 1, 7 | N-[4-(1H-pyrazol-1-yl)benzyl]- | 477.2 | 1.6 | I |
| | | 1-(7H-pyrrolo[2,3-d]pyrimidin- | | | |
| | | 4-yl)-1,2-dihydrospiro[indole- | | | |
| | | 3,4′-piperidin]-5-amine | | | |
| 136 | 1, 7 | N-(2-chlorobenzyl)-1-(7H- | 445.4 | 1.9 | I |
| | | pyrrolo[2,3-d]pyrimidin-4-yl)- | | | |
| | | 1,2-dihydrospiro[indole-3,4′- | | | |
| | | piperidin]-5-amine | | | |
| 137 | 1, 7 | N-[3-(1H-pyrazol-1-yl)benzyl]- | 477.5 | 1.5 | I |
| | | 1-(7H-pyrrolo[2,3-d]pyrimidin- | | | |
| | | 4-yl)-1,2-dihydrospiro[indole- | | | |
| | | 3,4′-piperidin]-5-amine | | | |
| 138 | 1, 7 | N-(2-methylbenzyl)-1-(7H- | 425.5 | 1.7 | I |
| | | pyrrolo[2,3-d]pyrimidin-4-yl)- | | | |
| | | 1,2-dihydrospiro[indole-3,4′- | | | |
| | | piperidin]-5-amine | | | |
| 139 | 1, 7 | 1-(7H-pyrrolo[2,3-d]pyrimidin- | 495.4 | 2 | I |
| | | 4-yl)-N-[3- | | | |
| | | (trifluoromethoxy)benzyl]-1,2- | | | |
| | | dihydrospiro[indole-3,4′- | | | |
| | | piperidin]-5-amine | | | |
| 140 | 1, 7 | N-(4-methylbenzyl)-1-(7H- | 425.4 | 1.6 | I |
| | | pyrrolo[2,3-d]pyrimidin-4-yl)- | | | |
| | | 1,2-dihydrospiro[indole-3,4′- | | | |
| | | piperidin]-5-amine | | | |
| 141 | 1, 7 | N-(4-morpholin-4-ylbenzyl)-1- | 496.5 | 1 | I |
| | | (7H-pyrrolo[2,3-d]pyrimidin-4- | | | |
| | | yl)-1,2-dihydrospiro[indole-3,4′- | | | |
| | | piperidin]-5-amine | | | |
| 142 | 1, 7 | N-(4-phenoxybenzyl)-1-(7H- | 503.5 | 2.1 | I |
| | | pyrrolo[2,3-d]pyrimidin-4-yl)- | | | |
| | | 1,2-dihydrospiro[indole-3,4′- | | | |
| | | piperidin]-5-amine | | | |
| 143 | 1, 7 | N-(biphenyl-4-ylmethyl)-1-(7H- | 487.5 | 1.9 | I |
| | | pyrrolo[2,3-d]pyrimidin-4-yl)- | | | |
| | | 1,2-dihydrospiro[indole-3,4′- | | | |
| | | piperidin]-5-amine | | | |
| 144 | 1, 7 | N-(3-methoxybenzyl)-1-(7H- | 441.5 | 1.6 | I |
| | | pyrrolo[2,3-d]pyrimidin-4-yl)- | | | |
| | | 1,2-dihydrospiro[indole-3,4′- | | | |
| | | piperidin]-5-amine | | | |
| 145 | 1, 7 | N-(biphenyl-3-ylmethyl)-1-(7H- | 487.5 | 1.9 | I |
| | | pyrrolo[2,3-d]pyrimidin-4-yl)- | | | |
| | | 1,2-dihydrospiro[indole-3,4′- | | | |
| | | piperidin]-5-amine | | | |
| 146 | 10 | 1-(5-chloro-7H-pyrrolo[2,3- | 368.1 | 2.24 | E |
| | | d]pyrimidin-4-yl)-1′,5-dimethyl- | | | |
| | | 1,2-dihydrospiro[indole-3,4′- | | | |
| | | piperidine] | | | |
| 147 | 10 | 1′,5-dimethyl-1-(7H- | 334.14 | 2.08 | E |
| | | pyrrolo[2,3-d]pyrimidin-4-yl)- | | | |
| | | 1,2-dihydrospiro[indole-3,4′- | | | |
| | | piperidine] | | | |
| 148 | 10 | 5-fluoro-1′-methyl-1-(7H- | 338.16 | 2.01 | E |
| | | pyrrolo[2,3-d]pyrimidin-4-yl)- | | | |
| | | 1,2-dihydrospiro[indole-3,4′- | | | |
| | | piperidine] | | | |
| 149 | 10 | 1-(5-chloro-7H-pyrrolo[2,3- | 372.05 | 2.17 | E |
| | | d]pyrimidin-4-yl)-5-fluoro-1′- | | | |
| | | methyl-1,2-dihydrospiro[indole- | | | |
| | | 3,4′-piperidine] | | | |
| 150 | 10 | 1-(5-chloro-7H-pyrrolo[2,3- | 354.08 | 2.06 | E |
| | | d]pyrimidin-4-yl)-1′-methyl-1,2- | | | |
| | | dihydrospiro[indole-3,4′- | | | |
| | | piperidine] | | | |
| 151 | 11 | 1′-(4-chlorobenzyl)-5-methoxy- | 460.4 | 1.7 | I |
| | | 1-(7H-pyrrolo[2,3-d]pyrimidin- | | | |
| | | 4-yl)-1,2-dihydrospiro[indole- | | | |
| | | 3,4′-piperidine] | | | |
| 152 | 11 | 1′-(4-chlorobenzyl)-1-(7H- | 498.1 | 1.98 | I |
| | | pyrrolo[2,3-d]pyrimidin-4-yl)-5- | | | |
| | | (trifluoromethyl)-1,2- | | | |
| | | dihydrospiro[indole-3,4′- | | | |
| | | piperidine] | | | |
| 153 | 11 | 1′-(4-chlorobenzyl)-1-(5- | 478.04 | 4.95 | D |
| | | chloro-7H-pyrrolo[2,3- | | | |
| | | d]pyrimidin-4-yl)-5-methyl-1,2- | | | |
| | | dihydrospiro[indole-3,4′- | | | |
| | | piperidine] | | | |
| 154 | 11 | 1′-(4-chlorobenzyl)-1-(7H- | 430.08 | 4 | D |
| | | pyrrolo[2,3-d]pyrimidin-4-yl)- | | | |
| | | 1,2-dihydrospiro[indole-3,4′- | | | |
| | | piperidine] | | | |
| 155 | 11 | 1′-(4-chlorobenzyl)-5,7- | 458.11 | 4.21 | D |
| | | dimethyl-1-(7H-pyrrolo[2,3- | | | |
| | | d]pyrimidin-4-yl)-1,2- | | | |
| | | dihydrospiro[indole-3,4′- | | | |
| | | piperidine] | | | |
| 156 | 11 | 1′-(4-chlorobenzyl)-1-(5- | 481.99 | 4.95 | D |
| | | chloro-7H-pyrrolo[2,3- | | | |
| | | d]pyrimidin-4-yl)-5-fluoro-1,2- | | | |
| | | dihydrospiro[indole-3,4′- | | | |
| | | piperidine] | | | |
| 157 | 11 | 1′-(4-chlorobenzyl)-5-methyl-1- | 444.05 | 4.22 | D |
| | | (7H-pyrrolo[2,3-d]pyrimidin-4- | | | |
| | | yl)-1,2-dihydrospiro[indole-3,4′- | | | |
| | | piperidine] | | | |
| 158 | 11 | 1′-(4-chlorobenzyl)-5-fluoro-1- | 448.04 | 4.21 | D |
| | | (7H-pyrrolo[2,3-d]pyrimidin-4- | | | |
| | | yl)-1,2-dihydrospiro[indole-3,4′- | | | |
| | | piperidine] | | | |
| 159 | 11 | 1′-(4-chlorobenzyl)-1-(5- | 464.03 | 4.74 | D |
| | | chloro-7H-pyrrolo[2,3- | | | |
| | | d]pyrimidin-4-yl)-1,2- | | | |
| | | dihydrospiro[indole-3,4′- | | | |
| | | piperidine] | | | |
| 160 | 10 | 1′-methyl-1-(7H-pyrrolo[2,3- | 345.3 | 1.12 | I |
| | | d]pyrimidin-4-yl)-1,2- | | | |
| | | dihydrospiro[indole-3,4′- | | | |
| | | piperidine]-5-carbonitrile | | | |
| 161 | 11 | 1′-(4-methylbenzyl)-1-(7H- | 410.08 | 1.75 | C |
| | | pyrrolo[2,3-d]pyrimidin-4-yl)- | | | |
| | | 1,2-dihydrospiro[indole-3,4′- | | | |
| | | piperidine] | | | |
| 162 | 11 | methyl 4-{[1-(7H-pyrrolo[2,3- | 454.04 | 1.67 | C |
| | | d]pyrimidin-4-yl)-1,2-dihydro- | | | |
| | | 1′H-spiro[indole-3,4′-piperidin]- | | | |
| | | 1′-yl]methyl}benzoate | | | |
| 163 | 11 | 1′-(cyclohexylmethyl)-1-(7H- | 402.11 | 1.73 | C |
| | | pyrrolo[2,3-d]pyrimidin-4-yl)- | | | |
| | | 1,2-dihydrospiro[indole-3,4′- | | | |
| | | piperidine] | | | |
| 164 | 11 | 1′-(3-methylbutyl)-1-(7H- | 376.12 | 1.6 | C |
| | | pyrrolo[2,3-d]pyrimidin-4-yl)- | | | |
| | | 1,2-dihydrospiro[indole-3,4′- | | | |
| | | piperidine] | | | |
| 165 | 11 | 1′-(cyclopropylmethyl)-1-(7H- | 360.08 | 1.43 | C |
| | | pyrrolo[2,3-d]pyrimidin-4-yl)- | | | |
| | | 1,2-dihydrospiro[indole-3,4′- | | | |
| | | piperidine] | | | |
| 166 | 10 | 1′,4-dimethyl-1-(7H- | 334.3 | 1.8 | I |
| | | pyrrolo[2,3-d]pyrimidin-4-yl)- | | | |
| | | 1,2-dihydrospiro[indole-3,4′- | | | |
| | | piperidine] | | | |
| 167 | 10 | 1′,6-dimethyl-1-(7H- | 334.3 | 2 | I |
| | | pyrrolo[2,3-d]pyrimidin-4-yl)- | | | |
| | | 1,2-dihydrospiro[indole-3,4′- | | | |
| | | piperidine] | | | |
| 168 | 11 | 5-chloro-1′-(cyclohexylmethyl)- | 436.17 | 2.03 | B |
| | | 1-(7H-pyrrolo[2,3-d]pyrimidin- | | | |
| | | 4-yl)-1,2-dihydrospiro[indole- | | | |
| | | 3,4′-piperidine] | | | |
| 169 | 11 | 5-chloro-1′-(2-phenylethyl)-1- | 444.17 | 1.98 | B |
| | | (7H-pyrrolo[2,3-d]pyrimidin-4- | | | |
| | | yl)-1,2-dihydrospiro[indole-3,4′- | | | |
| | | piperidine] | | | |
| 170 | 11 | 5-chloro-1′-[(3-phenyl-1H- | 496.16 | 1.87 | B |
| | | pyrazol-4-yl)methyl]-1-(7H- | | | |
| | | pyrrolo[2,3-d]pyrimidin-4-yl)- | | | |
| | | 1,2-dihydrospiro[indole-3,4′- | | | |
| | | piperidine] | | | |
| 171 | 11 | 5-chloro-1′-(4-methoxybenzyl)- | 460.15 | 1.96 | B |
| | | 1-(7H-pyrrolo[2,3-d]pyrimidin- | | | |
| | | 4-yl)-1,2-dihydrospiro[indole- | | | |
| | | 3,4′-piperidine] | | | |
| 172 | 11 | 1′-butyl-5-chloro-1-(7H- | 396.14 | 1.79 | B |
| | | pyrrolo[2,3-d]pyrimidin-4-yl)- | | | |
| | | 1,2-dihydrospiro[indole-3,4′- | | | |
| | | piperidine] | | | |
| 173 | 11 | 5-chloro-1′-(2,4- | 466.14 | 1.95 | B |
| | | difluorobenzyl)-1-(7H- | | | |
| | | pyrrolo[2,3-d]pyrimidin-4-yl)- | | | |
| | | 1,2-dihydrospiro[indole-3,4′- | | | |
| | | piperidine] | | | |
| 174 | 11 | 5-chloro-1-(7H-pyrrolo[2,3- | 437.1 | 1.7 | B |
| | | d]pyrimidin-4-yl)-1′-(1,3- | | | |
| | | thiazol-2-ylmethyl)-1,2- | | | |
| | | dihydrospiro[indole-3,4′- | | | |
| | | piperidine] | | | |
| 175 | 11 | 5-chloro-1-(7H-pyrrolo[2,3- | 498.13 | 2.17 | B |
| | | d]pyrimidin-4-yl)-1′-[4- | | | |
| | | (trifluoromethyl)benzyl]-1,2- | | | |
| | | dihydrospiro[indole-3,4′- | | | |
| | | piperidine] | | | |
| 176 | 11 | 5-chloro-1′-(4-chlorobenzyl)-1- | 464.1 | 2.07 | B |
| | | (7H-pyrrolo[2,3-d]pyrimidin-4- | | | |
| | | yl)-1,2-dihydrospiro[indole-3,4′- | | | |
| | | piperidine] | | | |
| 177 | 11 | 5-chloro-1′-(3-fluoro-4- | 478.12 | 2 | B |
| | | methoxybenzyl)-1-(7H- | | | |
| | | pyrrolo[2,3-d]pyrimidin-4-yl)- | | | |
| | | 1,2-dihydrospiro[indole-3,4′- | | | |
| | | piperidine] | | | |
| 178 | 11 | 5-chloro-1′-(2-chlorobenzyl)-1- | 464.11 | 1.97 | B |
| | | (7H-pyrrolo[2,3-d]pyrimidin-4- | | | |
| | | yl)-1,2-dihydrospiro[indole-3,4′- | | | |
| | | piperidine] | | | |
| 179 | 11 | 1′-[2-(benzyloxy)ethyl]-5- | 474.16 | 2.06 | B |
| | | chloro-1-(7H-pyrrolo[2,3- | | | |
| | | d]pyrimidin-4-yl)-1,2- | | | |
| | | dihydrospiro[indole-3,4′- | | | |
| | | piperidine] | | | |
| 180 | 11 | 5-chloro-1′-[(2-ethyl-1H- | 448.18 | 1.55 | B |
| | | imidazol-5-yl)methyl]-1-(7H- | | | |
| | | pyrrolo[2,3-d]pyrimidin-4-yl)- | | | |
| | | 1,2-dihydrospiro[indole-3,4′- | | | |
| | | piperidine] | | | |
| 181 | 11 | 5-chloro-1′-(3,4- | 466.13 | 2.02 | B |
| | | difluorobenzyl)-1-(7H- | | | |
| | | pyrrolo[2,3-d]pyrimidin-4-yl)- | | | |
| | | 1,2-dihydrospiro[indole-3,4′- | | | |
| | | piperidine] | | | |
| 182 | 11 | 5-chloro-1′-[(4-methyl-1H- | 434.18 | 1.52 | B |
| | | imidazol-5-yl)methyl]-1-(7H- | | | |
| | | pyrrolo[2,3-d]pyrimidin-4-yl)- | | | |
| | | 1,2-dihydrospiro[indole-3,4′- | | | |
| | | piperidine] | | | |
| 183 | 11 | 1′-(1H-benzimidazol-2- | 470.15 | 1.87 | B |
| | | ylmethyl)-5-chloro-1-(7H- | | | |
| | | pyrrolo[2,3-d]pyrimidin-4-yl)- | | | |
| | | 1,2-dihydrospiro[indole-3,4′- | | | |
| | | piperidine] | | | |
| 184 | 11 | 5-chloro-1′-(4-fluorobenzyl)-1- | 448.15 | 1.96 | B |
| | | (7H-pyrrolo[2,3-d]pyrimidin-4- | | | |
| | | yl)-1,2-dihydrospiro[indole-3,4′- | | | |
| | | piperidine] | | | |
| 185 | 11 | 5-chloro-1′-(4-chlorobenzyl)-1- | 500.07 | 2.35 | B |
| | | (5-chloro-7H-pyrrolo[2,3- | | | |
| | | d]pyrimidin-4-yl)-1,2- | | | |
| | | dihydrospiro[indole-3,4′- | | | |
| | | piperidine] | | | |
| 186 | 11 | 5-chloro-1-(5-chloro-7H- | 530.12 | 2.12 | B |
| | | pyrrolo[2,3-d]pyrimidin-4-yl)-1′- | | | |
| | | [(3-phenyl-1H-pyrazol-4- | | | |
| | | yl)methyl]-1,2- | | | |
| | | dihydrospiro[indole-3,4′- | | | |
| | | piperidine] | | | |
| 187 | 11 | 5-chloro-1′-[(3-methyl-1H- | 434.17 | 1.7 | B |
| | | pyrazol-5-yl)methyl]-1-(7H- | | | |
| | | pyrrolo[2,3-d]pyrimidin-4-yl)- | | | |
| | | 1,2-dihydrospiro[indole-3,4′- | | | |
| | | piperidine] | | | |
| 188 | 11 | 5-chloro-1-(5-chloro-7H- | 478.12 | 2.3 | B |
| | | pyrrolo[2,3-d]pyrimidin-4-yl)-1′- | | | |
| | | (2-phenylethyl)-1,2- | | | |
| | | dihydrospiro[indole-3,4′- | | | |
| | | piperidine] | | | |
| 189 | 11 | 5-chloro-1-(5-chloro-7H- | 470.16 | 2.35 | B |
| | | pyrrolo[2,3-d]pyrimidin-4-yl)-1′- | | | |
| | | (cyclohexylmethyl)-1,2- | | | |
| | | dihydrospiro[indole-3,4′- | | | |
| | | piperidine] | | | |
| 190 | 11 | 5-chloro-1-(7H-pyrrolo[2,3- | 419.14 | 1.82 | B |
| | | d]pyrimidin-4-yl)-1′-(1H-pyrrol- | | | |
| | | 2-ylmethyl)-1,2- | | | |
| | | dihydrospiro[indole-3,4′- | | | |
| | | piperidine] | | | |
| 191 | 11 | 5-chloro-1-(5-chloro-7H- | 465.11 | 1.78 | B |
| | | pyrrolo[2,3-d]pyrimidin-4-yl)-1′- | | | |
| | | (pyridin-3-ylmethyl)-1,2- | | | |
| | | dihydrospiro[indole-3,4′- | | | |
| | | piperidine] | | | |
| 192 | 11 | 1′-butyl-5-chloro-1-(5-chloro- | 430.11 | 2.12 | B |
| | | 7H-pyrrolo[2,3-d]pyrimidin-4- | | | |
| | | yl)-1,2-dihydrospiro[indole-3,4′- | | | |
| | | piperidine] | | | |
| 193 | 11 | 5-chloro-1-(5-chloro-7H- | 494.11 | 2.26 | B |
| | | pyrrolo[2,3-d]pyrimidin-4-yl)-1′- | | | |
| | | (4-methoxybenzyl)-1,2- | | | |
| | | dihydrospiro[indole-3,4′- | | | |
| | | piperidine] | | | |
| 194 | 11 | 5-chloro-1-(5-chloro-7H- | 512.11 | 2.28 | B |
| | | pyrrolo[2,3-d]pyrimidin-4-yl)-1′- | | | |
| | | (3-fluoro-4-methoxybenzyl)- | | | |
| | | 1,2-dihydrospiro[indole-3,4′- | | | |
| | | piperidine] | | | |
| 195 | 11 | 5-chloro-1-(5-chloro-7H- | 532.08 | 2.46 | B |
| | | pyrrolo[2,3-d]pyrimidin-4-yl)-1′- | | | |
| | | [4-(trifluoromethyl)benzyl]-1,2- | | | |
| | | dihydrospiro[indole-3,4′- | | | |
| | | piperidine] | | | |
| 196 | 11 | 1′-(1H-benzimidazol-2- | 504.13 | 2.1 | B |
| | | ylmethyl)-5-chloro-1-(5-chloro- | | | |
| | | 7H-pyrrolo[2,3-d]pyrimidin-4- | | | |
| | | yl)-1,2-dihydrospiro[indole-3,4′- | | | |
| | | piperidine] | | | |
| 197 | 11 | 5-chloro-1-(5-chloro-7H- | 482.11 | 2.26 | B |
| | | pyrrolo[2,3-d]pyrimidin-4-yl)-1′- | | | |
| | | (4-fluorobenzyl)-1,2- | | | |
| | | dihydrospiro[indole-3,4′- | | | |
| | | piperidine] | | | |
| 198 | 11 | 5-chloro-1-(5-chloro-7H- | 468.11 | 1.97 | B |
| | | pyrrolo[2,3-d]pyrimidin-4-yl)-1′- | | | |
| | | [(3-methyl-1H-pyrazol-5- | | | |
| | | yl)methyl]-1,2- | | | |
| | | dihydrospiro[indole-3,4′- | | | |
| | | piperidine] | | | |
| 199 | 11 | 5-chloro-1-(5-chloro-7H- | 453.11 | 2.13 | B |
| | | pyrrolo[2,3-d]pyrimidin-4-yl)-1′- | | | |
| | | (1H-pyrrol-2-ylmethyl)-1,2- | | | |
| | | dihydrospiro[indole-3,4′- | | | |
| | | piperidine] | | | |
| 200 | 11 | 1′-[2-(benzyloxy)ethyl]-5- | 508.13 | 2.36 | B |
| | | chloro-1-(5-chloro-7H- | | | |
| | | pyrrolo[2,3-d]pyrimidin-4-yl)- | | | |
| | | 1,2-dihydrospiro[indole-3,4′- | | | |
| | | piperidine] | | | |
| 201 | 11 | 5-chloro-1-(5-chloro-7H- | 500.1 | 2.24 | B |
| | | pyrrolo[2,3-d]pyrimidin-4-yl)-1′- | | | |
| | | (2,4-difluorobenzyl)-1,2- | | | |
| | | dihydrospiro[indole-3,4′- | | | |
| | | piperidine] | | | |
| 202 | 11 | 5-chloro-1′-(2-chlorobenzyl)-1- | 500.04 | 2.26 | B |
| | | (5-chloro-7H-pyrrolo[2,3- | | | |
| | | d]pyrimidin-4-yl)-1,2- | | | |
| | | dihydrospiro[indole-3,4′- | | | |
| | | piperidine] | | | |
| 203 | 11 | 5-chloro-1-(5-chloro-7H- | 482.12 | 1.76 | B |
| | | pyrrolo[2,3-d]pyrimidin-4-yl)-1′- | | | |
| | | [(2-ethyl-1H-imidazol-5- | | | |
| | | yl)methyl]-1,2- | | | |
| | | dihydrospiro[indole-3,4′- | | | |
| | | piperidine] | | | |
| 204 | 11 | 1′-(cyclohexylmethyl)-5- | 416.21 | 2.02 | B |
| | | methyl-1-(7H-pyrrolo[2,3- | | | |
| | | d]pyrimidin-4-yl)-1,2- | | | |
| | | dihydrospiro[indole-3,4′- | | | |
| | | piperidine] | | | |
| 205 | 11 | 1′-(cyclohexylmethyl)-5-fluoro- | 420.18 | 2.01 | B |
| | | 1-(7H-pyrrolo[2,3-d]pyrimidin- | | | |
| | | 4-yl)-1,2-dihydrospiro[indole- | | | |
| | | 3,4′-piperidine] | | | |
| 206 | 11 | 1-(5-chloro-7H-pyrrolo[2,3- | 436.15 | 2.28 | B |
| | | d]pyrimidin-4-yl)-1′- | | | |
| | | (cyclohexylmethyl)-1,2- | | | |
| | | dihydrospiro[indole-3,4′- | | | |
| | | piperidine] | | | |
| 207 | 11 | 5-fluoro-1′-(3-methylbutyl)-1- | 394.16 | 1.88 | B |
| | | (7H-pyrrolo[2,3-d]pyrimidin-4- | | | |
| | | yl)-1,2-dihydrospiro[indole-3,4′- | | | |
| | | piperidine] | | | |
| 208 | 11 | 1-(5-chloro-7H-pyrrolo[2,3- | 450.22 | 2.37 | B |
| | | d]pyrimidin-4-yl)-1′- | | | |
| | | (cyclohexylmethyl)-5-methyl- | | | |
| | | 1,2-dihydrospiro[indole-3,4′- | | | |
| | | piperidine] | | | |
| 209 | 11 | 5-chloro-1′-(3-methylbutyl)-1- | 410.15 | 2.05 | B |
| | | (7H-pyrrolo[2,3-d]pyrimidin-4- | | | |
| | | yl)-1,2-dihydrospiro[indole-3,4′- | | | |
| | | piperidine] | | | |
| 210 | 11 | 1-(5-chloro-7H-pyrrolo[2,3- | 424.17 | 2.24 | B |
| | | d]pyrimidin-4-yl)-5-methyl-1′- | | | |
| | | (3-methylbutyl)-1,2- | | | |
| | | dihydrospiro[indole-3,4′- | | | |
| | | piperidine] | | | |
| 211 | 11 | 1-(5-chloro-7H-pyrrolo[2,3- | 454.17 | 2.38 | B |
| | | d]pyrimidin-4-yl)-1′- | | | |
| | | (cyclohexylmethyl)-5-fluoro- | | | |
| | | 1,2-dihydrospiro[indole-3,4′- | | | |
| | | piperidine] | | | |
| 212 | 11 | 1-(5-chloro-7H-pyrrolo[2,3- | 410.15 | 2.16 | B |
| | | d]pyrimidin-4-yl)-1′-(3- | | | |
| | | methylbutyl)-1,2- | | | |
| | | dihydrospiro[indole-3,4′- | | | |
| | | piperidine] | | | |
| 213 | 11 | 1-(5-chloro-7H-pyrrolo[2,3- | 428.18 | 2.25 | B |
| | | d]pyrimidin-4-yl)-5-fluoro-1′-(3- | | | |
| | | methylbutyl)-1,2- | | | |
| | | dihydrospiro[indole-3,4′- | | | |
| | | piperidine] | | | |
| 214 | 11 | 5-methyl-1′-(3-methylbutyl)-1- | 390.22 | 1.88 | B |
| | | (7H-pyrrolo[2,3-d]pyrimidin-4- | | | |
| | | yl)-1,2-dihydrospiro[indole-3,4′- | | | |
| | | piperidine] | | | |
| 215 | 10 | 5-chloro-1′-methyl-1-(7H- | 354.09 | 1.69 | B |
| | | pyrrolo[2,3-d]pyrimidin-4-yl)- | | | |
| | | 1,2-dihydrospiro[indole-3,4′- | | | |
| | | piperidine] | | | |
| 216 | 11 | 5-chloro-1-(5-chloro-7H- | 444.11 | 2.41 | B |
| | | pyrrolo[2,3-d]pyrimidin-4-yl)-1′- | | | |
| | | (3-methylbutyl)-1,2- | | | |
| | | dihydrospiro[indole-3,4′- | | | |
| | | piperidine] | | | |
| 217 | 11 | 5-methyl-1′-(2-methylbutyl)-1- | 390.22 | 1.87 | B |
| | | (7H-pyrrolo[2,3-d]pyrimidin-4- | | | |
| | | yl)-1,2-dihydrospiro[indole-3,4′- | | | |
| | | piperidine] | | | |
| 218 | 10 | 5-chloro-1-(5-chloro-7H- | 388.05 | 2.01 | B |
| | | pyrrolo[2,3-d]pyrimidin-4-yl)-1′- | | | |
| | | methyl-1,2-dihydrospiro[indole- | | | |
| | | 3,4′-piperidine] | | | |
| 219 | 11 | 5-fluoro-1′-(2-methylbutyl)-1- | 394.16 | 1.86 | B |
| | | (7H-pyrrolo[2,3-d]pyrimidin-4- | | | |
| | | yl)-1,2-dihydrospiro[indole-3,4′- | | | |
| | | piperidine] | | | |
| 220 | 11 | 1′-(2-methylbutyl)-1-(7H- | 376.2 | 1.75 | B |
| | | pyrrolo[2,3-d]pyrimidin-4-yl)- | | | |
| | | 1,2-dihydrospiro[indole-3,4′- | | | |
| | | piperidine] | | | |
| 221 | 11 | 1′-butyl-1-(5-chloro-7H- | 414.1 | 2.13 | B |
| | | pyrrolo[2,3-d]pyrimidin-4-yl)-5- | | | |
| | | fluoro-1,2-dihydrospiro[indole- | | | |
| | | 3,4′-piperidine] | | | |
| 222 | 11 | 1-(5-chloro-7H-pyrrolo[2,3- | 410.15 | 2.11 | B |
| | | d]pyrimidin-4-yl)-1′-(2- | | | |
| | | methylbutyl)-1,2- | | | |
| | | dihydrospiro[indole-3,4′- | | | |
| | | piperidine] | | | |
| 223 | 11 | 1′-butyl-1-(7H-pyrrolo[2,3- | 362.18 | 1.65 | B |
| | | d]pyrimidin-4-yl)-1,2- | | | |
| | | dihydrospiro[indole-3,4′- | | | |
| | | piperidine] | | | |
| 224 | 11 | 1′-butyl-5-methyl-1-(7H- | 376.2 | 1.77 | B |
| | | pyrrolo[2,3-d]pyrimidin-4-yl)- | | | |
| | | 1,2-dihydrospiro[indole-3,4′- | | | |
| | | piperidine] | | | |
| 225 | 11 | 1-(5-chloro-7H-pyrrolo[2,3- | 428.12 | 2.22 | B |
| | | d]pyrimidin-4-yl)-5-fluoro-1′-(2- | | | |
| | | methylbutyl)-1,2- | | | |
| | | dihydrospiro[indole-3,4′- | | | |
| | | piperidine] | | | |
| 226 | 11 | 1′-butyl-1-(5-chloro-7H- | 396.14 | 2.02 | B |
| | | pyrrolo[2,3-d]pyrimidin-4-yl)- | | | |
| | | 1,2-dihydrospiro[indole-3,4′- | | | |
| | | piperidine] | | | |
| 227 | 11 | 5-chloro-1-(5-chloro-7H- | 444.11 | 2.38 | B |
| | | pyrrolo[2,3-d]pyrimidin-4-yl)-1′- | | | |
| | | (2-methylbutyl)-1,2- | | | |
| | | dihydrospiro[indole-3,4′- | | | |
| | | piperidine] | | | |
| 228 | 11 | 1′-propyl-1-(7H-pyrrolo[2,3- | 348.17 | 1.53 | B |
| | | d]pyrimidin-4-yl)-1,2- | | | |
| | | dihydrospiro[indole-3,4′- | | | |
| | | piperidine] | | | |
| 229 | 11 | 1′-butyl-1-(5-chloro-7H- | 410.15 | 2.13 | B |
| | | pyrrolo[2,3-d]pyrimidin-4-yl)-5- | | | |
| | | methyl-1,2-dihydrospiro[indole- | | | |
| | | 3,4′-piperidine] | | | |
| 230 | 11 | 1-(5-chloro-7H-pyrrolo[2,3- | 382.12 | 1.89 | B |
| | | d]pyrimidin-4-yl)-1′-propyl-1,2- | | | |
| | | dihydrospiro[indole-3,4′- | | | |
| | | piperidine] | | | |
| 231 | 11 | 1′-butyl-5-fluoro-1-(7H- | 380.15 | 1.75 | B |
| | | pyrrolo[2,3-d]pyrimidin-4-yl)- | | | |
| | | 1,2-dihydrospiro[indole-3,4′- | | | |
| | | piperidine] | | | |
| 232 | 11 | 5-methyl-1′-propyl-1-(7H- | 362.18 | 1.65 | B |
| | | pyrrolo[2,3-d]pyrimidin-4-yl)- | | | |
| | | 1,2-dihydrospiro[indole-3,4′- | | | |
| | | piperidine] | | | |
| 233 | 11 | 5-chloro-1-(5-chloro-7H- | 416.08 | 2.16 | B |
| | | pyrrolo[2,3-d]pyrimidin-4-yl)-1′- | | | |
| | | propyl-1,2-dihydrospiro[indole- | | | |
| | | 3,4′-piperidine] | | | |
| 234 | 11 | 1-(5-chloro-7H-pyrrolo[2,3- | 396.14 | 1.99 | B |
| | | d]pyrimidin-4-yl)-5-methyl-1′- | | | |
| | | propyl-1,2-dihydrospiro[indole- | | | |
| | | 3,4′-piperidine] | | | |
| 235 | 11 | 5-fluoro-1′-propyl-1-(7H- | 366.13 | 1.63 | B |
| | | pyrrolo[2,3-d]pyrimidin-4-yl)- | | | |
| | | 1,2-dihydrospiro[indole-3,4′- | | | |
| | | piperidine] | | | |
| 236 | 11 | 1′-(cyclopropylmethyl)-5- | 378.17 | 1.67 | B |
| | | fluoro-1-(7H-pyrrolo[2,3- | | | |
| | | d]pyrimidin-4-yl)-1,2- | | | |
| | | dihydrospiro[indole-3,4′- | | | |
| | | piperidine] | | | |
| 237 | 11 | 1-(5-chloro-7H-pyrrolo[2,3- | 400.09 | 2 | B |
| | | d]pyrimidin-4-yl)-5-fluoro-1′- | | | |
| | | propyl-1,2-dihydrospiro[indole- | | | |
| | | 3,4′-piperidine] | | | |
| 238 | 11 | 5-chloro-1′- | 394.1 | 1.83 | B |
| | | (cyclopropylmethyl)-1-(7H- | | | |
| | | pyrrolo[2,3-d]pyrimidin-4-yl)- | | | |
| | | 1,2-dihydrospiro[indole-3,4′- | | | |
| | | piperidine] | | | |
| 239 | 11 | 5-chloro-1′-propyl-1-(7H- | 382.12 | 1.81 | B |
| | | pyrrolo[2,3-d]pyrimidin-4-yl)- | | | |
| | | 1,2-dihydrospiro[indole-3,4′- | | | |
| | | piperidine] | | | |
| 240 | 11 | 1′-(cyclopropylmethyl)-5- | 374.16 | 1.71 | B |
| | | methyl-1-(7H-pyrrolo[2,3- | | | |
| | | d]pyrimidin-4-yl)-1,2- | | | |
| | | dihydrospiro[indole-3,4′- | | | |
| | | piperidine] | | | |
| 241 | 11 | 1′-(2-methoxyethyl)-1-(7H- | 364.16 | 1.51 | B |
| | | pyrrolo[2,3-d]pyrimidin-4-yl)- | | | |
| | | 1,2-dihydrospiro[indole-3,4′- | | | |
| | | piperidine] | | | |
| 242 | 11 | 1-(5-chloro-7H-pyrrolo[2,3- | 394.16 | 1.93 | B |
| | | d]pyrimidin-4-yl)-1′- | | | |
| | | (cyclopropylmethyl)-1,2- | | | |
| | | dihydrospiro[indole-3,4′- | | | |
| | | piperidine] | | | |
| 243 | 11 | 1-(5-chloro-7H-pyrrolo[2,3- | 408.12 | 2.05 | B |
| | | d]pyrimidin-4-yl)-1′- | | | |
| | | (cyclopropylmethyl)-5-methyl- | | | |
| | | 1,2-dihydrospiro[indole-3,4′- | | | |
| | | piperidine] | | | |
| 244 | 11 | 1-(5-chloro-7H-pyrrolo[2,3- | 412.13 | 2.03 | B |
| | | d]pyrimidin-4-yl)-1′- | | | |
| | | (cyclopropylmethyl)-5-fluoro- | | | |
| | | 1,2-dihydrospiro[indole-3,4′- | | | |
| | | piperidine] | | | |
| 245 | 11 | N,N,2,2-tetramethyl-3-[1-(7H- | 419.26 | 1.39 | B |
| | | pyrrolo[2,3-d]pyrimidin-4-yl)- | | | |
| | | 1,2-dihydro-1′H-spiro[indole- | | | |
| | | 3,4′-piperidin]-1′-yl]propan-1- | | | |
| | | amine | | | |
| 246 | 11 | 3-[5-chloro-1-(7H-pyrrolo[2,3- | 453.22 | 1.62 | B |
| | | d]pyrimidin-4-yl)-1,2-dihydro- | | | |
| | | 1′H-spiro[indole-3,4′-piperidin]- | | | |
| | | 1′-yl]-N,N,2,2- | | | |
| | | tetramethylpropan-1-amine | | | |
| 247 | 11 | 1′-(2-methoxyethyl)-5-methyl- | 378.17 | 1.62 | B |
| | | 1-(7H-pyrrolo[2,3-d]pyrimidin- | | | |
| | | 4-yl)-1,2-dihydrospiro[indole- | | | |
| | | 3,4′-piperidine] | | | |
| 248 | 11 | 5-chloro-1-(5-chloro-7H- | 428.12 | 2.21 | B |
| | | pyrrolo[2,3-d]pyrimidin-4-yl)-1′- | | | |
| | | (cyclopropylmethyl)-1,2- | | | |
| | | dihydrospiro[indole-3,4′- | | | |
| | | piperidine] | | | |
| 249 | 11 | N,N,2,2-tetramethyl-3-[5- | 433.28 | 1.49 | B |
| | | methyl-1-(7H-pyrrolo[2,3- | | | |
| | | d]pyrimidin-4-yl)-1,2-dihydro- | | | |
| | | 1′H-spiro[indole-3,4′-piperidin]- | | | |
| | | 1′-yl]propan-1-amine | | | |
| 250 | 11 | 3-[1-(5-chloro-7H-pyrrolo[2,3- | 453.22 | 1.65 | B |
| | | d]pyrimidin-4-yl)-1,2-dihydro- | | | |
| | | 1′H-spiro[indole-3,4′-piperidin]- | | | |
| | | 1′-yl]-N,N,2,2- | | | |
| | | tetramethylpropan-1-amine | | | |
| 251 | 11 | 5-fluoro-1′-(2-methoxyethyl)-1- | 382.12 | 1.6 | B |
| | | (7H-pyrrolo[2,3-d]pyrimidin-4- | | | |
| | | yl)-1,2-dihydrospiro[indole-3,4′- | | | |
| | | piperidine] | | | |
| 252 | 11 | 3-[1-(5-chloro-7H-pyrrolo[2,3- | 471.24 | 1.73 | B |
| | | d]pyrimidin-4-yl)-5-fluoro-1,2- | | | |
| | | dihydro-1′H-spiro[indole-3,4′- | | | |
| | | piperidin]-1′-yl]-N,N,2,2- | | | |
| | | tetramethylpropan-1-amine | | | |
| 253 | 11 | 3-[5-fluoro-1-(7H-pyrrolo[2,3- | 437.23 | 1.46 | B |
| | | d]pyrimidin-4-yl)-1,2-dihydro- | | | |
| | | 1′H-spiro[indole-3,4′-piperidin]- | | | |
| | | 1′-yl]-N,N,2,2- | | | |
| | | tetramethylpropan-1-amine | | | |
| 254 | 11 | 1′-(1H-imidazol-4-ylmethyl)-5- | 400.15 | 1.47 | B |
| | | methyl-1-(7H-pyrrolo[2,3- | | | |
| | | d]pyrimidin-4-yl)-1,2- | | | |
| | | dihydrospiro[indole-3,4′- | | | |
| | | piperidine] | | | |
| 255 | 11 | 3-[1-(5-chloro-7H-pyrrolo[2,3- | 467.23 | 1.75 | B |
| | | d]pyrimidin-4-yl)-5-methyl-1,2- | | | |
| | | dihydro-1′H-spiro[indole-3,4′- | | | |
| | | piperidin]-1′-yl]-N,N,2,2- | | | |
| | | tetramethylpropan-1-amine | | | |
| 256 | 11 | 3-[5-chloro-1-(5-chloro-7H- | 487.17 | 1.85 | B |
| | | pyrrolo[2,3-d]pyrimidin-4-yl)- | | | |
| | | 1,2-dihydro-1′H-spiro[indole- | | | |
| | | 3,4′-piperidin]-1′-yl]-N,N,2,2- | | | |
| | | tetramethylpropan-1-amine | | | |
| 257 | 11 | 1-(5-chloro-7H-pyrrolo[2,3- | 434.17 | 1.71 | B |
| | | d]pyrimidin-4-yl)-1′-(1H- | | | |
| | | imidazol-4-ylmethyl)-5-methyl- | | | |
| | | 1,2-dihydrospiro[indole-3,4′- | | | |
| | | piperidine] | | | |
| 258 | 11 | 5-fluoro-1′-(1H-imidazol-4- | 404.17 | 1.42 | B |
| | | ylmethyl)-1-(7H-pyrrolo[2,3- | | | |
| | | d]pyrimidin-4-yl)-1,2- | | | |
| | | dihydrospiro[indole-3,4′- | | | |
| | | piperidine] | | | |
| 259 | 11 | 1′-ethyl-1-(7H-pyrrolo[2,3- | 334.15 | 1.44 | B |
| | | d]pyrimidin-4-yl)-1,2- | | | |
| | | dihydrospiro[indole-3,4′- | | | |
| | | piperidine] | | | |
| 260 | 11 | 1′-(1H-imidazol-4-ylmethyl)-1- | 386.14 | 1.35 | B |
| | | (7H-pyrrolo[2,3-d]pyrimidin-4- | | | |
| | | yl)-1,2-dihydrospiro[indole-3,4′- | | | |
| | | piperidine] | | | |
| 261 | 11 | 1-(5-chloro-7H-pyrrolo[2,3- | 438.12 | 1.69 | B |
| | | d]pyrimidin-4-yl)-5-fluoro-1′- | | | |
| | | (1H-imidazol-4-ylmethyl)-1,2- | | | |
| | | dihydrospiro[indole-3,4′- | | | |
| | | piperidine] | | | |
| 262 | 11 | 1′-ethyl-5-methyl-1-(7H- | 348.17 | 1.58 | B |
| | | pyrrolo[2,3-d]pyrimidin-4-yl)- | | | |
| | | 1,2-dihydrospiro[indole-3,4′- | | | |
| | | piperidine] | | | |
| 263 | 11 | 5-chloro-1′-(1H-imidazol-4- | 420.16 | 1.59 | B |
| | | ylmethyl)-1-(7H-pyrrolo[2,3- | | | |
| | | d]pyrimidin-4-yl)-1,2- | | | |
| | | dihydrospiro[indole-3,4′- | | | |
| | | piperidine] | | | |
| 264 | 11 | 5-chloro-1-(5-chloro-7H- | 454.07 | 1.83 | B |
| | | pyrrolo[2,3-d]pyrimidin-4-yl)-1′- | | | |
| | | (1H-imidazol-4-ylmethyl)-1,2- | | | |
| | | dihydrospiro[indole-3,4′- | | | |
| | | piperidine] | | | |
| 265 | 11 | 5-chloro-1′-ethyl-1-(7H- | 368.11 | 1.72 | B |
| | | pyrrolo[2,3-d]pyrimidin-4-yl)- | | | |
| | | 1,2-dihydrospiro[indole-3,4′- | | | |
| | | piperidine] | | | |
| 266 | 11 | 1′-ethyl-5-fluoro-1-(7H- | 352.12 | 1.54 | B |
| | | pyrrolo[2,3-d]pyrimidin-4-yl)- | | | |
| | | 1,2-dihydrospiro[indole-3,4′- | | | |
| | | piperidine] | | | |
| 267 | 11 | 1-(5-chloro-7H-pyrrolo[2,3- | 438.12 | 2.11 | B |
| | | d]pyrimidin-4-yl)-5-fluoro-1′-(3- | | | |
| | | furylmethyl)-1,2- | | | |
| | | dihydrospiro[indole-3,4′- | | | |
| | | piperidine] | | | |
| 268 | 11 | 1-(5-chloro-7H-pyrrolo[2,3- | 420.09 | 2 | B |
| | | d]pyrimidin-4-yl)-1′-(3- | | | |
| | | furylmethyl)-1,2- | | | |
| | | dihydrospiro[indole-3,4′- | | | |
| | | piperidine] | | | |
| 269 | 11 | 1-(5-chloro-7H-pyrrolo[2,3- | 434.11 | 2.11 | B |
| | | d]pyrimidin-4-yl)-1′-(3- | | | |
| | | furylmethyl)-5-methyl-1,2- | | | |
| | | dihydrospiro[indole-3,4′- | | | |
| | | piperidine] | | | |
| 270 | 11 | 1′-[1-(4-chlorophenyl)ethyl]-5- | 462.3 | 1.9 | I |
| | | fluoro-1-(7H-pyrrolo[2,3- | | | |
| | | d]pyrimidin-4-yl)-1,2- | | | |
| | | dihydrospiro[indole-3,4′- | | | |
| | | piperidine] | | | |
| 271 | 14 | tert-butyl 5-bromo-1-(7H- | 484.25 | 3.55 | G |
| | | pyrrolo[2,3-d]pyrimidin-4-yl)- | | | |
| | | 1,2-dihydro-1′H-spiro[indole- | | | |
| | | 3,4′-piperidine]-1′-carboxylate | | | |
| 272 | 14 | tert-butyl 5-chloro-1-(7H- | 440.3 | 3.47 | G |
| | | pyrrolo[2,3-d]pyrimidin-4-yl)- | | | |
| | | 1,2-dihydro-1′H-spiro[indole- | | | |
| | | 3,4′-piperidine]-1′-carboxylate | | | |
| 273 | 14 | benzyl 1-(7H-pyrrolo[2,3- | 440.13 | 1.71 | E |
| | | d]pyrimidin-4-yl)-1,2-dihydro- | | | |
| | | 1′H-spiro[indole-3,4′- | | | |
| | | piperidine]-1′-carboxylate | | | |
| 274 | 14 | benzyl 1-(5-chloro-7H- | 488.11 | 2.03 | E |
| | | pyrrolo[2,3-d]pyrimidin-4-yl)-5- | | | |
| | | methyl-1,2-dihydro-1′H- | | | |
| | | spiro[indole-3,4′-piperidine]-1′- | | | |
| | | carboxylate | | | |
| 275 | 14 | benzyl 5,7-dimethyl-1-(7H- | 468.17 | 1.82 | E |
| | | pyrrolo[2,3-d]pyrimidin-4-yl)- | | | |
| | | 1,2-dihydro-1′H-spiro[indole- | | | |
| | | 3,4′-piperidine]-1′-carboxylate | | | |
| 276 | 14 | benzyl 1-(5-chloro-7H- | 492.06 | 1.91 | E |
| | | pyrrolo[2,3-d]pyrimidin-4-yl)-5- | | | |
| | | fluoro-1,2-dihydro-1′H- | | | |
| | | spiro[indole-3,4′-piperidine]-1′- | | | |
| | | carboxylate | | | |
| 277 | 14 | benzyl 5-methyl-1-(7H- | 454.15 | 1.89 | E |
| | | pyrrolo[2,3-d]pyrimidin-4-yl)- | | | |
| | | 1,2-dihydro-1′H-spiro[indole- | | | |
| | | 3,4′-piperidine]-1′-carboxylate | | | |
| 278 | 14 | benzyl 1-(5-chloro-7H- | 502.12 | 2.2 | E |
| | | pyrrolo[2,3-d]pyrimidin-4-yl)- | | | |
| | | 5,7-dimethyl-1,2-dihydro-1′H- | | | |
| | | spiro[indole-3,4′-piperidine]-1′- | | | |
| | | carboxylate | | | |
| 279 | 14 | benzyl 1-(5-chloro-7H- | 474.1 | 1.84 | E |
| | | pyrrolo[2,3-d]pyrimidin-4-yl)- | | | |
| | | 1,2-dihydro-1′H-spiro[indole- | | | |
| | | 3,4′-piperidine]-1′-carboxylate | | | |
| 280 | 14 | benzyl 5-fluoro-1-(7H- | 458.11 | 1.79 | E |
| | | pyrrolo[2,3-d]pyrimidin-4-yl)- | | | |
| | | 1,2-dihydro-1′H-spiro[indole- | | | |
| | | 3,4′-piperidine]-1′-carboxylate | | | |
| 281 | 14 | benzyl 5-methoxy-1-(7H- | 470.3 | 2.6 | I |
| | | pyrrolo[2,3-d]pyrimidin-4-yl)- | | | |
| | | 1,2-dihydro-1′H-spiro[indole- | | | |
| | | 3,4′-piperidine]-1′-carboxylate | | | |
| 282 | 12 | 1′-(4-chlorobenzoyl)-1-(7H- | 443.98 | 2.33 | C |
| | | pyrrolo[2,3-d]pyrimidin-4-yl)- | | | |
| | | 1,2-dihydrospiro[indole-3,4′- | | | |
| | | piperidine] | | | |
| 283 | 14 | 2-methoxyethyl 1-(7H- | 408.04 | 1.97 | C |
| | | pyrrolo[2,3-d]pyrimidin-4-yl)- | | | |
| | | 1,2-dihydro-1′H-spiro[indole- | | | |
| | | 3,4′-piperidine]-1′-carboxylate | | | |
| 284 | 12 | N-(4-chlorophenyl)-1-(7H- | 459 | 2.42 | C |
| | | pyrrolo[2,3-d]pyrimidin-4-yl)- | | | |
| | | 1,2-dihydro-1′H-spiro[indole- | | | |
| | | 3,4′-piperidine]-1′-carboxamide | | | |
| 285 | 15 | N-ethyl-1-(7H-pyrrolo[2,3- | 377.08 | 1.75 | C |
| | | d]pyrimidin-4-yl)-1,2-dihydro- | | | |
| | | 1′H-spiro[indole-3,4′- | | | |
| | | piperidine]-1′-carboxamide | | | |
| 286 | 15 | 5-methyl-1′-(morpholin-4- | 433.07 | 1.93 | C |
| | | ylcarbonyl)-1-(7H-pyrrolo[2,3- | | | |
| | | d]pyrimidin-4-yl)-1,2- | | | |
| | | dihydrospiro[indole-3,4′- | | | |
| | | piperidine] | | | |
| 287 | 15 | 5-methyl-1′-[(4- | 446.09 | 1.51 | C |
| | | methylpiperazin-1-yl)carbonyl]- | | | |
| | | 1-(7H-pyrrolo[2,3-d]pyrimidin- | | | |
| | | 4-yl)-1,2-dihydrospiro[indole- | | | |
| | | 3,4′-piperidine] | | | |
| 288 | 15 | 5-fluoro-1′-(morpholin-4- | 437.03 | 1.9 | C |
| | | ylcarbonyl)-1-(7H-pyrrolo[2,3- | | | |
| | | d]pyrimidin-4-yl)-1,2- | | | |
| | | dihydrospiro[indole-3,4′- | | | |
| | | piperidine] | | | |
| 289 | 15 | 5-fluoro-1′-[(4-methylpiperazin- | 450.08 | 1.5 | C |
| | | 1-yl)carbonyl]-1-(7H- | | | |
| | | pyrrolo[2,3-d]pyrimidin-4-yl)- | | | |
| | | 1,2-dihydrospiro[indole-3,4′- | | | |
| | | piperidine] | | | |
| 290 | 15 | 1′-[(4-methylpiperazin-1- | 432.09 | 1.43 | C |
| | | yl)carbonyl]-1-(7H-pyrrolo[2,3- | | | |
| | | d]pyrimidin-4-yl)-1,2- | | | |
| | | dihydrospiro[indole-3,4′- | | | |
| | | piperidine] | | | |
| 291 | 15 | 1-(5-chloro-7H-pyrrolo[2,3- | 471.02 | 2.31 | C |
| | | d]pyrimidin-4-yl)-5-fluoro-1′- | | | |
| | | (morpholin-4-ylcarbonyl)-1,2- | | | |
| | | dihydrospiro[indole-3,4′- | | | |
| | | piperidine] | | | |
| 292 | 15 | 1-(5-chloro-7H-pyrrolo[2,3- | 484.04 | 1.77 | C |
| | | d]pyrimidin-4-yl)-5-fluoro-1′- | | | |
| | | [(4-methylpiperazin-1- | | | |
| | | yl)carbonyl]-1,2- | | | |
| | | dihydrospiro[indole-3,4′- | | | |
| | | piperidine] | | | |
| 293 | 15 | 1-(5-chloro-7H-pyrrolo[2,3- | 466.06 | 1.69 | C |
| | | d]pyrimidin-4-yl)-1′-[(4- | | | |
| | | methylpiperazin-1-yl)carbonyl]- | | | |
| | | 1,2-dihydrospiro[indole-3,4′- | | | |
| | | piperidine] | | | |
| 294 | 15 | 1-(5-chloro-7H-pyrrolo[2,3- | 453.01 | 2.16 | C |
| | | d]pyrimidin-4-yl)-1′-(morpholin- | | | |
| | | 4-ylcarbonyl)-1,2- | | | |
| | | dihydrospiro[indole-3,4′- | | | |
| | | piperidine] | | | |
| 295 | 15 | 1-(5-chloro-7H-pyrrolo[2,3- | 480.08 | 1.76 | C |
| | | d]pyrimidin-4-yl)-5-methyl-1′- | | | |
| | | [(4-methylpiperazin-1- | | | |
| | | yl)carbonyl]-1,2- | | | |
| | | dihydrospiro[indole-3,4′- | | | |
| | | piperidine] | | | |
| 296 | 15 | 1-(5-chloro-7H-pyrrolo[2,3- | 467.02 | 2.28 | C |
| | | d]pyrimidin-4-yl)-5-methyl-1′- | | | |
| | | (morpholin-4-ylcarbonyl)-1,2- | | | |
| | | dihydrospiro[indole-3,4′- | | | |
| | | piperidine] | | | |
| 297 | 12 | 1′-acetyl-5-fluoro-1-(7H- | 366.2 | 1.8 | I |
| | | pyrrolo[2,3-d]pyrimidin-4-yl)- | | | |
| | | 1,2-dihydrospiro[indole-3,4′- | | | |
| | | piperidine] | | | |
| 298 | 12 | 5-fluoro-1′-(methoxyacetyl)-1- | 396.3 | 1.84 | I |
| | | (7H-pyrrolo[2,3-d]pyrimidin-4- | | | |
| | | yl)-1,2-dihydrospiro[indole-3,4′- | | | |
| | | piperidine] | | | |
| 299 | 15 | N-allyl-1-(7H-pyrrolo[2,3- | 389.3 | 1.9 | A |
| | | d]pyrimidin-4-yl)-1,2-dihydro- | | | |
| | | 1′H-spiro[indole-3,4′- | | | |
| | | piperidine]-1′-carboxamide | | | |
| 300 | 15 | N-pentyl-1-(7H-pyrrolo[2,3- | 419.3 | 2.3 | A |
| | | d]pyrimidin-4-yl)-1,2-dihydro- | | | |
| | | 1′H-spiro[indole-3,4′- | | | |
| | | piperidine]-1′-carboxamide | | | |
| 301 | 15 | N-isopropyl-1-(7H-pyrrolo[2,3- | 391.28 | 1.96 | A |
| | | d]pyrimidin-4-yl)-1,2-dihydro- | | | |
| | | 1′H-spiro[indole-3,4′- | | | |
| | | piperidine]-1′-carboxamide | | | |
| 302 | 15 | methyl N-{[1-(7H-pyrrolo[2,3- | 477.33 | 2.3 | A |
| | | d]pyrimidin-4-yl)-1,2-dihydro- | | | |
| | | 1′H-spiro[indole-3,4′-piperidin]- | | | |
| | | 1′-yl]carbonyl}-L-isoleucinate | | | |
| 303 | 15 | N-cyclohexyl-1-(7H- | 431.33 | 2.27 | A |
| | | pyrrolo[2,3-d]pyrimidin-4-yl)- | | | |
| | | 1,2-dihydro-1′H-spiro[indole- | | | |
| | | 3,4′-piperidine]-1′-carboxamide | | | |
| 304 | 15 | methyl N-{[1-(7H-pyrrolo[2,3- | 435.28 | 1.88 | A |
| | | d]pyrimidin-4-yl)-1,2-dihydro- | | | |
| | | 1′H-spiro[indole-3,4′-piperidin]- | | | |
| | | 1′-yl]carbonyl}-L-alaninate | | | |
| 305 | 15 | N-propyl-1-(7H-pyrrolo[2,3- | 391.29 | 1.96 | A |
| | | d]pyrimidin-4-yl)-1,2-dihydro- | | | |
| | | 1′H-spiro[indole-3,4′- | | | |
| | | piperidine]-1′-carboxamide | | | |
| 306 | 15 | N-benzyl-1-(7H-pyrrolo[2,3- | 439.3 | 2.19 | A |
| | | d]pyrimidin-4-yl)-1,2-dihydro- | | | |
| | | 1′H-spiro[indole-3,4′- | | | |
| | | piperidine]-1′-carboxamide | | | |
| 307 | 15 | ethyl N-{[1-(7H-pyrrolo[2,3- | 435.28 | 1.88 | A |
| | | d]pyrimidin-4-yl)-1,2-dihydro- | | | |
| | | 1′H-spiro[indole-3,4′-piperidin]- | | | |
| | | 1′-yl]carbonyl}glycinate | | | |
| 308 | 15 | N-(2-phenylethyl)-1-(7H- | 453.31 | 2.28 | A |
| | | pyrrolo[2,3-d]pyrimidin-4-yl)- | | | |
| | | 1,2-dihydro-1′H-spiro[indole- | | | |
| | | 3,4′-piperidine]-1′-carboxamide | | | |
| 309 | 15 | N-butyl-1-(7H-pyrrolo[2,3- | 405.29 | 2.13 | A |
| | | d]pyrimidin-4-yl)-1,2-dihydro- | | | |
| | | 1′H-spiro[indole-3,4′- | | | |
| | | piperidine]-1′-carboxamide | | | |
| 310 | 15 | N-(3,5-dimethylisoxazol-4-yl)- | 444.27 | 1.89 | A |
| | | 1-(7H-pyrrolo[2,3-d]pyrimidin- | | | |
| | | 4-yl)-1,2-dihydro-1′H- | | | |
| | | spiro[indole-3,4′-piperidine]-1′- | | | |
| | | carboxamide | | | |
| 311 | 15 | N-(2-furylmethyl)-1-(7H- | 429.24 | 2.07 | A |
| | | pyrrolo[2,3-d]pyrimidin-4-yl)- | | | |
| | | 1,2-dihydro-1′H-spiro[indole- | | | |
| | | 3,4′-piperidine]-1′-carboxamide | | | |
| 312 | 15 | N-benzoyl-1-(7H-pyrrolo[2,3- | 453.25 | 2.04 | A |
| | | d]pyrimidin-4-yl)-1,2-dihydro- | | | |
| | | 1′H-spiro[indole-3,4′- | | | |
| | | piperidine]-1′-carboxamide | | | |
| 313 | 15 | N-allyl-5-methyl-1-(7H- | 403.31 | 2.02 | A |
| | | pyrrolo[2,3-d]pyrimidin-4-yl)- | | | |
| | | 1,2-dihydro-1′H-spiro[indole- | | | |
| | | 3,4′-piperidine]-1′-carboxamide | | | |
| 314 | 15 | N-[(1S)-1-phenylethyl]-1-(7H- | 453.31 | 2.31 | A |
| | | pyrrolo[2,3-d]pyrimidin-4-yl)- | | | |
| | | 1,2-dihydro-1′H-spiro[indole- | | | |
| | | 3,4′-piperidine]-1′-carboxamide | | | |
| 315 | 15 | ethyl {[1-(7H-pyrrolo[2,3- | 421.27 | 1.83 | A |
| | | d]pyrimidin-4-yl)-1,2-dihydro- | | | |
| | | 1′H-spiro[indole-3,4′-piperidin]- | | | |
| | | 1′-yl]carbonyl}carbamate | | | |
| 316 | 15 | N-(4-cyanophenyl)-1-(7H- | 450.25 | 2.27 | A |
| | | pyrrolo[2,3-d]pyrimidin-4-yl)- | | | |
| | | 1,2-dihydro-1′H-spiro[indole- | | | |
| | | 3,4′-piperidine]-1′-carboxamide | | | |
| 317 | 15 | N-1H-indol-3-yl-1-(7H- | 464.3 | 2.15 | A |
| | | pyrrolo[2,3-d]pyrimidin-4-yl)- | | | |
| | | 1,2-dihydro-1′H-spiro[indole- | | | |
| | | 3,4′-piperidine]-1′-carboxamide | | | |
| 318 | 15 | N-ethyl-5-methyl-1-(7H- | 391.27 | 1.95 | A |
| | | pyrrolo[2,3-d]pyrimidin-4-yl)- | | | |
| | | 1,2-dihydro-1′H-spiro[indole- | | | |
| | | 3,4′-piperidine]-1′-carboxamide | | | |
| 319 | 15 | ethyl 4-({[1-(7H-pyrrolo[2,3- | 463.31 | 2.03 | A |
| | | d]pyrimidin-4-yl)-1,2-dihydro- | | | |
| | | 1′H-spiro[indole-3,4′-piperidin]- | | | |
| | | 1′-yl]carbonyl}amino)butanoate | | | |
| 320 | 15 | N-phenyl-1-(7H-pyrrolo[2,3- | 425.28 | 2.23 | A |
| | | d]pyrimidin-4-yl)-1,2-dihydro- | | | |
| | | 1′H-spiro[indole-3,4′- | | | |
| | | piperidine]-1′-carboxamide | | | |
| 321 | 15 | N-cyclohexyl-5-methyl-1-(7H- | 445.35 | 2.38 | A |
| | | pyrrolo[2,3-d]pyrimidin-4-yl)- | | | |
| | | 1,2-dihydro-1′H-spiro[indole- | | | |
| | | 3,4′-piperidine]-1′-carboxamide | | | |
| 322 | 15 | N-cyclopentyl-1-(7H- | 417.32 | 2.15 | A |
| | | pyrrolo[2,3-d]pyrimidin-4-yl)- | | | |
| | | 1,2-dihydro-1′H-spiro[indole- | | | |
| | | 3,4′-piperidine]-1′-carboxamide | | | |
| 323 | 15 | ethyl N-{[5-methyl-1-(7H- | 449.3 | 2 | A |
| | | pyrrolo[2,3-d]pyrimidin-4-yl)- | | | |
| | | 1,2-dihydro-1′H-spiro[indole- | | | |
| | | 3,4′-piperidin]-1′- | | | |
| | | yl]carbonyl}glycinate | | | |
| 324 | 15 | N-isopropyl-5-methyl-1-(7H- | 405.29 | 2.08 | A |
| | | pyrrolo[2,3-d]pyrimidin-4-yl)- | | | |
| | | 1,2-dihydro-1′H-spiro[indole- | | | |
| | | 3,4′-piperidine]-1′-carboxamide | | | |
| 325 | 15 | methyl N-{[5-methyl-1-(7H- | 449.3 | 1.99 | A |
| | | pyrrolo[2,3-d]pyrimidin-4-yl)- | | | |
| | | 1,2-dihydro-1′H-spiro[indole- | | | |
| | | 3,4′-piperidin]-1′-yl]carbonyl}-L- | | | |
| | | alaninate | | | |
| 326 | 15 | 5-methyl-N-propyl-1-(7H- | 405.29 | 2.08 | A |
| | | pyrrolo[2,3-d]pyrimidin-4-yl)- | | | |
| | | 1,2-dihydro-1′H-spiro[indole- | | | |
| | | 3,4′-piperidine]-1′-carboxamide | | | |
| 327 | 15 | methyl N-{[5-methyl-1-(7H- | 491.34 | 2.41 | A |
| | | pyrrolo[2,3-d]pyrimidin-4-yl)- | | | |
| | | 1,2-dihydro-1′H-spiro[indole- | | | |
| | | 3,4′-piperidin]-1′-yl]carbonyl}- | | | |
| | | D-leucinate | | | |
| 328 | 15 | N-benzyl-5-methyl-1-(7H- | 453.31 | 2.32 | A |
| | | pyrrolo[2,3-d]pyrimidin-4-yl)- | | | |
| | | 1,2-dihydro-1′H-spiro[indole- | | | |
| | | 3,4′-piperidine]-1′-carboxamide | | | |
| 329 | 15 | ethyl 4-({[5-methyl-1-(7H- | 477.33 | 2.13 | A |
| | | pyrrolo[2,3-d]pyrimidin-4-yl)- | | | |
| | | 1,2-dihydro-1′H-spiro[indole- | | | |
| | | 3,4′-piperidin]-1′- | | | |
| | | yl]carbonyl}amino)butanoate | | | |
| 330 | 15 | methyl N-{[5-methyl-1-(7H- | 491.34 | 2.4 | A |
| | | pyrrolo[2,3-d]pyrimidin-4-yl)- | | | |
| | | 1,2-dihydro-1′H-spiro[indole- | | | |
| | | 3,4′-piperidin]-1′-yl]carbonyl}-L- | | | |
| | | isoleucinate | | | |
| 331 | 15 | N-cyclopentyl-5-methyl-1-(7H- | 431.34 | 2.26 | A |
| | | pyrrolo[2,3-d]pyrimidin-4-yl)- | | | |
| | | 1,2-dihydro-1′H-spiro[indole- | | | |
| | | 3,4′-piperidine]-1′-carboxamide | | | |
| 332 | 15 | 5-methyl-N-phenyl-1-(7H- | 439.3 | 2.34 | A |
| | | pyrrolo[2,3-d]pyrimidin-4-yl)- | | | |
| | | 1,2-dihydro-1′H-spiro[indole- | | | |
| | | 3,4′-piperidine]-1′-carboxamide | | | |
| 333 | 15 | 5-methyl-N-pentyl-1-(7H- | 433.31 | 2.4 | A |
| | | pyrrolo[2,3-d]pyrimidin-4-yl)- | | | |
| | | 1,2-dihydro-1′H-spiro[indole- | | | |
| | | 3,4′-piperidine]-1′-carboxamide | | | |
| 334 | 15 | N-butyl-5-methyl-1-(7H- | 419.31 | 2.24 | A |
| | | pyrrolo[2,3-d]pyrimidin-4-yl)- | | | |
| | | 1,2-dihydro-1′H-spiro[indole- | | | |
| | | 3,4′-piperidine]-1′-carboxamide | | | |
| 335 | 15 | ethyl {[5-methyl-1-(7H- | 435.28 | 1.95 | A |
| | | pyrrolo[2,3-d]pyrimidin-4-yl)- | | | |
| | | 1,2-dihydro-1′H-spiro[indole- | | | |
| | | 3,4′-piperidin]-1′- | | | |
| | | yl]carbonyl}carbamate | | | |
| 336 | 15 | N-(4-cyanophenyl)-5-methyl-1- | 464.29 | 2.36 | A |
| | | (7H-pyrrolo[2,3-d]pyrimidin-4- | | | |
| | | yl)-1,2-dihydro-1′H- | | | |
| | | spiro[indole-3,4′-piperidine]-1′- | | | |
| | | carboxamide | | | |
| 337 | 15 | N-benzoyl-5-methyl-1-(7H- | 467.28 | 2.15 | A |
| | | pyrrolo[2,3-d]pyrimidin-4-yl)- | | | |
| | | 1,2-dihydro-1′H-spiro[indole- | | | |
| | | 3,4′-piperidine]-1′-carboxamide | | | |
| 338 | 15 | 5-methyl-N-(2-phenylethyl)-1- | 467.32 | 2.37 | A |
| | | (7H-pyrrolo[2,3-d]pyrimidin-4- | | | |
| | | yl)-1,2-dihydro-1′H- | | | |
| | | spiro[indole-3,4′-piperidine]-1′- | | | |
| | | carboxamide | | | |
| 339 | 15 | N-(3,5-dimethylisoxazol-4-yl)- | 458.28 | 2 | A |
| | | 5-methyl-1-(7H-pyrrolo[2,3- | | | |
| | | d]pyrimidin-4-yl)-1,2-dihydro- | | | |
| | | 1′H-spiro[indole-3,4′- | | | |
| | | piperidine]-1′-carboxamide | | | |
| 340 | 15 | N-1H-indol-3-yl-5-methyl-1- | 478.31 | 2.26 | A |
| | | (7H-pyrrolo[2,3-d]pyrimidin-4- | | | |
| | | yl)-1,2-dihydro-1′H- | | | |
| | | spiro[indole-3,4′-piperidine]-1′- | | | |
| | | carboxamide | | | |
| 341 | 15 | N-allyl-1-(5-chloro-7H- | 423.23 | 2.27 | A |
| | | pyrrolo[2,3-d]pyrimidin-4-yl)- | | | |
| | | 1,2-dihydro-1′H-spiro[indole- | | | |
| | | 3,4′-piperidine]-1′-carboxamide | | | |
| 342 | 15 | 1-(5-chloro-7H-pyrrolo[2,3- | 453.3 | 2.42 | A |
| | | d]pyrimidin-4-yl)-N-pentyl-1,2- | | | |
| | | dihydro-1′H-spiro[indole-3,4′- | | | |
| | | piperidine]-1′-carboxamide | | | |
| 343 | 15 | 5-methyl-N-[(1S)-1- | 467.32 | 2.4 | A |
| | | phenylethyl]-1-(7H-pyrrolo[2,3- | | | |
| | | d]pyrimidin-4-yl)-1,2-dihydro- | | | |
| | | 1′H-spiro[indole-3,4′- | | | |
| | | piperidine]-1′-carboxamide | | | |
| 344 | 15 | 1-(5-chloro-7H-pyrrolo[2,3- | 465.29 | 2.4 | A |
| | | d]pyrimidin-4-yl)-N-cyclohexyl- | | | |
| | | 1,2-dihydro-1′H-spiro[indole- | | | |
| | | 3,4′-piperidine]-1′-carboxamide | | | |
| 345 | 15 | methyl N-{[1-(5-chloro-7H- | 469.24 | 2.23 | A |
| | | pyrrolo[2,3-d]pyrimidin-4-yl)- | | | |
| | | 1,2-dihydro-1′H-spiro[indole- | | | |
| | | 3,4′-piperidin]-1′-yl]carbonyl}-L- | | | |
| | | alaninate | | | |
| 346 | 15 | 1-(5-chloro-7H-pyrrolo[2,3- | 425.23 | 2.35 | A |
| | | d]pyrimidin-4-yl)-N-isopropyl- | | | |
| | | 1,2-dihydro-1′H-spiro[indole- | | | |
| | | 3,4′-piperidine]-1′-carboxamide | | | |
| 347 | 15 | methyl N-{[1-(5-chloro-7H- | 511.31 | 2.44 | A |
| | | pyrrolo[2,3-d]pyrimidin-4-yl)- | | | |
| | | 1,2-dihydro-1′H-spiro[indole- | | | |
| | | 3,4′-piperidin]-1′-yl]carbonyl}- | | | |
| | | D-leucinate | | | |
| 348 | 15 | N-benzyl-1-(5-chloro-7H- | 473.25 | 2.3 | A |
| | | pyrrolo[2,3-d]pyrimidin-4-yl)- | | | |
| | | 1,2-dihydro-1′H-spiro[indole- | | | |
| | | 3,4′-piperidine]-1′-carboxamide | | | |
| 349 | 15 | ethyl N-{[1-(5-chloro-7H- | 469.24 | 2.23 | A |
| | | pyrrolo[2,3-d]pyrimidin-4-yl)- | | | |
| | | 1,2-dihydro-1′H-spiro[indole- | | | |
| | | 3,4′-piperidin]-1′- | | | |
| | | yl]carbonyl}glycinate | | | |
| 350 | 15 | N-(2-furylmethyl)-5-methyl-1- | 443.28 | 2.16 | A |
| | | (7H-pyrrolo[2,3-d]pyrimidin-4- | | | |
| | | yl)-1,2-dihydro-1′H- | | | |
| | | spiro[indole-3,4′-piperidine]-1′- | | | |
| | | carboxamide | | | |
| 351 | 15 | 1-(5-chloro-7H-pyrrolo[2,3- | 487.26 | 2.43 | A |
| | | d]pyrimidin-4-yl)-N-[(1S)-1- | | | |
| | | phenylethyl]-1,2-dihydro-1′H- | | | |
| | | spiro[indole-3,4′-piperidine]-1′- | | | |
| | | carboxamide | | | |
| 352 | 15 | 1-(5-chloro-7H-pyrrolo[2,3- | 484.25 | 2.41 | A |
| | | d]pyrimidin-4-yl)-N-(4- | | | |
| | | cyanophenyl)-1,2-dihydro-1′H- | | | |
| | | spiro[indole-3,4′-piperidine]-1′- | | | |
| | | carboxamide | | | |
| 353 | 15 | 1-(5-chloro-7H-pyrrolo[2,3- | 487.26 | 2.38 | A |
| | | d]pyrimidin-4-yl)-N-(2- | | | |
| | | phenylethyl)-1,2-dihydro-1′H- | | | |
| | | spiro[indole-3,4′-piperidine]-1′- | | | |
| | | carboxamide | | | |
| 354 | 15 | 1-(5-chloro-7H-pyrrolo[2,3- | 459.24 | 2.35 | A |
| | | d]pyrimidin-4-yl)-N-phenyl-1,2- | | | |
| | | dihydro-1′H-spiro[indole-3,4′- | | | |
| | | piperidine]-1′-carboxamide | | | |
| 355 | 15 | 1-(5-chloro-7H-pyrrolo[2,3- | 463.25 | 2.43 | A |
| | | d]pyrimidin-4-yl)-N-(2- | | | |
| | | furylmethyl)-1,2-dihydro-1′H- | | | |
| | | spiro[indole-3,4′-piperidine]-1′- | | | |
| | | carboxamide | | | |
| 356 | 15 | methyl N-{[1-(5-chloro-7H- | 511.3 | 2.45 | A |
| | | pyrrolo[2,3-d]pyrimidin-4-yl)- | | | |
| | | 1,2-dihydro-1′H-spiro[indole- | | | |
| | | 3,4′-piperidin]-1′-yl]carbonyl}-L- | | | |
| | | isoleucinate | | | |
| 357 | 15 | ethyl 4-({[1-(5-chloro-7H- | 497.27 | 2.39 | A |
| | | pyrrolo[2,3-d]pyrimidin-4-yl)- | | | |
| | | 1,2-dihydro-1′H-spiro[indole- | | | |
| | | 3,4′-piperidin]-1′- | | | |
| | | yl]carbonyl}amino)butanoate | | | |
| 358 | 15 | N-benzoyl-1-(5-chloro-7H- | 487.26 | 2.43 | A |
| | | pyrrolo[2,3-d]pyrimidin-4-yl)- | | | |
| | | 1,2-dihydro-1′H-spiro[indole- | | | |
| | | 3,4′-piperidine]-1′-carboxamide | | | |
| 359 | 15 | 1-(5-chloro-7H-pyrrolo[2,3- | 498.29 | 2.53 | A |
| | | d]pyrimidin-4-yl)-N-1H-indol-3- | | | |
| | | yl-1,2-dihydro-1′H-spiro[indole- | | | |
| | | 3,4′-piperidine]-1′-carboxamide | | | |
| 360 | 15 | 1-(5-chloro-7H-pyrrolo[2,3- | 451.27 | 2.25 | A |
| | | d]pyrimidin-4-yl)-N-cyclopentyl- | | | |
| | | 1,2-dihydro-1′H-spiro[indole- | | | |
| | | 3,4′-piperidine]-1′-carboxamide | | | |
| 361 | 15 | 1-(5-chloro-7H-pyrrolo[2,3- | 425.23 | 2.34 | A |
| | | d]pyrimidin-4-yl)-N-propyl-1,2- | | | |
| | | dihydro-1′H-spiro[indole-3,4′- | | | |
| | | piperidine]-1′-carboxamide | | | |
| 362 | 15 | 1-(5-chloro-7H-pyrrolo[2,3- | 467.32 | 2.5 | A |
| | | d]pyrimidin-4-yl)-5-methyl-N- | | | |
| | | pentyl-1,2-dihydro-1′H- | | | |
| | | spiro[indole-3,4′-piperidine]-1′- | | | |
| | | carboxamide | | | |
| 363 | 15 | methyl N-{[1-(5-chloro-7H- | 525.35 | 2.52 | A |
| | | pyrrolo[2,3-d]pyrimidin-4-yl)-5- | | | |
| | | methyl-1,2-dihydro-1′H- | | | |
| | | spiro[indole-3,4′-piperidin]-1′- | | | |
| | | yl]carbonyl}-L-isoleucinate | | | |
| 364 | 15 | N-butyl-1-(5-chloro-7H- | 439.24 | 2.22 | A |
| | | pyrrolo[2,3-d]pyrimidin-4-yl)- | | | |
| | | 1,2-dihydro-1′H-spiro[indole- | | | |
| | | 3,4′-piperidine]-1′-carboxamide | | | |
| 365 | 15 | 1-(5-chloro-7H-pyrrolo[2,3- | 479.3 | 2.47 | A |
| | | d]pyrimidin-4-yl)-N-cyclohexyl- | | | |
| | | 5-methyl-1,2-dihydro-1′H- | | | |
| | | spiro[indole-3,4′-piperidine]-1′- | | | |
| | | carboxamide | | | |
| 366 | 15 | 1-(5-chloro-7H-pyrrolo[2,3- | 439.24 | 2.42 | A |
| | | d]pyrimidin-4-yl)-5-methyl-N- | | | |
| | | propyl-1,2-dihydro-1′H- | | | |
| | | spiro[indole-3,4′-piperidine]-1′- | | | |
| | | carboxamide | | | |
| 367 | 15 | methyl N-{[1-(5-chloro-7H- | 483.25 | 2.32 | A |
| | | pyrrolo[2,3-d]pyrimidin-4-yl)-5- | | | |
| | | methyl-1,2-dihydro-1′H- | | | |
| | | spiro[indole-3,4′-piperidin]-1′- | | | |
| | | yl]carbonyl}-L-alaninate | | | |
| 368 | 15 | 1-(5-chloro-7H-pyrrolo[2,3- | 501.3 | 2.45 | A |
| | | d]pyrimidin-4-yl)-5-methyl-N- | | | |
| | | (2-phenylethyl)-1,2-dihydro- | | | |
| | | 1′H-spiro[indole-3,4′- | | | |
| | | piperidine]-1′-carboxamide | | | |
| 369 | 15 | 1-(5-chloro-7H-pyrrolo[2,3- | 439.25 | 2.42 | A |
| | | d]pyrimidin-4-yl)-N-isopropyl-5- | | | |
| | | methyl-1,2-dihydro-1′H- | | | |
| | | spiro[indole-3,4′-piperidine]-1′- | | | |
| | | carboxamide | | | |
| 370 | 15 | N-butyl-1-(5-chloro-7H- | 453.31 | 2.31 | A |
| | | pyrrolo[2,3-d]pyrimidin-4-yl)-5- | | | |
| | | methyl-1,2-dihydro-1′H- | | | |
| | | spiro[indole-3,4′-piperidine]-1′- | | | |
| | | carboxamide | | | |
| 371 | 15 | methyl N-{[1-(5-chloro-7H- | 525.35 | 2.5 | A |
| | | pyrrolo[2,3-d]pyrimidin-4-yl)-5- | | | |
| | | methyl-1,2-dihydro-1′H- | | | |
| | | spiro[indole-3,4′-piperidin]-1′- | | | |
| | | yl]carbonyl}-D-leucinate | | | |
| 372 | 15 | ethyl 4-({[1-(5-chloro-7H- | 511.3 | 2.46 | A |
| | | pyrrolo[2,3-d]pyrimidin-4-yl)-5- | | | |
| | | methyl-1,2-dihydro-1′H- | | | |
| | | spiro[indole-3,4′-piperidin]-1′- | | | |
| | | yl]carbonyl}amino)butanoate | | | |
| 373 | 15 | 1-(5-chloro-7H-pyrrolo[2,3- | 498.28 | 2.47 | A |
| | | d]pyrimidin-4-yl)-N-(4- | | | |
| | | cyanophenyl)-5-methyl-1,2- | | | |
| | | dihydro-1′H-spiro[indole-3,4′- | | | |
| | | piperidine]-1′-carboxamide | | | |
| 374 | 15 | N-benzyl-1-(5-chloro-7H- | 487.27 | 2.37 | A |
| | | pyrrolo[2,3-d]pyrimidin-4-yl)-5- | | | |
| | | methyl-1,2-dihydro-1′H- | | | |
| | | spiro[indole-3,4′-piperidine]-1′- | | | |
| | | carboxamide | | | |
| 375 | 15 | 1-(5-chloro-7H-pyrrolo[2,3- | 473.25 | 2.43 | A |
| | | d]pyrimidin-4-yl)-5-methyl-N- | | | |
| | | phenyl-1,2-dihydro-1′H- | | | |
| | | spiro[indole-3,4′-piperidine]-1′- | | | |
| | | carboxamide | | | |
| 376 | 15 | 1-(5-chloro-7H-pyrrolo[2,3- | 512.3 | 2.29 | A |
| | | d]pyrimidin-4-yl)-N-1H-indol-3- | | | |
| | | yl-5-methyl-1,2-dihydro-1′H- | | | |
| | | spiro[indole-3,4′-piperidine]-1′- | | | |
| | | carboxamide | | | |
| 377 | 15 | N-benzoyl-1-(5-chloro-7H- | 501.28 | 2.51 | A |
| | | pyrrolo[2,3-d]pyrimidin-4-yl)-5- | | | |
| | | methyl-1,2-dihydro-1′H- | | | |
| | | spiro[indole-3,4′-piperidine]-1′- | | | |
| | | carboxamide | | | |
| 378 | 15 | 1-(5-chloro-7H-pyrrolo[2,3- | 465.29 | 2.33 | A |
| | | d]pyrimidin-4-yl)-N-cyclopentyl- | | | |
| | | 5-methyl-1,2-dihydro-1′H- | | | |
| | | spiro[indole-3,4′-piperidine]-1′- | | | |
| | | carboxamide | | | |
| 379 | 15 | 1-(5-chloro-7H-pyrrolo[2,3- | 492.28 | 2.33 | A |
| | | d]pyrimidin-4-yl)-N-(3,5- | | | |
| | | dimethylisoxazol-4-yl)-5- | | | |
| | | methyl-1,2-dihydro-1′H- | | | |
| | | spiro[indole-3,4′-piperidine]-1′- | | | |
| | | carboxamide | | | |
| 380 | 15 | 1-(5-chloro-7H-pyrrolo[2,3- | 501.29 | 2.49 | A |
| | | d]pyrimidin-4-yl)-5-methyl-N- | | | |
| | | [(1S)-1-phenylethyl]-1,2- | | | |
| | | dihydro-1′H-spiro[indole-3,4′- | | | |
| | | piperidine]-1′-carboxamide | | | |
| 381 | 15 | 1-(5-chloro-7H-pyrrolo[2,3- | 477.26 | 2.5 | A |
| | | d]pyrimidin-4-yl)-N-(2- | | | |
| | | furylmethyl)-5-methyl-1,2- | | | |
| | | dihydro-1′H-spiro[indole-3,4′- | | | |
| | | piperidine]-1′-carboxamide | | | |
| 382 | 15 | methyl N-{[1-(7H-pyrrolo[2,3- | 477.33 | 2.32 | A |
| | | d]pyrimidin-4-yl)-1,2-dihydro- | | | |
| | | 1′H-spiro[indole-3,4′-piperidin]- | | | |
| | | 1′-yl]carbonyl}-L-leucinate | | | |
| 383 | 15 | N-methyl-1-(7H-pyrrolo[2,3- | 363.19 | 1.78 | B |
| | | d]pyrimidin-4-yl)-1,2-dihydro- | | | |
| | | 1′H-spiro[indole-3,4′- | | | |
| | | piperidine]-1′-carboxamide | | | |
| 384 | 14 | methyl 5-chloro-1-(7H- | 398.14 | 2.59 | B |
| | | pyrrolo[2,3-d]pyrimidin-4-yl)- | | | |
| | | 1,2-dihydro-1′H-spiro[indole- | | | |
| | | 3,4′-piperidine]-1′-carboxylate | | | |
| 385 | 15 | 5-chloro-N-methyl-1-(7H- | 397.16 | 2.13 | B |
| | | pyrrolo[2,3-d]pyrimidin-4-yl)- | | | |
| | | 1,2-dihydro-1′H-spiro[indole- | | | |
| | | 3,4′-piperidine]-1′-carboxamide | | | |
| 386 | 14 | methyl 1-(7H-pyrrolo[2,3- | 364.18 | 2.19 | B |
| | | d]pyrimidin-4-yl)-1,2-dihydro- | | | |
| | | 1′H-spiro[indole-3,4′- | | | |
| | | piperidine]-1′-carboxylate | | | |
| 387 | 14 | N-ethyl-1-(5-methyl-7H- | 390.4 | 5.34 | H |
| | | pyrrolo[2,3-d]pyrimidin-4-yl)- | | | |
| | | 1,2-dihydro-1′H-spiro[indole- | | | |
| | | 3,4′-piperidine]-1′-carboxamide | | | |
| 388 | 15 | 1-(5-chloro-7H-pyrrolo[2,3- | 478.17 | 2.3 | E |
| | | d]pyrimidin-4-yl)-N-(3,5- | | | |
| | | dimethylisoxazol-4-yl)-1,2- | | | |
| | | dihydro-1′H-spiro[indole-3,4′- | | | |
| | | piperidine]-1′-carboxamide | | | |
| 389 | 15 | ethyl N-{[1-(5-chloro-7H- | 483.18 | 2.46 | E |
| | | pyrrolo[2,3-d]pyrimidin-4-yl)-5- | | | |
| | | methyl-1,2-dihydro-1′H- | | | |
| | | spiro[indole-3,4′-piperidin]-1′- | | | |
| | | yl]carbonyl}glycinate | | | |
| 390 | 15 | N-allyl-1-(5-chloro-7H- | 437.18 | 2.49 | E |
| | | pyrrolo[2,3-d]pyrimidin-4-yl)-5- | | | |
| | | methyl-1,2-dihydro-1′H- | | | |
| | | spiro[indole-3,4′-piperidine]-1′- | | | |
| | | carboxamide | | | |
| 391 | 15 | N-(2-morpholin-4-ylethyl)-1- | 462.27 | 2.08 | C |
| | | (7H-pyrrolo[2,3-d]pyrimidin-4- | | | |
| | | yl)-1,2-dihydro-1′H- | | | |
| | | spiro[indole-3,4′-piperidine]-1′- | | | |
| | | carboxamide | | | |
| 392 | 15 | N-[2-(dimethylamino)ethyl]-1- | 420.25 | 2 | C |
| | | (7H-pyrrolo[2,3-d]pyrimidin-4- | | | |
| | | yl)-1,2-dihydro-1′H- | | | |
| | | spiro[indole-3,4′-piperidine]-1′- | | | |
| | | carboxamide | | | |
| 393 | 15 | N-(3-morpholin-4-ylpropyl)-1- | 476.3 | 2.09 | C |
| | | (7H-pyrrolo[2,3-d]pyrimidin-4- | | | |
| | | yl)-1,2-dihydro-1′H- | | | |
| | | spiro[indole-3,4′-piperidine]-1′- | | | |
| | | carboxamide | | | |
| 394 | 15 | N-[3-(dimethylamino)propyl]-1- | 434.28 | 2.05 | C |
| | | (7H-pyrrolo[2,3-d]pyrimidin-4- | | | |
| | | yl)-1,2-dihydro-1′H- | | | |
| | | spiro[indole-3,4′-piperidine]-1′- | | | |
| | | carboxamide | | | |
| 395 | 15 | N-(2-hydroxyethyl)-1-(7H- | 393.21 | 2.25 | C |
| | | pyrrolo[2,3-d]pyrimidin-4-yl)- | | | |
| | | 1,2-dihydro-1′H-spiro[indole- | | | |
| | | 3,4′-piperidine]-1′-carboxamide | | | |
| 396 | 15 | N-(2-pyrrolidin-1-ylethyl)-1- | 446.27 | 2.08 | C |
| | | (7H-pyrrolo[2,3-d]pyrimidin-4- | | | |
| | | yl)-1,2-dihydro-1′H- | | | |
| | | spiro[indole-3,4′-piperidine]-1′- | | | |
| | | carboxamide | | | |
| 397 | 15 | N-[2-(dimethylamino)ethyl]-N- | 434.29 | 2.16 | C |
| | | methyl-1-(7H-pyrrolo[2,3- | | | |
| | | d]pyrimidin-4-yl)-1,2-dihydro- | | | |
| | | 1′H-spiro[indole-3,4′- | | | |
| | | piperidine]-1′-carboxamide | | | |
| 398 | 15 | N-[2-(1-methylpyrrolidin-2- | 460.29 | 2.1 | C |
| | | yl)ethyl]-1-(7H-pyrrolo[2,3- | | | |
| | | d]pyrimidin-4-yl)-1,2-dihydro- | | | |
| | | 1′H-spiro[indole-3,4′- | | | |
| | | piperidine]-1′-carboxamide | | | |
| 399 | 15 | (1-{[1-(7H-pyrrolo[2,3- | 433.23 | 2.01 | C |
| | | d]pyrimidin-4-yl)-1,2-dihydro- | | | |
| | | 1′H-spiro[indole-3,4′-piperidin]- | | | |
| | | 1′-yl]carbonyl}pyrrolidin-2- | | | |
| | | yl)methanol | | | |
| 400 | 15 | N-(pyridin-3-ylmethyl)-1-(7H- | 440.22 | 2.05 | C |
| | | pyrrolo[2,3-d]pyrimidin-4-yl)- | | | |
| | | 1,2-dihydro-1′H-spiro[indole- | | | |
| | | 3,4′-piperidine]-1′-carboxamide | | | |
| 401 | 15 | N-{[(2S)-1-ethylpyrrolidin-2- | 460.29 | 2.22 | C |
| | | yl]methyl}-1-(7H-pyrrolo[2,3- | | | |
| | | d]pyrimidin-4-yl)-1,2-dihydro- | | | |
| | | 1′H-spiro[indole-3,4′- | | | |
| | | piperidine]-1′-carboxamide | | | |
| 402 | 15 | N-[3-(dimethylamino)propyl]- | 448.31 | 2.24 | C |
| | | N-methyl-1-(7H-pyrrolo[2,3- | | | |
| | | d]pyrimidin-4-yl)-1,2-dihydro- | | | |
| | | 1′H-spiro[indole-3,4′- | | | |
| | | piperidine]-1′-carboxamide | | | |
| 403 | 15 | N-(2-methoxyethyl)-1-(7H- | 407.22 | 1.86 | C |
| | | pyrrolo[2,3-d]pyrimidin-4-yl)- | | | |
| | | 1,2-dihydro-1′H-spiro[indole- | | | |
| | | 3,4′-piperidine]-1′-carboxamide | | | |
| 404 | 15 | (1-{[1-(7H-pyrrolo[2,3- | 447.29 | 1.94 | C |
| | | d]pyrimidin-4-yl)-1,2-dihydro- | | | |
| | | 1′H-spiro[indole-3,4′-piperidin]- | | | |
| | | 1′-yl]carbonyl}piperidin-4- | | | |
| | | yl)methanol | | | |
| 405 | 15 | N-[4-(dimethylamino)butyl]-1- | 448.31 | 2.08 | C |
| | | (7H-pyrrolo[2,3-d]pyrimidin-4- | | | |
| | | yl)-1,2-dihydro-1′H- | | | |
| | | spiro[indole-3,4′-piperidine]-1′- | | | |
| | | carboxamide | | | |
| 406 | 15 | 1′-[(4-ethylpiperazin-1- | 446.28 | 2.07 | C |
| | | yl)carbonyl]-1-(7H-pyrrolo[2,3- | | | |
| | | d]pyrimidin-4-yl)-1,2- | | | |
| | | dihydrospiro[indole-3,4′- | | | |
| | | piperidine] | | | |
| 407 | 15 | 2-(1-{[1-(7H-pyrrolo[2,3- | 461.29 | 2.04 | C |
| | | d]pyrimidin-4-yl)-1,2-dihydro- | | | |
| | | 1′H-spiro[indole-3,4′-piperidin]- | | | |
| | | 1′-yl]carbonyl}piperidin-4- | | | |
| | | yl)ethanol | | | |
| 408 | 15 | N-[3-(dimethylamino)-2,2- | 462.33 | 2.24 | C |
| | | dimethylpropyl]-1-(7H- | | | |
| | | pyrrolo[2,3-d]pyrimidin-4-yl)- | | | |
| | | 1,2-dihydro-1′H-spiro[indole- | | | |
| | | 3,4′-piperidine]-1′-carboxamide | | | |
| 409 | 15 | N-[(1R,2R)-2- | 505.39 | 2.09 | C |
| | | hydroxycyclohexyl]-N-(2- | | | |
| | | methoxyethyl)-1-(7H- | | | |
| | | pyrrolo[2,3-d]pyrimidin-4-yl)- | | | |
| | | 1,2-dihydro-1′H-spiro[indole- | | | |
| | | 3,4′-piperidine]-1′-carboxamide | | | |
| 410 | 15 | N-[(1-methylpiperidin-3- | 460.31 | 2.1 | C |
| | | yl)methyl]-1-(7H-pyrrolo[2,3- | | | |
| | | d]pyrimidin-4-yl)-1,2-dihydro- | | | |
| | | 1′H-spiro[indole-3,4′- | | | |
| | | piperidine]-1′-carboxamide | | | |
| 411 | 15 | (3R)—N,N-dimethyl-1-{[1-(7H- | 446.27 | 2.06 | C |
| | | pyrrolo[2,3-d]pyrimidin-4-yl)- | | | |
| | | 1,2-dihydro-1′H-spiro[indole- | | | |
| | | 3,4′-piperidin]-1′- | | | |
| | | yl]carbonyl}pyrrolidin-3-amine | | | |
| 412 | 15 | N-[(2R)-2-hydroxypropyl]-1- | 407.22 | 2.34 | C |
| | | (7H-pyrrolo[2,3-d]pyrimidin-4- | | | |
| | | yl)-1,2-dihydro-1′H- | | | |
| | | spiro[indole-3,4′-piperidine]-1′- | | | |
| | | carboxamide | | | |
| 413 | 15 | N-[(1R,2R)-2-(4- | 515.38 | 2.02 | C |
| | | methylpiperazin-1- | | | |
| | | yl)cyclopentyl]-1-(7H- | | | |
| | | pyrrolo[2,3-d]pyrimidin-4-yl)- | | | |
| | | 1,2-dihydro-1′H-spiro[indole- | | | |
| | | 3,4′-piperidine]-1′-carboxamide | | | |
| 414 | 15 | (1R,2R)-2-(4-{[1-(7H- | 502.33 | 2.13 | C |
| | | pyrrolo[2,3-d]pyrimidin-4-yl)- | | | |
| | | 1,2-dihydro-1′H-spiro[indole- | | | |
| | | 3,4′-piperidin]-1′- | | | |
| | | yl]carbonyl}piperazin-1- | | | |
| | | yl)cyclopentanol | | | |
| 415 | 15 | N-{[1-(2- | 504.35 | 2.14 | C |
| | | methoxyethyl)piperidin-4- | | | |
| | | yl]methyl}-1-(7H-pyrrolo[2,3- | | | |
| | | d]pyrimidin-4-yl)-1,2-dihydro- | | | |
| | | 1′H-spiro[indole-3,4′- | | | |
| | | piperidine]-1′-carboxamide | | | |
| 416 | 15 | N-{[1-(2- | 518.36 | 2.35 | C |
| | | methoxyethyl)piperidin-4- | | | |
| | | yl]methyl}-N-methyl-1-(7H- | | | |
| | | pyrrolo[2,3-d]pyrimidin-4-yl)- | | | |
| | | 1,2-dihydro-1′H-spiro[indole- | | | |
| | | 3,4′-piperidine]-1′-carboxamide | | | |
| 417 | 15 | (3S)-1-{[1-(7H-pyrrolo[2,3- | 419.21 | 1.82 | C |
| | | d]pyrimidin-4-yl)-1,2-dihydro- | | | |
| | | 1′H-spiro[indole-3,4′-piperidin]- | | | |
| | | 1′-yl]carbonyl}pyrrolidin-3-ol | | | |
| 418 | 15 | 1-{[1-(7H-pyrrolo[2,3- | 446.25 | 1.84 | C |
| | | d]pyrimidin-4-yl)-1,2-dihydro- | | | |
| | | 1′H-spiro[indole-3,4′-piperidin]- | | | |
| | | 1′-yl]carbonyl}-L-prolinamide | | | |
| 419 | 15 | N-{[1-(2- | 504.36 | 2.18 | C |
| | | methoxyethyl)piperidin-3- | | | |
| | | yl]methyl}-1-(7H-pyrrolo[2,3- | | | |
| | | d]pyrimidin-4-yl)-1,2-dihydro- | | | |
| | | 1′H-spiro[indole-3,4′- | | | |
| | | piperidine]-1′-carboxamide | | | |
| 420 | 15 | 5-chloro-N-(3-morpholin-4- | 510.3 | 2.49 | C |
| | | ylpropyl)-1-(7H-pyrrolo[2,3- | | | |
| | | d]pyrimidin-4-yl)-1,2-dihydro- | | | |
| | | 1′H-spiro[indole-3,4′- | | | |
| | | piperidine]-1′-carboxamide | | | |
| 421 | 15 | N-(3-pyrrolidin-1-ylpropyl)-1- | 460.31 | 2.13 | C |
| | | (7H-pyrrolo[2,3-d]pyrimidin-4- | | | |
| | | yl)-1,2-dihydro-1′H- | | | |
| | | spiro[indole-3,4′-piperidine]-1′- | | | |
| | | carboxamide | | | |
| 422 | 15 | N,N-dimethyl-1-(7H- | 377.22 | 2.05 | C |
| | | pyrrolo[2,3-d]pyrimidin-4-yl)- | | | |
| | | 1,2-dihydro-1′H-spiro[indole- | | | |
| | | 3,4′-piperidine]-1′-carboxamide | | | |
| 423 | 15 | 5-chloro-N-[3- | 468.25 | 2.44 | C |
| | | (dimethylamino)propyl]-1-(7H- | | | |
| | | pyrrolo[2,3-d]pyrimidin-4-yl)- | | | |
| | | 1,2-dihydro-1′H-spiro[indole- | | | |
| | | 3,4′-piperidine]-1′-carboxamide | | | |
| 424 | 15 | 5-chloro-N-{[(2S)-1- | 494.3 | 2.64 | C |
| | | ethylpyrrolidin-2-yl]methyl}-1- | | | |
| | | (7H-pyrrolo[2,3-d]pyrimidin-4- | | | |
| | | yl)-1,2-dihydro-1′H- | | | |
| | | spiro[indole-3,4′-piperidine]-1′- | | | |
| | | carboxamide | | | |
| 425 | 15 | 5-chloro-N-(2-morpholin-4- | 496.27 | 2.46 | C |
| | | ylethyl)-1-(7H-pyrrolo[2,3- | | | |
| | | d]pyrimidin-4-yl)-1,2-dihydro- | | | |
| | | 1′H-spiro[indole-3,4′- | | | |
| | | piperidine]-1′-carboxamide | | | |
| 426 | 15 | 5-chloro-N-[2- | 454.24 | 2.39 | C |
| | | (dimethylamino)ethyl]-1-(7H- | | | |
| | | pyrrolo[2,3-d]pyrimidin-4-yl)- | | | |
| | | 1,2-dihydro-1′H-spiro[indole- | | | |
| | | 3,4′-piperidine]-1′-carboxamide | | | |
| 427 | 15 | 5-chloro-N-(2-pyrrolidin-1- | 480.29 | 2.49 | C |
| | | ylethyl)-1-(7H-pyrrolo[2,3- | | | |
| | | d]pyrimidin-4-yl)-1,2-dihydro- | | | |
| | | 1′H-spiro[indole-3,4′- | | | |
| | | piperidine]-1′-carboxamide | | | |
| 428 | 15 | 1-(7H-pyrrolo[2,3-d]pyrimidin- | 349.19 | 2.25 | C |
| | | 4-yl)-1,2-dihydro-1′H- | | | |
| | | spiro[indole-3,4′-piperidine]-1′- | | | |
| | | carboxamide | | | |
| 429 | 15 | 5-chloro-N-[2- | 468.25 | 2.56 | C |
| | | (dimethylamino)ethyl]-N- | | | |
| | | methyl-1-(7H-pyrrolo[2,3- | | | |
| | | d]pyrimidin-4-yl)-1,2-dihydro- | | | |
| | | 1′H-spiro[indole-3,4′- | | | |
| | | piperidine]-1′-carboxamide | | | |
| 430 | 15 | 5-chloro-1′-[(4-ethylpiperazin- | 480.28 | 2.49 | C |
| | | 1-yl)carbonyl]-1-(7H- | | | |
| | | pyrrolo[2,3-d]pyrimidin-4-yl)- | | | |
| | | 1,2-dihydrospiro[indole-3,4′- | | | |
| | | piperidine] | | | |
| 431 | 15 | 5-chloro-N-[3- | 482.28 | 2.62 | C |
| | | (dimethylamino)propyl]-N- | | | |
| | | methyl-1-(7H-pyrrolo[2,3- | | | |
| | | d]pyrimidin-4-yl)-1,2-dihydro- | | | |
| | | 1′H-spiro[indole-3,4′- | | | |
| | | piperidine]-1′-carboxamide | | | |
| 432 | 15 | 5-chloro-N-[2-(1- | 494.3 | 2.49 | C |
| | | methylpyrrolidin-2-yl)ethyl]-1- | | | |
| | | (7H-pyrrolo[2,3-d]pyrimidin-4- | | | |
| | | yl)-1,2-dihydro-1′H- | | | |
| | | spiro[indole-3,4′-piperidine]-1′- | | | |
| | | carboxamide | | | |
| 433 | 15 | (3R)-1-{[5-chloro-1-(7H- | 480.28 | 2.46 | C |
| | | pyrrolo[2,3-d]pyrimidin-4-yl)- | | | |
| | | 1,2-dihydro-1′H-spiro[indole- | | | |
| | | 3,4′-piperidin]-1′-yl]carbonyl}- | | | |
| | | N,N-dimethylpyrrolidin-3- | | | |
| | | amine | | | |
| 434 | 15 | 5-chloro-N-[3-(dimethylamino)- | 496.28 | 2.66 | C |
| | | 2,2-dimethylpropyl]-1-(7H- | | | |
| | | pyrrolo[2,3-d]pyrimidin-4-yl)- | | | |
| | | 1,2-dihydro-1′H-spiro[indole- | | | |
| | | 3,4′-piperidine]-1′-carboxamide | | | |
| 435 | 15 | 5-chloro-N-pyridin-3-yl-1-(7H- | 460.22 | 2.1 | C |
| | | pyrrolo[2,3-d]pyrimidin-4-yl)- | | | |
| | | 1,2-dihydro-1′H-spiro[indole- | | | |
| | | 3,4′-piperidine]-1′-carboxamide | | | |
| 436 | 15 | 5-chloro-N-[(1-methylpiperidin- | 494.29 | 2.49 | C |
| | | 3-yl)methyl]-1-(7H-pyrrolo[2,3- | | | |
| | | d]pyrimidin-4-yl)-1,2-dihydro- | | | |
| | | 1′H-spiro[indole-3,4′- | | | |
| | | piperidine]-1′-carboxamide | | | |
| 437 | 15 | (3S)-1-{[5-chloro-1-(7H- | 453.22 | 2.08 | C |
| | | pyrrolo[2,3-d]pyrimidin-4-yl)- | | | |
| | | 1,2-dihydro-1′H-spiro[indole- | | | |
| | | 3,4′-piperidin]-1′- | | | |
| | | yl]carbonyl}pyrrolidin-3-ol | | | |
| 438 | 15 | (1R,2R)-2-(4-{[5-chloro-1-(7H- | 536.33 | 2.52 | C |
| | | pyrrolo[2,3-d]pyrimidin-4-yl)- | | | |
| | | 1,2-dihydro-1′H-spiro[indole- | | | |
| | | 3,4′-piperidin]-1′- | | | |
| | | yl]carbonyl}piperazin-1- | | | |
| | | yl)cyclopentanol | | | |
| 439 | 15 | 5-chloro-N-(3-pyrrolidin-1- | 494.28 | 2.52 | C |
| | | ylpropyl)-1-(7H-pyrrolo[2,3- | | | |
| | | d]pyrimidin-4-yl)-1,2-dihydro- | | | |
| | | 1′H-spiro[indole-3,4′- | | | |
| | | piperidine]-1′-carboxamide | | | |
| 440 | 15 | 1-{[5-chloro-1-(7H-pyrrolo[2,3- | 480.23 | 2.1 | C |
| | | d]pyrimidin-4-yl)-1,2-dihydro- | | | |
| | | 1′H-spiro[indole-3,4′-piperidin]- | | | |
| | | 1′-yl]carbonyl}-L-prolinamide | | | |
| 441 | 15 | 5-chloro-N-{[1-(2- | 538.34 | 2.57 | C |
| | | methoxyethyl)piperidin-3- | | | |
| | | yl]methyl}-1-(7H-pyrrolo[2,3- | | | |
| | | d]pyrimidin-4-yl)-1,2-dihydro- | | | |
| | | 1′H-spiro[indole-3,4′- | | | |
| | | piperidine]-1′-carboxamide | | | |
| 442 | 15 | 5-bromo-N-[2- | 500.2 | 1.72 | I |
| | | (dimethylamino)ethyl]-1-(7H- | | | |
| | | pyrrolo[2,3-d]pyrimidin-4-yl)- | | | |
| | | 1,2-dihydro-1′H-spiro[indole- | | | |
| | | 3,4′-piperidine]-1′-carboxamide | | | |
| 443 | 13 | 1′-[(4-chlorophenyl)sulfonyl]-1- | 479.96 | 2.57 | C |
| | | (7H-pyrrolo[2,3-d]pyrimidin-4- | | | |
| | | yl)-1,2-dihydrospiro[indole-3,4′- | | | |
| | | piperidine] | | | |
| 444 | 13 | 5-fluoro-1′-(methylsulfonyl)-1- | 402.2 | 2.09 | I |
| | | (7H-pyrrolo[2,3-d]pyrimidin-4- | | | |
| | | yl)-1,2-dihydrospiro[indole-3,4′- | | | |
| | | piperidine] | | | |
| 445 | 14, 19 | benzyl (3S)-1-(7H-pyrrolo[2,3- | 426.11 | 1.51 | E |
| | | d]pyrimidin-4-yl)-1,2-dihydro- | | | |
| | | 1′H-spiro[indole-3,3′- | | | |
| | | pyrrolidine]-1′-carboxylate | | | |
| 446 | 14, 19 | benzyl (3S)-1-(5-chloro-7H- | 460.07 | 1.61 | E |
| | | pyrrolo[2,3-d]pyrimidin-4-yl)- | | | |
| | | 1,2-dihydro-1′H-spiro[indole- | | | |
| | | 3,3′-pyrrolidine]-1′-carboxylate | | | |
| 447 | 19, 10 | (3R)-1′-methyl-1-(7H- | 306.12 | 1.97 | E |
| | | pyrrolo[2,3-d]pyrimidin-4-yl)- | | | |
| | | 1,2-dihydrospiro[indole-3,3′- | | | |
| | | pyrrolidine] | | | |
| 448 | 19 | (3R)-1-(7H-pyrrolo[2,3- | 292.1 | 2.73 | D |
| | | d]pyrimidin-4-yl)-1,2- | | | |
| | | dihydrospiro[indole-3,3′- | | | |
| | | pyrrolidine] | | | |
| 449 | 19 | (3R)-1-(5-chloro-7H- | 326.05 | 3.46 | D |
| | | pyrrolo[2,3-d]pyrimidin-4-yl)- | | | |
| | | 1,2-dihydrospiro[indole-3,3′- | | | |
| | | pyrrolidine] | | | |
| 450 | 19, 11 | (3R)-1′-(4-chlorobenzyl)-1-(7H- | 416.06 | 4.02 | D |
| | | pyrrolo[2,3-d]pyrimidin-4-yl)- | | | |
| | | 1,2-dihydrospiro[indole-3,3′- | | | |
| | | pyrrolidine] | | | |
| 451 | 19, 11 | (3R)-1′-(4-chlorobenzyl)-1-(5- | 449.95 | 4.75 | D |
| | | chloro-7H-pyrrolo[2,3- | | | |
| | | d]pyrimidin-4-yl)-1,2- | | | |
| | | dihydrospiro[indole-3,3′- | | | |
| | | pyrrolidine] | | | |
| 452 | 19, 2 | (3R)-1-(5-methyl-7H- | 305.4 | 3.82 | H |
| | | pyrrolo[2,3-d]pyrimidin-4-yl)- | | | |
| | | 1,2-dihydrospiro[indole-3,3′- | | | |
| | | pyrrolidine] | | | |
| 453 | 19, 2 | (3S)-1-(7H-pyrrolo[2,3- | 292.2 | 0.91 | I |
| | | d]pyrimidin-4-yl)-1,2- | | | |
| | | dihydrospiro[indole-3,3′- | | | |
| | | pyrrolidine] | | | |
| 454 | 19, 1 | (3S)-1′-methyl-1-(7H- | 306.3 | 0.86 | I |
| | | pyrrolo[2,3-d]pyrimidin-4-yl)- | | | |
| | | 1,2-dihydrospiro[indole-3,3′- | | | |
| | | pyrrolidine] | | | |
| 455 | 19 | (3S)-1-(5-chloro-7H- | 326.2 | 1.22 | I |
| | | pyrrolo[2,3-d]pyrimidin-4-yl)- | | | |
| | | 1,2-dihydrospiro[indole-3,3′- | | | |
| | | pyrrolidine] | | | |
| 456 | 19, 12 | 1′-methyl-1-(7H-pyrrolo[2,3- | 306.12 | 1.17 | B |
| | | d]pyrimidin-4-yl)-1,2- | | | |
| | | dihydrospiro[indole-3,3′- | | | |
| | | pyrrolidine] | | | |
| 457 | 19, 12 | 1-(5-chloro-7H-pyrrolo[2,3- | 340.08 | 1.58 | B |
| | | d]pyrimidin-4-yl)-1′-methyl-1,2- | | | |
| | | dihydrospiro[indole-3,3′- | | | |
| | | pyrrolidine] | | | |
| 458 | 19, 11 | (3R)-1′-(cyclohexylmethyl)-1- | 388.21 | 1.77 | A |
| | | (7H-pyrrolo[2,3-d]pyrimidin-4- | | | |
| | | yl)-1,2-dihydrospiro[indole-3,3′- | | | |
| | | pyrrolidine] | | | |
| 459 | 19, 11 | (3R)-1-(7H-pyrrolo[2,3- | 376.14 | 1.35 | A |
| | | d]pyrimidin-4-yl)-1′- | | | |
| | | (tetrahydrofuran-2-ylmethyl)- | | | |
| | | 1,2-dihydrospiro[indole-3,3′- | | | |
| | | pyrrolidine] | | | |
| 460 | 19, 11 | (3R)-1′-(2,2-dimethylpropyl)-1- | 362.18 | 1.56 | A |
| | | (7H-pyrrolo[2,3-d]pyrimidin-4- | | | |
| | | yl)-1,2-dihydrospiro[indole-3,3′- | | | |
| | | pyrrolidine] | | | |
| 461 | 19, 11 | (3R)-1′-(1H-imidazol-4- | 372.14 | 1.21 | A |
| | | ylmethyl)-1-(7H-pyrrolo[2,3- | | | |
| | | d]pyrimidin-4-yl)-1,2- | | | |
| | | dihydrospiro[indole-3,3′- | | | |
| | | pyrrolidine] | | | |
| 462 | 19, 11 | (3R)-1′-butyl-1-(7H-pyrrolo[2,3- | 348.17 | 1.53 | A |
| | | d]pyrimidin-4-yl)-1,2- | | | |
| | | dihydrospiro[indole-3,3′- | | | |
| | | pyrrolidine] | | | |
| 463 | 19, 11 | (3R)-1′-isobutyl-1-(7H- | 348.17 | 1.48 | A |
| | | pyrrolo[2,3-d]pyrimidin-4-yl)- | | | |
| | | 1,2-dihydrospiro[indole-3,3′- | | | |
| | | pyrrolidine] | | | |
| 464 | 19, 11 | (3R)-1′-(3-furylmethyl)-1-(7H- | 372.12 | 1.51 | A |
| | | pyrrolo[2,3-d]pyrimidin-4-yl)- | | | |
| | | 1,2-dihydrospiro[indole-3,3′- | | | |
| | | pyrrolidine] | | | |
| 465 | 19, 11 | (3R)-1′-[2-(4- | 488.25 | 2.12 | A |
| | | methylphenoxy)benzyl]-1-(7H- | | | |
| | | pyrrolo[2,3-d]pyrimidin-4-yl)- | | | |
| | | 1,2-dihydrospiro[indole-3,3′- | | | |
| | | pyrrolidine] | | | |
| 466 | 19, 11 | N,N-dimethyl-3-(4-{[(3R)-1- | 483.28 | 1.37 | A |
| | | (7H-pyrrolo[2,3-d]pyrimidin-4- | | | |
| | | yl)-1,2-dihydro-1′H- | | | |
| | | spiro[indole-3,3′-pyrrolidin]-1′- | | | |
| | | yl]methyl}phenoxy)propan-1- | | | |
| | | amine | | | |
| 467 | 19, 11 | (3R)-1′-(3,3-dimethylbutyl)-1- | 376.2 | 1.77 | A |
| | | (7H-pyrrolo[2,3-d]pyrimidin-4- | | | |
| | | yl)-1,2-dihydrospiro[indole-3,3′- | | | |
| | | pyrrolidine] | | | |
| 468 | 19, 11 | N,N-diethyl-2-(4-{[(3R)-1-(7H- | 497.3 | 1.37 | A |
| | | pyrrolo[2,3-d]pyrimidin-4-yl)- | | | |
| | | 1,2-dihydro-1′H-spiro[indole- | | | |
| | | 3,3′-pyrrolidin]-1′- | | | |
| | | yl]methyl}phenoxy)ethanamine | | | |
| 469 | 19, 11 | 2,6-dimethyl-8-[(3R)-1-(7H- | 448.25 | 1.74 | A |
| | | pyrrolo[2,3-d]pyrimidin-4-yl)- | | | |
| | | 1,2-dihydro-1′H-spiro[indole- | | | |
| | | 3,3′-pyrrolidin]-1′-yl]octan-2-ol | | | |
| 470 | 19, 11 | (3R)-1′-[(3-methyl-1H-pyrazol- | 386.2 | 1.4 | A |
| | | 5-yl)methyl]-1-(7H-pyrrolo[2,3- | | | |
| | | d]pyrimidin-4-yl)-1,2- | | | |
| | | dihydrospiro[indole-3,3′- | | | |
| | | pyrrolidine] | | | |
| 471 | 19, 11 | (3R)-1-(5-chloro-7H- | 406.14 | 1.45 | A |
| | | pyrrolo[2,3-d]pyrimidin-4-yl)-1′- | | | |
| | | (1H-imidazol-4-ylmethyl)-1,2- | | | |
| | | dihydrospiro[indole-3,3′- | | | |
| | | pyrrolidine] | | | |
| 472 | 19, 11 | (3R)-1′-(3-methylbutyl)-1-(7H- | 362.18 | 1.66 | A |
| | | pyrrolo[2,3-d]pyrimidin-4-yl)- | | | |
| | | 1,2-dihydrospiro[indole-3,3′- | | | |
| | | pyrrolidine] | | | |
| 473 | 19, 11 | (3R)-1′-[(6-methylpyridin-2- | 397.2 | 1.52 | A |
| | | yl)methyl]-1-(7H-pyrrolo[2,3- | | | |
| | | d]pyrimidin-4-yl)-1,2- | | | |
| | | dihydrospiro[indole-3,3′- | | | |
| | | pyrrolidine] | | | |
| 474 | 19, 11 | (3R)-1-(5-chloro-7H- | 382.12 | 1.81 | A |
| | | pyrrolo[2,3-d]pyrimidin-4-yl)-1′- | | | |
| | | isobutyl-1,2- | | | |
| | | dihydrospiro[indole-3,3′- | | | |
| | | pyrrolidine] | | | |
| 475 | 19, 11 | (3R)-1-(5-chloro-7H- | 422.19 | 2.09 | A |
| | | pyrrolo[2,3-d]pyrimidin-4-yl)-1′- | | | |
| | | (cyclohexylmethyl)-1,2- | | | |
| | | dihydrospiro[indole-3,3′- | | | |
| | | pyrrolidine] | | | |
| 476 | 19, 11 | (3R)-1′-butyl-1-(5-chloro-7H- | 382.12 | 1.85 | A |
| | | pyrrolo[2,3-d]pyrimidin-4-yl)- | | | |
| | | 1,2-dihydrospiro[indole-3,3′- | | | |
| | | pyrrolidine] | | | |
| 477 | 19, 11 | (3R)-1′-(isoxazol-3-ylmethyl)- | 373.14 | 1.42 | A |
| | | 1-(7H-pyrrolo[2,3-d]pyrimdin- | | | |
| | | 4-yl)-1,2-dihydrospiro[indole- | | | |
| | | 3,3′-pyrrolidine] | | | |
| 478 | 19, 11 | (3R)-1-(5-chloro-7H- | 406.14 | 1.83 | A |
| | | pyrrolo[2,3-d]pyrimidin-4-yl)-1′- | | | |
| | | (3-furylmethyl)-1,2- | | | |
| | | dihydrospiro[indole-3,3′- | | | |
| | | pyrrolidine] | | | |
| 479 | 19, 11 | (3R)-1-(5-chloro-7H- | 396.14 | 1.87 | A |
| | | pyrrolo[2,3-d]pyrimidin-4-yl)-1′- | | | |
| | | (2,2-dimethylpropyl)-1,2- | | | |
| | | dihydrospiro[indole-3,3′- | | | |
| | | pyrrolidine] | | | |
| 480 | 19, 11 | (3R)-1-(5-chloro-7H- | 410.15 | 1.66 | A |
| | | pyrrolo[2,3-d]pyrimidin-4-yl)-1′- | | | |
| | | (tetrahydrofuran-2-ylmethyl)- | | | |
| | | 1,2-dihydrospiro[indole-3,3′- | | | |
| | | pyrrolidine] | | | |
| 481 | 19, 11 | (3R)-1-(5-chloro-7H- | 420.16 | 1.69 | A |
| | | pyrrolo[2,3-d]pyrimidin-4-yl)-1′- | | | |
| | | [(3-methyl-1H-pyrazol-5- | | | |
| | | yl)methyl]-1,2- | | | |
| | | dihydrospiro[indole-3,3′- | | | |
| | | pyrrolidine] | | | |
| 482 | 19, 11 | 2-(4-{[(3R)-1-(5-chloro-7H- | 531.25 | 1.59 | A |
| | | pyrrolo[2,3-d]pyrimidin-4-yl)- | | | |
| | | 1,2-dihydro-1′H-spiro[indole- | | | |
| | | 3,3′-pyrrolidin]-1′- | | | |
| | | yl]methyl}phenoxy)-N,N- | | | |
| | | diethylethanamine | | | |
| 483 | 19, 11 | (3R)-1-(5-chloro-7H- | 410.17 | 2.09 | A |
| | | pyrrolo[2,3-d]pyrimidin-4-yl)-1′- | | | |
| | | (3,3-dimethylbutyl)-1,2- | | | |
| | | dihydrospiro[indole-3,3′- | | | |
| | | pyrrolidine] | | | |
| 484 | 19, 11 | (3R)-1-(5-chloro-7H- | 431.18 | 1.81 | A |
| | | pyrrolo[2,3-d]pyrimidin-4-yl)-1′- | | | |
| | | [(6-methylpyridin-2-yl)methyl]- | | | |
| | | 1,2-dihydrospiro[indole-3,3′- | | | |
| | | pyrrolidine] | | | |
| 485 | 19, 11 | (3R)-1-(5-chloro-7H- | 396.14 | 1.98 | A |
| | | pyrrolo[2,3-d]pyrimidin-4-yl)-1′- | | | |
| | | (3-methylbutyl)-1,2- | | | |
| | | dihydrospiro[indole-3,3′- | | | |
| | | pyrrolidine] | | | |
| 486 | 19, 11 | (3R)-1-(5-chloro-7H- | 522.2 | 2.43 | A |
| | | pyrrolo[2,3-d]pyrimidin-4-yl)-1′- | | | |
| | | [2-(4-methylphenoxy)benzyl]- | | | |
| | | 1,2-dihydrospiro[indole-3,3′- | | | |
| | | pyrrolidine] | | | |
| 487 | 19, 11 | 8-[(3R)-1-(5-chloro-7H- | 482.26 | 2.02 | A |
| | | pyrrolo[2,3-d]pyrimidin-4-yl)- | | | |
| | | 1,2-dihydro-1′H-spiro[indole- | | | |
| | | 3,3′-pyrrolidin]-1′-yl]-2,6- | | | |
| | | dimethyloctan-2-ol | | | |
| 488 | 19, 11 | (3R)-1-(5-chloro-7H- | 407.1 | 1.73 | A |
| | | pyrrolo[2,3-d]pyrimidin-4-yl)-1′- | | | |
| | | (isoxazol-3-ylmethyl)-1,2- | | | |
| | | dihydrospiro[indole-3,3′- | | | |
| | | pyrrolidine] | | | |
| 489 | 19, 11 | (3R)-1′-(cyclopropylmethyl)-1- | 346.13 | 1.44 | A |
| | | (7H-pyrrolo[2,3-d]pyrimidin-4- | | | |
| | | yl)-1,2-dihydrospiro[indole-3,3′- | | | |
| | | pyrrolidine] | | | |
| 490 | 19, 11 | (3R)-1′-propyl-1-(7H- | 334.15 | 1.4 | A |
| | | pyrrolo[2,3-d]pyrimidin-4-yl)- | | | |
| | | 1,2-dihydrospiro[indole-3,3′- | | | |
| | | pyrrolidine] | | | |
| 491 | 19, 11 | (3R)-1-(5-chloro-7H- | 380.09 | 1.76 | A |
| | | pyrrolo[2,3-d]pyrimidin-4-yl)-1′- | | | |
| | | (cyclopropylmethyl)-1,2- | | | |
| | | dihydrospiro[indole-3,3′- | | | |
| | | pyrrolidine] | | | |
| 492 | 19, 10 | (3R)-1-(5-chloro-7H- | 340.08 | 1.58 | A |
| | | pyrrolo[2,3-d]pyrimidin-4-yl)-1′- | | | |
| | | methyl-1,2-dihydrospiro[indole- | | | |
| | | 3,3′-pyrrolidine] | | | |
| 493 | 19, 11 | (3R)-1′-ethyl-1-(7H-pyrrolo[2,3- | 320.14 | 1.32 | A |
| | | d]pyrimidin-4-yl)-1,2- | | | |
| | | dihydrospiro[indole-3,3′- | | | |
| | | pyrrolidine] | | | |
| 494 | 19, 11 | (3R)-1-(5-chloro-7H- | 354.09 | 1.64 | A |
| | | pyrrolo[2,3-d]pyrimidin-4-yl)-1′- | | | |
| | | ethyl-1,2-dihydrospiro[indole- | | | |
| | | 3,3′-pyrrolidine] | | | |
| 495 | 19, 15 | (3S)—N-allyl-1-(7H-pyrrolo[2,3- | 375.18 | 1.75 | A |
| | | d]pyrimidin-4-yl)-1,2-dihydro- | | | |
| | | 1′H-spiro[indole-3,3′- | | | |
| | | pyrrolidine]-1′-carboxamide | | | |
| 496 | 19, 15 | (3S)—N-pentyl-1-(7H- | 405.25 | 2.11 | A |
| | | pyrrolo[2,3-d]pyrimidin-4-yl)- | | | |
| | | 1,2-dihydro-1′H-spiro[indole- | | | |
| | | 3,3′-pyrrolidine]-1′- | | | |
| | | carboxamide | | | |
| 497 | 19, 15 | methyl N-{[(3S)-1-(7H- | 463.28 | 2.15 | A |
| | | pyrrolo[2,3-d]pyrimidin-4-yl)- | | | |
| | | 1,2-dihydro-1′H-spiro[indole- | | | |
| | | 3,3′-pyrrolidin]-1′-yl]carbonyl}- | | | |
| | | L-isoleucinate | | | |
| 498 | 19, 15 | (3S)—N-isopropyl-1-(7H- | 377.21 | 1.79 | A |
| | | pyrrolo[2,3-d]pyrimidin-4-yl)- | | | |
| | | 1,2-dihydro-1′H-spiro[indole- | | | |
| | | 3,3′-pyrrolidine]-1′- | | | |
| | | carboxamide | | | |
| 499 | 19, 15 | methyl N-{[(3S)-1-(7H- | 421.19 | 1.73 | A |
| | | pyrrolo[2,3-d]pyrimidin-4-yl)- | | | |
| | | 1,2-dihydro-1′H-spiro[indole- | | | |
| | | 3,3′-pyrrolidin]-1′-yl]carbonyl}- | | | |
| | | L-alaninate | | | |
| 500 | 19, 15 | (3S)—N-[(1R,2S)-2- | 451.24 | 2.19 | A |
| | | phenylcyclopropyl]-1-(7H- | | | |
| | | pyrrolo[2,3-d]pyrimidin-4-yl)- | | | |
| | | 1,2-dihydro-1′H-spiro[indole- | | | |
| | | 3,3′-pyrrolidine]-1′- | | | |
| | | carboxamide | | | |
| 501 | 19, 15 | (3S)—N-benzyl-1-(7H- | 425.19 | 2.04 | A |
| | | pyrrolo[2,3-d]pyrimidin-4-yl)- | | | |
| | | 1,2-dihydro-1′H-spiro[indole- | | | |
| | | 3,3′-pyrrolidine]-1′- | | | |
| | | carboxamide | | | |
| 502 | 19, 15 | (3S)—N-cyclohexyl-1-(7H- | 417.23 | 2.09 | A |
| | | pyrrolo[2,3-d]pyrimidin-4-yl)- | | | |
| | | 1,2-dihydro-1′H-spiro[indole- | | | |
| | | 3,3′-pyrrolidine]-1′- | | | |
| | | carboxamide | | | |
| 503 | 19, 15 | (3S)—N-propyl-1-(7H- | 377.21 | 1.8 | A |
| | | pyrrolo[2,3-d]pyrimidin-4-yl)- | | | |
| | | 1,2-dihydro-1′H-spiro[indole- | | | |
| | | 3,3′-pyrrolidine]-1′- | | | |
| | | carboxamide | | | |
| 504 | 19, 15 | methyl N-{[(3S)-1-(7H- | 463.27 | 2.13 | A |
| | | pyrrolo[2,3-d]pyrimidin-4-yl)- | | | |
| | | 1,2-dihydro-1′H-spiro[indole- | | | |
| | | 3,3′-pyrrolidin]-1′-yl]carbonyl}- | | | |
| | | L-leucinate | | | |
| 505 | 19, 15 | (3S)—N-ethyl-1-(7H-pyrrolo[2,3- | 363.18 | 1.66 | A |
| | | d]pyrimidin-4-yl)-1,2-dihydro- | | | |
| | | 1′H-spiro[indole-3,3′- | | | |
| | | pyrrolidine]-1′-carboxamide | | | |
| 506 | 19, 15 | (3S)—N-cyclopentyl-1-(7H- | 403.21 | 1.98 | A |
| | | pyrrolo[2,3-d]pyrimidin-4-yl)- | | | |
| | | 1,2-dihydro-1′H-spiro[indole- | | | |
| | | 3,3′-pyrrolidine]-1′- | | | |
| | | carboxamide | | | |
| 507 | 19, 15 | (3S)—N-butyl-1-(7H-pyrrolo[2,3- | 391.23 | 1.95 | A |
| | | d]pyrimidin-4-yl)-1,2-dihydro- | | | |
| | | 1′H-spiro[indole-3,3′- | | | |
| | | pyrrolidine]-1′-carboxamide | | | |
| 508 | 19, 15 | ethyl N-{[(3S)-1-(7H- | 421.18 | 1.73 | A |
| | | pyrrolo[2,3-d]pyrimidin-4-yl)- | | | |
| | | 1,2-dihydro-1′H-spiro[indole- | | | |
| | | 3,3′-pyrrolidin]-1′- | | | |
| | | yl]carbonyl}glycinate | | | |
| 509 | 19, 15 | (3S)—N-(4-cyanophenyl)-1-(7H- | 436.21 | 2.11 | A |
| | | pyrrolo[2,3-d]pyrimidin-4-yl)- | | | |
| | | 1,2-dihydro-1′H-spiro[indole- | | | |
| | | 3,3′-pyrrolidine]-1′- | | | |
| | | carboxamide | | | |
| 510 | 19, 15 | ethyl {[(3S)-1-(7H-pyrrolo[2,3- | 407.16 | 1.68 | A |
| | | d]pyrimidin-4-yl)-1,2-dihydro- | | | |
| | | 1′H-spiro[indole-3,3′- | | | |
| | | pyrrolidin]-1′- | | | |
| | | yl]carbonyl}carbamate | | | |
| 511 | 19, 15 | (3S)—N-(2-phenylethyl)-1-(7H- | 439.2 | 2.11 | A |
| | | pyrrolo[2,3-d]pyrimidin-4-yl)- | | | |
| | | 1,2-dihydro-1′H-spiro[indole- | | | |
| | | 3,3′-pyrrolidine]-1′- | | | |
| | | carboxamide | | | |
| 512 | 19, 15 | (3S)—N-(3,5-dimethylisoxazol- | 430.22 | 1.73 | A |
| | | 4-yl)-1-(7H-pyrrolo[2,3- | | | |
| | | d]pyrimidin-4-yl)-1,2-dihydro- | | | |
| | | 1′H-spiro[indole-3,3′- | | | |
| | | pyrrolidine]-1′-carboxamide | | | |
| 513 | 19, 15 | (3S)—N-phenyl-1-(7H- | 411.17 | 2.05 | A |
| | | pyrrolo[2,3-d]pyrimidin-4-yl)- | | | |
| | | 1,2-dihydro-1′H-spiro[indole- | | | |
| | | 3,3′-pyrrolidine]-1′- | | | |
| | | carboxamide | | | |
| 514 | 19, 15 | ethyl 4-({[(3S)-1-(7H- | 449.25 | 1.88 | A |
| | | pyrrolo[2,3-d]pyrimidin-4-yl)- | | | |
| | | 1,2-dihydro-1′H-spiro[indole- | | | |
| | | 3,3′-pyrrolidin]-1′- | | | |
| | | yl]carbonyl}amino)butanoate | | | |
| 515 | 19, 15 | (3S)—N-[(1S)-1-phenylethyl]-1- | 439.2 | 2.13 | A |
| | | (7H-pyrrolo[2,3-d]pyrimidin-4- | | | |
| | | yl)-1,2-dihydro-1′H- | | | |
| | | spiro[indole-3,3′-pyrrolidine]-1′- | | | |
| | | carboxamide | | | |
| 516 | 19, 15 | (3S)—N-allyl-1-(5-chloro-7H- | 409.14 | 2.11 | A |
| | | pyrrolo[2,3-d]pyrimidin-4-yl)- | | | |
| | | 1,2-dihydro-1′H-spiro[indole | | | |
| | | 3,3′-pyrrolidine]-1′- | | | |
| | | carboxamide | | | |
| 517 | 19, 15 | (3S)-1-(5-chloro-7H- | 451.21 | 2.52 | A |
| | | pyrrolo[2,3-d]pyrimidin-4-yl)-N- | | | |
| | | cyclohexyl-1,2-dihydro-1′H- | | | |
| | | spiro[indole-3,3′-pyrrolidine]-1′- | | | |
| | | carboxamide | | | |
| 518 | 19, 15 | (3S)—N-benzoyl-1-(7H- | 439.2 | 1.93 | A |
| | | pyrrolo[2,3-d]pyrimidin-4-yl)- | | | |
| | | 1,2-dihydro-1′H-spiro[indole- | | | |
| | | 3,3′-pyrrolidine]-1′- | | | |
| | | carboxamide | | | |
| 519 | 19, 15 | (3S)—N-(2-furylmethyl)-1-(7H- | 415.19 | 1.89 | A |
| | | pyrrolo[2,3-d]pyrimidin-4-yl)- | | | |
| | | 1,2-dihydro-1′H-spiro[indole- | | | |
| | | 3,3′-pyrrolidine]-1′- | | | |
| | | carboxamide | | | |
| 520 | 19, 15 | (3S)-1-(5-chloro-7H- | 411.15 | 2.18 | A |
| | | pyrrolo[2,3-d]pyrimidin-4-yl)-N- | | | |
| | | isopropyl-1,2-dihydro-1′H- | | | |
| | | spiro[indole-3,3′-pyrrolidine]-1′- | | | |
| | | carboxamide | | | |
| 521 | 19, 15 | (3S)-1-(5-chloro-7H- | 397.16 | 2.02 | A |
| | | pyrrolo[2,3-d]pyrimidin-4-yl)-N- | | | |
| | | ethyl-1,2-dihydro-1′H- | | | |
| | | spiro[indole-3,3′-pyrrolidine]-1′- | | | |
| | | carboxamide | | | |
| 522 | 19, 15 | methyl N-{[(3S)-1-(5-chloro- | 455.19 | 2.1 | A |
| | | 7H-pyrrolo[2,3-d]pyrimidin-4- | | | |
| | | yl)-1,2-dihydro-1′H- | | | |
| | | spiro[indole-3,3′-pyrrolidin]-1′- | | | |
| | | yl]carbonyl}-L-alaninate | | | |
| 523 | 19, 15 | (3S)—N-1H-indol-3-yl-1-(7H- | 450.22 | 2.02 | A |
| | | pyrrolo[2,3-d]pyrimidin-4-yl)- | | | |
| | | 1,2-dihydro-1′H-spiro[indole- | | | |
| | | 3,3′-pyrrolidine]-1′- | | | |
| | | carboxamide | | | |
| 524 | 19, 15 | (3S)-1-(5-chloro-7H- | 439.2 | 2.53 | A |
| | | pyrrolo[2,3-d]pyrimidin-4-yl)-N- | | | |
| | | pentyl-1,2-dihydro-1′H- | | | |
| | | spiro[indole-3,3′-pyrrolidine]-1′- | | | |
| | | carboxamide | | | |
| 525 | 19, 15 | (3S)-1-(5-chloro-7H- | 411.17 | 2.17 | A |
| | | pyrrolo[2,3-d]pyrimidin-4-yl)-N- | | | |
| | | propyl-1,2-dihydro-1′H- | | | |
| | | spiro[indole-3,3′-pyrrolidine]-1′- | | | |
| | | carboxamide | | | |
| 526 | 19, 15 | methyl N-{[(3S)-1-(5-chloro- | 497.23 | 2.54 | A |
| | | 7H-pyrrolo[2,3-d]pyrimidin-4- | | | |
| | | yl)-1,2-dihydro-1′H- | | | |
| | | spiro[indole-3,3′-pyrrolidin]-1′- | | | |
| | | yl]carbonyl}-D-leucinate | | | |
| 527 | 19, 15 | ethyl 4-({[(3S)-1-(5-chloro-7H- | 483.22 | 2.23 | A |
| | | pyrrolo[2,3-d]pyrimidin-4-yl)- | | | |
| | | 1,2-dihydro-1′H-spiro[indole- | | | |
| | | 3,3′-pyrrolidin]-1′- | | | |
| | | yl]carbonyl}amino)butanoate | | | |
| 528 | 19, 15 | (3S)—N-butyl-1-(5-chloro-7H- | 425.19 | 2.36 | A |
| | | pyrrolo[2,3-d]pyrimidin-4-yl)- | | | |
| | | 1,2-dihydro-1′H-spiro[indole- | | | |
| | | 3,3′-pyrrolidine]-1′- | | | |
| | | carboxamide | | | |
| 529 | 19, 15 | ethyl N-{[(3S)-1-(5-chloro-7H- | 455.19 | 2.08 | A |
| | | pyrrolo[2,3-d]pyrimidin-4-yl)- | | | |
| | | 1,2-dihydro-1′H-spiro[indole- | | | |
| | | 3,3′-pyrrolidin]-1′- | | | |
| | | yl]carbonyl}glycinate | | | |
| 530 | 19, 15 | methyl N-{[(3S)-1-(5-chloro- | 497.23 | 2.57 | A |
| | | 7H-pyrrolo[2,3-d]pyrimidin-4- | | | |
| | | yl)-1,2-dihydro-1′H- | | | |
| | | spiro[indole-3,3′-pyrrolidin]-1′- | | | |
| | | yl]carbonyl}-L-isoleucinate | | | |
| 531 | 19, 15 | (3S)-1-(5-chloro-7H- | 437.18 | 2.39 | A |
| | | pyrrolo[2,3-d]pyrimidin-4-yl)-N- | | | |
| | | cyclopentyl-1,2-dihydro-1′H- | | | |
| | | spiro[indole-3,3′-pyrrolidine]-1′- | | | |
| | | carboxamide | | | |
| 532 | 19, 15 | ethyl {[(3S)-1-(5-chloro-7H- | 441.15 | 2.05 | A |
| | | pyrrolo[2,3-d]pyrimidin-4-yl)- | | | |
| | | 1,2-dihydro-1′H-spiro[indole- | | | |
| | | 3,3′-pyrrolidin]-1′- | | | |
| | | yl]carbonyl}carbamate | | | |
| 533 | 19, 15 | (3S)-1-(5-chloro-7H- | 473.22 | 2.51 | A |
| | | pyrrolo[2,3-d]pyrimidin-4-yl)-N- | | | |
| | | (2-phenylethyl)-1,2-dihydro- | | | |
| | | 1′H-spiro[indole-3,3′- | | | |
| | | pyrrolidine]-1′-carboxamide | | | |
| 534 | 19, 15 | (3S)-1-(5-chloro-7H- | 449.14 | 2.25 | A |
| | | pyrrolo[2,3-d]pyrimidin-4-yl)-N- | | | |
| | | (2-furylmethyl)-1,2-dihydro- | | | |
| | | 1′H-spiro[indole-3,3′- | | | |
| | | pyrrolidine]-1′-carboxamide | | | |
| 535 | 19, 15 | (3S)-1-(5-chloro-7H- | 470.17 | 2.54 | A |
| | | pyrrolo[2,3-d]pyrimidin-4-yl)-N- | | | |
| | | (4-cyanophenyl)-1,2-dihydro- | | | |
| | | 1′H-spiro[indole-3,3′- | | | |
| | | pyrrolidine]-1′-carboxamide | | | |
| 536 | 19, 15 | (3S)-1-(5-chloro-7H- | 464.17 | 2.09 | A |
| | | pyrrolo[2,3-d]pyrimidin-4-yl)-N- | | | |
| | | (3,5-dimethylisoxazol-4-yl)-1,2- | | | |
| | | dihydro-1′H-spiro[indole-3,3′- | | | |
| | | pyrrolidine]-1′-carboxamide | | | |
| 537 | 19, 15 | (3S)—N-benzoyl-1-(5-chloro- | 473.16 | 2.32 | A |
| | | 7H-pyrrolo[2,3-d]pyrimidin-4- | | | |
| | | yl)-1,2-dihydro-1′H- | | | |
| | | spiro[indole-3,3′-pyrrolidine]-1′- | | | |
| | | carboxamide | | | |
| 538 | 1 | 5-methyl-1-(9H-purin-6-yl)-1,2- | 321.15 | 3.27 | D |
| | | dihydrospiro[indole-3,4′- | | | |
| | | piperidine] | | | |
| 539 | 1 | 1-(9H-purin-6-yl)-1,2- | 307.13 | 3.01 | D |
| | | dihydrospiro[indole-3,4′- | | | |
| | | piperidine] | | | |
| 540 | 1 | 5-fluoro-1-(9H-purin-6-yl)-1,2- | 325.1 | 3.24 | D |
| | | dihydrospiro[indole-3,4′- | | | |
| | | piperidine] | | | |
| 541 | 1 | 5,7-dimethyl-1-(9H-purin-6-yl)- | 335.16 | 3.35 | D |
| | | 1,2-dihydrospiro[indole-3,4′- | | | |
| | | piperidine] | | | |
| 542 | 1, 8 | 2-methyl-1-(9H-purin-6-yl)-1,2- | 321.3 | 1.06 | I |
| | | dihydrospiro[indole-3,4′- | | | |
| | | piperidine] | | | |
| 543 | 1, 10 | 1′-methyl-1-(9H-purin-6-yl)- | 321.15 | 1.38 | E |
| | | 1,2-dihydrospiro[indole-3,4′- | | | |
| | | piperidine] | | | |
| 544 | 1, 10 | 1′,5-dimethyl-1-(9H-purin-6-yl)- | 335.16 | 1.58 | E |
| | | 1,2-dihydrospiro[indole-3,4′- | | | |
| | | piperidine] | | | |
| 545 | 1, 10 | 5-fluoro-1′-methyl-1-(9H-purin- | 339.11 | 1.49 | E |
| | | 6-yl)-1,2-dihydrospiro[indole- | | | |
| | | 3,4′-piperidine] | | | |
| 546 | 1, 11 | 1′-(4-chlorobenzyl)-1-(9H- | 431.03 | 4.19 | D |
| | | purin-6-yl)-1,2- | | | |
| | | dihydrospiro[indole-3,4′- | | | |
| | | piperidine] | | | |
| 547 | 1, 11 | 1′-(4-chlorobenzyl)-5-fluoro-1- | 449 | 4.41 | D |
| | | (9H-purin-6-yl)-1,2- | | | |
| | | dihydrospiro[indole-3,4′- | | | |
| | | piperidine] | | | |
| 548 | 1, 11 | 1′-(4-chlorobenzyl)-5-methyl-1- | 445.05 | 4.4 | D |
| | | (9H-purin-6-yl)-1,2- | | | |
| | | dihydrospiro[indole-3,4′- | | | |
| | | piperidine] | | | |
| 549 | 1, 11 | 5-chloro-1′-(cyclohexylmethyl)- | 437.21 | 2.1 | B |
| | | 1-(9H-purin-6-yl)-1,2- | | | |
| | | dihydrospiro[indole-3,4′- | | | |
| | | piperidine] | | | |
| 550 | 1, 11 | 5-chloro-1-(9H-purin-6-yl)-1′- | 438.11 | 1.8 | B |
| | | (1,3-thiazol-2-ylmethyl)-1,2- | | | |
| | | dihydrospiro[indole-3,4′- | | | |
| | | piperidine] | | | |
| 551 | 1, 11 | 5-chloro-1′-(4-methoxybenzyl)- | 461.17 | 2.05 | B |
| | | 1-(9H-purin-6-yl)-1,2- | | | |
| | | dihydrospiro[indole-3,4′- | | | |
| | | piperidine] | | | |
| 552 | 1, 11 | 5-chloro-1′-(2,4- | 467.13 | 2.02 | B |
| | | difluorobenzyl)-1-(9H-purin-6- | | | |
| | | yl)-1,2-dihydrospiro[indole-3,4′- | | | |
| | | piperidine] | | | |
| 553 | 1, 11 | 5-chloro-1′-(2-phenylethyl)-1- | 445.18 | 2.08 | B |
| | | (9H-purin-6-yl)-1,2- | | | |
| | | dihydrospiro[indole-3,4′- | | | |
| | | piperidine] | | | |
| 554 | 1, 11 | 1′-[2-(benzyloxy)ethyl]-5- | 475.16 | 2.17 | B |
| | | chloro-1-(9H-purin-6-yl)-1,2- | | | |
| | | dihydrospiro[indole-3,4′- | | | |
| | | piperidine] | | | |
| 555 | 1, 11 | 5-chloro-1′-(3-fluoro-4- | 479.14 | 2.08 | B |
| | | methoxybenzyl)-1-(9H-purin-6- | | | |
| | | yl)-1,2-dihydrospiro[indole-3,4′- | | | |
| | | piperidine] | | | |
| 556 | 1, 11 | 5-chloro-1′-[(2-methyl-1H- | 435.13 | 1.61 | B |
| | | imidazol-4-yl)methyl]-1-(9H- | | | |
| | | purin-6-yl)-1,2- | | | |
| | | dihydrospiro[indole-3,4′- | | | |
| | | piperidine] | | | |
| 557 | 1, 11 | 1′-butyl-5-chloro-1-(9H-purin- | 397.16 | 1.89 | B |
| | | 6-yl)-1,2-dihydrospiro[indole- | | | |
| | | 3,4′-piperidine] | | | |
| 558 | 1, 11 | 5-chloro-1′-(3,4- | 499.06 | 2.27 | B |
| | | dichlorobenzyl)-1-(9H-purin-6- | | | |
| | | yl)-1,2-dihydrospiro[indole-3,4′- | | | |
| | | piperidine] | | | |
| 559 | 1, 11 | 5-chloro-1′-[(4-methyl-1H- | 435.13 | 1.61 | B |
| | | imidazol-5-yl)methyl]-1-(9H- | | | |
| | | purin-6-yl)-1,2- | | | |
| | | dihydrospiro[indole-3,4′- | | | |
| | | piperidine] | | | |
| 560 | 1, 11 | 5-chloro-1-(9H-purin-6-yl)-1′- | 499.15 | 2.27 | B |
| | | [4-(trifluoromethyl)benzyl]-1,2- | | | |
| | | dihydrospiro[indole-3,4′- | | | |
| | | piperidine] | | | |
| 561 | 1, 11 | 5-chloro-1′-(3,4- | 467.12 | 2.12 | B |
| | | difluorobenzyl)-1-(9H-purin-6- | | | |
| | | yl)-1,2-dihydrospiro[indole-3,4′- | | | |
| | | piperidine] | | | |
| 562 | 1, 11 | 5-chloro-1′-[(3-methyl-1H- | 435.17 | 1.81 | B |
| | | pyrazol-5-yl)methyl]-1-(9H- | | | |
| | | purin-6-yl)-1,2- | | | |
| | | dihydrospiro[indole-3,4′- | | | |
| | | piperidine] | | | |
| 563 | 1, 11 | 5-chloro-1′-(4-chlorobenzyl)-1- | 465.13 | 2.15 | B |
| | | (9H-purin-6-yl)-1,2- | | | |
| | | dihydrospiro[indole-3,4′- | | | |
| | | piperidine] | | | |
| 564 | 1, 11 | 5-chloro-1-(9H-purin-6-yl)-1′- | 420.12 | 1.92 | B |
| | | (1H-pyrrol-2-ylmethyl)-1,2- | | | |
| | | dihydrospiro[indole-3,4′- | | | |
| | | piperidine] | | | |
| 565 | 1, 11 | 5-chloro-1′-(2-chlorobenzyl)-1- | 465.11 | 2.04 | B |
| | | (9H-purin-6-yl)-1,2- | | | |
| | | dihydrospiro[indole-3,4′- | | | |
| | | piperidine] | | | |
| 566 | 1, 11 | 5-chloro-1′-[(2-ethyl-1H- | 449.15 | 1.65 | B |
| | | imidazol-5-yl)methyl]-1-(9H- | | | |
| | | purin-6-yl)-1,2- | | | |
| | | dihydrospiro[indole-3,4′- | | | |
| | | piperidine] | | | |
| 567 | 1, 11 | 5-chloro-1′-(4-fluorobenzyl)-1- | 449.14 | 2.05 | B |
| | | (9H-purin-6-yl)-1,2- | | | |
| | | dihydrospiro[indole-3,4′- | | | |
| | | piperidine] | | | |
| 568 | 1, 11 | 1′-(1H-benzimidazol-2- | 471.15 | 1.97 | B |
| | | ylmethyl)-5-chloro-1-(9H-purin- | | | |
| | | 6-yl)-1,2-dihydrospiro[indole- | | | |
| | | 3,4′-piperidine] | | | |
| 569 | 1, 14 | benzyl 1-(9H-purin-6-yl)-1,2- | 441.1 | 2.37 | E |
| | | dihydro-1′H-spiro[indole-3,4′- | | | |
| | | piperidine]-1′-carboxylate | | | |
| 570 | 1, 14 | benzyl 5-fluoro-1-(9H-purin-6- | 459.11 | 2.44 | E |
| | | yl)-1,2-dihydro-1′H- | | | |
| | | spiro[indole-3,4′-piperidine]-1′- | | | |
| | | carboxylate | | | |
| 571 | 1, 14 | benzyl 5-methyl-1-(9H-purin-6- | 455.11 | 2.54 | E |
| | | yl)-1,2-dihydro-1′H- | | | |
| | | spiro[indole-3,4′-piperidine]-1′- | | | |
| | | carboxylate | | | |
| 572 | 1, 15 | 5-methyl-1′-(morpholin-4- | 434.08 | 1.96 | C |
| | | ylcarbonyl)-1-(9H-purin-6-yl)- | | | |
| | | 1,2-dihydrospiro[indole-3,4′- | | | |
| | | piperidine] | | | |
| 573 | 1, 15 | 5-fluoro-1′-(morpholin-4- | 438.04 | 1.95 | C |
| | | ylcarbonyl)-1-(9H-purin-6-yl)- | | | |
| | | 1,2-dihydrospiro[indole-3,4′- | | | |
| | | piperidine] | | | |
| 574 | 1, 15 | 1′-[(4-methylpiperazin-1- | 433.08 | 1.5 | C |
| | | yl)carbonyl]-1-(9H-purin-6-yl)- | | | |
| | | 1,2-dihydrospiro[indole-3,4′- | | | |
| | | piperidine] | | | |
| 575 | 1, 15 | 1′-(morpholin-4-ylcarbonyl)-1- | 420.08 | 1.84 | C |
| | | (9H-purin-6-yl)-1,2- | | | |
| | | dihydrospiro[indole-3,4′- | | | |
| | | piperidine] | | | |
| 576 | 1, 15 | 5-methyl-1′-[(4- | 447.09 | 1.57 | C |
| | | methylpiperazin-1-yl)carbonyl]- | | | |
| | | 1-(9H-purin-6-yl)-1,2- | | | |
| | | dihydrospiro[indole-3,4′- | | | |
| | | piperidine] | | | |
| 577 | 1, 15 | 5-fluoro-1′-[(4-methylpiperazin- | 451.07 | 1.57 | C |
| | | 1-yl)carbonyl]-1-(9H-purin-6- | | | |
| | | yl)-1,2-dihydrospiro[indole-3,4′- | | | |
| | | piperidine] | | | |
| 578 | 19, 14 | benzyl (3S)-1-(9H-purin-6-yl)- | 427.09 | 2.23 | E |
| | | 1,2-dihydro-1′H-spiro[indole- | | | |
| | | 3,3′-pyrrolidine]-1′-carboxylate | | | |
| 579 | 19, 10 | (3R)-1′-methyl-1-(9H-purin-6- | 307.13 | 1.52 | E |
| | | yl)-1,2-dihydrospiro[indole-3,3′- | | | |
| | | pyrrolidine] | | | |
| 580 | 19 | (3R)-1-(9H-purin-6-yl)-1,2- | 293.12 | 3 | D |
| | | dihydrospiro[indole-3,3′- | | | |
| | | pyrrolidine] | | | |
| 581 | 19, 11 | (3R)-1′-isobutyl-1-(9H-purin-6- | 349.19 | 1.61 | A |
| | | yl)-1,2-dihydrospiro[indole-3,3′- | | | |
| | | pyrrolidine] | | | |
| 582 | 19, 11 | (3R)-1′-(cyclohexylmethyl)-1- | 389.2 | 1.89 | A |
| | | (9H-purin-6-yl)-1,2- | | | |
| | | dihydrospiro[indole-3,3′- | | | |
| | | pyrrolidine] | | | |
| 583 | 19, 11 | (3R)-1′-(2,2-dimethylpropyl)-1- | 363.2 | 1.68 | A |
| | | (9H-purin-6-yl)-1,2- | | | |
| | | dihydrospiro[indole-3,3′- | | | |
| | | pyrrolidine] | | | |
| 584 | 19, 11 | N,N-diethyl-2-(4-{[(3R)-1-(9H- | 498.25 | 1.46 | A |
| | | purin-6-yl)-1,2-dihydro-1′H- | | | |
| | | spiro[indole-3,3′-pyrrolidin]-1′- | | | |
| | | yl]methyl}phenoxy)ethanamine | | | |
| 585 | 19, 11 | (3R)-1′-butyl-1-(9H-purin-6-yl)- | 349.19 | 1.64 | A |
| | | 1,2-dihydrospiro[indole-3,3′- | | | |
| | | pyrrolidine] | | | |
| 586 | 19, 11 | 2,6-dimethyl-8-[(3R)-1-(9H- | 449.27 | 1.85 | A |
| | | purin-6-yl)-1,2-dihydro-1′H- | | | |
| | | spiro[indole-3,3′-pyrrolidin]-1′- | | | |
| | | yl]octan-2-ol | | | |
| 587 | 19, 11 | (3R)-1′-(1H-imidazol-4- | 373.14 | 1.28 | A |
| | | ylmethyl)-1-(9H-purin-6-yl)-1,2- | | | |
| | | dihydrospiro[indole-3,3′- | | | |
| | | pyrrolidine] | | | |
| 588 | 19, 11 | (3R)-1′-(3,3-dimethylbutyl)-1- | 377.22 | 1.89 | A |
| | | (9H-purin-6-yl)-1,2- | | | |
| | | dihydrospiro[indole-3,3′- | | | |
| | | pyrrolidine] | | | |
| 589 | 19, 11 | N,N-dimethyl-3-(4-{[(3R)-1- | 484.3 | 1.44 | A |
| | | (9H-purin-6-yl)-1,2-dihydro- | | | |
| | | 1′H-spiro[indole-3,3′- | | | |
| | | pyrrolidin]-1′- | | | |
| | | yl]methyl}phenoxy)propan-1- | | | |
| | | amine | | | |
| 590 | 19, 11 | (3R)-1′-[(3-methyl-1H-pyrazol- | 387.16 | 1.51 | A |
| | | 5-yl)methyl]-1-(9H-purin-6-yl)- | | | |
| | | 1,2-dihydrospiro[indole-3,3′- | | | |
| | | pyrrolidine] | | | |
| 591 | 19, 11 | (3R)-1-(9H-purin-6-yl)-1′- | 377.16 | 1.46 | A |
| | | (tetrahydrofuran-2-ylmethyl)- | | | |
| | | 1,2-dihydrospiro[indole-3,3′- | | | |
| | | pyrrolidine] | | | |
| 592 | 19, 11 | (3R)-1′-(3-furylmethyl)-1-(9H- | 373.14 | 1.63 | A |
| | | purin-6-yl)-1,2- | | | |
| | | dihydrospiro[indole-3,3′- | | | |
| | | pyrrolidine] | | | |
| 593 | 19, 11 | (3R)-1′-(isoxazol-3-ylmethyl)- | 374.14 | 1.52 | A |
| | | 1-(9H-purin-6-yl)-1,2- | | | |
| | | dihydrospiro[indole-3,3′- | | | |
| | | pyrrolidine] | | | |
| 594 | 19, 11 | (3R)-1′-[(6-methylpyridin-2- | 398.18 | 1.63 | A |
| | | yl)methyl]-1-(9H-purin-6-yl)- | | | |
| | | 1,2-dihydrospiro[indole-3,3′- | | | |
| | | pyrrolidine] | | | |
| 595 | 19, 11 | (3R)-1′-(3-methylbutyl)-1-(9H- | 363.2 | 1.78 | A |
| | | purin-6-yl)-1,2- | | | |
| | | dihydrospiro[indole-3,3′- | | | |
| | | pyrrolidine] | | | |
| 596 | 19, 11 | (3R)-1′-[2-(4- | 489.21 | 2.25 | A |
| | | methylphenoxy)benzyl]-1-(9H- | | | |
| | | purin-6-yl)-1,2- | | | |
| | | dihydrospiro[indole-3,3′- | | | |
| | | pyrrolidine] | | | |
| 597 | 19, 11 | (3R)-1′-propyl-1-(9H-purin-6- | 346.13 | 1.44 | A |
| | | yl)-1,2-dihydrospiro[indole-3,3′- | | | |
| | | pyrrolidine] | | | |
| 598 | 19, 11 | (3R)-1′-ethyl-1-(9H-purin-6-yl)- | 335.17 | 1.52 | A |
| | | 1,2-dihydrospiro[indole-3,3′- | | | |
| | | pyrrolidine] | | | |
| 599 | 19, 11 | (3R)-1′-(cyclopropylmethyl)-1- | 347.15 | 1.56 | A |
| | | (9H-purin-6-yl)-1,2- | | | |
| | | dihydrospiro[indole-3,3′- | | | |
| | | pyrrolidine] | | | |
| 600 | 19, 15 | (3S)—N-cyclohexyl-1-(9H-purin- | 418.24 | 2.16 | A |
| | | 6-yl)-1,2-dihydro-1′H- | | | |
| | | spiro[indole-3,3′-pyrrolidine]-1′- | | | |
| | | carboxamide | | | |
| 601 | 19, 15 | ethyl N-{[(3S)-1-(9H-purin-6- | 422.19 | 1.77 | A |
| | | yl)-1,2-dihydro-1′H- | | | |
| | | spiro[indole-3,3′-pyrrolidin]-1′- | | | |
| | | yl]carbonyl}glycinate | | | |
| 602 | 19, 15 | (3S)—N-benzyl-1-(9H-purin-6- | 426.21 | 2.1 | A |
| | | yl)-1,2-dihydro-1′H- | | | |
| | | spiro[indole-3,3′-pyrrolidine]-1′- | | | |
| | | carboxamide | | | |
| 603 | 19, 15 | (3S)—N-ethyl-1-(9H-purin-6-yl)- | 364.16 | 1.72 | A |
| | | 1,2-dihydro-1′H-spiro[indole- | | | |
| | | 3,3′-pyrrolidine]-1′- | | | |
| | | carboxamide | | | |
| 604 | 19, 15 | (3S)—N-allyl-1-(9H-purin-6-yl)- | 376.18 | 1.81 | A |
| | | 1,2-dihydro-1′H-spiro[indole- | | | |
| | | 3,3′-pyrrolidine]-1′- | | | |
| | | carboxamide | | | |
| 605 | 19, 15 | methyl N-{[(3S)-1-(9H-purin-6- | 464.24 | 2.2 | A |
| | | yl)-1,2-dihydro-1′H- | | | |
| | | spiro[indole-3,3′-pyrrolidin]-1′- | | | |
| | | yl]carbonyl}-L-leucinate | | | |
| 606 | 19, 15 | (3S)—N-propyl-1-(9H-purin-6- | 378.17 | 1.85 | A |
| | | yl)-1,2-dihydro-1′H- | | | |
| | | spiro[indole-3,3′-pyrrolidine]-1′- | | | |
| | | carboxamide | | | |
| 607 | 19, 15 | (3S)—N-butyl-1-(9H-purin-6-yl)- | 392.19 | 2.03 | A |
| | | 1,2-dihydro-1′H-spiro[indole- | | | |
| | | 3,3′-pyrrolidine]-1′- | | | |
| | | carboxamide | | | |
| 608 | 19, 15 | (3S)—N-isopropyl-1-(9H-purin- | 378.17 | 1.85 | A |
| | | 6-yl)-1,2-dihydro-1′H- | | | |
| | | spiro[indole-3,3′-pyrrolidine]-1′- | | | |
| | | carboxamide | | | |
| 609 | 19, 15 | (3S)—N-pentyl-1-(9H-purin-6- | 406.2 | 2.2 | A |
| | | yl)-1,2-dihydro-1′H- | | | |
| | | spiro[indole-3,3′-pyrrolidine]-1′- | | | |
| | | carboxamide | | | |
| 610 | 19, 15 | methyl N-{[(3S)-1-(9H-purin-6- | 464.24 | 2.21 | A |
| | | yl)-1,2-dihydro-1′H- | | | |
| | | spiro[indole-3,3′-pyrrolidin]-1′- | | | |
| | | yl]carbonyl}-L-isoleucinate | | | |
| 611 | 19, 15 | (3S)—N-(2-phenylethyl)-1-(9H- | 440.22 | 2.19 | A |
| | | purin-6-yl)-1,2-dihydro-1′H- | | | |
| | | spiro[indole-3,3′-pyrrolidine]-1′- | | | |
| | | carboxamide | | | |
| 612 | 19, 15 | ethyl 4-({[(3S)-1-(9H-purin-6- | 450.22 | 1.94 | A |
| | | yl)-1,2-dihydro-1′H- | | | |
| | | spiro[indole-3,3′-pyrrolidin]-1′- | | | |
| | | yl]carbonyl}amino)butanoate | | | |
| 613 | 19, 15 | (3S)—N-[(1R,2S)-2- | 452.26 | 4.06 | A |
| | | phenylcyclopropyl]-1-(9H- | | | |
| | | purin-6-yl)-1,2-dihydro-1′H- | | | |
| | | spiro[indole-3,3′-pyrrolidine]-1′- | | | |
| | | carboxamide | | | |
| 614 | 19, 15 | methyl N-{[(3S)-1-(9H-purin-6- | 422.19 | 1.78 | A |
| | | yl)-1,2-dihydro-1′H- | | | |
| | | spiro[indole-3,3′-pyrrolidin]-1′- | | | |
| | | yl]carbonyl}-L-alaninate | | | |
| 615 | 19, 15 | ethyl {[(3S)-1-(9H-purin-6-yl)- | 408.18 | 1.75 | A |
| | | 1,2-dihydro-1′H-spiro[indole- | | | |
| | | 3,3′-pyrrolidin]-1′- | | | |
| | | yl]carbonyl}carbamate | | | |
| 616 | 19, 15 | (3S)—N-[(1S)-1-phenylethyl]-1- | 440.22 | 2.21 | A |
| | | (9H-purin-6-yl)-1,2-dihydro- | | | |
| | | 1′H-spiro[indole-3,3′- | | | |
| | | pyrrolidine]-1′-carboxamide | | | |
| 617 | 19, 15 | (3S)—N-cyclopentyl-1-(9H- | 404.23 | 2.04 | A |
| | | purin-6-yl)-1,2-dihydro-1′H- | | | |
| | | spiro[indole-3,3′-pyrrolidine]-1′- | | | |
| | | carboxamide | | | |
| 618 | 19, 15 | (3S)—N-(2-furylmethyl)-1-(9H- | 416.2 | 1.93 | A |
| | | purin-6-yl)-1,2-dihydro-1′H- | | | |
| | | spiro[indole-3,3′-pyrrolidine]-1′- | | | |
| | | carboxamide | | | |
| 619 | 19, 15 | (3S)—N-(3,5-dimethylisoxazol- | 431.19 | 1.8 | A |
| | | 4-yl)-1-(9H-purin-6-yl)-1,2- | | | |
| | | dihydro-1′H-spiro[indole-3,3′- | | | |
| | | pyrrolidine]-1′-carboxamide | | | |
| 620 | 19, 15 | (3S)—N-phenyl-1-(9H-purin-6- | 412.19 | 2.12 | A |
| | | yl)-1,2-dihydro-1′H- | | | |
| | | spiro[indole-3,3′-pyrrolidine]-1′- | | | |
| | | carboxamide | | | |
| 621 | 19, 15 | (3S)—N-benzoyl-1-(9H-purin-6- | 440.16 | 2 | A |
| | | yl)-1,2-dihydro-1′H- | | | |
| | | spiro[indole-3,3′-pyrrolidine]-1′- | | | |
| | | carboxamide | | | |
| 622 | 19 | (3S)-1-(9H-purin-6-yl)-1,2- | 293.2 | 0.98 | I |
| | | dihydrospiro[indole-3,3′- | | | |
| | | pyrrolidine] | | | |
| 623 | 16 | 5-fluoro-1-(1H-pyrrolo[2,3- | 323.3 | 0.4 | I |
| | | b]pyridin-4-yl)-1,2- | | | |
| | | dihydrospiro[indole-3,4′- | | | |
| | | piperidine] | | | |
| 624 | 16 | 5-methyl-1-(1H-pyrrolo[2,3- | 319.3 | 0.51 | I |
| | | b]pyridin-4-yl)-1,2- | | | |
| | | dihydrospiro[indole-3,4′- | | | |
| | | piperidine] | | | |
| 625 | 16, 15 | N-ethyl-1-(1H-pyrrolo[2,3- | 375.2 | 4.9 | H |
| | | b]pyridin-4-yl)-1,2-dihydro-1′H- | | | |
| | | spiro[indole-3,4′-piperidine]-1′- | | | |
| | | carboxamide | | | |
| 626 | 16, 15 | N-cyclohexyl-1-(1H- | 429.3 | 6.54 | H |
| | | pyrrolo[2,3-b]pyridin-4-yl)-1,2- | | | |
| | | dihydro-1′H-spiro[indole-3,4′- | | | |
| | | piperidine]-1′-carboxamide | | | |
| 627 | 8 | 1-(1H-pyrazolo[3,4- | 306.4 | 3.59 | H |
| | | d]pyrimidin-4-yl)-1,2- | | | |
| | | dihydrospiro[indole-3,4′- | | | |
| | | piperidine] | | | |
| 628 | 8 | 5-chloro-1-(1H-pyrazolo[3,4- | 340.2 | 4.25 | H |
| | | d]pyrimidin-4-yl)-1,2- | | | |
| | | dihydrospiro[indole-3,4′- | | | |
| | | piperidine] | | | |
| 629 | 20, 15 | N-ethyl-1-(3-methyl-1H- | 391.4 | 4.83 | H |
| | | pyrazolo[3,4-d]pyrimidin-4-yl)- | | | |
| | | 1,2-dihydro-1′H-spiro[indole- | | | |
| | | 3,4′-piperidine]-1′-carboxamide | | | |
| 630 | 20, 15 | N-cyclohexyl-1-(3-methyl-1H- | 445.4 | 6.15 | H |
| | | pyrazolo[3,4-d]pyrimidin-4-yl)- | | | |
| | | 1,2-dihydro-1′H-spiro[indole- | | | |
| | | 3,4′-piperidine]-1′-carboxamide | | | |
| 631 | 20, 12 | 1′-acetyl-1-(3-methyl-1H- | 362.3 | 1.67 | H |
| | | pyrazolo[3,4-d]pyrimidin-4-yl)- | | | |
| | | 1,2-dihydrospiro[indole-3,4′- | | | |
| | | piperidine] | | | |
| 632 | 20, 13 | 1-(3-methyl-1H-pyrazolo[3,4- | 398.3 | 5.32 | H |
| | | d]pyrimidin-4-yl)-1′- | | | |
| | | (methylsulfonyl)-1,2- | | | |
| | | dihydrospiro[indole-3,4′- | | | |
| | | piperidine] | | | |
| 633 | 8 | 5-fluoro-1-(1H-pyrazolo[3,4- | 324.3 | 3.89 | H |
| | | d]pyrimidin-4-yl)-1,2- | | | |
| | | dihydrospiro[indole-3,4′- | | | |
| | | piperidine] | | | |
| 634 | 8 | 2-methyl-1-(1H-pyrazolo[3,4- | 321.3 | 1.18 | I |
| | | d]pyrimidin-4-yl)-1,2- | | | |
| | | dihydrospiro[indole-3,4′- | | | |
| | | piperidine] | | | |
| 635 | 8 | 2-phenyl-1-(1H-pyrazolo[3,4- | 383.2 | 1.56 | I |
| | | d]pyrimidin-4-yl)-1,2- | | | |
| | | dihydrospiro[indole-3,4′- | | | |
| | | piperidine] | | | |
| 636 | 20 | 5-fluoro-1-(3-methyl-1H- | 338.1 | 3.71 | H |
| | | pyrazolo[3,4-d]pyrimidin-4-yl)- | | | |
| | | 1,2-dihydrospiro[indole-3,4′- | | | |
| | | piperidine] | | | |
| 637 | 8 | 2-propyl-1-(1H-pyrazolo[3,4- | 349.2 | 1.57 | I |
| | | d]pyrimidin-4-yl)-1,2- | | | |
| | | dihydrospiro[indole-3,4′- | | | |
| | | piperidine] | | | |
| 638 | 1, 20 | 6-(3-methyl-1H-pyrazolo[3,4- | 361.1 | 1 | I |
| | | d]pyrimidin-4-yl)-6,7-dihydro- | | | |
| | | 3H-spiro[imidazo[4,5-e]indole- | | | |
| | | 8,4′-piperidine] | | | |
| 639 | 19, 8 | (3S)-1-(1H-pyrazolo[3,4- | 293.2 | 0.85 | I |
| | | d]pyrimidin-4-yl)-1,2- | | | |
| | | dihydrospiro[indole-3,3′- | | | |
| | | pyrrolidine] | | | |
| 640 | 19, 20 | (3S)-1-(3-methyl-1H- | 306.4 | 3.28 | H |
| | | pyrazolo[3,4-d]pyrimidin-4-yl)- | | | |
| | | 1,2-dihydrospiro[indole-3,3′- | | | |
| | | pyrrolidine] | | | |

1. A compound of formula I: 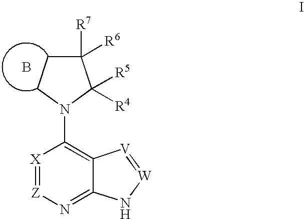
wherein X, Z, V and W are independently selected from the group consisting of N or CR¹;
each R¹ is independently selected from H, halo, cyano, nitro, azido, trifluoromethyl, trifluoromethoxy, —(CH₂)ₙNR⁸R⁹, —(CH₂)ₙOC(O)NR⁸R⁹, —NHC(═NCN)NHR¹⁰, —(C₁-C₁₀)alkyl, —(C₂-C₆)alkenyl, —(C₂-C₆)alkynyl, —O(CH₂)ₙR¹, —O(CH₂)ₙNR⁸R⁹, —(CH₂)ₙC(O)R¹⁰, —(CH₂)ₙNR¹⁰C(O)R¹⁰—(CH₂)ₙNR¹⁰SO₂R¹⁰—(CH₂)ₙC(O)OR¹⁰, —(CH₂)ₙOC(O)R¹⁰, —(CH₂)ₙC(O)NR⁸R⁹, —(CH₂)ₙSO₂NR⁸R⁹, —(CH₂)ₙS(O)ⱼR¹⁰—(CH₂)ₙNR¹⁰C(O)NR⁸R⁹, —(CH₂)ₙNR¹⁰C(O)OR¹⁰, —(CR¹¹R¹²)ₜ(C₆-C₁₀)aryl, —(CR¹¹R¹²)ₜ(4 to 10 membered heterocyclic), —(CR¹¹R¹²)_{q}C(O)(CR¹¹R¹²)ₜ(C₆-C₁₀)aryl, —(CR¹¹R¹²)_{q}C(O)(CR¹¹R¹²)ₜ(4 to 10 membered heterocyclic), —(CR¹¹R¹²)ₜO(CR¹²R¹²)_{q}(C₆-C₁₀)aryl, —(CR¹¹R¹²)ₜO(CR¹¹R¹²)_{q}(4 to 10 membered heterocyclic), —(CR¹¹R¹²)_{q}S(O)ⱼ(CR¹¹R¹²)ₜ(C₆-C₁₀)aryl, and —(CR¹¹R¹²)_{q}S(O)ⱼ(CR¹¹R¹²)ₜ(4 to 10 membered heterocyclic), wherein 1 or 2 ring carbon atoms of the heterocyclic moieties of the foregoing R¹ groups are optionally substituted with an oxo moiety, and the alkyl, alkenyl, alkynyl, aryl and heterocyclic moieties of the foregoing R¹ groups are optionally substituted with 1 to 3 substituents independently selected from halo, hydroxy, cyano, nitro, trifluoromethyl, trifluoromethoxy, azido, —OR¹⁰, —C(O)R¹⁰, —C(O)OR¹⁰, —OC(O)R¹⁰, —NR¹⁰C(O)R¹⁰, —C(O)NR¹⁰R¹¹, —NR⁸R⁹, —NR¹⁰R¹⁰, —(C₁-C₁₀)alkyl, —(C₂-C₆)alkenyl, —(C₂-C₆)alkynyl, —(CR¹¹R¹²)ₜ(C₆-C₁₀)aryl, and —(CR¹¹R¹²)ₜ(4 to 10 membered heterocyclic);
n is an integer selected from 0 to 4;
j is an integer selected from 0 to 2;
q and t are each independently an integer from 0 to 5;
R⁴ is selected from H, (C₁-C₁₀)alkyl, —(CR¹¹R¹²)ₜ(C₆-C₁₀)aryl, —(CR¹¹R¹²)ₜ(4 to 10 membered heterocyclic), wherein the alkyl, aryl and heterocyclic moieties of the foregoing R⁴ groups are optionally substituted with 1 to 3 substituents independently selected from halo, cyano, nitro, trifluoromethyl, trifluoromethoxy, azido, —OR¹³, —C(O)R¹³, —C(O)OR¹³, —OC(O)R¹³, —NR¹³C(O)R¹³, —C(O)NR¹⁴R¹⁵NR¹²OR¹², —(C₁-C₁₀)alkyl, —(C₂-C₆)alkenyl, —(C₂-C₆)alkynyl, —(CR¹¹R¹²)ₜ(C₆-C₁₀)aryl, and —(CR¹¹R¹²)ₜ(4 to 10 membered heterocyclic);
R⁵ is selected from H, —(C₁-C₁₀)alkyl, or wherein R⁴ and R⁵ when taken together form an oxo moiety;
R⁶ and R⁷ are taken together to form a 4 to 10-membered cyclic, bicyclic, heterocyclic or heterobicyclic ring system, said heterocyclic and heterobicyclic ring system containing 1 to 3 heteroatoms independently selected from N, O, or S, wherein each N atom present in the heterocyclic and heterobicyclic ring system is optionally substituted with a substituent selected from —(C₁-C₁₀)alkyl, —R¹⁰—C(O)R¹⁰—SO₂R¹⁰—C(O)NR¹⁰C(O)R¹⁰—C(O)NR¹⁰C(O)OR¹⁰, —C(O)NR⁸R⁹, —C(O)OR¹⁰ and each carbon atom in the heterocyclic and heterobicyclic ring system is independently optionally substituted by 1 to 2 substituents selected from R¹, and each carbon atom in the cyclic and bicyclic ring system is independently optionally substituted by 1 to 2 substituents selected from R¹;
R⁸ and R⁹ are independently selected from H, —(C₁-C₁₀)alkyl, —(CR¹¹R¹²)ₜ(C₆-C₁₀)aryl, —(CR¹¹R¹²)ₜ(4 to 10 membered heterocyclic), or wherein R⁸ and R⁹ when attached to the same N may be taken together to form a 3 to 11 membered mono or bicyclic ring containing an additional 1 to 2 heteroatoms independently selected from N, S or O, wherein each carbon atom of mono or bicyclic ring are optionally substituted with 1 to 2-(C₁-C₁₀)alkyl groups, or an oxo moiety and each additional N atom of the mono or bicyclic ring when present is optionally substituted with a substituent selected from —(C₁-C₁₀)alkyl, —R¹⁰, —C(O)R¹⁰, —SO₂R¹, —C(O)NR¹¹R¹², —C(O)OR¹⁰, wherein 1 or 2 ring carbon atoms of the heterocyclic moieties of the foregoing R⁸ and R⁹ groups are optionally substituted with an oxo moiety, and the alkyl, aryl and heterocyclic moieties of the foregoing R⁸ and R⁹ groups are optionally substituted with 1 to 3 substituents independently selected from halo, cyano, nitro, trifluoromethyl, trifluoromethoxy, azido, —OR¹², —C(O)R¹², —C(O)OR¹², —OC(O)R¹², —NR¹²C(O)R¹², —C(O)NR¹⁴R¹⁵, —(CH₂)ₙNR¹⁰C(O)NR¹⁴R¹⁵, O(CH₂)ₙNR¹⁴R¹⁵, —NR¹⁴R¹⁵, —NR¹²OR¹², —(CH₂)ₙSOⱼR¹⁰, —(C₁-C₁₀)alkyl, —(C₂-C₆)alkenyl, —(C₂-C₆)alkynyl, —(CR¹¹R¹²)ₜ(C₆-C₁₀)aryl, and —(CR¹¹R¹²)ₜ(4 to 10 membered heterocyclic);
R¹⁰ is selected from H, —(C₁-C₁₀)alkyl, —(CR¹¹R¹²)ₜ(C₆-C₁₀)aryl, —(CR¹¹R¹²)ₜ(4 to 10 membered heterocyclic), —(CR¹¹R¹²)_{q}S(O)ⱼ(CR¹¹R¹²)ₜ(C₆-C₁₀)aryl and —(CR¹¹R¹²)_{q}S(O)ⱼ(CR¹¹R¹²)ₜ(4 to 10 membered heterocyclic), wherein the alkyl, aryl and heterocyclic moieties of the foregoing R¹⁰ groups are optionally substituted with 1 to 3 substituents independently selected from halo, cyano, nitro, trifluoromethyl, trifluoromethoxy, azido, —OR¹², —C(O)R¹², —C(O)OR¹², —OC(O)R¹², —NR¹²C(O)R¹², —C(O)NR¹⁴R¹⁵, —O(CH₂)ₙNR¹⁴R¹⁵, —NR¹⁴R¹⁵, —NR¹²OR¹², —(C₁-C₁₀)alkyl, —(C₂-C₆)alkenyl, —(C₂-C₆)alkynyl, —(CR¹¹R¹²)ₜ(C₆-C₁₀)aryl, and —(CR¹¹R¹²)ₜ(4 to 10 membered heterocyclic);
R¹¹ and R¹² are independently selected from H and —(C₁-C₁₀)alkyl;
R¹³ is selected from H, —(C₁-C₁₀)alkyl, —(CR¹¹R¹²)ₜ(C₆-C₁₀ aryl), and —(CR¹¹R¹²)ₜ(4 to 10 membered heterocyclic);
R¹⁴ and R¹⁵ are independently selected from H and —(C₁-C₁₀)alkyl or R¹⁴ and R¹⁵ may be taken together with the N atom they are attached to form a 3 to 11 membered mono- or bicyclic ring optionally containing 1 to 2 additional heteroatoms independently selected from N, O or S(O)ⱼ, wherein the C atoms of said mono- or bicyclic ring are optionally substituted with a substituent selected from oxo or —(C₁-C₁₀)alkyl, and wherein each N atom present in the mono- or bicyclic ring is optionally substituted with a substituent independently selected from —(C₁-C₁₀)alkyl;
B represents a fused 5 or 6-membered aromatic ring containing 0 to 2 heteroatoms, independently selected from N, O or S(O)ⱼ, with the proviso the fused ring B does not contain two adjacent O or S(O)ⱼ atoms, wherein the carbon atoms of the fused ring B may be optionally substituted with 1 to 3 substituents independently selected from R¹, wherein the N atoms of the fused ring B may be optionally substituted with 1 to 2 substituents independently selected from R¹⁰ and ring B may optionally be fused to ring C;
C represents a 5 to 7-membered mono or bicyclic ring, optionally containing 0 to 3 heteroatoms, independently selected from N, O, and S(O)ⱼ, with the proviso the fused ring C does not contain two adjacent O or S(O)ⱼ atoms, and wherein the carbon atoms of fused ring C are optionally substituted with 1 to 3 substituents independently selected from R¹³, wherein the N atoms of the fused ring C may be optionally substituted with 1 to 2 substituents independently selected from R¹¹;
or a pharmaceutically acceptable salt thereof.

2. The compound according to claim 1, or a pharmaceutically acceptable salt thereof, wherein X is N and Z, V and W are CR¹.

3. The compound according to claim 1, or a pharmaceutically acceptable salt thereof, wherein X and V are N and Z and W are CR¹.

4. The compound of claim 1, or a pharmaceutically acceptable salt thereof, wherein each R¹ is independently selected from H, halo, cyano, nitro, azido, trifluoromethyl, trifluoromethoxy, —(CH₂)ₙNR⁸R⁹, —(CH₂)ₙOC(O)NR⁸R⁹, —NHC(═NCN)NHR¹⁰, —(C₁-C₁₀)alkyl, —(C₂-C₆)alkenyl, —(C₂-C₆)alkynyl, —O(CH₂)ₙR¹⁰—O(CH₂)ₙNR⁸R⁹, —(CH₂)ₙC(O)R¹⁰, —(CH₂)ₙNR¹⁰C(O)R¹⁰, —(CH₂)ₙNR¹⁰SO₂R¹⁰, —(CH₂)ₙC(O)OR¹⁰, —(CH₂)ₙOC(O)R¹¹, —(CH₂)ₙC(O)NR⁸R⁹, —(CH₂)ₙSO₂NR⁸R⁹, —(CH₂)ₙSOⱼR¹⁰, —(CH₂)ₙNR¹⁰C(O)NR⁸R⁹, —(CH₂)ₙNR¹⁰C(O)OR¹⁰—(CR¹¹R¹²)ₜ(C₆-C₁₀)aryl, —(CR¹¹R¹²)ₜ(4 to 10 membered heterocyclic), —(CR¹¹R¹²)_{q}C(O)(CR¹¹R¹²)ₜ(C₆-C₁₀)aryl —(CR¹¹R¹²)_{q}C(O)(CR¹¹R¹²)ₜ(4 to 10 membered heterocyclic), —(CR¹¹R¹²)ₜO(CR¹¹R¹²)_{q}(C₆-C₁₀)aryl, —(CR¹¹R¹²)ₜO(CR¹¹R¹²)_{q}(4 to 10 membered heterocyclic), —(CR¹¹R¹²)_{q}S(O)ⱼ(CR¹¹R¹²)ₜ(C₆-C₁₀)aryl, and —(CR¹¹R¹²)_{q}S(O)ⱼ(CR¹¹R¹²)ₜ(4 to 10 membered heterocyclic), wherein 1 or 2 ring carbon atoms of the heterocyclic moieties of the foregoing R¹ groups are optionally substituted with an oxo moiety, and the alkyl, alkenyl, alkynyl, aryl and heterocyclic moieties of the foregoing R¹ groups are optionally substituted with 1 to 3 substituents independently selected from halo, hydroxy, cyano, nitro, trifluoromethyl, trifluoromethoxy, azido, —OR¹⁰, —C(O)R¹⁰, —C(O)OR¹⁰, —OC(O)R¹⁰, —NR¹⁰C(O)NR¹⁰, —C(O)NR¹¹, —NR⁸R⁹, —NR¹⁰R¹⁰, —(C₁-C₁₀)alkyl, —(C₂-C₆)alkenyl, —(C₂-C₆)alkynyl, —(CR¹¹R¹²)ₜ(C₆-C₁₀)aryl, and —(CR¹¹R¹²)ₜ(4 to 10 membered heterocyclic).

5. The compound of claim 1, or a pharmaceutically acceptable salt thereof, wherein ring B represents a fused 5-membered aromatic ring containing 0 to 2 heteroatoms, independently selected from N, O or S(O)ⱼ, with the proviso the fused ring B does not contain two adjacent O or S(O)ⱼ atoms, wherein the carbon atoms of the fused ring B may be optionally substituted with 1 to 3 substituents independently selected from R¹, wherein the N atoms of the fused ring B may be optionally substituted with 1 to 2 substituents independently selected from R¹⁰ and ring B may optionally be fused to ring C.

6. The compound of claim 1, or a pharmaceutically acceptable salt thereof, wherein R⁴ is selected from H and (C₁-C₁₀)alkyl, wherein the alkyl moiety of the foregoing R⁴ group is optionally substituted with 1 to 3 substituents independently selected from halo, cyano, nitro, trifluoromethyl, trifluoromethoxy, azido, —OR¹³, —C(O)R¹³, —C(O)OR¹³, —OC(O)R¹³, —NR¹³C(O)R¹³, —C(O)NR¹⁴R¹⁵, —NR¹²R¹², —(C₁-C₁₀)alkyl, —(C₂-C₆)alkenyl, —(C₂-C₆)alkynyl, —(CR¹¹R¹²)ₜ(C₆-C₁₀)aryl, and —(CR¹¹R¹²)ₜ(4 to 10 membered heterocyclic).

7. The compound of claim 1, or a pharmaceutically acceptable salt thereof, wherein R⁵ is H.

8. The compound of claim 1, or a pharmaceutically acceptable salt thereof, wherein R⁵ is —(C₁-C₁₀)alkyl.

9. The compound of claim 1, or a pharmaceutically acceptable salt thereof, wherein R⁶ and R⁷ are taken together to form a 4 to 10-membered cyclic or bicyclic ring and each carbon atom in the cyclic and bicyclic ring system is independently optionally substituted by 1 to 2 substituents selected from R¹.

10. The compound of claim 1, or a pharmaceutically acceptable salt thereof, wherein R⁶ and R⁷ are taken together to form a 4 to 10-membered heterocyclic or heterobicyclic ring system, said heterocyclic and heterobicyclic ring system containing 1 to 3 heteroatoms independently selected from N, O, or S, wherein each N atom present in the heterocyclic and heterobicyclic ring system is optionally substituted with a substituent selected from —(C₁-C₁₀)alkyl, —R¹⁰—C(O)R¹⁰—SO₂R¹⁰—C(O)NR¹⁰C(O)R¹⁰—C(O)NR¹⁰C(O)OR¹⁰—C(O)NR⁸R⁹, —C(O)OR¹⁰ and each carbon atom in the heterocyclic and heterobicyclic ring system is independently optionally substituted by 1 to 2 substituents selected from R¹.

11. The compound according to claim 1, or a pharmaceutically acceptable salt thereof, wherein said compound is selected from the group consisting of:
(3R)-1-(1H-pyrrolo[2,3-b]pyridin-4-yl)-1,2-dihydrospiro[indole-3,3′-pyrrolidine];
(3R)-1′-(3-furylmethyl)-1-(7H-pyrrolo[2,3-d]pyrimidin-4-yl)-1,2-dihydrospiro[indole-3,3′-pyrrolidine];
(3R)-1′-(3-methylbutyl)-1-(7H-pyrrolo[2,3-d]pyrimidin-4-yl)-1,2-dihydrospiro[indole-3,3′-pyrrolidine];
(3R)-1′-(4-chlorobenzyl)-1-(7H-pyrrolo[2,3-d]pyrimidin-4-yl)-1,2-dihydrospiro[indole-3,3′-pyrrolidine];
(3R)-1-(5-chloro-7H-pyrrolo[2,3-d]pyrimidin-4-yl)-1′-(cyclopropylmethyl)-1,2-dihydrospiro[indole-3,3′-pyrrolidine];
(3R)-1-(5-chloro-7H-pyrrolo[2,3-d]pyrimidin-4-yl)-1,2-dihydrospiro[indole-3,3′-pyrrolidine];
(3R)-1-(5-chloro-7H-pyrrolo[2,3-d]pyrimidin-4-yl)-1′-ethyl-1,2-dihydrospiro[indole-3,3′-pyrrolidine];
(3R)-1-(5-chloro-7H-pyrrolo[2,3-d]pyrimidin-4-yl)-1′-methyl-1,2-dihydrospiro[indole-3,3′-pyrrolidine];
(3R)-1-(5-methyl-7H-pyrrolo[2,3-d]pyrimidin-4-yl)-1,2-dihydrospiro[indole-3,3′-pyrrolidine];
(3R)-1-(7H-pyrrolo[2,3-d]pyrimidin-4-yl)-1,2-dihydrospiro[indole-3,3′-pyrrolidine];
(3R)-1′-(cyclopropylmethyl)-1-(7H-pyrrolo[2,3-d]pyrimidin-4-yl)-1,2-dihydrospiro[indole-3,3′-pyrrolidine];
(3R)-1′-butyl-1-(5-chloro-7H-pyrrolo[2,3-d]pyrimidin-4-yl)-1,2-dihydrospiro[indole-3,3′-pyrrolidine];
(3R)-1′-butyl-1-(7H-pyrrolo[2,3-d]pyrimidin-4-yl)-1,2-dihydrospiro[indole-3,3′-pyrrolidine];
(3R)-1′-ethyl-1-(7H-pyrrolo[2,3-d]pyrimidin-4-yl)-1,2-dihydrospiro[indole-3,3′-pyrrolidine];
(3R)-1′-methyl-1-(7H-pyrrolo[2,3-d]pyrimidin-4-yl)-1,2-dihydrospiro[indole-3,3′-pyrrolidine];
(3R)-1′-propyl-1-(7H-pyrrolo[2,3-d]pyrimidin-4-yl)-1,2-dihydrospiro[indole-3,3′-pyrrolidine];
(3S)-1-(5-chloro-7H-pyrrolo[2,3-d]pyrimidin-4-yl)-1,2-dihydrospiro[indole-3,3′-pyrrolidine];
(3S)-1-(7H-pyrrolo[2,3-d]pyrimidin-4-yl)-1,2-dihydrospiro[indole-3,3′-pyrrolidine];
(3S)-1-(7H-pyrrolo[2,3-d]pyrimidin-4-yl)-1,2-dihydrospiro[indole-3,3′-pyrrolidine];
(3S)-1′-methyl-1-(7H-pyrrolo[2,3-d]pyrimidin-4-yl)-1,2-dihydrospiro[indole-3,3′-pyrrolidine];
1-(1H-pyrrolo[2,3-b]pyridin-4-yl)-1,2-dihydrospiro[indole-3,4′-piperidine];
1-(3-chloro-1H-pyrrolo[2,3-b]pyridin-4-yl)-1,2-dihydrospiro[indole-3,4′-piperidine];
1′-(4-chlorobenzyl)-5-methoxy-1-(7H-pyrrolo[2,3-d]pyrimidin-4-yl)-1,2-dihydrospiro[indole-3,4′-piperidine];
1-(5-chloro-7H-pyrrolo[2,3-d]pyrimidin-4-yl)-1,2-dihydrospiro[indole-3,4′-piperidine];
1-(5-chloro-7H-pyrrolo[2,3-d]pyrimidin-4-yl)-5-fluoro-1′-(morpholin-4-ylcarbonyl)-1,2-dihydrospiro[indole-3,4′-piperidine];
1-(5-chloro-7H-pyrrolo[2,3-d]pyrimidin-4-yl)-5-fluoro-1,2-dihydrospiro[indole-3,4′-piperidine];
1-(5-chloro-7H-pyrrolo[2,3-d]pyrimidin-4-yl)-5-methyl-1,2-dihydrospiro[indole-3,4′-piperidine];
1-(5-chloro-7H-pyrrolo[2,3-d]pyrimidin-4-yl)-5-methyl-1′-[(4-methylpiperazin-1-yl)carbonyl]-1,2-dihydrospiro[indole-3,4′-piperidine];
1-(5-methyl-7H-pyrrolo[2,3-d]pyrimidin-4-yl)-1,2-dihydrospiro[indole-3,4′-piperidine];
1-(7H-pyrrolo[2,3-d]pyrimidin-4-yl)-1,2-dihydrospiro[indole-3,4′-piperidine];
1-(7H-pyrrolo[2,3-d]pyrimidin-4-yl)-1,2-dihydrospiro[indole-3,4′-piperidine]-4-carbonitrile;
1-(7H-pyrrolo[2,3-d]pyrimidin-4-yl)-1,2-dihydrospiro[indole-3,4′-piperidine]-5-carbonitrile;
1-(7H-pyrrolo[2,3-d]pyrimidin-4-yl)-1,2-dihydrospiro[indole-3,4′-piperidine]-5-sulfonamide;
1-(7H-pyrrolo[2,3-d]pyrimidin-4-yl)-5-(2-thienyl)-1,2-dihydrospiro[indole-3,4′-piperidine];
1-(7H-pyrrolo[2,3-d]pyrimidin-4-yl)-5-(3-thienyl)-1,2-dihydrospiro[indole-3,4′-piperidine];
1-(7H-pyrrolo[2,3-d]pyrimidin-4-yl)-5-(trifluoromethyl)-1,2-dihydrospiro[indole-3,4′-piperidine];
1-(7H-pyrrolo[2,3-d]pyrimidin-4-yl)-N-[3-(trifluoromethoxy)benzyl]-1,2-dihydrospiro[indole-3,4′-piperidin]-5-amine;
1-(9H-purin-6-yl)-1,2-dihydrospiro[indole-3,4′-piperidine];
1′-(isopropylsulfonyl)-1-(7H-pyrrolo[2,3-d]pyrimidin-4-yl)-1,2-dihydrospiro[indole-3,4′-piperidine];
1′,5-dimethyl-1-(7H-pyrrolo[2,3-d]pyrimidin-4-yl)-1,2-dihydrospiro[indole-3,4′-piperidine];
1′-[1-(4-chlorophenyl)ethyl]-5-fluoro-1-(7H-pyrrolo[2,3-d]pyrimidin-4-yl)-1,2-dihydrospiro[indole-3,4′-piperidine];
1′-methyl-1-(1H-pyrrolo[2,3-b]pyridin-4-yl)-1,2-dihydrospiro[indole-3,4′-piperidine];
2-[1-(7H-pyrrolo[2,3-d]pyrimidin-4-yl)-1,2-dihydrospiro[indole-3,4′-piperidin]-5-yl]benzonitrile;
2-cyclopropyl-1-(1H-pyrazolo[3,4-d]pyrimidin-4-yl)-1,2-dihydrospiro[indole-3,4′-piperidine];
2-methyl-1-(7H-pyrrolo[2,3-d]pyrimidin-4-yl)-1,2-dihydrospiro[indole-3,4′-piperidine];
2-methyl-1-(9H-purin-6-yl)-1,2-dihydrospiro[indole-3,4′-piperidine];
2-phenyl-1-(1H-pyrazolo[3,4-d]pyrimidin-4-yl)-1,2-dihydrospiro[indole-3,4′-piperidine];
2-propyl-1-(1H-pyrazolo[3,4-d]pyrimidin-4-yl)-1,2-dihydrospiro[indole-3,4′-piperidine];
3-(7H-pyrrolo[2,3-d]pyrimidin-4-yl)-2,3-dihydrospiro[benzo[e]indole-1,4′-piperidine];
3-[1-(5-chloro-7H-pyrrolo[2,3-d]pyrimidin-4-yl)-5-methyl-1,2-dihydro-1′H-spiro[indole-3,4′-piperidin]-1-yl]-N,N,2,2-tetramethylpropan-1-amine;
3-[1-(7H-pyrrolo[2,3-d]pyrimidin-4-yl)-1,2-dihydrospiro[indole-3,4′-piperidin]-5-yl]benzonitrile;
4-(5-chlorospiro[indole-3,4′-piperidin]-1(2H)-yl)-7H-pyrrolo[2,3-d]pyrimidine-5-carbonitrile;
4-(5-fluorospiro[indole-3,4′-piperidin]-1(2H)-yl)-7H-pyrrolo[2,3-d]pyrimidine-5-carbonitrile;
4,5-dichloro-1-(7H-pyrrolo[2,3-d]pyrimidin-4-yl)-1,2-dihydrospiro[indole-3,4′-piperidine];
4,5-dimethyl-1-(7H-pyrrolo[2,3-d]pyrimidin-4-yl)-1,2-dihydrospiro[indole-3,4′-piperidine];
4-chloro-1-(7H-pyrrolo[2,3-d]pyrimidin-4-yl)-1,2-dihydrospiro[indole-3,4′-piperidine];
4-chloro-5-methyl-1-(7H-pyrrolo[2,3-d]pyrimidin-4-yl)-1,2-dihydrospiro[indole-3,4′-piperidine];
4-fluoro-1-(7H-pyrrolo[2,3-d]pyrimidin-4-yl)-1,2-dihydrospiro[indole-3,4′-piperidine];
4-methyl-1-(7H-pyrrolo[2,3-d]pyrimidin-4-yl)-1,2-dihydrospiro[indole-3,4′-piperidine];
4-spiro[indole-3,4′-piperidin]-1(2H)-yl-7H-pyrrolo[2,3-d]pyrimidine-5-carbonitrile;
55-(1,3-benzodioxol-5-yl)-1-(7H-pyrrolo[2,3-d]pyrimidin-4-yl)-1,2-dihydrospiro[indole-3,4′-piperidine];
5-(2-methylphenyl)-1-(7H-pyrrolo[2,3-d]pyrimidin-4-yl)-1,2-dihydrospiro[indole-3,4′-piperidine];
5-(2-phenoxyphenyl)-1-(7H-pyrrolo[2,3-d]pyrimidin-4-yl)-1,2-dihydrospiro[indole-3,4′-piperidine];
5-(3,4-dihydroquinolin-1(2H)-yl)-1-(7H-pyrrolo[2,3-d]pyrimidin-4-yl)-1,2-dihydrospiro[indole-3,4′-piperidine];
5-(3,5-dimethylisoxazol-4-yl)-1-(7H-pyrrolo[2,3-d]pyrimidin-4-yl)-1,2-dihydrospiro[indole-3,4′-piperidine];
5-(3-furyl)-1-(7H-pyrrolo[2,3-d]pyrimidin-4-yl)-1,2-dihydrospiro[indole-3,4′-piperidine];
5-(4-methylpiperazin-1-yl)-1-(7H-pyrrolo[2,3-d]pyrimidin-4-yl)-1,2-dihydrospiro[indole-3,4′-piperidine];
5-(5-methyl-2-furyl)-1-(7H-pyrrolo[2,3-d]pyrimidin-4-yl)-1,2-dihydrospiro[indole-3,4′-piperidine];
5-(methylsulfonyl)-1-(7H-pyrrolo[2,3-d]pyrimidin-4-yl)-1,2-dihydrospiro[indole-3,4′-piperidine];
5-biphenyl-2-yl-1-(7H-pyrrolo[2,3-d]pyrimidin-4-yl)-1,2-dihydrospiro[indole-3,4′-piperidine];
5-chloro-1-(5-chloro-7H-pyrrolo[2,3-d]pyrimidin-4-yl)-1′-(1H-imidazol-4-ylmethyl)-1,2-dihydrospiro[indole-3,4′-piperidine];
5-chloro-1-(5-chloro-7H-pyrrolo[2,3-d]pyrimidin-4-yl)-1,2-dihydrospiro[indole-3,4′-piperidine];
5-chloro-1-(7H-pyrrolo[2,3-d]pyrimidin-4-yl)-1,2-dihydrospiro[indole-3,4′-piperidine];
5-fluoro-1-(1H-pyrrolo[2,3-b]pyridin-4-yl)-1,2-dihydrospiro[indole-3,4′-piperidine];
5-fluoro-1-(3-methyl-1H-pyrazolo[3,4-d]pyrimidin-4-yl)-1,2-dihydrospiro[indole-3,4′-piperidine];
5-fluoro-1-(5-methyl-7H-pyrrolo[2,3-d]pyrimidin-4-yl)-1,2-dihydrospiro[indole-3,4′-piperidine];
5-fluoro-1-(7H-pyrrolo[2,3-d]pyrimidin-4-yl)-1,2-dihydrospiro[indole-3,4′-piperidine];
5-fluoro-1′-methyl-1-(7H-pyrrolo[2,3-d]pyrimidin-4-yl)-1,2-dihydrospiro[indole-3,4′-piperidine];
5-isopropyl-1-(7H-pyrrolo[2,3-d]pyrimidin-4-yl)-1,2-dihydrospiro[indole-3,4′-piperidine];
5-methoxy-1-(7H-pyrrolo[2,3-d]pyrimidin-4-yl)-1,2-dihydrospiro[indole-3,4′-piperidine];
5-methyl-1-(5-methyl-7H-pyrrolo[2,3-d]pyrimidin-4-yl)-1,2-dihydrospiro[indole-3,4′-piperidine];
5-methyl-1-(9H-purin-6-yl)-1,2-dihydrospiro[indole-3,4′-piperidine];
5-morpholin-4-yl-1-(7H-pyrrolo[2,3-d]pyrimidin-4-yl)-1,2-dihydrospiro[indole-3,4′-piperidine];
5-phenoxy-1-(7H-pyrrolo[2,3-d]pyrimidin-4-yl)-1,2-dihydrospiro[indole-3,4′-piperidine];
5-phenyl-1-(7H-pyrrolo[2,3-d]pyrimidin-4-yl)-1,2-dihydrospiro[indole-3,4′-piperidine];
5-pyridin-3-yl-1-(7H-pyrrolo[2,3-d]pyrimidin-4-yl)-1,2-dihydrospiro[indole-3,4′-piperidine];
5-pyridin-4-yl-1-(7H-pyrrolo[2,3-d]pyrimidin-4-yl)-1,2-dihydrospiro[indole-3,4′-piperidine];
5-pyrimidin-5-yl-1-(7H-pyrrolo[2,3-d]pyrimidin-4-yl)-1,2-dihydrospiro[indole-3,4′-piperidine];
6-(3-methyl-1H-pyrazolo[3,4-d]pyrimidin-4-yl)-6,7-dihydro-3H-spiro[imidazo[4,5-e]indole-8,4′-piperidine];
6-(7H-pyrrolo[2,3-d]pyrimidin-4-yl)-6,7-dihydro-3H-spiro[imidazo[4,5-e]indole-8,4′-piperidine];
6-chloro-5-methyl-1-(7H-pyrrolo[2,3-d]pyrimidin-4-yl)-1,2-dihydrospiro[indole-3,4′-piperidine];
methyl 4-{[1-(7H-pyrrolo[2,3-d]pyrimidin-4-yl)-1,2-dihydro-1′H-spiro[indole-3,4′-piperidin]-1′-yl]methyl}benzoate;
N-(2,2-dimethylpropyl)-1-(7H-pyrrolo[2,3-d]pyrimidin-4-yl)-1,2-dihydrospiro[indole-3,4′-piperidin]-5-amine;
N-(2-fluorobenzyl)-1-(7H-pyrrolo[2,3-d]pyrimidin-4-yl)-1,2-dihydrospiro[indole-3,4′-piperidin]-5-amine;
N-(2-methoxybenzyl)-1-(7H-pyrrolo[2,3-d]pyrimidin-4-yl)-1,2-dihydrospiro[indole-3,4′-piperidin]-5-amine;
N-(2-methoxyethyl)-1-(7H-pyrrolo[2,3-d]pyrimidin-4-yl)-1,2-dihydrospiro[indole-3,4′-piperidin]-5-amine;
N-(2-methylbenzyl)-1-(7H-pyrrolo[2,3-d]pyrimidin-4-yl)-1,2-dihydrospiro[indole-3,4′-piperidin]-5-amine;
N-(2-phenylethyl)-1-(7H-pyrrolo[2,3-d]pyrimidin-4-yl)-1,2-dihydrospiro[indole-3,4′-piperidin]-5-amine;
N-(3-chlorophenyl)-1-(7H-pyrrolo[2,3-d]pyrimidin-4-yl)-1,2-dihydrospiro[indole-3,4′-piperidin]-5-amine;
N-(3-methoxybenzyl)-1-(7H-pyrrolo[2,3-d]pyrimidin-4-yl)-1,2-dihydrospiro[indole-3,4′-piperidin]-5-amine;
N-(3-methylbenzyl)-1-(7H-pyrrolo[2,3-d]pyrimidin-4-yl)-1,2-dihydrospiro[indole-3,4′-piperidin]-5-amine;
N-(3-methylphenyl)-1-(7H-pyrrolo[2,3-d]pyrimidin-4-yl)-1,2-dihydrospiro[indole-3,4′-piperidin]-5-amine;
N-(4-chlorobenzyl)-1-(7H-pyrrolo[2,3-d]pyrimidin-4-yl)-1,2-dihydrospiro[indole-3,4′-piperidin]-5-amine;
N-(4-chlorophenyl)-1-(7H-pyrrolo[2,3-d]pyrimidin-4-yl)-1,2-dihydrospiro[indole-3,4′-piperidin]-5-amine;
N-(4-methylbenzyl)-1-(7H-pyrrolo[2,3-d]pyrimidin-4-yl)-1,2-dihydrospiro[indole-3,4′-piperidin]-5-amine;
N-(4-methylphenyl)-1-(7H-pyrrolo[2,3-d]pyrimidin-4-yl)-1,2-dihydrospiro[indole-3,4′-piperidin]-5-amine;
N-(4-phenoxybenzyl)-1-(7H-pyrrolo[2,3-d]pyrimidin-4-yl)-1,2-dihydrospiro[indole-3,4′-piperidin]-5-amine;
N-(biphenyl-3-ylmethyl)-1-(7H-pyrrolo[2,3-d]pyrimidin-4-yl)-1,2-dihydrospiro[indole-3,4′-piperidin]-5-amine;
N-(biphenyl-4-ylmethyl)-1-(7H-pyrrolo[2,3-d]pyrimidin-4-yl)-1,2-dihydrospiro[indole-3,4′-piperidin]-5-amine;
N,N′-dimethyl-N-[1-(7H-pyrrolo[2,3-d]pyrimidin-4-yl)-1,2-dihydrospiro[indole-3,4′-piperidin]-5-yl]ethane-1,2-diamine;
N-[3-(1H-pyrazol-1-yl)benzyl]-1-(7H-pyrrolo[2,3-d]pyrimidin-4-yl)-1,2-dihydrospiro[indole-3,4′-piperidin]-5-amine;
N-[4-(1H-pyrazol-1-yl)benzyl]-1-(7H-pyrrolo[2,3-d]pyrimidin-4-yl)-1,2-dihydrospiro[indole-3,4′-piperidin]-5-amine;
N-{4-[1-(7H-pyrrolo[2,3-d]pyrimidin-4-yl)-1,2-dihydrospiro[indole-3,4′-piperidin]-5-yl]phenyl}acetamide;
N-benzyl-1-(7H-pyrrolo[2,3-d]pyrimidin-4-yl)-1,2-dihydrospiro[indole-3,4′-piperidin]-5-amine;
N-cyclobutyl-1-(7H-pyrrolo[2,3-d]pyrimidin-4-yl)-1,2-dihydrospiro[indole-3,4′-piperidin]-5-amine;
N-cyclopropyl-1-(7H-pyrrolo[2,3-d]pyrimidin-4-yl)-1,2-dihydrospiro[indole-3,4′-piperidine]-5-carboxamide;
N-phenyl-1-(7H-pyrrolo[2,3-d]pyrimidin-4-yl)-1,2-dihydrospiro[indole-3,4′-piperidin]-5-amine; and
N-phenyl-1-(7H-pyrrolo[2,3-d]pyrimidin-4-yl)-1,2-dihydrospiro[indole-3,4′-piperidine]-5-carboxamide.

12. The compound according to claim 1, or a pharmaceutically acceptable salt thereof, wherein said compound is selected from the group consisting of:
(3R)-1-(5-methyl-7H-pyrrolo[2,3-d]pyrimidin-4-yl)-1,2-dihydrospiro[indole-3,3′-pyrrolidine];
(3R)-1-(7H-pyrrolo[2,3-d]pyrimidin-4-yl)-1,2-dihydrospiro[indole-3,3′-pyrrolidine];
(3S)-1-(5-chloro-7H-pyrrolo[2,3-d]pyrimidin-4-yl)-1,2-dihydrospiro[indole-3,3′-pyrrolidine];
(3S)-1-(7H-pyrrolo[2,3-d]pyrimidin-4-yl)-1,2-dihydrospiro[indole-3,3′-pyrrolidine];
(3S)-1′-methyl-1-(7H-pyrrolo[2,3-d]pyrimidin-4-yl)-1,2-dihydrospiro[indole-3,3′-pyrrolidine];
1-(1H-pyrrolo[2,3-b]pyridin-4-yl)-1,2-dihydrospiro[indole-3,4′-piperidine];
1-(3-chloro-1H-pyrrolo[2,3-b]pyridin-4-yl)-1,2-dihydrospiro[indole-3,4′-piperidine];
1-(5-chloro-7H-pyrrolo[2,3-d]pyrimidin-4-yl)-5-fluoro-1′-(morpholin-4-ylcarbonyl)-1,2-dihydrospiro[indole-3,4′-piperidine];
1-(5-methyl-7H-pyrrolo[2,3-d]pyrimidin-4-yl)-1,2-dihydrospiro[indole-3,4′-piperidine];
1-(7H-pyrrolo[2,3-d]pyrimidin-4-yl)-1,2-dihydrospiro[indole-3,4′-piperidine];
1-(7H-pyrrolo[2,3-d]pyrimidin-4-yl)-1,2-dihydrospiro[indole-3,4′-piperidine]-4-carbonitrile;
1-(7H-pyrrolo[2,3-d]pyrimidin-4-yl)-1,2-dihydrospiro[indole-3,4′-piperidine]-5-carbonitrile;
1-(7H-pyrrolo[2,3-d]pyrimidin-4-yl)-5-(trifluoromethyl)-1,2-dihydrospiro[indole-3,4′-piperidine];
1-(9H-purin-6-yl)-1,2-dihydrospiro[indole-3,4′-piperidine];
1′-(isopropylsulfonyl)-1-(7H-pyrrolo[2,3-d]pyrimidin-4-yl)-1,2-dihydrospiro[indole-3,4′-piperidine];
1′-[1-(4-chlorophenyl)ethyl]-5-fluoro-1-(7H-pyrrolo[2,3-d]pyrimidin-4-yl)-1,2-dihydrospiro[indole-3,4′-piperidine];
1′-methyl-1-(1H-pyrrolo[2,3-b]pyridin-4-yl)-1,2-dihydrospiro[indole-3,4′-piperidine];
2-methyl-1-(7H-pyrrolo[2,3-d]pyrimidin-4-yl)-1,2-dihydrospiro[indole-3,4′-piperidine];
2-methyl-1-(9H-purin-6-yl)-1,2-dihydrospiro[indole-3,4′-piperidine];
3-(7H-pyrrolo[2,3-d]pyrimidin-4-yl)-2,3-dihydrospiro[benzo[e]indole-1,4′-piperidine];
3-[1-(5-chloro-7H-pyrrolo[2,3-d]pyrimidin-4-yl)-5-methyl-1,2-dihydro-1′H-spiro[indole-3,4′-piperidin]-1-yl]-N,N,2,2-tetramethylpropan-1-amine;
4-(5-chlorospiro[indole-3,4′-piperidin]-1(2H)-yl)-7H-pyrrolo[2,3-d]pyrimidine-5-carbonitrile;
4-(5-fluorospiro[indole-3,4′-piperidin]-1(2H)-yl)-7H-pyrrolo[2,3-d]pyrimidine-5-carbonitrile;
4-spiro[indole-3,4′-piperidin]-1(2H)-yl-7H-pyrrolo[2,3-d]pyrimidine-5-carbonitrile;
5-(3,5-dimethylisoxazol-4-yl)-1-(7H-pyrrolo[2,3-d]pyrimidin-4-yl)-1,2-dihydrospiro[indole-3,4′-piperidine];
5-(4-methylpiperazin-1-yl)-1-(7H-pyrrolo[2,3-d]pyrimidin-4-yl)-1,2-dihydrospiro[indole-3,4′-piperidine];
5-chloro-1-(5-chloro-7H-pyrrolo[2,3-d]pyrimidin-4-yl)-1′-(1H-imidazol-4-ylmethyl)-1,2-dihydrospiro[indole-3,4′-piperidine];
5-chloro-1-(5-chloro-7H-pyrrolo[2,3-d]pyrimidin-4-yl)-1,2-dihydrospiro[indole-3,4′-piperidine];
5-chloro-1-(7H-pyrrolo[2,3-d]pyrimidin-4-yl)-1,2-dihydrospiro[indole-3,4′-piperidine];
5-fluoro-1-(1H-pyrrolo[2,3-b]pyridin-4-yl)-1,2-dihydrospiro[indole-3,4′-piperidine];
5-fluoro-1-(7H-pyrrolo[2,3-d]pyrimidin-4-yl)-1,2-dihydrospiro[indole-3,4′-piperidine];
5-methyl-1-(9H-purin-6-yl)-1,2-dihydrospiro[indole-3,4′-piperidine];
5-phenoxy-1-(7H-pyrrolo[2,3-d]pyrimidin-4-yl)-1,2-dihydrospiro[indole-3,4′-piperidine];
6-(3-methyl-1H-pyrazolo[3,4-d]pyrimidin-4-yl)-6,7-dihydro-3H-spiro[imidazo[4,5-e]indole-8,4′-piperidine];
6-(7H-pyrrolo[2,3-d]pyrimidin-4-yl)-6,7-dihydro-3H-spiro[imidazo[4,5-e]indole-8,4′-piperidine];
methyl 4-{[1-(7H-pyrrolo[2,3-d]pyrimidin-4-yl)-1,2-dihydro-1′H-spiro[indole-3,4′-piperidin]-1′-yl]methyl}benzoate;
N-(3-chlorophenyl)-1-(7H-pyrrolo[2,3-d]pyrimidin-4-yl)-1,2-dihydrospiro[indole-3,4′-piperidin]-5-amine; and
N-cyclopropyl-1-(7H-pyrrolo[2,3-d]pyrimidin-4-yl)-1,2-dihydrospiro[indole-3,4′-piperidine]-5-carboxamide.

13. A method for the treatment of abnormal cell growth in a mammal comprising administering to said mammal an amount of a compound of claim 1, or a pharmaceutically acceptable salt thereof, that is effective in treating abnormal cell growth.

14. The method according to claim 13, wherein said abnormal cell growth is cancer.

15. The method according to claim 14, wherein said cancer is selected from mesothelioma, hepatobilliary (hepatic and billiary duct), a primary or secondary CNS tumor, a primary or secondary brain tumor, lung cancer (NSCLC and SCLC), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, non hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.
